SEQUENCE LISTING (236 sequences)

SEQ ID NO: 1 - DNA, 357 bp, Homo sapiens
  source 1..357: mol_type = genomic DNA
caggtgcagctggtgcagtctggggctgaactgaagaagcctggggcgtcagtgaaggtctcatgcaaggcttctggatacactttcaccggcaactatatccactggatgcgacaggtccctggacaagggcttgagtggatggggtggatcaacccaagaactggtgacacacatcatgcacagaagtttcagggcagggtcgacatgaccagggacacctccatcaacacagcctacctggaactgaccaggctggaatctgacgacacagccctttattactgtgcgcgatgtgtcttcgcaacttcgcaattcgacccctggggccagggaaccctggtcaccgtctcctca

SEQ ID NO: 2 - DNA, 330 bp, Homo sapiens
  source 1..330: mol_type = genomic DNA
cagtctgccctgactcagcctgcctccgtgtctgggtctcctggacagtcgatcaccatctcctgcactggaaccaacagtgatattgggagtcataaccttgtctcctggtaccaacaacacccaggcaaagcccccaaagtcatgatttatgacgacagtaagcggccctcaggagtttctaatcgcttctctggctccaagtctggcagcacggcctccctgacaatctctgggctccagtctgaggacgaggctgattattattgctgctcatatgcaggtagtagcaactgggtgttcggcggagggaccaagctgacactccta

SEQ ID NO: 3 - DNA, 357 bp, Homo sapiens
  source 1..357: mol_type = genomic DNA
caggtgcagctggtgcagtctggggctgaggtgaagaagcctggggcctcagtgagggtctcctgcagggcctctggatacagcttcaccgacaactatattcactgggtgcggcaggcccctggacaagggcttgagtggatgggatggatcaaccctgacagtggtgccacaagctacgaacataagtttcagggcagggtcaccatgaccagggacacgtccatcagcacagccttcatggaactgggcagtctgagatctgacgacacggccgtttattactgtgcgagggtgcggagggggacagcaggacttgactactggggccagggaaccctggtcaccgtctcctca

SEQ ID NO: 4 - DNA, 330 bp, Homo sapiens
  source 1..330: mol_type = genomic DNA
cagtctgtgctgactcagcctgcctccgtgtctgggtctcttggacagtcgatcaccatctcctgcactggaaccagcagtgatattgggaattataaccttgtctcctggtaccaacagcacccaggccaagcccccaaactcatcatttatgaggtcaataggcggccctcaggggtttctgatcgcttctctgactccaagtctggcaacacggcctccctgacaatctctgggctccaggctgaggacgagggtgattattactgcttttcatatgcaggtggtagtacttgggtgttcggcggagggaccaaggtgaccgtcctc

SEQ ID NO: 5 - protein, 119 aa, synthetic construct
QVQLVQSGAELKKPGASVKVSCKASGYTFTGNYIHWMRQVPGQGLEWMGWINPRTGDTHHAQKFQGRVDMTRDTSINTAYLELTRLESDDTALYYCARCVFATSQFDPWGQGTLVTVSS

SEQ ID NO: 6 - protein, 119 aa, synthetic construct
EVQLVQSGAEVKKPGASVRVSCRASGYSFTDNYIHWVRQAPGQGLEWMGWINPDSGATSYEHKFQGRVTMTRDTSISTAFMELGSLRSDDTAVYYCARVRRGTAGLDYWGQGTLVTVSS

SEQ ID NO: 7 - protein, 119 aa, synthetic construct
QVQLVQSGAEVKKPGASVKVSCKASGYTFTGYYMHWVRQAPGQGLEWMGWINPNTGDTKYAQKFQDWVTMTRDTSISTAYMELNRLRSGDTAVYYCARIISRSSGLDVWGQGTTVTVSS

SEQ ID NO: 8 - protein, 118 aa, synthetic construct
QVQLVQSGAEVKKPGASVKVSCRASGYTFTANYMHWVRQAPGQGLEWMGWINPKSGDTSSTEKFQGRVTMTRDTSITTAYMELSRLRSDDTAVYYCARVPYGSSPDFWGQGTLVTVSS

SEQ ID NO: 9 - protein, 118 aa, synthetic construct
QVQLVQSGAEVKKPGASVKVSCRASGYTFTANYMHWVRQAPGQGLEWMGWINPNSGDTSSAQKFQGRVTMTRDTSISTAYMELSRLRSDDTAVYYCARVPYGSSPDFWGQGTLVTVSS

SEQ ID NO: 10 - protein, 120 aa, synthetic construct
QVQLVQSGAEVKKPGASMKVSCKASGYTFTRYYVHWVRQAPGQGLEWMGWMDPNSGGTNSAQNFQGRVTMTRDTSINTAYMEVSGLRSDDTGVYYCARITVGYFPYFDFWGQGTLVTVSS

SEQ ID NO: 11 - protein, 119 aa, synthetic construct
QVQLVESGAEVKRPGASVKVACKASEYRFAASYIHWVRQAPGQGLEWMGWINLNSGDTKSSQKFQGWVTLTRDTSINTVYMEMTNLKFDDTAIYYCAKIILGGGGLDVWGQGTTVTVSS

SEQ ID NO: 12 - protein, 119 aa, synthetic construct
QVQLVQSGAEVKKPGASVKVSCKASGYTFTGNYMHWVRQAPGQGLEWMGWINPDSGGTNYAQRFQGRVTMTRDTSISTAYMELSRLTSDDTAMYYCARVMLRSSGFDSWGQGTLVTVSS

SEQ ID NO: 13 - protein, 117 aa, synthetic construct
QVQLVQSGAEMKNPGASVKVSCKASGYTFSGYYIHWVRQAPGQGLEWMAWIDPNSGATNYPEKFQGRVTMTTDTSITTTYMELTSLSSDDTAVYYCARVRYGSGFHWGQGTLVTVSS

SEQ ID NO: 14 - protein, 120 aa, synthetic construct
QVQLAQSGAEVRKPGASVKVSCKASGYTFTDYYIHWVRQAPGQGLEWMGWINPDSGRTNYAQRFQGRVSMTSDTSISTAYMDLSRLRSDDTAVYFCAREGAHNYQHFDHWGQGTLVTVSA

SEQ ID NO: 15 - protein, 117 aa, synthetic construct
QVQLVQSGAEVKKPGASVRVSCKASGFTFTTHYIHWVRQAPGQGLEWMGWIDPSSGGTKFAQHFQGRVTMTTDASINTAFMDLNRLRSDDTAMYYCARVRFGSGWYWGQGTLVTVSS

SEQ ID NO: 16 - protein, 124 aa, synthetic construct
QVQLVQSGAEVKRPGSSVKVSCKASGVTFSGYIMSWVRQAPGQGFEWIGGIIPIFGKTTYAQKFQGRVTITADESTTTAYMELNSLKSEDTAIYYCARGGCSSSGCYARPFDPWGQGTLVTVSS

SEQ ID NO: 17 - protein, 110 aa, synthetic construct
QSALTQPASVSGSPGQSITISCTGTNSDIGSHNLVSWYQQHPGKAPKVMIYDDSKRPSGVSNRFSGSKSGSTASLTISGLQSEDEADYYCCSYAGSSNWVFGGGTKLTLL

SEQ ID NO: 18 - protein, 110 aa, synthetic construct
QSVLTQPASVSGSLGQSITISCTGTSSDIGNYNLVSWYQQHPGQAPKLIIYEVNRRPSGVSDRFSDSKSGNTASLTISGLQAEDEGDYYCFSYAGGSTWVFGGGTKVTVL

SEQ ID NO: 19 - protein, 111 aa, synthetic construct
QSALTQPRSVSGSPGQSVTISCAGTSSDVGGYNYVSWYQHHPGKAPKLMIYDVFKRPSGVPDRFSGSKSGNAASLIISGLQAEDEADYYCYSYSGSYTFWVFGGGTKLTVL

SEQ ID NO: 20 - protein, 110 aa, synthetic construct
QSALTQPASVSGSPGQSITISCTGTSSDVGRYKFVSWYQQHPGKAPKVMIFEVTKRPSGVSDRFSGSKSANTASLTISGLQAEDEADYYCLSYAGSSNWVFGGGTRLTVL

SEQ ID NO: 21 - protein, 110 aa, synthetic construct
QSALTQPASVSGSPGQSITISCTGTSSDVGSYKFVSWYQQHPGKAPKVMIFEVTKRPSGVSDRFSGSKSANTASLTISGLQAEDEADYYCFSYAGSSTWVFGGGTRLTVL

SEQ ID NO: 22 - protein, 110 aa, synthetic construct
QSALIQPRSVSGSPGQSITISCSGTSSDVGNYNYISWYQQHPGKAPKVMIYDVNKRPSGVPDRFSGSKSGNTASLTISGLQAEDEADYYCCSYAGSSSWVFGGGTKLTVL

SEQ ID NO: 23 - protein, 111 aa, synthetic construct
QSVLTQPRSVSGSLGQSVTISCSGTSTDVGSYEFVSWYQHHPGKAPKLIIYDVSKRPSGVPDRFSGSKSGNTASLAVSGLQSDDEADYYCSSYADTSNTWVFGGGTKLTVL

SEQ ID NO: 24 - protein, 110 aa, synthetic construct
QSALIQPASVSGSPGQSITISCTGTSSDVGTYNLVSWYQQHPGKVPKLMIYEASERPSGVSNRFSGSKSGNTASLTISGLQAEDEADYYCCSYAGNSAWVFGGGTKLTVL

SEQ ID NO: 25 - protein, 109 aa, synthetic construct
QAVVTQEPSLTVSPGGTVTLTCGSSTGAVTSGHYPYWFQQKAGQAPRTLIYDTGNKHSSTPARFSGSLLGGKAALTLSGAQPEDEAEYYCLLSHSGPWVFGGGTKLTVL

SEQ ID NO: 26 - protein, 108 aa, synthetic construct
EIVLTQSPDTLSLSPGESATLFCRASQSVRSSYLAWYQQTPGQAPRLLIYGASSRATGIPDRFSGSGSGTDFTLTISRLEPEDFAVYYCQQFGRSSWTFGQGTKVEIK

SEQ ID NO: 27 - protein, 109 aa, synthetic construct
QAVVTQEPSLTVSPGGTVTLTCGSTTGPVTSGHYPYWFQQKPGQAPRTVIYDTTNKHSWTPAWFSGSLLGGKAALTLSGAQPEDEAEYYCLLAYSGTWVFGGGTKLTVL

SEQ ID NO: 28 - protein, 115 aa, synthetic construct
QPVVTQPSSLSASPGASASLTCALRSDINVGTYRIYWYQQKSGSPPQFLLRYRSDSDKDLGSGVPSRFSGSKDASANAGILLISGLQSDDEGDYYCMIWHSSAVIFGGGTKLTVL

SEQ ID NO: 29 - protein, 81 aa, synthetic construct
NRMENLNKKVDDGFLDIWTYNAELLVLLENERTLDFHDSNVKNLYEKVKSQLKNNAKEIGNGSFEFYHKCNNECMESVKNG

SEQ ID NO: 30 - protein, 81 aa, synthetic construct
NRMENLNKKVDDGFIDIWTYNAELLVLLENERTLDFHDSNVKNLYEKVKSQLKNNAKEIGNGSFEFYHKCNDECMESVKNG

SEQ ID NO: 31 - protein, 81 aa, synthetic construct
NRIENLNKKVDDGFLDIWTYNAELLVLLENERTLDYHDSNVKNLYEKVRSQLKNNAKEIGNGSFEFYHKCDNTCMESVKNG

SEQ ID NO: 32 - protein, 81 aa, synthetic construct
NRIENLNKKVDDGFLDIWTYNAELLVLLENERTLDYHDSNVKNLYEKVRNQLKNNAKEIGNGSFEFYHKCDNTCMESVKNG

SEQ ID NO: 33 - protein, 81 aa, synthetic construct
NRIENLNKKVDDGFLDVWTYNAELLVLLENERTLDYHDSNVKNLYEKVRHQLKNNAKEIGNGSFEFYHKCDNTCMESVKNG

SEQ ID NO: 34 - protein, 81 aa, synthetic construct
NRLENLNKKMEDGFLDVWTYNAELLVLMENERTLDFHDSNVKNLYDKVRMQLRDNVKELGNGSFEFYHKCDDECMNSVKNG

SEQ ID NO: 35 - protein, 81 aa, synthetic construct
NRIQDLEKYVEDTKIDLWSYNAELLVALENQHTIDLTDSEMNKLFEKTRRQLRENAEDMGNGSFKIYHKCDNACIESIRNG

SEQ ID NO: 36 - protein, 81 aa, synthetic construct
NRIQDLEKYVEDTKIDLWSYNAELLVALENQHTIDLTDSEMNKLFEKTKKQLRENAEDMGNGSFKIYHKCDNACIGSIRNG

SEQ ID NO: 37 - protein, 81 aa, synthetic construct
NRVQDLEKYVEDTKIDLWSYNAELLVALENQHTIDLTDSEMNKLFEKTKKQLRENAEDMGNGSFKIYHKCDNACIGSIRNE

SEQ ID NO: 38 - protein, 81 aa, synthetic construct
NRIQDLEKYVEDTKIDLWSYNAELLVALENQHTIDVTDSEMNKLFERVRRQLRENAEDKGNGSFEIFHQCDNNCIESIRNG

SEQ ID NO: 39 - protein, 81 aa, synthetic construct
NRIENLNKKMEDGFLDVWTYNAELLVLMENERTLDFHDSNVKNLYDKVRLQLRDNAKELGNGCFEFYHKCDNECMESVRNG

SEQ ID NO: 40 - protein, 81 aa, synthetic construct
NRIGNLNKRMEDGFLDVWTYNAELLVLLENERTLDLHDANVKNLYEKVKSQLRDNANDLGNGSFEFWHKCDNECMESVKNG

SEQ ID NO: 41 - protein, 81 aa, synthetic construct
NQIGNVINWTRDSITEVWSYNAELLVAMENQHTIDLADSEMDKLYERVKRQLRENAEEDGTGSFEIFHKCDDDCMASIRNN

SEQ ID NO: 42 - protein, 81 aa, synthetic construct
NRINMINDKIDDQIEGLWAYNAELLVLLENQKTLDEHDSNVKNLFDEVKRRLSTNAMDAGNGSFDILHKCNNECMETIKNG

SEQ ID NO: 43 - protein, 81 aa, synthetic construct
NRLNMINNKIDDQIQDIWAYNAELLVLLENQKTLDEHDANVNNLYNKVKRALGSNAVEDGKGSFELYHKCDDQCMETIRNG

SEQ ID NO: 44 - protein, 81 aa, synthetic construct
NQIGNVINWTKDSITDIWTYQAELLVAMENQHTIDMADSEMLNLYERVRKQLRQNAEEDGKGSFEIYHACDDSCMESIRNN

SEQ ID NO: 45 - protein, 81 aa, synthetic construct
NRINQLSKHVDDSVVDIWSYNAQLLVLLENEKTLDLHDSNVRNLHEKVRRMLKDNAKDEGNGSFTFYHKCDNECIERVRNG

SEQ ID NO: 46 - protein, 81 aa, synthetic construct; identical to SEQ ID NO: 45

SEQ ID NO: 47 - protein, 81 aa, synthetic construct
NRINMLANRIDDAVTDVWSYNAKLLVLLENDKTLDMHDANVRNLHDQVRRVLKTNAIDEGNGSFELLHKCNDSCMETIRNG

SEQ ID NO: 48 - protein, 81 aa, synthetic construct
NRIQDLEKYVEDTKIDLWSYNAELLVALENQHTIDVTDSEMNKLFERVRRQLRENAEDQGNGSFEIFHQCDNNCIESIRNG

SEQ ID NO: 49 - protein, 81 aa, synthetic construct
NQIGNVINWTRDSLTEIWSYNAELLVAMENQHTIDLADSEMNKLYERVRRQLRENAEEDGTGSFEIFHRCDDQCMESIRNN

SEQ ID NO: 50 - protein, 81 aa, synthetic construct
NRINMLADRVDDAVTDVWSYNAKLLVLLENGRTLDLHDANVRNLHDQIKRTLKNNAIDEGDGSFNLLHKCNDSCMEAIRNG

SEQ ID NO: 51 - protein, 81 aa, synthetic construct
NRIQHLSDRVDDALLDIWSYNTELLVLLENERTLDFHDANVKNLFEKVKAQLKDNAIDEGNGSFLLLHKCNNSCMDDIKNG

SEQ ID NO: 52 - protein, 81 aa, synthetic construct
NRIKHLSDRVDDGFLDVWSYNAELLVLLENERTLDFHDANVNNLYQKVKVQLKDNAIDMGNGSFKILHKCNNTCMDDIKNG

SEQ ID NO: 53 - protein, 81 aa, synthetic construct
NEILELDEKVDDLRADTISSQIELAVLLSNEGIINSEDEHLLALERKLKKMLGPSAVEIGNGSFETKHKCNQTCLDRIAAG

SEQ ID NO: 54 - protein, 81 aa, synthetic construct
NEILELDEKVDDLRADTISSQIELAVLLSNEGIINSEDEHLLALERKLKKMLGPSAVDIGNGSFETKHKCNQTCLDRIAAG

SEQ ID NO: 55 - protein, 81 aa, synthetic construct; identical to SEQ ID NO: 53

SEQ ID NO: 56 - protein, 55 aa, synthetic construct
RIQDLEKYVEDTKIDLWSYNAELLVALENQHTIDLTDSEMNKLFEKTRRQLRENA

SEQ ID NO: 57 - protein, 57 aa, synthetic construct
ERRIENLNKKMEDGFLDVWTYNAELLVLMENERTLDFHDSNVKNLYDKVRLQLRDNA

SEQ ID NO: 58 - protein, 56 aa, synthetic construct
HNEILELDEKVDDLRADTISSQIELAVLLSNEGIINSEDEHLLALERKLKKMLGPS

SEQ ID NO: 59 - protein, 8 aa, synthetic construct
GYTFTGNY

SEQ ID NO: 60 - protein, 7 aa, synthetic construct
GYTFTGN

SEQ ID NO: 61 - protein, 8 aa, synthetic construct
INPRTGDT

SEQ ID NO: 62 - protein, 6 aa, synthetic construct
NPRTGD

SEQ ID NO: 63 - protein, 12 aa, synthetic construct
ARCVFATSQFDP

SEQ ID NO: 64 - protein, 9 aa, synthetic construct
NSDIGSHNL

SEQ ID NO: 65 - protein, 14 aa, synthetic construct
TGTNSDIGSHNLVS

SEQ ID NO: 66 - skipped

SEQ ID NO: 67 - protein, 7 aa, synthetic construct
DDSKRPS

SEQ ID NO: 68 - protein, 10 aa, synthetic construct
CSYAGSSNWV

SEQ ID NO: 69 - protein, 10 aa, synthetic construct
CSYAGSSNWV

SEQ ID NO: 70 - protein, 8 aa, synthetic construct
GYSFTDNY

SEQ ID NO: 71 - protein, 7 aa, synthetic construct
GYSFTDN

SEQ ID NO: 72 - protein, 8 aa, synthetic construct
INPDSGAT

SEQ ID NO: 73 - protein, 6 aa, synthetic construct
NPDSGA

SEQ ID NO: 74 - protein, 12 aa, synthetic construct
ARVRRGTAGLDY

SEQ ID NO: 75 - protein, 9 aa, synthetic construct
SSDIGNYNL

SEQ ID NO: 76 - protein, 14 aa, synthetic construct
TGTSSDIGNYNLVS

SEQ ID NO: 77 - skipped

SEQ ID NO: 78 - protein, 7 aa, synthetic construct
EVNRRPS

SEQ ID NO: 79 - protein, 10 aa, synthetic construct
FSYAGGSTWV

SEQ ID NO: 80 - protein, 10 aa, synthetic construct
FSYAGGSTWV

SEQ ID NO: 81 - protein, 8 aa, synthetic construct
GYTFTGYY

SEQ ID NO: 82 - protein, 7 aa, synthetic construct
GYTFTGY

SEQ ID NO: 83 - protein, 8 aa, synthetic construct
INPNTGDT

SEQ ID NO: 84 - protein, 6 aa, synthetic construct
NPNTGD

SEQ ID NO: 85 - protein, 12 aa, synthetic construct
ARIISRSSGLDV

SEQ ID NO: 86 - protein, 9 aa, synthetic construct
SSDVGGYNY

SEQ ID NO: 87 - protein, 14 aa, synthetic construct
AGTSSDVGGYNYVS

SEQ ID NO: 88 - skipped

SEQ ID NO: 89 - protein, 7 aa, synthetic construct
DVFKRPS

SEQ ID NO: 90 - protein, 11 aa, synthetic construct
YSYSGSYTFWV

SEQ ID NO: 91 - protein, 11 aa, synthetic construct
YSYSGSYTFWV

SEQ ID NO: 92 - protein, 8 aa, synthetic construct
GYTFTANY

SEQ ID NO: 93 - protein, 7 aa, synthetic construct
GYTFTAN

SEQ ID NO: 94 - protein, 8 aa, synthetic construct
INPKSGDT

SEQ ID NO: 95 - protein, 6 aa, synthetic construct
NPKSGD

SEQ ID NO: 96 - protein, 11 aa, synthetic construct
ARVPYGSSPDF

SEQ ID NO: 97 - protein, 9 aa, synthetic construct
SSDVGRYKF

SEQ ID NO: 98 - protein, 14 aa, synthetic construct
TGTSSDVGRYKFVS

SEQ ID NO: 99 - skipped

SEQ ID NO: 100 - protein, 7 aa, synthetic construct
EVTKRPS

SEQ ID NO: 101 - protein, 10 aa, synthetic construct
LSYAGSSNWV

SEQ ID NO: 102 - protein, 10 aa, synthetic construct
LSYAGSSNWV

SEQ ID NO: 103 - protein, 8 aa, synthetic construct
GYTFTANY

SEQ ID NO: 104 - protein, 7 aa, synthetic construct
GYTFTAN

SEQ ID NO: 105 - protein, 8 aa, synthetic construct
INPNSGDT

SEQ ID NO: 106 - protein, 6 aa, synthetic construct
NPNSGD

SEQ ID NO: 107 - protein, 11 aa, synthetic construct
ARVPYGSSPDF

SEQ ID NO: 108 - protein, 9 aa, synthetic construct
SSDVGSYKF

SEQ ID NO: 109 - protein, 14 aa, synthetic construct
TGTSSDVGSYKFVS

SEQ ID NO: 110 - skipped

SEQ ID NO: 111 - protein, 7 aa, synthetic construct
EVTKRPS

SEQ ID NO: 112 - protein, 10 aa, synthetic construct
FSYAGSSTWV

SEQ ID NO: 113 - protein, 10 aa, synthetic construct
FSYAGSSTWV

SEQ ID NO: 114 - protein, 8 aa, synthetic construct
GYTFTRYY

SEQ ID NO: 115 - protein, 7 aa, synthetic construct
GYTFTRY

SEQ ID NO: 116 - protein, 8 aa, synthetic construct
MDPNSGGT

SEQ ID NO: 117 - protein, 6 aa, synthetic construct
DPNSGG

SEQ ID NO: 118 - protein, 13 aa, synthetic construct
ARITVGYFPYFDF

SEQ ID NO: 119 - protein, 9 aa, synthetic construct
SSDVGNYNY

SEQ ID NO: 120 - protein, 14 aa, synthetic construct
SGTSSDVGNYNYIS

SEQ ID NO: 121 - skipped

SEQ ID NO: 122 - protein, 7 aa, synthetic construct
DVNKRPS

SEQ ID NO: 123 - protein, 10 aa, synthetic construct
CSYAGSSSWV

SEQ ID NO: 124 - protein, 10 aa, synthetic construct
CSYAGSSSWV

SEQ ID NO: 125 - protein, 8 aa, synthetic construct
EYRFAASY

SEQ ID NO: 126 - protein, 7 aa, synthetic construct
EYRFAAS

SEQ ID NO: 127 - protein, 8 aa, synthetic construct
INLNSGDT

SEQ ID NO: 128 - protein, 6 aa, synthetic construct
NLNSGD

SEQ ID NO: 129 - protein, 12 aa, synthetic construct
AKIILGGGGLDV

SEQ ID NO: 130 - protein, 9 aa, synthetic construct
STDVGSYEF

SEQ ID NO: 131 - protein, 14 aa, synthetic construct
SGTSTDVGSYEFVS

SEQ ID NO: 132 - skipped

SEQ ID NO: 133 - protein, 7 aa, synthetic construct
DVSKRPS

SEQ ID NO: 134 - protein, 11 aa, synthetic construct
SSYADTSNTWV

SEQ ID NO: 135 - protein, 11 aa, synthetic construct
SSYADTSNTWV

SEQ ID NO: 136 - protein, 8 aa, synthetic construct
GYTFTGNY

SEQ ID NO: 137 - protein, 7 aa, synthetic construct
GYTFTGN

SEQ ID NO: 138 - protein, 8 aa, synthetic construct
INPDSGGT

SEQ ID NO: 139 - protein, 6 aa, synthetic construct
NPDSGG

SEQ ID NO: 140 - protein, 12 aa, synthetic construct
ARVMLRSSGFDS

SEQ ID NO: 141 - protein, 9 aa, synthetic construct
SSDVGTYNL

SEQ ID NO: 142 - protein, 14 aa, synthetic construct
TGTSSDVGTYNLVS

SEQ ID NO: 143 - skipped

SEQ ID NO: 144 - protein, 7 aa, synthetic construct
EASERPS

SEQ ID NO: 145 - protein, 10 aa, synthetic construct
CSYAGNSAWV

SEQ ID NO: 146 - protein, 10 aa, synthetic construct
CSYAGNSAWV

SEQ ID NO: 147 - protein, 8 aa, synthetic construct
GYTFSGYY

SEQ ID NO: 148 - protein, 7 aa, synthetic construct
GYTFSGY

SEQ ID NO: 149 - protein, 8 aa, synthetic construct
IDPNSGAT

SEQ ID NO: 150 - protein, 6 aa, synthetic construct
DPNSGA

SEQ ID NO: 151 - protein, 10 aa, synthetic construct
ARVRYGSGFH

SEQ ID NO: 152 - protein, 9 aa, synthetic construct
TGAVTSGHY

SEQ ID NO: 153 - protein, 14 aa, synthetic construct
GSSTGAVTSGHYPY

SEQ ID NO: 154 - skipped

SEQ ID NO: 155 - protein, 7 aa, synthetic construct
DTGNKHS

SEQ ID NO: 156 - protein, 9 aa, synthetic construct
LLSHSGPWV

SEQ ID NO: 157 - protein, 9 aa, synthetic construct
LLSHSGPWV

SEQ ID NO: 158 - protein, 8 aa, synthetic construct
GFTFTTHY

SEQ ID NO: 159 - protein, 7 aa, synthetic construct
GFTFTTH

SEQ ID NO: 160 - protein, 8 aa, synthetic construct
IDPSSGGT

SEQ ID NO: 161 - protein, 6 aa, synthetic construct
DPSSGG

SEQ ID NO: 162 - protein, 10 aa, synthetic construct
ARVRFGSGWY

SEQ ID NO: 163 - protein, 9 aa, synthetic construct
TGPVTSGHY

SEQ ID NO: 164 - protein, 14 aa, synthetic construct
GSTTGPVTSGHYPY

SEQ ID NO: 165 - skipped

SEQ ID NO: 166 - protein, 7 aa, synthetic construct
DTTNKHS

SEQ ID NO: 167 - protein, 9 aa, synthetic construct
LLAYSGTWV

SEQ ID NO: 168 - protein, 9 aa, synthetic construct
LLAYSGTWV

SEQ ID NO: 169 - protein, 8 aa, synthetic construct
GYTFTDYY

SEQ ID NO: 170 - protein, 7 aa, synthetic construct
GYTFTDY

SEQ ID NO: 171 - protein, 8 aa, synthetic construct
INPDSGRT

SEQ ID NO: 172 - protein, 6 aa, synthetic construct
NPDSGR

SEQ ID NO: 173 - protein, 13 aa, synthetic construct
AREGAHNYQHFDH

SEQ ID NO: 174 - protein, 7 aa, synthetic construct
QSVRSSY

SEQ ID NO: 175 - protein, 12 aa, synthetic construct
RASQSVRSSYLA

SEQ ID NO: 176 - skipped

SEQ ID NO: 177 - protein, 7 aa, synthetic construct
GASSRAT

SEQ ID NO: 178 - protein, 9 aa, synthetic construct
QQFGRSSWT

SEQ ID NO: 179 - protein, 9 aa, synthetic construct
QQFGRSSWT

SEQ ID NO: 180 - protein, 8 aa, synthetic construct
GVTFSGYI

SEQ ID NO: 181 - protein, 7 aa, synthetic construct
GVTFSGY

SEQ ID NO: 182 - protein, 8 aa, synthetic construct
IIPIFGKT

SEQ ID NO: 183 - protein, 6 aa, synthetic construct
IPIFGK

SEQ ID NO: 184 - protein, 17 aa, synthetic construct
ARGGCSSSGCYARPFDP

SEQ ID NO: 185 - protein, 9 aa, synthetic construct
SDINVGTYR

SEQ ID NO: 186 - protein, 14 aa, synthetic construct
ALRSDINVGTYRIY

SEQ ID NO: 187 - protein, 7 aa, synthetic construct
YRSDSDK

SEQ ID NO: 188 - protein, 11 aa, synthetic construct
YRSDSDKDLGS

SEQ ID NO: 189 - protein, 9 aa, synthetic construct
MIWHSSAVI

SEQ ID NO: 190 - protein, 9 aa, synthetic construct
MIWHSSAVI

SEQ ID NO: 191 - protein, 11 aa, synthetic construct
  MOD_RES 2: note = X = any amino acid
  MOD_RES 4: note = X = any amino acid
  MOD_RES 11: note = X = any amino acid
AXDXGNGCFEX

SEQ ID NO: 192 - protein, 159 aa, synthetic construct
GYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGSFEFYHKCNNECMESVKNGTYDYPKYSEESKLNREKIDGVKLESM

SEQ ID NO: 193 - protein, 159 aa, synthetic construct
GYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGSFEFYHKCNDECMESVKNGTYDYPKYSEESKLNREKIDGVKLESM

SEQ ID NO: 194 - protein, 160 aa, synthetic construct
GYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGSFEFYHKCDNTCMESVKNGTYDYPKYSEEAKLNREEIDGVKLESTR

SEQ ID NO: 195 - protein, 160 aa, synthetic construct
GYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGSFEFYHKCDNTCMESVKNGTYDYPKYSEEAKLNREKIDGVKLESTR

SEQ ID NO: 196 - protein, 163 aa, synthetic construct
GYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGSFEFYHKCDNTCMESVKNGTYDYPKYSEEAKLNREKIDGVKLDSTRIYQ

SEQ ID NO: 197 - protein, 163 aa, synthetic construct
GYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGSFEFYHKCDDECMNSVKNGTYDYPKYEEESKLNRNEIKGVKLSSMGVYQ

SEQ ID NO: 198 - protein, 162 aa, synthetic construct
GFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGSFKIYHKCDNACIESIRNGTYDHDVYRDEALNNRFQIKGVELKSGYKD

SEQ ID NO: 199 - protein, 162 aa, synthetic construct
GFRHQNSEGRGQAADLKSTQAAIDQINGKLNRLIGETNEKFHQIEKEFSEVEGRIQDLEKYVEDTKIDLWSYNAELLVALENQHTIDLTDSEMNKLFEKTKKQLRENAEDMGNGSFKIYHKCDNACIGSIRNGTYDHDVYRDEALNNRFQIKGVELKSGYKD

SEQ ID NO: 200 - protein, 162 aa, synthetic construct
GFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGSFKIYHKCDNACIGSIRNETYDHNVYRDEALNNRFQIKGVELKSGYKD

SEQ ID NO: 201 - protein, 162 aa, synthetic construct
GFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDKGNGSFEIFHQCDNNCIESIRNGTYDHDIYRDEAINNRFQIQGVKLTQGYKD

SEQ ID NO: 202 - protein, 163 aa, synthetic construct
GYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQ

SEQ ID NO: 203 - protein, 163 aa, synthetic construct
GYHHENSQGSGYAADRESTQKAIDGITNKVNSIINKMNTQFEAVDHEFSNLERRIGNLNKRMEDGFLDVWTYNAELLVLLENERTLDLHDANVKNLYEKVKSQLRDNANDLGNGSFEFWHKCDNECMESVKNGTYDYPKYQKESKLNRQGIESVKLENLGVYQ

SEQ ID NO: 204 - protein, 162 aa, synthetic construct
GFRHQNAQGEGTAADYKSTQSAIDQITGKLNRLIAKTNQQFKLIDNEFNEVEKQIGNVINWTRDSITEVWSYNAELLVAMENQHTIDLADSEMDKLYERVKRQLRENAEEDGTGSFEIFHKCDDDCMASIRNNTYDHRKYREEAMQNRIQIDPVKLSSGYKD

SEQ ID NO: 205 - protein, 163 aa, synthetic construct
GFHHSNSEGTGMAADQKSTQEAIDKITNKVNNIVDKMNREFEVVNHEFSEVEKRINMINDKIDDQIEGLWAYNAELLVLLENQKTLDEHDSNVKNLFDEVKRRLSTNAMDAGNGSFDILHKCNNECMETIKNGTYNHKEYEEEAKLERSKINGVKLEENTTYK

SEQ ID NO: 206 - protein, 163 aa, synthetic construct
GFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGSNAVEDGKGSFELYHKCDDQCMETIRNGTYNRRKYQEESKLERQKIEGVKLESEGTYK

SEQ ID NO: 207 - protein, 162 aa, synthetic construct
GFRHQNAQGTGQAADYKSTQAAIDQITGKLNRLVEKTNTEFESIESEFSEIEHQIGNVINWTKDSITDIWTYQAELLVAMENQHTIDMADSEMLNLYERVRKQLRQNAEEDGKGSFEIYHACDDSCMESIRNNTYDHSQYREEALLNRLNINPVTLSSGYKD

SEQ ID NO: 208 - protein, 164 aa, synthetic construct
GFQHRNDEGTGIAADKESTQKAIDQITSKVNNIVDRMNTNFESVQHEFSEIEERINQLSKHVDDSVVDIWSYNAQLLVLLENEKTLDLHDSNVRNLHEKVRRMLKDNAKDEGNGSFTFYHKCDNECIERVRNGTYDHKEFEEESKINRQEIEGVKLDSSGNVYK

SEQ ID NO: 209 - protein, 163 aa, synthetic construct
GFQHQNAEGTGIAADRDSTQKAIDNMQNKLNNVIDKMNKQFDVVNHEFSEVESRINMINSKVDDQITDIWAYNAELLVLLENQKTLDEHDANVRNLHDRVRRVLRENAIDTGDGSFEILHKCDNNCMDTIRNGTYDHKEYEEESKIERQKINGVKLEENSTYK

SEQ ID NO: 210 - protein, 164 aa, synthetic construct
GFQHQNEQGVGIAADKESTQKAINQITTKINNIIEKMNGNYDSIRGEFNQVEQRINMLANRIDDAVTDVWSYNAKLLVLLENDKTLDMHDANVRNLHDQVRRVLKTNAIDEGNGSFELLHKCNDSCMETIRNGTYNHTEYEEESKLKRQEIEGIKLKSDDNVYK

SEQ ID NO: 211 - protein, 162 aa, synthetic construct
GFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDQGNGSFEIFHQCDNNCIESIRNGTYDHNIYRDEAINNRIKINPVNLTMGYKD

SEQ ID NO: 212 - protein, 162 aa, synthetic construct
GFRHQNAQGQGTAADYKSTQAAIDQITGKLNRLIEKTNKQFELIDNEFTEVEQQIGNVINWTRDSLTEIWSYNAELLVAMENQHTIDLADSEMNKLYERVRRQLRENAEEDGTGSFEIFHRCDDQCMESIRNNTYNHTEYRQEALQNRIMINPVKLSSGYKD

SEQ ID NO: 213 - protein, 164 aa, synthetic construct
GFQHQNEQGTGIAADKASTQKAIDEITTKINNIIEKMNGNYDSIRGEFNQVEKRINMLADRVDDAVTDVWSYNAKLLVLLENGRTLDLHDANVRNLHDQIKRTLKNNAIDEGDGSFNLLHKCNDSCMEAIRNGTYNHEDYKEESQLKRQEIEGIKLKTEDNVYK

SEQ ID NO: 214 - protein, 166 aa, synthetic construct
GYHHENQEGSGYAADKEATQKAVDAITNKVNSIIDKMNSQFESNIKEFNRLELRIQHLSDRVDDALLDIWSYNTELLVLLENERTLDFHDANVKNLFEKVKAQLKDNAIDEGNGSFLLLHKCNNSCMDDIKNGTYKYMDYREESHIEKQKIDGVKLTDYSRYYTMT

SEQ ID NO: 215 - protein, 165 aa, synthetic construct
GYHHQNSEGSGYAADKEATQKAVDAITTKVNNIIDKMNTQFESTAKEFNKIEMRIKHLSDRVDDGFLDVWSYNAELLVLLENERTLDFHDANVNNLYQKVKVQLKDNAIDMGNGSFKILHKCNNTCMDDIKNGTYNYYEYRKESHLEKQKIDGVKLSENSSYKIM

SEQ ID NO: 216 - protein, 165 aa, synthetic construct
GYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGSFETKHKCNQTCLDRIAAGTFDAGEFSLPTFDSLNITAASLNDDGLDNHTI

SEQ ID NO: 217 - protein, 164 aa, synthetic construct
GYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGSFETKHKCNQTCLDRIAAGTFNAGEFSLPTFDSLNITAASLNDDGLDNHT

SEQ ID NO: 218 - protein, 165 aa, synthetic construct; identical to SEQ ID NO: 216

SEQ ID NO: 219 - DNA, 358 bp, synthetic construct
  source 1..358: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaatactggtgacacaaagtatgcacagaagtttcaggactgggtcaccatgaccagggacacgtccatcagtacagcctacatggagctgaacaggctgagatctggcgacacggccgtctattattgtgcgaggataattagcaggtccagcggtctggacgtctggggccaagggaccacggtcaccgtctcctcag

SEQ ID NO: 220 - DNA, 334 bp, synthetic construct
  source 1..334: mol_type = other DNA
cagtctgccctgactcagcctcgctcagtgtccgggtctcctggacagtcagtcaccatctcctgcgctggaaccagcagtgatgttggtggttataactatgtctcctggtaccaacaccaccccggcaaagcccccaaactcatgatttatgatgtctttaagcggccctcaggggtccctgatcgcttctctggctccaagtctggcaacgcggcctccctgatcatctctgggctccaggctgaggatgaggctgattattactgctactcatattcaggcagctacactttttgggtgttcggcggagggaccaagctgaccgtcctag

SEQ ID NO: 221 - DNA, 355 bp, synthetic construct
  source 1..355: mol_type = other DNA
caggtgcagctggtgcagtctggggctgaggtgaagaagcctggggcctcagtgaaggtgtcctgcagggcttctggatacaccttcaccgccaattatatgcactgggtgcgacaggcccctggacaagggcttgagtggatgggatggatcaaccctaaaagtggtgacacaagctctacagagaagtttcagggcagggtcaccatgaccagggacacgtccatcaccacagcctacatggaactgagtaggttgagatctgacgacacggccgtgtattactgtgcgagagtcccctatggcagcagccccgacttttggggccagggaaccctggtcaccgtctcctcag

SEQ ID NO: 222 - DNA, 331 bp, synthetic construct
  source 1..331: mol_type = other DNA
cagtctgccctgactcagcctgcctccgtgtctgggtctcctggacagtcgatcaccatctcctgcactggaaccagcagtgatgttgggaggtataagtttgtctcctggtaccaacagcacccaggcaaagcccccaaagtcatgatttttgaggtcactaagcggccctcaggggtttctgatcgcttctctggctccaagtctgccaacacggcctccctgacaatctctgggctccaggctgaggacgaggctgattattactgcttgtcatatgccggcagtagcaattgggtcttcggcggagggaccaggctgaccgtcctag

SEQ ID NO: 223 - DNA, 355 bp, synthetic construct
  source 1..355: mol_type = other DNA
caggtgcagctggtgcagtctggggctgaggtgaagaagcctggggcctcagtgaaggtctcctgcagggcttctggatacaccttcaccgccaattatatgcactgggtgcgacaggcccctggacaagggcttgagtggatgggatggatcaaccctaacagtggtgacacaagctctgcacagaagtttcagggcagggtcaccatgaccagggacacgtccatcagcacagcctacatggagctgagtagactgagatctgacgacacggccgtgtattactgtgcgagagtcccctatggcagcagccccgacttttggggccagggaaccctggtcaccgtctcctcag

SEQ ID NO: 224 - DNA, 331 bp, synthetic construct
  source 1..331: mol_type = other DNA
cagtctgccctgactcagcctgcctccgtgtctgggtctcctggacagtcgatcaccatctcctgcactggaaccagcagtgatgttgggagttataagtttgtctcctggtaccaacagcacccaggcaaagcccccaaagtcatgatttttgaggtcactaagcggccctcaggggtttctgatcgcttctctggctccaagtctgccaacacggcctccctgacaatctctgggctccaggctgaggacgaggctgattattactgcttctcatatgcaggtagtagcacttgggtattcggcggagggaccaggctgaccgtcctag

SEQ ID NO: 225 - DNA, 361 bp, synthetic construct
  source 1..361: mol_type = other DNA
caggtgcagctggtgcaatctggggctgaggtgaagaagcctggggcctcaatgaaggtctcctgcaaggcttctggatacaccttcacccgctactatgtgcactgggtgcgacaggcccctggacaagggcttgagtggatgggatggatggaccctaatagtggtggcacaaactctgcacagaactttcagggtagggtaaccatgaccagggacacgtccatcaacacggcctacatggaggtgagcgggctgagatctgacgacacgggcgtttattactgtgcgaggattacagtgggttatttcccatactttgacttctggggccagggaaccctggtcaccgtctcctcag

SEQ ID NO: 226 - DNA, 331 bp, synthetic construct
  source 1..331: mol_type = other DNA
cagtctgccctgattcagcctcgctcagtgtccgggtctcctggacagtcaatcaccatctcctgcagtggaaccagcagtgatgttggtaattataactatatttcctggtaccaacagcacccaggcaaagcccccaaagtcatgatttatgatgtcaataagcggccctcaggggtccctgatcgcttctctggctccaagtctggcaacacggcctccctgaccatctctgggctccaggctgaagatgaggctgattactactgctgctcatatgcaggcagctcctcttgggtgttcggcggagggaccaaactgaccgtcctag

SEQ ID NO: 227 - DNA, 376 bp, synthetic construct
  source 1..376: mol_type = other DNA
caggtgcagctggtggagtctggggctgaggtgaagaggcctggggcgtcagtgaaggtcgcctgcaaggcttctgaatacagatttgccgcctcctatatacactgggtgcgacaggccccaggacaagggcttgagtggatgggctggataaacctcaacagtggtgacacaaagtcttcacagaagtttcagggctgggtcaccctgacccgcgacacgtccatcaacacagtctacatggagatgactaatctgaaatttgacgacacggccatttattactgtgcgaagatcatcctgggggggggcggtctagacgtctggggccaagggaccacggtcaccgtctcctcagcctccaccaagggcccat

SEQ ID NO: 228 - DNA, 392 bp, synthetic construct
  source 1..392: mol_type = other DNA
gctagcccagtctgttctgactcagcctcgctcagtgtccgggtctcttggacagtcagtcaccatctcctgcagtggaaccagcactgatgttggtagttatgagtttgtctcctggtaccaacaccacccaggcaaagcccccaaactcattatttatgatgtctctaagcggccctcaggagtccctgatcgcttctctggctcaaagtctggcaacacggcctccctggccgtctctgggctccagtctgacgatgaggctgactattattgctcgtcatatgctgacacctccaatacttgggtgttcggcggagggacgaagctgaccgtcctaggtcagcccaaggctgccccctcggtcactctgttcccaccctcgcggccgc

SEQ ID NO: 229 - DNA, 388 bp, synthetic construct
  source 1..388: mol_type = other DNA
caggtgcagctggtgcagtctggggctgaggtgaagaagcctggggcctcagtgaaggtctcctgcaaggcttctggatacaccttcaccggcaactatatgcactgggtgcgacaggcccctggacaagggcttgagtggatgggctggatcaaccctgacagtggtggcacaaactatgcacagaggtttcagggcagggtcaccatgaccagggacacgtccatcagcacagcctacatggagctgagcaggctgacatctgacgacacggccatgtattactgtgcgagagtgatgctgaggagtagtggatttgactcctggggccagggaaccctggtcaccgtctcctcagcctccaccaagggcccatcccagggcccat

SEQ ID NO: 230 - DNA, 392 bp, synthetic construct
  source 1..392: mol_type = other DNA
gctagcccagtctgccctgattcagcctgcctccgtgtctgggtctcctggacagtcgatcaccatctcctgcactggaaccagcagtgatgtcgggacttataaccttgtctcctggtatcaacagcacccaggcaaagtccccaaactcatgatttatgaggccagtgagcggccctcaggggtttctaatcgcttctctggctccaagtctggcaacacggcctccttgacaatctctgggctccaggctgaggacgaggctgactattactgctgctcatatgcagggaatagcgcttgggtgttcggcggagggaccaagctgaccgtcctaggtcagcccaaggctgccccctcggtcactctgttcccaccctcgcggcccgcac

SEQ ID NO: 231 - DNA, 352 bp, synthetic construct
  source 1..352: mol_type = other DNA
caggtgcagctggtgcagtctggggctgagatgaagaaccctggggcctcagtgaaggtctcctgcaaggcttctggatacaccttcagcggctactatatacactgggtgcgacaggcccctggacaagggcttgagtggatggcatggatcgaccctaatagtggtgccacaaactatccagagaagtttcagggcagggtcaccatgaccacggacacgtccatcaccacaacctacatggagctgaccagcctgagttctgacgacacggccgtgtattactgtgcgagagttcgatatggcagtggcttccactggggccagggaaccctggtcaccgtctcctcag

SEQ ID NO: 232 - DNA, 328 bp, synthetic construct
  source 1..328: mol_type = other DNA
caggctgtggtgactcaggagccctcactgactgtgtccccaggagggacagtcactctcacctgtggctccagcactggagctgtcaccagtggtcattatccctactggttccagcagaaggctggccaagcccccaggacactgatttatgatacaggcaacaaacactcctcgacacctgcccggttctcaggctccctccttgggggcaaagctgccctgaccctttcgggtgcgcagcctgaggatgaggctgaatattactgcttgctctcccatagtggtccgtgggtgttcggcggagggaccaagctgaccgtcctag

SEQ ID NO: 233 - DNA, 360 bp, synthetic construct
  source 1..360: mol_type = other DNA
caggtgcagctggcgcagtctggggctgaggtgaggaagcctggggcctcagtgaaagtctcctgcaaggcttctggatacaccttcaccgactactatatacactgggtgcgacaggcccctggacaagggcttgagtggatgggatggatcaaccctgacagtggcaggacaaactatgcacagagatttcagggcagggtcagtatgaccagcgacacgtccatcagcacagcctacatggacctgagcagactgagatctgacgacacggccgtgtatttctgtgcgagagagggggctcataattaccaacactttgaccactggggccagggaaccctggtcaccgtgtccgca

SEQ ID NO: 234 - DNA, 324 bp, synthetic construct
  source 1..324: mol_type = other DNA
gaaattgtgttgacgcagtctccagacaccctgtctttgtctccaggggaaagcgccaccctcttctgcagggccagtcagagtgttaggagcagttacttagcctggtaccagcagacacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtaggtcgtcgtggacgttcggccaagggaccaaggtggaaatcaaa

SEQ ID NO: 235 - DNA, 351 bp, synthetic construct
  source 1..351: mol_type = other DNA
caggtgcaactggtgcagtctggggctgaggtgaagaagcctggggcctcagtgagggtctcctgcaaggcttctggattcaccttcaccacacactacatacactgggtgcgacaggcccctggacaagggcttgagtggatgggatggatcgaccctagcagtggtggcacaaagtttgcacagcactttcagggcagggtcaccatgaccacggacgcgtccatcaacacagccttcatggacctgaacaggctgagatctgacgacacggccatgtattactgtgcgagagtgcgctttggaagtggctggtactggggccagggaaccctggtcaccgtctcctca

SEQ ID NO: 236 - DNA, 327 bp, synthetic construct
  source 1..327: mol_type = other DNA
caggctgtggtgactcaggagccctcactgactgtgtccccaggagggacggtcactctcacctgtggttccaccactggacctgtcaccagtggtcattatccctactggttccaacagaagcctggccaagcccccaggacagtgatttatgatacaaccaacaaacactcctggacacctgcctggttctcaggctccctccttgggggcaaagctgccctgaccctttcgggtgcacagcctgaggatgaggctgagtattactgcttgctcgcctatagtggtacttgggtattcggcggagggaccaagctgaccgtcctt

(19) United States
(12) Patent Application Publication
(10) Pub. No.: US 2025/0084152 A1
(43) Pub. Date: Mar. 13, 2025

(54) Compositions For and Methods of Treating Influenza

(71) Applicants: Duke University, Durham, NC (US); President and Fellows of Harvard College, Cambridge, MA (US)

(72) Inventors: Garnett Kelsoe, Durham, NC (US); Joel Finney, Durham, NC (US); Stephen Harrison, Cambridge, MA (US)

(21) Appl. No.: 18/883,669

(22) Filed: Sep. 12, 2024

Related U.S. Application Data
(60) Provisional application No. 63/582,181, filed on Sep. 12, 2023.

(51) Int. Cl. C07K 16/10

(52) U.S. Cl. CPC C07K 16/1018; C07K 2317/33; C07K 2317/55; C07K 2317/565; C07K 2317/76

(57) ABSTRACT

Disclosed herein are binding molecules and/or antibodies targeting a universal influenza antigen and methods of using the same to treat a subject having an influenza infection and to minimize the progression of an influenza infection in a subject.

### CROSS-REFERENCE TO RELATED APPLICATION

This application claims the benefit of priority to U.S. Provisional Application No. 63/582,181 filed 12 Sep. 2023, which is incorporated by reference herein in its entirety.

### STATEMENT REGARDING FEDERALLY SPONSORED RESEARCH OR DEVELOPMENT

This invention was made with government support under Federal Grant No. AI089618 awarded by the National Institute of Health. The Federal Government has certain rights to this invention.

### REFERENCE TO THE SEQUENCE LISTING

The Sequence Listing submitted 12 Sep. 2024 as an XML file named ‘23-2098-US_Sequence Listing’, created on 12 Sep. 2024 and having a size of 215,690 bytes is hereby incorporated by reference pursuant to 37 C.F.R. § 1.52 (e) (5).

### BACKGROUND

Influenza A and B viruses (IAVs and IBVs) comprise multiple phylogenetically and antigenically distinct groups, of which two A lineages (H1N1 and H3N2) and two B lineages (Victoria and Yamagata) currently circulate among humans. Antibody (Ab) responses to IAV/IBV infection or vaccination are largely directed toward the viral hemagglutinin (HA) glycoprotein, which mediates viral attachment and membrane fusion with the host cell. Population-level immune pressure drives selection of HA mutations that promote viral escape from immune control. Consequently, annually updated, licensed influenza vaccines typically protect only against seasonal strains and closely related subtypes. Moreover, additional IAVs circulating in non-human animal populations have the potential to spill over into human populations and cause influenza pandemics. Thus, there remains an unmet medical need for identifying a universal HA antigen and developing antibodies and vaccines targeting that universal antigen.

### BRIEF DESCRIPTION OF THE FIGURES

FIG. 1A-FIG. 1G show S1V2-72 and K06.18 are broadly binding HA stem mAbs whose epitope is distinct from those of other standard stem mAbs. FIG. 1A shows the results of a Luminex multiplex binding assay conducted with culture supernatant IgG from human Bmem clones S1V2-72 and K06.18. Binding mean fluorescence intensities (MFIs) are color-coded by magnitude: yellow (≥1×10³), orange (≥1×104), red (≥1×105), black (not done). LOD: below limit of detection. FIG. 1B shows serially diluted recombinant IgG forms of S1V2-72, K06.18, their UAs, CR9114 and an irrelevant mAb were screened by Luminex assay for binding to full-length, trimeric, soluble prefusion rHA ectodomains (FLsE). FIG. 1C shows flow cytometry histograms depicting recombinant IgG binding to K530 cell lines expressing recombinant, native HA on the cell surface. FIG. 1D-FIG. 1F show Ab epitope overlap determined by competitive inhibition of binding in a Luminex assay. Each curve depicts the binding MFI of K06.18 (FIG. 1D), or S1V2-72 (FIG. 1E, FIG. 1F) to HA-conjugated microbeads in the presence of serially diluted competitor mAbs. HAs used: H1/X-181 (FIG. 1D), H1/CA09 (FIG. 1E; for CR9114, 222-1C06, and S1V2-72 filled diamonds) or H3/X-31 (FIG. 1E; for CR8020 and S1V2-72 empty diamonds), or H5/VN04 (FIG. 1F). FIG. 1G provides ELISA measurement of recombinant IgG binding to a truncated peptide encompassing the LAH of prefusion HA2. LAH5 and LAH31 are mAbs reported (Adachi Y, et al. (2019) Nat. Commun. 10:3883; Tonouchi K, et al. (2020) Int. Immunol. 32:613-621) to bind the LAH.

FIG. 2A-FIG. 2E show that S1V2-72 binds a conserved β-hairpin exposed in EHA2. FIG. 2A shows 5 Å-resolution cryo-EM map for S1V2-72 Fab bound to B/MY04 EHA2. FIG. 2B is a docked model showing the AF2-predicted structures (ribbon models) of two S1V2-72 Fabs and trimeric B/MY04 EHA2 docked into the cryo-EM map shown in (FIG. 2A). FIG. 2C shows a ribbon model showing S1V2-72 Fab (heavy chain in orange, light chain in yellow) complexed with B/MY04 EHA2 monomer (cyan) (left side). FIG. 2C also shows a rotated, close-up view of the S1V2-72: EHA2 interface (right side). Also shown is the position of the HA1 strand (dashed green line) that would be linked by disulfide bond (black line) to HA2 in the postfusion structure of authentic HA1+HA2. FIG. 2D shows a simplified close-up view of the S1V2-72: EHA2 interface, showing only EHA2, the HCDRs and LCDR3. FIG. 2E shows the alignment of the amino acid sequences encoding the B-hairpin in different HA subtypes. The boxed residues form the loop of the hairpin. Dashed positions indicate identity with the consensus residue. CON (consensus sequence). B-V (Victoria lineage of IBV). B-Y (Yamagata lineage of IBV).

FIG. 3A-FIG. 3C shows that S1V2-72 protects against lethal IAV or IAB challenge by IgG subtype-dependent mechanisms. FIG. 3A-FIG. 3B show the weight loss and survival of mAb-infused mice after infection with H3N2 (FIG. 3A) or HBNB (FIG. 3B) influenza virus. Asterisks denote significant differences (P<0.05) in survival at day 13 compared to 222-1C06-treated controls. FIG. 3C shows the in vitro microneutralization IC50 values. FIG. 3D shows the results of an in vitro ADCC proxy assay. Mouse FcγRIV-expressing effector cells and HA-expressing target cells were co-cultured in the presence of serially diluted IgG2c mAbs. FcγRIV activation was measured as luminescence output, as described in EXAMPLES (infra).

FIG. 4A-FIG. 4D show the characterization of mAbs identified by their S1V2-72-like genetic signature. FIG. 4A-FIG. 4B show ELISA results showing rIgG binding to EHA2s (FIG. 4A) or HA0s (FIG. 4B). FIG. 4C provides flow cytometry histograms depicting rIgG binding to K530 cell lines expressing recombinant, native HA on the cell surface. FIG. 4D shows the Ab epitope overlap determined by competitive inhibition of binding in an ELISA. Each curve depicts the binding OD of S1V2-72 to B/MY04 EHA2-coated microplates in the presence of serially diluted competitor mAbs.

FIG. 5A-FIG. 5J show that B/MY04 EHA2 vaccination elicits potent GC and serum Ab responses against the S1V2-72 epitope. FIG. 5A provides a schematic depicting the schedule for vaccinating mice and analyzing their immune response. FIG. 5B is a representative flow cytometry dot plots depicting the frequencies of GC B cells and PCs in draining popliteal LNs from naïve, primed (18 d post-prime), primed and boosted (8 d post-boost), or primed but unboosted (same timepoint as boosted) mice. FIG. 5C-FIG. 5D show the aggregate frequencies and absolute numbers of GC B cells (FIG. 5C) and PCs (FIG. 5D) from mice treated as described in (FIG. 5A) and (FIG. 5B). Data were pooled from 3 independent experiments with 2-3 animals per group per experiment. FIG. 5E shows Luminex MFI values for culture supernatant IgG binding. Each symbol represents clonal IgG from a single GC B cell isolated 18 d post-prime with B/MY04 EHA2. Data are from one experiment with three mice, representative of two independent experiments comprising five mice total. Horizontal red lines denote the binding threshold (mean plus 6 SD of signal from control wells containing no B cells). FIG. 5F shows the competitive inhibition by S1V2-72. S1V2-72 human IgG or irrelevant human IgG was pre-incubated with B/MY04 EHA2 Luminex beads, followed by addition of Nojima culture supernatant (mouse) IgGs. Competitive inhibition was calculated as mouse IgG binding in the presence of S1V2-72 as a percentage of mouse IgG binding in the presence of irrelevant human IgG. Values below the dotted horizontal line are >90% inhibited. Only clonal supernatant IgGs whose (uninhibited) binding MFI for B/MY04 EHA2 was ≥10³ (FIG. 5E) were tested. FIG. 5G shows the Luminex MFI values for B/MY04 EHA2 binding by serially diluted serum IgG from mice treated as in (FIG. 5A). Each curve shows the geometric mean±SEM. Binding by recombinant S1V2-72 mouse IgG1 standard (2 μg/mL initial, then serially 3-fold diluted) is shown for comparison. Data are from experiment, representative of three independent experiments with similar results.

FIG. 5H shows the concentrations of B/MY04 EHA2-reactive serum IgG, normalized to S1V2-72 mouse IgG1 standard Ab (as shown in [FIG. 5G]). Data were pooled from 3 independent experiments with 2-3 mice per group per experiment. FIG. 5I shows the competitive inhibition by immune serum. Serially diluted serum was pre-incubated with B/MY04 EHA2 or H3/X31 EHA2 Luminex beads, then S1V2-72 human IgG was added. Inhibition was calculated as the percentage of human IgG binding signal relative to control samples lacking mouse Ig. Each curve depicts the geometric mean±SEM of the groups shown in (FIG. 5G). Values below the dotted horizontal line are >90% inhibited. Inhibition by recombinant S1V2-72 mouse IgG1 standard (2 g/mL initial, then serially 3-fold diluted) is shown for comparison. FIG. 5J shows total S1V2-72-competing B/MY04 EHA2-reactive serum Ig, normalized to S1V2-72 mouse IgG1 standard Ab (as shown in [FIG. 5I]). Each symbol represents a single animal (FIG. 5C, FIG. 5D, FIG. 5F, FIG. 5H, FIG. 5J). Solid black horizontal lines denote geometric means (FIG. 5C, FIG. 5D, FIG. 5H, FIG. 5J). Asterisks (FIG. 5C, FIG. 5D, FIG. 5H, FIG. 5J) denote statistically significant differences at P<0.05 (*), P<0.01 (**), P<0.001 (***). See METHODS for details of statistical analyses.

FIG. 6A-FIG. 6G shows the identification and characterization of broadly reactive HA mAbs. FIG. 6A is an example of a flow cytometry gating strategy for isolation of HA-binding IgG+Bmem from human donor S1 before (d0) or one week after (d7) immunization with seasonal influenza vaccine. FIG. 6B shows the MFI values of Nojima culture supernatant IgGs binding to antigen beads in a Luminex assay. Each symbol represents clonal IgG from a single Bmem cell. Horizontal red lines denote the binding threshold (mean plus 6 SD of signal from control wells containing no B cells). FIG. 6C-FIG. 6G provides the Luminex assay results depicting rIgG binding to full-length soluble ectodomain (FLsE) of prefusion H1 HA (FIG. 6C), FLsE prefusion H3 HA (FIG. 6D), FLSE prefusion B HA (FIG. 6E), H3/X31 trimeric head (i.e., lacking the stem domain; FIG. 6F), or FLsE prefusion wildtype (WT) and mutant forms of H3/KS17 (FIG. 6G). The mutants, mInterface, mRBS, mCR9114, and mCR8020, have clusters of point mutations designed (28) to eliminate binding of head interface, receptor binding site, CR9114-like, and CR8020-like Abs, respectively.

FIG. 7A-FIG. 7C show the S1V2-72 binds postfusion HA more avidly than prefusion HA. FIG. 7A show BLI traces showing the association of B/MY04 FLSE HA0 trimers with IgG-loaded BLI sensors. The IAV-specific IgG K03.12 (11) served as a negative control for HA binding. FIG. 7B-FIG. 7C show BLI traces showing the association of S1V2-72 Fab with B/MY04 FLsE-loaded (FIG. 7B) or B/MY04 EHA2-loaded (FIG. 7C) BLI sensors. In all traces, protein binding to the loaded sensor was measured as wavelength shift (DI), in nm.

FIG. 8 provides a schematic depicting the strategy for processing cryo-EM data.

FIG. 9A shows a representative micrograph of S1V2-72 Fab bound to B/MY04 EHA2, embedded in vitreous ice (scale bar=300 Å), low pass filtered for clarity. FIG. 9B shows the selected 2D class averages of S1V2-72 Fab: EHA2 complexes. FIG. 9C shows the reconstruction of two S1V2-72 Fabs bound to B/MY04 EHA2, filtered and colored by local resolution. FIG. 9D shows the gold-standard Fourier shell correlation (GSFSC) curves from CryoSPARC. FIG. 9E shows the viewing direction distribution plot.

FIG. 10A-FIG. 10B shows S1V2-72 protects mice from lethal infection with a high dose of IAV. FIG. 10A shows mice were injected with mAbs and subsequently infected with H3N2 IAV. Post-infection weight loss (mean±SEM) and survival were tracked over time. FIG. 10B shows the results of an in vitro ADCC proxy assay. Mouse FcgRIV-expressing effector cells and HA-expressing target cells were co-cultured in the presence of serially diluted mouse IgG2c or mouse IgG1 mAbs. FcgRIV activation was measured as luminescence output (mean±S.D.).

FIG. 11A-FIG. 11D shows the characterization of human mAbs that compete with S1V2-72 for HA binding. FIG. 11A shows the inhibition of S1V2-72 binding to HAs by Nojima culture supernatant IgGs. Samples selected for further characterization as rigGs are marked with unique colored symbols. Dotted horizontal lines denote 80% and 90% inhibition. FIG. 11B shows luminex MFI values for Nojima culture supernatant IgGs binding to antigen beads. Results are shown for the IgGs that inhibited S1V2-72 binding by >90%, and color-coded as in FIG. 1A, except that blank white cells denote values below the limit of detection, and green cells denote MFI values <1000. Black cells were not measured. FIG. 11C shows ELISA OD values for culture supernatant IgGs binding to LAH peptides. Values above background are highlighted red. FIG. 11D shows Luminex assay results for rIgG binding to HA-conjugated beads. FIG. 11E shows the results of a Luminex competitive inhibition assay. Each curve depicts S1V2-72 binding to HA in the presence of serially diluted competitor rigGs. HAs used were B/MY04 EHA2 (for irrelevant IgG, CR9114, S1V2-72, S5V6-P7F12), H3/X31 EHA2 (for S1V5-P4E9, S8V5-P5F7), or H5/VN04 FLsE (for KEL03-P12C9). FIG. 11F shows flow cytometry histograms depicting recombinant IgG binding to K530 cell lines expressing recombinant, native HA on the cell surface.

FIG. 12A-FIG. 12D shows B/MY04 EHA2 vaccination elicits potent GC and serum Ab responses against the S1V2-72 epitope. FIG. 12A shows Luminex MFI values for culture supernatant IgG binding. Each symbol represents clonal IgG from a single mature follicular B cell. Horizontal red lines denote the binding threshold (mean plus 6 SD of signal from control wells containing no B cells). Data are from one mouse; they are representative of three independent experiments with three mice total. FIG. 12B (top) shows Luminex MFI values for culture supernatant IgGs from GC B cells. FIG. 12B (bottom left) shows Luminex MFI values for supernatant IgGs that bind B/MY04 EHA2, but not H3/X31 EHA2. FIG. 12B (bottom right) shows Luminex MFI values for IgGs that bind both B/MY04 EHA2 and H3/X31 EHA2. Each symbol represents a clonal IgG from a single B cell isolated 18 d post-prime with B/MY04 EHA2. Data were pooled from 3 mice from one independent experiment, and are representative of two independent experiments with 5 mice total. Solid red lines denote the binding thresholds, as in (FIG. 12A). FIG. 12C shows Luminex MFI values for HA binding by serially diluted serum IgG from mice treated as in FIG. 5A. Each curve depicts the geometric mean±SEM. Binding by recombinant S1V2-72 mouse IgG1 standard (2 μg/mL initial, then serially 3-fold diluted) is shown for comparison. Data are representative of three independent experiments. FIG. 12D shows the concentration of HA-binding serum IgG, normalized to S1V2-72 mouse IgG1 standard Ab (as shown in [FIG. 12C]). Each symbol represents one mouse. Data were pooled from 2-3 independent experiments with 2-3 animals per group per experiment. Horizontal lines depict geometric means. Asterisks (FIG. 12C, FIG. 12D) denote statistically significant differences at P<0.05 (*), P<0.01 (**).

FIG. 13 shows that serially diluted recombinant IgG forms of CR9114, irrelevant mAb 319-319-D28P, several β-hairpin antibodies, and S5V6-P7F12 were screened by ELISA for binding to EHA2s from various IAVs and IBV.

FIG. 14A-FIG. 14D show conserved features of β-hairpin Ab heavy- and light-chains. FIG. 14A shows the alignment of β-hairpin antibody heavy-chain amino acid sequences. CDRs are annotated according to the IMGT method. There are many widely conserved or universally conserved residues in the CDRs. FIG. 14B-FIG. 14D show sequence logograms depicting the relative frequencies of the amino acids in β-hairpin antibody light-chains (FIG. 14B), in human germline IGLV sequences (n=38) deposited in the IMGT database (FIG. 14C), or in human germline IGKV sequences (n=66) deposited in the IMGT database (FIG. 14D). Numbers along the x-axis denote the order of the residue after conserved Cys104 (IMGT numbering), i.e., “1” denotes the residue immediately C-terminal to Cys104, “2” denotes the second residue, etc. The height of each letter reflects the relative abundance of the corresponding amino acid among all the aligned sequences. The black box circumscribes the residues at the tip of the β-hairpin antibody light-chains (FIG. 14B) or at comparable positions in the germline sequences (FIG. 14C-FIG. 14D). Inset text denotes the absolute frequencies of serine residues at specific positions (FIG. 14C-FIG. 14D).

FIG. 15 shows a flow cytometry gating strategy for isolation of EHA2-binding or -nonbinding mature follicular (MF) B cells from the spleens of β-hairpin antibody knockin mice.

FIG. 16 shows ELISA OD values for culture supernatant IgG (diluted 1:5) binding to B/MY04 EHA2. Each symbol represents clonal IgG from a single MF B cell isolated as described in FIG. 15, except in the column denoted “feeder cells only”, where each symbol represents a culture supernatant from a well into which no B cell was sorted. The horizontal dashed line denotes the binding threshold (mean plus 6 SD of signal from control wells containing no B cells). “Probe (−)” denotes MF B cells that did not bind EHA-PE in FIG. 15; “probe (+)” denotes MF B cells that bound EHA-PE.

FIG. 17A-FIG. 17B show ELISA OD values for culture supernatant IgG binding (diluted 1:10) to anti-Ig LC and various EHA2s. Each symbol represents clonal IgG from a single, B/MY04 EHA2-binding MF B cell isolated from β-hairpin LC⁺⁺ (FIG. 17A) or β-hairpin HC^{+/+} β-hairpin LC⁺⁺ (FIG. 17B) mice, as described in FIG. 15 and FIG. 16. Certain clonal IgGs are depicted by symbols with unique colors and shapes (e.g., red circle) to enable comparison of the binding of those IgGs to several different antigens. The horizontal dashed line denotes the binding threshold (mean plus 6 SD of signal from control wells containing no B cells).

FIG. 18 shows a flow cytometry gating strategy for isolation of GC B cells elicited by immunization with EHA2. Mice were immunized with B/MY04 EHA2 (10 μg)+alum in the hind footpad, and the draining popliteal lymph node was dissected 8 days post-immunization for recovery of GC B cells.

FIG. 19A-FIG. 19B show ELISA OD values for culture supernatant IgG binding (diluted 1:10) to anti-Ig LC and various EHA2s. Each symbol represents clonal IgG from a single GC B cell isolated from β-hairpin LC⁺⁺ (FIG. 19A) or β-hairpin HC^{+/+} β-hairpin LC⁺⁺ (FIG. 19B) mice, as described in FIG. 18. Certain clonal IgGs are depicted by symbols with unique colors and shapes (e.g., red circle) to enable comparison of the binding of those IgGs to several different antigens. The horizontal dashed line denotes the binding threshold (mean plus 6 SD of signal from control wells containing no B cells).

FIG. 20A-FIG. 20B show ELISA data depicting the binding of serially diluted, recombinant IgG forms of S1V2-72 (FIG. 20A) or S5V6-P7F12 (FIG. 20B) to various EHA2s.

FIG. 21 shows full-length EHA2 and the truncated, “core” HA2 sequence containing the β-hairpin epitope. The left most panel shows full-length B/MY04 EHA2, the middle panel shows B/MY04 EHA2 (pink) bound to S1V2-72 Fab (red/blue), and the right most panel shows a proposed truncated form of EHA2 (HA2 residues 75-155) containing the β-hairpin epitope.

### BRIEF SUMMARY

Disclosed herein is a binding molecule and/or antibody capable of specifically binding to postfusion hemagglutinin (HA) of influenza. Disclosed herein is a binding molecule and/or antibody capable of specifically binding to postfusion hemagglutinin (HA) of influenza A virus strains. Disclosed herein is a binding molecule and/or antibody capable of specifically binding to postfusion hemagglutinin (HA) of influenza B virus strains. Disclosed herein is a binding molecule and/or antibody capable of specifically binding to postfusion hemagglutinin (HA) of influenza A virus strains and influenza B virus strains.

Disclosed herein is a nucleic acid molecule encoding a disclosed binding molecule and/or antibody. Disclosed herein is a nucleic acid molecule encoding a disclosed antibody.

Disclosed herein is a nucleic acid molecule comprising IGHV1-2*02/IGHD5-12*01/IGHJ5*02 and IGLV2-23*01/IGLJ3*02. Disclosed herein is nucleic acid molecule comprising IGHV1-2*02/IGHD5-12*01/IGHJ5*02 encoding a heavy chain, and IGLV2-23*01/IGLJ3*02 encoding a light chain. Disclosed herein is a nucleic acid molecule comprising IGHV1-2*02/IGHD5-12*01/IGHJ5*02 encoding a heavy chain, and IGLV2-23*01/IGLJ3*02 encoding a light chain, wherein the heavy chain can comprise about 1% to about 20% somatic mutations, and/or wherein the light chain can comprise about 1% to about 20% somatic mutations. Disclosed herein is a nucleic acid molecule comprising IGHV1-2*02/IGHD5-12*01/IGHJ5*02 encoding a heavy chain and IGLV2-23*01/IGLJ3*02 encoding a light chain, wherein the heavy chain can comprise about 1% to about 20% somatic mutations, and/or wherein the light chain can comprise about 1% to about 20% somatic mutations.

Disclosed herein is a universal influenza vaccine antigen. Disclosed herein is a universal influenza vaccine antigen comprising a β-hairpin epitope of HA2.

Disclosed herein is a composition comprising a universal influenza vaccine antigen. Disclosed herein is a composition comprising a universal influenza vaccine antigen and a vaccine adjuvant. Disclosed herein is a postfusion HA protein and an adjuvant. Disclosed herein is a pharmaceutical formulation comprising a disclosed binding molecule and/or antibody and one or more pharmaceutically acceptable carriers and/or excipients. Disclosed herein is a pharmaceutical formulation comprising a disclosed antibody and one or more pharmaceutically acceptable carriers and/or excipients.

Disclosed herein is a pharmaceutical formulation comprising a disclosed universal influenza antigen and one or more pharmaceutically acceptable carriers and/or excipients.

Disclosed herein is a pharmaceutical formulation comprising a disclosed nucleic acid molecule and one or more pharmaceutically acceptable carriers and/or excipients. Disclosed herein is a pharmaceutical formulation comprising one or more disclosed binding molecules and/or antibodies and one or more pharmaceutically acceptable carriers and/or excipients. Disclosed herein is a pharmaceutical formulation comprising one or more disclosed antibodies and one or more pharmaceutically acceptable carriers and/or excipients. Disclosed herein is a pharmaceutical formulation comprising one or more disclosed universal influenza antigens and one or more pharmaceutically acceptable carriers and/or excipients.

Disclosed herein is a pharmaceutical formulation comprising one or more disclosed nucleic acid molecules and one or more pharmaceutically acceptable carriers and/or excipients.

Disclosed herein is a method of treating a subject having an influenza infection, the method comprising administering to a subject in need thereof a therapeutically effective amount of a disclosed binding molecule and/or a disclosed antibody. Disclosed herein is a method of slowing progression of an influenza infection, the method comprising administering to a subject in need thereof a therapeutically effective amount of a disclosed binding molecule and/or a disclosed antibody. Disclosed herein is a method of reducing the symptoms of an influenza infection, the method comprising administering to a subject in need thereof a therapeutically effective amount of a disclosed binding molecule and/or a disclosed antibody.

Disclosed herein is a method of minimizing the morbidity and/or the risk of mortality due to an influenza infection, the method comprising administering to a subject in need thereof a therapeutically effective amount of a disclosed binding molecule and/or a disclosed antibody.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase ‘consisting essentially of’ limits the scope of a claim to the recited components in a composition or the recited steps in a method as well as those that do not materially affect the basic and novel characteristic or characteristics of the claimed composition or claimed method. The phrase ‘consisting of’ excludes any component, step, or element that is not recited in the claim. The phrase ‘comprising’ is synonymous with ‘including’, ‘containing’, or ‘characterized by’, and is inclusive or open-ended. ‘Comprising’ does not exclude additional, unrecited components or steps.

In an aspect, when referring to any numerical value, the term ‘about’ means a value falling within a range that is +10% of the stated value.

Ranges can be expressed herein as from ‘about’ one particular value, and/or to ‘about’ another particular value. When such a range is expressed, a further aspect includes from the one particular value and/or to the other particular value. Similarly, when values are expressed as approximations, by use of the antecedent ‘about,’ it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as ‘about’ that particular value in addition to the value itself. For example, if the value ‘10’ is disclosed, then ‘about 10’ is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

In an aspect, the terms ‘optional’ or ‘optionally’ means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

In an aspect, the term ‘subject’ refers to the target of administration, e.g., a human being. The term ‘subject’ also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have an influenza infection, be suspected of having an influenza infection, or be at risk of developing an influenza infection.

In an aspect, the term ‘diagnosed’ means having been subjected to an examination by a person of skill, for example, a physician, and found to have a condition that can be diagnosed or treated by one or more disclosed binding molecules and/or disclosed antibodies, the disclosed isolated nucleic acid molecules, the disclosed vectors, the disclosed cells, the disclosed pharmaceutical formulations, or a combination thereof, or by one or more disclosed methods. For example, ‘diagnosed with a disease or disorder’ means having been subjected to an examination by a person of skill, for example, a physician, and found to have a condition (such as an influenza infection) that can be treated by one or more disclosed binding molecules and/or disclosed antibodies, the disclosed isolated nucleic acid molecules, the disclosed vectors, the disclosed cells, the disclosed pharmaceutical formulations, or a combination thereof, or by one or more disclosed methods. For example, “suspected of having a disease or disorder” can mean having been subjected to an examination by a person of skill, for example, a physician, and found to have a condition (such as an influenza infection) that can likely be treated by one or more disclosed binding molecules and/or disclosed antibodies, the disclosed isolated nucleic acid molecules, the disclosed vectors, the disclosed cells, the disclosed pharmaceutical formulations, or a combination thereof, or by one or more disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject afflicted with a disease or disorder (e.g., an influenza infection). In an aspect, a patient can refer to a subject that has been diagnosed with or is suspected of having a disease or disorder such as an influenza infection. In an aspect, a patient can refer to a subject that has been diagnosed with or is suspected of having a disease or disorder and is seeking treatment or receiving treatment for a disease or disorder (such as an influenza infection).

In an aspect, the phrase “identified to be in need of treatment for a disease or disorder,” or the like, refers to selection of a subject based upon need for treatment of the disease or disorder. For example, a subject can be identified as having a need for treatment of a disease or disorder (e.g., an influenza infection) based upon an earlier diagnosis by a person of skill and thereafter subjected to treatment for an influenza infection. In an aspect, the identification can be performed by a person different from the person making the diagnosis. In an aspect, the administration can be performed by one who performed the diagnosis.

In an aspect, “activated” and “activation” can refer to the state of a T cell that has been sufficiently stimulated to induce detectable cellular proliferation. Activation can also be associated with induced cytokine production and detectable effector functions. The term “activated T cells” can refer to T cells that are proliferating. Signals generated through the TCR alone may be insufficient for full activation of the T cell and one or more secondary or costimulatory signals may also be required. Thus, T cell activation can comprise a primary stimulation signal through the TCR/CD3 complex and one or more secondary costimulatory signals. Costimulation can be evidenced by proliferation and/or cytokine production by T cells that have received a primary activation signal, such as stimulation through the TCR/CD3 complex.

In an aspect, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having received one or more disclosed binding molecules and/or disclosed antibodies, the disclosed isolated nucleic acid molecules, the disclosed vectors, the disclosed cells, the disclosed pharmaceutical formulations, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not having received one or more of the disclosed binding molecule and/or disclosed antibodies, the disclosed isolated nucleic acid molecules, the disclosed vectors, the disclosed cells, the disclosed pharmaceutical formulations, or a combination thereof). In an aspect, the inhibition or reduction can be 0-25%, 25-50%, 50-75%, or 75-100% as compared to native or control levels. In an aspect, a native or control level can be a pre-disease or pre-disorder level (such as a pre-influenza infection state).

The words “treat” or “treating” or “treatment” include palliative treatment, that is, treatment designed for the relief of symptoms rather than the curing of the disease, pathological condition, or disorder (e.g., an influenza infection); preventative treatment, that is, treatment directed to minimizing or partially or completely inhibiting the development of the associated disease, pathological condition, or disorder (e.g., an influenza infection); and supportive treatment, that is, treatment employed to supplement another specific therapy directed toward the improvement of the associated disease, pathological condition, or disorder (e.g., an influenza infection). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having an influenza infection). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (e.g., an influenza infection). For example, treating a disease or disorder can reduce one or more symptoms of a disease or disorder in a subject by 1%-100% as compared to a control (such as, for example, an individual not having an influenza infection). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder (e.g., an influenza infection). It is understood that treatment does not necessarily refer to a cure or complete ablation or eradication of a disease or disorder. However, in an aspect, treatment can refer to a cure or complete ablation or eradication of a disease or disorder (such as an influenza infection).

In an aspect, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as an influenza infection) or related complication from progressing to that complication. In an aspect, preventing metastasis is intended.

In an aspect, the terms “administering” and “administration” refer to any method of providing one or more disclosed binding molecules and/or disclosed antibodies, the disclosed isolated nucleic acid molecules, the disclosed vectors, the disclosed cells, the disclosed pharmaceutical formulations, or a combination thereof, or by one or more disclosed methods to a subject. Such methods are well known to those skilled in the art and include, but are not limited to, the following: oral administration, transdermal administration, administration by inhalation, nasal administration, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of one or more disclosed binding molecules and/or disclosed antibodies, the disclosed isolated nucleic acid molecules, the disclosed vectors, the disclosed cells, the disclosed pharmaceutical formulations, or a combination thereof can comprise administration directly into the CNS or the PNS. Administration can be continuous or intermittent. Administration can comprise a combination of one or more routes.

In an aspect, the skilled person can determine an efficacious dose, an efficacious schedule, and an efficacious route of administration for the one or more disclosed binding molecules and/or disclosed antibodies, the disclosed isolated nucleic acid molecules, the disclosed vectors, the disclosed cells, the disclosed pharmaceutical formulations, or a combination thereof to treat or prevent a disease or disorder (such as an influenza infection). In an aspect, the skilled person can also alter, change, or modify an aspect of an administering step to improve efficacy of one or more disclosed binding molecules and/or disclosed antibodies, the disclosed isolated nucleic acid molecules, the disclosed vectors, the disclosed cells, the disclosed pharmaceutical formulations, or a combination thereof.

By “determining the amount” is meant both an absolute quantification of a particular analyte (e.g., biomarker for an influenza infection, for example) or a determination of the relative abundance of a particular analyte (e.g., an influenza infection biomarker). The phrase includes both direct or indirect measurements of abundance or both.

In an aspect, “modifying the method” can comprise modifying or changing one or more features or aspects of one or more steps of a disclosed method. In an aspect, a method can be altered by changing the amount of the one or more disclosed binding molecules and/or disclosed antibodies, the disclosed isolated nucleic acid molecules, the disclosed vectors, the disclosed cells, the disclosed pharmaceutical formulations, or a combination thereof administered to a subject, or by changing the frequency of administration of the one or more disclosed binding molecules and/or disclosed antibodies, the disclosed isolated nucleic acid molecules, the disclosed vectors, the disclosed cells, the disclosed pharmaceutical formulations, or a combination thereof to a subject, by changing the duration of time that one or more disclosed binding molecules and/or disclosed antibodies, the disclosed isolated nucleic acid molecules, the disclosed vectors, the disclosed cells, the disclosed pharmaceutical formulations, or a combination thereof is administered to a subject, or by substituting for one or more of the disclosed components and/or reagents with a similar or equivalent component and/or reagent. The same applies to all disclosed binding molecule and/or disclosed antibodies, disclosed isolated nucleic acid molecules, disclosed vectors disclosed cells, disclosed pharmaceutical formulations, and any combination thereof.

In an aspect, the term “pharmaceutically acceptable carrier” refers to sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, “concurrently” means (1) simultaneously in time, or (2) at different times during the course of a common treatment schedule.

In an aspect, the term “contacting” refers to bringing one or more disclosed binding molecules and/or disclosed antibodies, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof together with a target area or intended target area in such a manner that the disclosed binding molecule and/or disclosed antibodies, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof can exert an effect on the intended target or targeted area either directly or indirectly. A target area or intended target area can be one or more of a subject's organs (e.g., lungs, heart, liver, kidney, brain, etc.) hosting cells infected by influenza. In an aspect, a target area or intended target area can be any cell or any organ infected by a disease or disorder (such as an influenza infection). In an aspect, a target area or intended target area can be any organ, tissue, or cells that are affected by a disease or disorder (such as an influenza infection).

In an aspect, “determining” can refer to measuring or ascertaining the presence and severity of a disease or disorder, such as, for example, influenza infection. Methods and techniques used to determine the presence and/or severity of a disease or disorder are typically known to the medical arts. For example, the art is familiar with the ways to identify and/or diagnose the presence, severity, or both of a disease or disorder (such as, for example, an influenza infection).

In an aspect, “effective amount” and “amount effective” can refer to an amount that is sufficient to achieve the desired result such as, for example, the treatment and/or prevention of a disease or disorder (e.g., an influenza infection) or a suspected disease or disorder (e.g., an influenza infection). In an aspect, the terms “effective amount” and “amount effective” can refer to an amount that is sufficient to achieve the desired an effect on an undesired condition (e.g., an influenza infection). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of one or more disclosed binding disclosed vectors, the disclosed cells, the disclosed pharmaceutical formulations, the disclosed anti-chemokines, the disclosed anti-cancer agents, the disclosed chemotherapeutics, or any combination thereof that (i) treats the particular disease, condition, or disorder (e.g., an influenza infection), (ii) attenuates, ameliorates, or eliminates one or more symptoms of the particular disease, condition, or disorder e.g., an influenza infection), or (iii) delays the onset of one or more symptoms of the particular disease, condition, or disorder described herein (e.g., an influenza infection). The specific therapeutically effective dose level for any particular patient will depend upon a variety of factors including the disorder being treated and the severity of the disorder; one or more disclosed binding molecules and/or disclosed antibodies, the disclosed isolated nucleic acid molecules, the disclosed vectors, the disclosed cells, the disclosed pharmaceutical formulations, or any combination thereof employed; the disclosed methods employed; the age, body weight, general health, sex and diet of the patient; the time of administration; the route of administration; the rate of excretion of the one or more disclosed binding molecules and/or disclosed antibodies, the disclosed isolated nucleic acid molecules, the disclosed vectors, the disclosed cells, the disclosed pharmaceutical formulations, or any combination thereof employed; the duration of the treatment; drugs used in combination or coincidental with the one or more disclosed binding molecules and/or disclosed antibodies, the disclosed isolated nucleic acid molecules, the disclosed vectors, the disclosed cells, the disclosed pharmaceutical formulations, or any combination thereof employed, and other like factors well known in the medical arts. For example, it is well within the skill of the art to start doses of the one or more disclosed binding molecules and/or disclosed antibodies, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed binding molecule and/or disclosed antibodies, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, an influenza infection.

The term “antibody” (Ab) includes, without limitation, a glycoprotein immunoglobulin that binds specifically to an antigen. An antibody can comprise at least two heavy (H) chains and two light (L) chains interconnected by disulfide bonds, or an antigen-binding molecule and/or antibody thereof. Each H chain can comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions can be further subdivided into regions of hypervariability, termed complementarity determining regions (CDRs), interspersed with regions that are more conserved, termed framework regions (FR). Each VH and VL can comprise three CDRs and four FRs, arranged from amino-terminus to carboxy-terminus in the following order: FR1, CDR1, FR2, CDR2, FR3, CDR3, and FR4. The variable regions of the heavy and light chains can contain a binding domain that interacts with an antigen. The constant regions of the Abs can mediate the binding of the immunoglobulin to host tissues or factors, including various cells of the immune system (e.g., effector cells) and the first component (C1q) of the classical complement system. Generally, human antibodies can be approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains can be linked or connected to one another by a single disulfide bond and two other disulfide bonds can connect the heavy chain hinge regions to one another, so that the dimers can be connected to one another and the tetramer can be formed. Naturally produced antibodies are also glycosylated, e.g., on the CH₂ domain. The term “antibody” is used to mean an immunoglobulin molecule that recognizes and specifically binds to a target, such as a protein, polypeptide, peptide, carbohydrate, polynucleotide, lipid, or combinations of the foregoing etc., through at least one antigen recognition site within the variable region of the immunoglobulin molecule. In an aspect, the term encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). CDRs of the light and heavy chains are primarily responsible for the interaction and specificity of the antibody with antigen (such as a disclosed universal influenza antigen). In an aspect, the variable region can be a human variable region. In an aspect, the variable region can comprise rodent or murine CDRs and human framework regions (FRs). In an aspect, the variable region is a primate (e.g., non-human primate) variable region. In an aspect, the variable region can comprise rodent or murine CDRs and primate framework regions (FRs).

The terms “VL” and “VL domain” are used interchangeably to refer to the light chain variable region of an antibody or an antigen-binding molecule and/or antibody thereof. The terms “VH” and “VH domain” are used interchangeably to refer to the heavy chain variable region of an antibody or an antigen-binding molecule and/or antibody thereof.

In an aspect, “conjugate” or “conjugated” can be used to define the operative association of one disclosed component to another disclosed component. In an aspect, conjugated does not intend to refer solely to any type of operative association and is not particularly limited to chemical “conjugation”.

As known to the skilled person, certain IgG subtypes can bind to cell surface receptors on many types of cells to bring about an assortment of effects, for example, (i) the enabling of phagocytosis (e.g., monocytes, macrophages, neutrophils), (ii) antibody-dependent cellular cytotoxicity (monocytes, macrophages, and lymphocytes), or (iii) to effect feedback control on antibody synthesis (B and T lymphocytes). In an aspect, the properties of the IgG subclasses can very, and in most cases the Fc fragments can have the same property as the intact IgG; meaning that it does not appear to be modulated by the hinge or the Fab. In an aspect, cellular Fc receptors can be classified into three categories according to structure and affinity. All the sites on IgG that can interact with these separate receptors appear to be located in the Fc region, and, in the case of the FcγRI (which is the highest affinity receptor class), the site involves residues 233-237, at the N-terminal end of the Cy2 domain close to the hinge region but coded in the Cy2 exon. In humans, FcRg activation and opsonization is most evident in IgG1, IgG2, and IgG3 and negative for IgG4; C fixation is highest in IgG1 and IgG3, low in IgG2 and negative in IgG4. Human IgG1 exhibits greatest ADCC activity.

In an aspect, “effector function” can refer to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement mediated cytotoxicity (CMC). An effector function may be antigen binding dependent, antigen binding independent, or both. ADCC refers to lysis of antibody-bound target cells by immune effector cells. ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

The term “immunotherapy” refers to the treatment of a subject afflicted with, or at risk of contracting or suffering a recurrence of, a disease by a method comprising inducing, enhancing, suppressing, or otherwise modifying an immune response. Examples of immunotherapy can include, but are not limited to, NK cells and T cell therapies, NK CAR T-cell and CAR T-cells therapies, and vaccines. T cell therapy can include adoptive T cell therapy, tumor-infiltrating lymphocyte (TIL) immunotherapy, autologous cell therapy, engineered autologous cell therapy (eACTTM), and allogeneic T cell transplantation. However, one of skill in the art would recognize that the conditioning methods disclosed herein would enhance the effectiveness of any transplanted T cell therapy. The T cells or NK cells of the immunotherapy can come from any source known in the art. For example, T cells and NK cells can be differentiated in vitro from a hematopoietic stem cell population (for example iPSCs) or can be obtained from a subject. T cells and NK cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using techniques known to the skilled person.

In an aspect, the term “humanized antibody” refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues from the complementary determining region (CDR) are replaced by residues from the CDR of a non-human species (e.g., mouse, rat, rabbit, hamster, etc.) that have the desired specificity, affinity, and capability. In some instances, the Fv framework region (FR) residues of a human immunoglobulin are replaced with the corresponding residues in an antibody from a non-human species that has the desired specificity, affinity, and capability. The humanized antibody can be further modified by the substitution of additional residue either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin.

That an antibody “selectively binds” or “specifically binds” to an epitope or receptor means that the antibody reacts or associates more frequently, more rapidly, with greater duration, with greater affinity, or with some combination of the above to the epitope or receptor than with alternative substances, including unrelated proteins. “Selectively binds” or “specifically binds” means, for instance, that an antibody binds to a protein with a KD of about 0.1 mM or less, more usually about 1 μM or less. “Selectively binds” or “specifically binds” means at times that an antibody binds to a protein with a KD of about 0.1 mM or less, at times about 1 μM or less, at times about 0.1 μM or less, at times about 0.01 μM or less, and at times about 1 nM or less. It is understood that, in an aspect, an antibody or binding moiety that specifically binds to a first target may or may not specifically bind to a second target. As such, “specific binding” does not necessarily require (although it can include) exclusive binding, e.g., binding to a single target.

A “target” or “target antigen” is any molecule bound by a binding motif (e.g., a β-hairpin in postfusion HA2 or prefusion HA2). A disclosed target can be cells and/or tissues in a subject. A target, in an aspect, can be a β-hairpin in HA.

In an aspect, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

In an aspect, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis (octyloxy) butanoyl)oxy)-2-((((3-(diethylamino) propoxy) carbonyl)oxy) methyl) propyl octadeca-9,12-dienoate, also called 3-((4,4-bis (octyloxy) butanoyl)oxy)-2-((((3-(diethylamino) propoxy) carbonyl)oxy) methyl) propyl (9Z,12Z)-octadeca-9,12-dienoate) or another ionizable lipid. In an aspect, the term cationic and ionizable in the context of LNP lipids can be used interchangeably, e.g., wherein ionizable lipids are cationic depending on pH.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

In an aspect, “immune-modulating” refers to the ability of the one or more disclosed binding molecules and/or disclosed antibodies, the disclosed isolated nucleic acid molecules, the disclosed vectors, the disclosed cells, the disclosed pharmaceutical formulations, or a combination thereof to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

In an aspect, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGFIR antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

In an aspect, the term “package insert” is used to refer to instructions customarily included in commercial packages of therapeutic products, that contain information about the indications, usage, dosage, administration, contraindications and/or warnings concerning the use of such therapeutic products.

In an aspect, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., one or more disclosed binding disclosed vectors, the disclosed cells, the disclosed pharmaceutical formulations, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy to a subject having or diagnosed with an influenza infection.

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific claimed compositions and/or claimed methods.

### B. Influenza

Influenza viruses are emerging and re-emerging contagious pathogens that pose a significant threat to global health. Influenza viruses belong to the Orthomyxoviridae family and are classified as types A, B, C, and the recently identified type D. Influenza A viruses (IAVs) infect diverse host species, including birds, bats, pigs, and humans. IAVs are enveloped viruses, containing a 13.5 kb genome composed of eight segments of negative-sense single-stranded RNA, encoding different proteins, including RNA-dependent RNA polymerase subunits (PB1, PB2, PA), viral glycoproteins hemagglutinin (HA) and neuraminidase (NA), viral nucleoprotein (NP), matrix protein M1 and M2, non-structural protein (NS1), and NS2.

IAVs can be further characterized by their subtypes based on their surface glycoproteins, HA and NA. HA, but not NA, is essential to initiate an infection, while the inhibition of NA has been reported to enhance infection. HA and NA are antigenically distinct and are majorly targeted by influenza virus vaccines to produce neutralizing antibodies. To date, 18 HA and 11 NA subtypes have been identified in IAVs, and a combination of these HA and NA subtypes can generate many different strains. Vaccination remains the most effective intervention approach; however, mismatches between circulating and vaccine subtypes may reduce vaccine efficacy. Moreover, multiple strategies adopted by IA Vs can make it difficult to prevent successful infection and replication in the host. The innate immune system, an important component of host immunity, confers protection against invading pathogens, including viruses. The innate immune system detects conserved structures on microbes, such as pathogen-associated molecular patterns (PAMPs)/microbe-associated molecular patterns (MAMPs) and damage-associated molecular patterns (DAMPs), via the key innate immune-sensing receptors, germline-encoded pattern recognition receptors (PRRs). Different PRRs, such as Toll-like receptors (TLRs), retinoic acid-inducible gene I (RIG)-like receptors, nucleotide-binding oligomerization domain-containing protein-like receptors, C-type lectin receptors, AIM2-like receptors, and DNA-sensing receptors, are key innate immune components that recognize viral nucleic acids and proteins.

### C. Compositions

Binding Molecules and/or Antibodies

Disclosed herein is a binding molecule and/or a disclosed antibody capable of specifically binding to postfusion hemagglutinin (HA) of influenza. Disclosed herein is a binding molecule and/or a disclosed antibody capable of specifically binding to postfusion hemagglutinin (HA) of influenza A virus strains. Disclosed herein is a binding molecule and/or a disclosed antibody capable of specifically binding to postfusion hemagglutinin (HA) of influenza B virus strains. Disclosed herein is a binding molecule and/or a disclosed antibody capable of specifically binding to postfusion hemagglutinin (HA) of influenza A virus strains and influenza B virus strains.

In an aspect, a disclosed binding molecule and/or a disclosed antibody can be a recombinant binding molecule and/or recombinant antibody. Recombinant techniques are known to the skilled person in the art. In an aspect, a disclosed binding molecule and/or a disclosed antibody can be humanized. Techniques to humanize binding molecules and/or antibodies are known to the skilled person in the art.

In an aspect, a disclosed binding molecule and/or a disclosed antibody can be considered to universally bind to HA.

In an aspect, a disclosed binding molecule and/or a disclosed antibody can bind to the stem region of the HA protein. In an aspect, a disclosed stem region of the HA protein can comprise an epitope comprising the sequence set forth in SEQ ID NO:191. In an aspect, a disclosed binding molecule and/or antibody can comprise (i) a variable light chain region (VL) comprising 3 complementarity determining regions (CDRs), and (ii) a variable heavy chain region (VH) comprising 3 CDRs and a constant heavy chain region.

In an aspect of a disclosed binding molecule and/or a disclosed antibody, the VH can comprise at least one CDR having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184. In an aspect of a disclosed binding molecule and/or a disclosed antibody, the VH can comprise at least two CDRs having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect of a disclosed binding molecule and/or a disclosed antibody, the VH can comprise at least three CDRs having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184. In an aspect of a disclosed binding molecule and/or a disclosed antibody, the VL can comprise at least one CDR having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect of a disclosed binding molecule and/or a disclosed antibody, the VL can comprise at least two CDRs having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190. In an aspect of a disclosed binding molecule and/or a disclosed antibody, the VL can comprise at least three CDRs having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect of a disclosed binding molecule and/or a disclosed antibody, the VH can comprise at least three CDRs having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184, and wherein the VL can comprise at least three CDRs having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect of a disclosed binding molecule and/or a disclosed antibody, the VH can comprise at least one CDR having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect of a disclosed binding molecule and/or a disclosed antibody, the VH can comprise at least two CDRs having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect of a disclosed binding molecule and/or a disclosed antibody, the VH can comprise at least three CDRs having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect of a disclosed binding molecule and/or a disclosed antibody, the VL can comprise at least one CDR having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect of a disclosed binding molecule and/or a disclosed antibody, the VL can comprise at least two CDRs having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect of a disclosed binding molecule and/or a disclosed antibody, the VL can comprise at least three CDRs having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect of a disclosed binding molecule and/or a disclosed antibody, the VH can comprise at least three CDRs having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184, and wherein the VL can comprise at least three CDRs having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

TABLE 1
Exemplary Heavy Chain Variable Regions

| Antibody | Heavy Chain Variable Region |
|---|---|
| S1V2-72 | QVQLVQSGAELKKPGASVKVSCKASGYTFTGNYIHWMRQVPGQGLE |
| | WMGWINPRTGDTHHAQKFQGRVDMTRDTSINTAYLELTRLESDDTA |
| | LYYCARCVFATSQFDPWGQGTLVTVSS (SEQ ID NO: 05) |
| K06.18 | EVQLVQSGAEVKKPGASVRVSCRASGYSFTDNYIHWVRQAPGQGLE |
| | WMGWINPDSGATSYEHKFQGRVTMTRDTSISTAFMELGSLRSDDTA |
| | VYYCARVRRGTAGLDYWGQGTLVTVSS (SEQ ID NO: 06) |
| 334-100 | QVQLVQSGAEVKKPGASVKVSCKASGYTFTGYYMHWVRQAPGQGLE |
| | WMGWINPNTGDTKYAQKFQDWVTMTRDTSISTAYMELNRLRSGDTA |
| | VYYCARIISRSSGLDVWGQGTTVTVSS (SEQ ID NO: 07) |
| 350-310-D7B | QVQLVQSGAEVKKPGASVKVSCRASGYTFTANYMHWVRQAPGQGLE |
| | WMGWINPKSGDTSSTEKFQGRVTMTRDTSITTAYMELSRLRSDDTA |
| | VYYCARVPYGSSPDFWGQGTLVTVSS (SEQ ID NO: 08) |
| 350-315 | QVQLVQSGAEVKKPGASVKVSCRASGYTFTANYMHWVRQAPGQGLE |
| | WMGWINPNSGDTSSAQKFQGRVTMTRDTSISTAYMELSRLRSDDTA |
| | VYYCARVPYGSSPDFWGQGTLVTVSS (SEQ ID NO: 09) |
| 350-376 | QVQLVQSGAEVKKPGASMKVSCKASGYTFTRYYVHWVRQAPGQGLE |
| | WMGWMDPNSGGTNSAQNFQGRVTMTRDTSINTAYMEVSGLRSDDTG |
| | VYYCARITVGYFPYFDFWGQGTLVTVSS (SEQ ID NO: 10) |
| M94 | QVQLVESGAEVKRPGASVKVACKASEYRFAASYIHWVRQAPGQGLE |
| | WMGWINLNSGDTKSSQKFQGWVTLTRDTSINTVYMEMTNLKFDDTA |
| | IYYCAKIILGGGGLDVWGQGTTVTVSS (SEQ ID NO: 11) |
| C1_ID26079- | QVQLVQSGAEVKKPGASVKVSCKASGYTFTGNYMHWVRQAPGQGLE |
| 156194 | WMGWINPDSGGTNYAQRFQGRVTMTRDTSISTAYMELSRLTSDDTA |
| | MYYCARVMLRSSGFDSWGQGTLVTVSS (SEQ ID NO: 12) |
| FDA053- | QVQLVQSGAEMKNPGASVKVSCKASGYTFSGYYIHWVRQAPGQGLE |
| 87001 | WMAWIDPNSGATNYPEKFQGRVTMTTDTSITTTYMELTSLSSDDTA |
| | VYYCARVRYGSGFHWGQGTLVTVSS (SEQ ID NO: 13) |
| 351-52 | QVQLAQSGAEVRKPGASVKVSCKASGYTFTDYYIHWVRQAPGQGLE |
| | WMGWINPDSGRTNYAQRFQGRVSMTSDTSISTAYMDLSRLRSDDTA |
| | VYFCAREGAHNYQHFDHWGQGTLVTVSA (SEQ ID NO: 14) |
| 350-341 | QVQLVQSGAEVKKPGASVRVSCKASGFTFTTHYIHWVRQAPGQGLE |
| | WMGWIDPSSGGTKFAQHFQGRVTMTTDASINTAFMDLNRLRSDDTA |
| | MYYCARVRFGSGWYWGQGTLVTVSS (SEQ ID NO: 15) |
| S5V6-P7F12 | QVQLVQSGAEVKRPGSSVKVSCKASGVTFSGYIMSWVRQAPGQGFE |
| | WIGGIIPIFGKTTYAQKFQGRVTITADESTTTAYMELNSLKSEDTA |
| | IYYCARGGCSSSGCYARPFDPWGQGTLVTVSS (SEQ ID NO: |
| | 16) |

TABLE 2
Exemplary Light Chain Variable Regions

| Antibody | Light Chain Variable Region |
|---|---|
| S1V2-72 | QSALTQPASVSGSPGQSITISCTGTNSDIGSHNLVSWYQQHPGKAPKV |
| | MIYDDSKRPSGVSNRFSGSKSGSTASLTISGLQSEDEADYYCCSYAGS |
| | SNWVFGGGTKLTLL (SEQ ID NO: 17) |
| K06.18 | QSVLTQPASVSGSLGQSITISCTGTSSDIGNYNLVSWYQQHPGQAPKL |
| | IIYEVNRRPSGVSDRFSDSKSGNTASLTISGLQAEDEGDYYCFSYAGG |
| | STWVFGGGTKVTVL (SEQ ID NO: 18) |
| 334-100 | QSALTQPRSVSGSPGQSVTISCAGTSSDVGGYNYVSWYQHHPGKAPKL |
| | MIYDVFKRPSGVPDRFSGSKSGNAASLIISGLQAEDEADYYCYSYSGS |
| | YTFWVFGGGTKLTVL (SEQ ID NO: 19) |
| 350-310-D7B | QSALTQPASVSGSPGQSITISCTGTSSDVGRYKFVSWYQQHPGKAPKV |
| | MIFEVTKRPSGVSDRFSGSKSANTASLTISGLQAEDEADYYCLSYAGS |
| | SNWVFGGGTRLTVL (SEQ ID NO: 20) |
| 350-315 | QSALTQPASVSGSPGQSITISCTGTSSDVGSYKFVSWYQQHPGKAPKV |
| | MIFEVTKRPSGVSDRFSGSKSANTASLTISGLQAEDEADYYCFSYAGS |
| | STWVFGGGTRLTVL (SEQ ID NO: 21) |
| 350-376 | QSALIQPRSVSGSPGQSITISCSGTSSDVGNYNYISWYQQHPGKAPKV |
| | MIYDVNKRPSGVPDRFSGSKSGNTASLTISGLQAEDEADYYCCSYAGS |
| | SSWVFGGGTKLTVL (SEQ ID NO: 22) |
| M94 | QSVLTQPRSVSGSLGQSVTISCSGTSTDVGSYEFVSWYQHHPGKAPKL |
| | IIYDVSKRPSGVPDRESGSKSGNTASLAVSGLQSDDEADYYCSSYADT |
| | SNTWVFGGGTKLTVL (SEQ ID NO: 23) |
| C1_ID26079- | QSALIQPASVSGSPGQSITISCTGTSSDVGTYNLVSWYQQHPGKVPKL |
| 156194 | MIYEASERPSGVSNRFSGSKSGNTASLTISGLQAEDEADYYCCSYAGN |
| | SAWVFGGGTKLTVL (SEQ ID NO: 24) |
| FDA053- | QAVVTQEPSLTVSPGGTVTLTCGSSTGAVTSGHYPYWFQQKAGQAPRT |
| 87001 | LIYDTGNKHSSTPARFSGSLLGGKAALTLSGAQPEDEAEYYCLLSHSG |
| | PWVFGGGTKLTVL (SEQ ID NO: 25) |
| 351-52 | EIVLTQSPDTLSLSPGESATLFCRASQSVRSSYLAWYQQTPGQAPRLL |
| | IYGASSRATGIPDRFSGSGSGTDFTLTISRLEPEDFAVYYCQQFGRSS |
| | WTFGQGTKVEIK (SEQ ID NO: 26) |
| 350-341 | QAVVTQEPSLTVSPGGTVTLTCGSTTGPVTSGHYPYWFQQKPGQAPRT |
| | VIYDTTNKHSWTPAWFSGSLLGGKAALTLSGAQPEDEAEYYCLLAYSG |
| | TWVFGGGTKLTVL (SEQ ID NO: 27) |
| S5V6-P7F12 | QPVVTQPSSLSASPGASASLTCALRSDINVGTYRIYWYQQKSGSPPQF |
| | LLRYRSDSDKDLGSGVPSRFSGSKDASANAGILLISGLQSDDEGDYYC |
| | MIWHSSA VIFGGGTKLTVL (SEQ ID NO: 28) |

Disclosed herein is a binding molecule and/or a disclosed antibody referred to as S1V2-72.

In an aspect of a disclosed binding molecule and/or a disclosed antibody, the heavy chain variable region can comprise the amino acid sequence set forth in SEQ ID NO:05. In an aspect of a disclosed binding molecule and/or a disclosed antibody, the light chain variable region can comprise the amino acid sequence set forth in SEQ ID NO:17. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:05. In an aspect of a disclosed binding molecule and/or a disclosed antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:17.

In an aspect of a disclosed binding molecule and/or a disclosed antibody, the heavy chain variable region can comprise the amino acid sequence set forth in SEQ ID NO:05, and wherein the light chain variable region can comprise the amino acid sequence set forth in SEQ ID NO:17. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:05, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:17.

In an aspect of a disclosed binding molecule and/or a disclosed antibody, the VH can comprise at least one CDR having the sequence set forth in any one of SEQ ID NO:59-SEQ ID NO:63. In an aspect of a disclosed binding molecule and/or a disclosed antibody, the VL can comprise at least one CDR having the sequence set forth in any one of SEQ ID NO:64-SEQ ID NO:69. In an aspect of a disclosed binding molecule and/or a disclosed antibody, the VH can comprise at least one CDR having the sequence set forth in any one of SEQ ID NO:59-SEQ ID NO:63, and wherein the VL comprise at least one CDR having the sequence set forth in any one of SEQ ID NO:64-SEQ ID NO:69.

In an aspect of a disclosed binding molecule and/or a disclosed antibody, the VH can comprise 3 CDRs comprising the sequence set forth in any one of SEQ ID NO:59-SEQ ID NO:63, and wherein the VL can comprise 3 CDRs comprising the sequence set forth in any one of SEQ ID NO:64-SEQ ID NO:69.

In an aspect of a disclosed binding molecule and/or a disclosed antibody, the first CDR in the VH can comprise the sequence set forth in SEQ ID NO:59 or SEQ ID NO:60; the second CDR in the VH can comprise the sequence set forth in SEQ ID NO:61 or SEQ ID NO:62; and the third CDR in the VH can comprise the sequence set forth in SEQ ID NO:63. In an aspect of a disclosed binding molecule and/or a disclosed antibody, the first CDR in the VL can comprise the sequence set forth in SEQ ID NO:64 or SEQ ID NO:65; the second CDR in the VL can comprise the sequence set forth in SEQ ID NO:66 or SEQ ID NO:67; and the third CDR in the VL can comprise the sequence set forth in SEQ ID NO:68 or SEQ ID NO:69.

In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63. In an aspect of a disclosed binding molecule and/or a disclosed antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69. In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, and the VL can comprise at least one CDR having comprising a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69. In an aspect of a disclosed binding molecule and/or a disclosed antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in SEQ ID NO:59-SEQ ID NO:63, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69.

In an aspect of a disclosed binding molecule and/or a disclosed antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:59 or SEQ ID NO:60; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:61 or SEQ ID NO:62; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:63. In an aspect, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:64 or SEQ ID NO:65; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:66 or SEQ ID NO:67; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:68 or SEQ ID NO:69.

TABLE 3
Exemplary VH-CDRs and VL-CDRs for S1V2-72

| SEQ ID # | Description | Sequence |
|---|---|---|
| 59 | VH CDR1 | GYTFTGNY |
| 60 | VH CDR1 | GYTFTGN |
| 61 | VH CDR2 | INPRTGDT |
| 62 | VH CDR2 | NPRTGD |
| 63 | VH CDR3 | ARCVFATSQFDP |
| 64 | VL CDR1 | NSDIGSHNL |
| 65 | VL CDR1 | TGTNSDIGSHNLVS |
| 66 | VL CDR2 | DDS |
| 67 | VL CDR2 | DDSKRPS |
| 68 | VL CDR3 | CSYAGSSNWV |
| 69 | VL CDR3 | CSYAGSSNWV |

Disclosed herein is a binding molecule and/or a disclosed antibody referred to as K06.18.

In an aspect of a disclosed binding molecule and/or a disclosed antibody, the heavy chain variable region can comprise the amino acid sequence set forth in SEQ ID NO:06. In an aspect of a disclosed binding molecule and/or a disclosed antibody, the light chain variable region can comprise the amino acid sequence set forth in SEQ ID NO:18. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:06. In an aspect of a disclosed binding molecule and/or a disclosed antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:18.

In an aspect of a disclosed binding molecule and/or a disclosed antibody, the heavy chain variable region can comprise the amino acid sequence set forth in SEQ ID NO:06, and wherein the light chain variable region can comprise the amino acid sequence set forth in SEQ ID NO:18. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:06, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:18.

In an aspect of a disclosed binding molecule and/or a disclosed antibody, the VH can comprise at least one CDR having the sequence set forth in any one of SEQ ID NO:70-SEQ ID NO:74. In an aspect of a disclosed binding molecule and/or a disclosed antibody, the VL can comprise at least one CDR having the sequence set forth in any one of SEQ ID NO:75-SEQ ID NO:80. In an aspect of a disclosed binding molecule and/or a disclosed antibody, the VH can comprise at least one CDR having the sequence set forth in any one of SEQ ID NO:70-SEQ ID NO:74, and the VL can comprise at least one CDR having the sequence set forth in any one of SEQ ID NO:75-SEQ ID NO:80.

In an aspect of a disclosed binding molecule and/or a disclosed antibody, the VH can comprise 3 CDRs selected from SEQ ID NO:70-SEQ ID NO:74, and the VL can comprise 3 CDRs selected from SEQ ID NO:75-SEQ ID NO:80. In an aspect of a disclosed binding molecule and/or a disclosed antibody, a first CDR in the VH can comprise the sequence set forth in SEQ ID NO:70 or SEQ ID NO:71; a second CDR in the VH can comprise the sequence set forth in SEQ ID NO:72 or SEQ ID NO:73; and a third CDR in the VH can comprise the sequence set forth in SEQ ID NO:74. In an aspect of a disclosed binding molecule and/or a disclosed antibody, a first CDR in the VL can comprise the sequence set forth in SEQ ID NO:75 or SEQ ID NO:76; a second CDR in the VL can comprise the sequence set forth in SEQ ID NO:77 or SEQ ID NO:78; and a third CDR in the VL can comprise the sequence set forth in SEQ ID NO:79 or SEQ ID NO:80.

In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:70-SEQ ID NO:74. In an aspect of a disclosed binding molecule and/or a disclosed antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:75-SEQ ID NO:80. In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:70-SEQ ID NO:74, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:75-SEQ ID NO:80. In an aspect of a disclosed binding molecule and/or a disclosed antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in SEQ ID NO:70-SEQ ID NO:74, and the VL can comprise 3 CDRs comprising a sequence having at least about 80%, 85%, 90%, 91%, 92%, 93%, 94%, 95%, 96%, 97%, 98%, 99% or greater identity to the sequence set forth in SEQ ID NO:75-SEQ ID NO:80. In an aspect of a disclosed binding molecule and/or a disclosed antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:70 or SEQ ID NO:71; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:72 or SEQ ID NO:73; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:74. In an aspect of a disclosed binding molecule and/or a disclosed antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:75 or SEQ ID NO:76; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:77 or SEQ ID NO:78; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:79 or SEQ ID NO:80.

TABLE 4
Exemplary VH-CDRs and VL-CDRs for KEL06.18

| SEQ ID # | Description | Sequence |
|---|---|---|
| 70 | VH CDR1 | GYSFTDNY |
| 71 | VH CDR1 | GYSFTDN |
| 72 | VH CDR2 | INPDSGAT |
| 73 | VH CDR2 | NPDSGA |
| 74 | VH CDR3 | ARVRRGTAGLDY |
| 75 | VL CDR1 | SSDIGNYNL |
| 76 | VL CDR1 | TGTSSDIGNYNLVS |
| 77 | VL CDR2 | EVN |
| 78 | VL CDR2 | EVNRRPS |
| 79 | VL CDR3 | FSYAGGSTWV |
| 80 | VL CDR3 | FSYAGGSTWV |

Disclosed herein is a binding molecule and/or a disclosed antibody referred to as 334-100.

In an aspect of a disclosed binding molecule and/or a disclosed antibody, the heavy chain variable region can comprise the amino acid sequence set forth in SEQ ID NO:07. In an aspect of a disclosed binding molecule and/or a disclosed antibody, the light chain variable region can comprise the amino acid sequence set forth in SEQ ID NO:19. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:07. In an aspect of a disclosed binding molecule and/or a disclosed antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:19.

In an aspect of a disclosed binding molecule and/or a disclosed antibody, the heavy chain variable region can comprise the amino acid sequence set forth in SEQ ID NO:07, and wherein the light chain variable region can comprise the amino acid sequence set forth in SEQ ID NO:19. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:07, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:19.

In an aspect of a disclosed binding molecule and/or a disclosed antibody, the VH can comprise at least one CDR having the sequence set forth in any one of SEQ ID NO:81-SEQ ID NO:85. In an aspect of a disclosed binding molecule and/or a disclosed antibody, the VL can comprise at least one CDR having the sequence set forth in any one of SEQ ID NO:86-SEQ ID NO:91. In an aspect of a disclosed binding molecule and/or a disclosed antibody, the VH can comprise at least one CDR having the sequence set forth in any one of SEQ ID NO:81-SEQ ID NO:85, and the VL can comprise at least one CDR having the sequence set forth in any one of SEQ ID NO:86-SEQ ID NO:91. In an aspect of a disclosed binding molecule and/or a disclosed antibody, the VH can comprise 3 CDRs selected from SEQ ID NO:81-SEQ ID NO:85, and the VL can comprise 3 CDRs selected from SEQ ID NO:86-SEQ ID NO:91. In an aspect of a disclosed binding molecule and/or a disclosed antibody, a first CDR in the VH can comprise the sequence set forth in SEQ ID NO:81 or SEQ ID NO:82; a second CDR in the VH can comprise the sequence set forth in SEQ ID NO:83 or SEQ ID NO:84; and a third CDR in the VH can comprise the sequence set forth in SEQ ID NO:85.

In an aspect of a disclosed binding molecule and/or a disclosed antibody, a first CDR in the VL can comprise the sequence set forth in SEQ ID NO:86 or SEQ ID NO:87; a second CDR in the VL can comprise the sequence set forth in SEQ ID NO:88 or SEQ ID NO:89; and a third CDR in the VL can comprise the sequence set forth in SEQ ID NO:90 or SEQ ID NO:91.

In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:81-SEQ ID NO:85. In an aspect of a disclosed binding molecule and/or a disclosed antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:86-SEQ ID NO:91. In an aspect of a disclosed binding molecule and/or a disclosed antibody, the VH can comprise at least one comprising a sequence having at least about 80%, 85%, 90%, 91%, 92%, 93%, 94%, 95%, 96%, 97%, 98%, 99% or greater identity to the sequence set forth in any one of SEQ ID NO:81-SEQ ID NO:85, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:86-SEQ ID NO:91. In an aspect of a disclosed binding molecule and/or a disclosed antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:81-SEQ ID NO:85, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one SEQ ID NO:86-SEQ ID NO:91. In an aspect of a disclosed binding molecule and/or a disclosed antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:81 or SEQ ID NO:82; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:83 or SEQ ID NO:84; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:85. In an aspect of a disclosed binding molecule and/or a disclosed antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:86 or SEQ ID NO:87; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:88 or SEQ ID NO:89; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:90 or SEQ ID NO:91.

TABLE 5
Exemplary VH-CDRs and VL-CDRs for 334-100

| SEQ ID # | Description | Sequence |
|---|---|---|
| 81 | VH CDR1 | GYTFTGYY |
| 82 | VH CDR1 | GYTFTGY |
| 83 | VH CDR2 | INPNTGDT |
| 84 | VH CDR2 | NPNTGD |
| 85 | VH CDR3 | ARIISRSSGLDV |
| 86 | VL CDR1 | SSDVGGYNY |
| 87 | VL CDR1 | AGTSSDVGGYNYVS |
| 88 | VL CDR2 | DVF |
| 89 | VL CDR2 | DVFKRPS |
| 90 | VL CDR3 | YSYSGSYTFWV |
| 91 | VL CDR3 | YSYSGSYTFWV |

Disclosed herein is a binding molecule and/or a disclosed antibody referred to as 350-310-D7B.

In an aspect of a disclosed binding molecule and/or a disclosed antibody, the heavy chain variable region can comprise the amino acid sequence set forth in SEQ ID NO:08. In an aspect of a disclosed binding molecule and/or a disclosed antibody, the light chain variable region can comprise the amino acid sequence set forth in SEQ ID NO:20. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:08. In an aspect of a disclosed binding molecule and/or a disclosed antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:20.

In an aspect of a disclosed binding molecule and/or a disclosed antibody, the heavy chain variable region can comprise the amino acid sequence set forth in SEQ ID NO:08, and wherein the light chain variable region can comprise the amino acid sequence set forth in SEQ ID NO:20. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:08, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:20.

In an aspect of a disclosed binding molecule and/or a disclosed antibody, the VH can comprise at least one CDR having the sequence set forth in any one of SEQ ID NO:92-SEQ ID NO:96. In an aspect of a disclosed binding molecule and/or a disclosed antibody, the VL can comprise at least one CDR having the sequence set forth in any one of SEQ ID NO:97-SEQ ID NO:102. In an aspect of a disclosed binding molecule and/or a disclosed antibody, the VH can comprise at least one CDR having the sequence set forth in any one of SEQ ID NO:92-SEQ ID NO:96, and the VL can comprise at least one CDR having the sequence set forth in any one of SEQ ID NO:97-SEQ ID NO:102. In an aspect of a disclosed binding molecule and/or a disclosed antibody, the VH can comprise 3 CDRs selected from SEQ ID NO:92-SEQ ID NO:96, and the VL can comprise 3 CDRs selected from SEQ ID NO:97-SEQ ID NO:102.

In an aspect of a disclosed binding molecule and/or a disclosed antibody, a first CDR in the VH can comprise the sequence set forth in SEQ ID NO:92 or SEQ ID NO:93; a second CDR in the VH can comprise the sequence set forth in SEQ ID NO:94 or SEQ ID NO:695; and a third CDR in the VH can comprise the sequence set forth in SEQ ID NO:96. In an aspect of a disclosed binding molecule and/or a disclosed antibody, a first CDR in the VL can comprise the sequence set forth in SEQ ID NO:97 or SEQ ID NO:98; a second CDR in the VL can comprise the sequence set forth in SEQ ID NO:99 or SEQ ID NO:100; and a third CDR in the VL can comprise the sequence set forth in SEQ ID NO:101 or SEQ ID NO:102.

In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:92-SEQ ID NO:96. In an aspect of a disclosed binding molecule and/or a disclosed antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:97-SEQ ID NO:102. In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:92-SEQ ID NO:96, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:97-SEQ ID NO:102.

In an aspect of a disclosed binding molecule and/or a disclosed antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:92-SEQ ID NO:96, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:97-SEQ ID NO:102. In an aspect of a disclosed binding molecule and/or a disclosed antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:92 or SEQ ID NO:93; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:94 or SEQ ID NO:695; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:96. In an aspect of a disclosed binding molecule and/or a disclosed antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:97 or SEQ ID NO:98; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:99 or SEQ ID NO:100; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:101 or SEQ ID NO:102.

TABLE 6
Exemplary VH-CDRs and VL-CDRs for 350-310-D7B

| SEQ ID # | Description | Sequence |
|---|---|---|
| 92 | VH CDR1 | GYTFTANY |
| 93 | VH CDR1 | GYTFTAN |
| 94 | VH CDR2 | INPKSGDT |
| 95 | VH CDR2 | NPKSGD |
| 96 | VH CDR3 | ARVPYGSSPDF |
| 97 | VL CDR1 | SSDVGRYKF |
| 98 | VL CDR1 | TGTSSDVGRYKFVS |
| 99 | VL CDR2 | EVT |
| 100 | VL CDR2 | EVTKRPS |
| 101 | VL CDR3 | LSYAGSSNWV |
| 102 | VL CDR3 | LSYAGSSNWV |

Disclosed herein is a binding molecule and/or a disclosed antibody referred to as 350-315.

In an aspect of a disclosed binding molecule and/or a disclosed antibody, the heavy chain variable region can comprise the amino acid sequence set forth in SEQ ID NO:09. In an aspect of a disclosed binding molecule and/or a disclosed antibody, the light chain variable region can comprise the amino acid sequence set forth in SEQ ID NO:21. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:09. In an aspect of a disclosed binding molecule and/or a disclosed antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:21.

In an aspect of a disclosed binding molecule and/or a disclosed antibody, the heavy chain variable region can comprise the amino acid sequence set forth in SEQ ID NO:09, and wherein the light chain variable region can comprise the amino acid sequence set forth in SEQ ID NO:21. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:09, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:21.

In an aspect of a disclosed binding molecule and/or a disclosed antibody, the VH can comprise at least one CDR having the sequence set forth in any one of SEQ ID NO:103-SEQ ID NO:107. In an aspect of a disclosed binding molecule and/or a disclosed antibody, the VL can comprise at least one CDR having the sequence set forth in any one of SEQ ID NO:108-SEQ ID NO:113. In an aspect of a disclosed binding molecule and/or a disclosed antibody, the VH can comprise at least one CDR having the sequence set forth in any one of SEQ ID NO:103-SEQ ID NO:107, and the VL can comprise at least one CDR having the sequence set forth in any one of SEQ ID NO:108-SEQ ID NO:113.

In an aspect of a disclosed binding molecule and/or a disclosed antibody, the VH can comprise 3 CDRs selected from SEQ ID NO:103-SEQ ID NO:107, and the VL can comprise 3 CDRs selected from SEQ ID NO:108-SEQ ID NO:113. In an aspect of a disclosed binding molecule and/or a disclosed antibody, a first CDR in the VH can comprise the sequence set forth in SEQ ID NO:103 or SEQ ID NO:104; a second CDR in the VH can comprise the sequence set forth in SEQ ID NO:105 or SEQ ID NO:106; and a third CDR in the VH can comprise the sequence set forth in SEQ ID NO:107. In an aspect of a disclosed binding molecule and/or a disclosed antibody, a first CDR in the VL can comprise the sequence set forth in SEQ ID NO:108 or SEQ ID NO:109; a second CDR in the VL can comprise the sequence set forth in SEQ ID NO:110 or SEQ ID NO:111; and a third CDR in the VL can comprise the sequence set forth in SEQ ID NO:112 or SEQ ID NO:113.

In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:103-SEQ ID NO:107. In an aspect of a disclosed binding molecule and/or a disclosed antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:108-SEQ ID NO:113. In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:103-SEQ ID NO:107, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:108-SEQ ID NO:113. In an aspect of a disclosed binding molecule and/or a disclosed antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:103-SEQ ID NO:107, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:108-SEQ ID NO:113.

In an aspect of a disclosed binding molecule and/or a disclosed antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:103 or SEQ ID NO:104; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:105 or SEQ ID NO:106; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:107. In an aspect of a disclosed binding molecule and/or a disclosed antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:108 or SEQ ID NO:109; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:110 or SEQ ID NO:111; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:112 or SEQ ID NO:113.

TABLE 7
Exemplary VH-CDRs and VL-CDRs for 350-315

| SEQ ID # | Description | Sequence |
|---|---|---|
| 103 | VH CDR1 | GYTFTANY |
| 104 | VH CDR1 | GYTFTAN |
| 105 | VH CDR2 | INPNSGDT |
| 106 | VH CDR2 | NPNSGD |
| 107 | VH CDR3 | ARVPYGSSPDF |
| 108 | VL CDR1 | SSDVGSYKF |
| 109 | VL CDR1 | TGTSSDVGSYKFVS |
| 110 | VL CDR2 | EVT |
| 111 | VL CDR2 | EVTKRPS |
| 112 | VL CDR3 | FSYAGSSTWV |
| 113 | VL CDR3 | FSYAGSSTWV |

Disclosed herein is a binding molecule and/or a disclosed antibody referred to as 350-376.

In an aspect of a disclosed binding molecule and/or a disclosed antibody, the heavy chain variable region can comprise the amino acid sequence set forth in SEQ ID NO:10. In an aspect of a disclosed binding molecule and/or a disclosed antibody, the light chain variable region can comprise the amino acid sequence set forth in SEQ ID NO:22. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:10. In an aspect of a disclosed binding molecule and/or a disclosed antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:22.

In an aspect of a disclosed binding molecule and/or a disclosed antibody, the heavy chain variable region can comprise the amino acid sequence set forth in SEQ ID NO:10, and wherein the light chain variable region can comprise the amino acid sequence set forth in SEQ ID NO:22. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:10, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:22.

In an aspect of a disclosed binding molecule and/or a disclosed antibody, the VH can comprise at least one CDR having the sequence set forth in any one of SEQ ID NO:114-SEQ ID NO:118. In an aspect of a disclosed binding molecule and/or a disclosed antibody, the VL can comprise at least one CDR having the sequence set forth in any one of SEQ ID NO:119-SEQ ID NO:124. In an aspect of a disclosed binding molecule and/or a disclosed antibody, the VH can comprise at least one CDR having the sequence set forth in any one of SEQ ID NO:114-SEQ ID NO:118, and the VL can comprise at least one CDR having the sequence set forth in any one of SEQ ID NO:119-SEQ ID NO:124. In an aspect of a disclosed binding molecule and/or a disclosed antibody, the first CDR in the VH can comprise the sequence set forth in SEQ ID NO:114 or SEQ ID NO:115; a second CDR in the VH can comprise the sequence set forth in SEQ ID NO:116 or SEQ ID NO:117; and a third CDR in the VH can comprise the sequence set forth in SEQ ID NO:118. In an aspect of a disclosed binding molecule and/or a disclosed antibody, a first CDR in the VL can comprise the sequence set forth in SEQ ID NO:119 or SEQ ID NO:120; a second CDR in the VL can comprise the sequence set forth in SEQ ID NO:121 or SEQ ID NO:122; and a third CDR in the VL can comprise the sequence set forth in SEQ ID NO:123 or SEQ ID NO:124.

In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:114-SEQ ID NO:118. In an aspect of a disclosed binding molecule and/or a disclosed antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:119-SEQ ID NO:124. In an aspect of a disclosed binding molecule and/or a disclosed antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:114-SEQ ID NO:118, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:119-SEQ ID NO:124.

In an aspect of a disclosed binding molecule and/or a disclosed antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:114 or SEQ ID NO:115; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:116 or SEQ ID NO:117; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:118. In an aspect of a disclosed binding molecule and/or a disclosed antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:119 or SEQ ID NO:120; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:121 or SEQ ID NO:122; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:123 or SEQ ID NO:124.

TABLE 8
Exemplary VH-CDRs and VL-CDRs for 350-376

| SEQ ID # | Description | Sequence |
|---|---|---|
| 114 | VH CDR1 | GYTFTRYY |
| 115 | VH CDR1 | GYTFTRY |
| 116 | VH CDR2 | MDPNSGGT |
| 117 | VH CDR2 | DPNSGG |
| 118 | VH CDR3 | ARITVGYFPYFDF |
| 119 | VL CDR1 | SSDVGNYNY |
| 120 | VL CDR1 | SGTSSDVGNYNYIS |
| 121 | VL CDR2 | DVN |
| 122 | VL CDR2 | DVNKRPS |
| 123 | VL CDR3 | CSYAGSSSWV |
| 124 | VL CDR3 | CSYAGSSSWV |

Disclosed herein is a binding molecule and/or a disclosed antibody referred to as 351-52.

In an aspect of a disclosed binding molecule and/or a disclosed antibody, the heavy chain variable region can comprise the amino acid sequence set forth in SEQ ID NO:14. In an aspect of a disclosed binding molecule and/or a disclosed antibody, the light chain variable region can comprise the amino acid sequence set forth in SEQ ID NO:26. In an aspect of a disclosed binding molecule and/or a disclosed antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:14. In an aspect of a disclosed binding molecule and/or antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:26.

In an aspect of a disclosed binding molecule and/or antibody, the heavy chain variable region can comprise the amino acid sequence set forth in SEQ ID NO:14, and wherein the light chain variable region can comprise the amino acid sequence set forth in SEQ ID NO:26. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:14, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:26.

In an aspect of a disclosed binding molecule and/or antibody, the VH can comprise at least one CDR having the sequence set forth in any one of SEQ ID NO:169-SEQ ID NO:173. In an aspect of a disclosed binding molecule and/or antibody, the VL can comprise at least one CDR having the sequence set forth in any one of SEQ ID NO:174-SEQ ID NO:179. In an aspect of a disclosed binding molecule and/or antibody, the VH can comprise at least one CDR having the sequence set forth in any one of SEQ ID NO:169-SEQ ID NO:173, and the VL can comprise at least one CDR having the sequence set forth in any one of SEQ ID NO:174-SEQ ID NO:179.

In an aspect of a disclosed binding molecule and/or antibody, the VH can comprise 3 CDRs selected from SEQ ID NO:169-SEQ ID NO:173, and the VL can comprise 3 CDRs having the sequence set froth in any one of SEQ ID NO:174-SEQ ID NO:179. In an aspect of a disclosed binding molecule and/or antibody, a first CDR in the VH can comprise the sequence set forth in SEQ ID NO:169 or SEQ ID NO:170; a second CDR in the VH can comprise the sequence set forth in SEQ ID NO:171 or SEQ ID NO:172; and a third CDR in the VH can comprise the sequence set forth in SEQ ID NO:173. In an aspect of a disclosed binding molecule and/or antibody, a first CDR in the VL can comprise the sequence set forth in SEQ ID NO:174 or SEQ ID NO:175; a second CDR in the VL can comprise the sequence set forth in SEQ ID NO:176 or SEQ ID NO:177; and a third CDR in the VL can comprise the sequence set forth in SEQ ID NO:178 or SEQ ID NO:179.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:169-SEQ ID NO:173. In an aspect of a disclosed binding molecule and/or antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:174-SEQ ID NO:179. In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:169-SEQ ID NO:173, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:174-SEQ ID NO:179.

In an aspect of a disclosed binding molecule and/or antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:169-SEQ ID NO:173, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:174-SEQ ID NO:179.

In an aspect of a disclosed binding molecule and/or antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:169 or SEQ ID NO:170; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:171 or SEQ ID NO:172; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:173.

In an aspect of a disclosed binding molecule and/or antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:174 or SEQ ID NO:175; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:176 or SEQ ID NO:177; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:178 or SEQ ID NO:179.

TABLE 9
Exemplary VH-CDRs and VL-CDRs for 351-352

| SEQ ID # | Description | Sequence |
|---|---|---|
| 169 | VH CDR1 | GYTFTDYY |
| 170 | VH CDR1 | GYTFTDY |
| 171 | VH CDR2 | INPDSGRT |
| 172 | VH CDR2 | NPDSGR |
| 173 | VH CDR3 | AREGAHNYQHFDH |
| 174 | VL CDR1 | QSVRSSY |
| 175 | VL CDR1 | RASQSVRSSYLA |
| 176 | VL CDR2 | GAS |
| 177 | VL CDR2 | GASSRAT |
| 178 | VL CDR3 | QQFGRSSWT |
| 179 | VL CDR3 | QQFGRSSWT |

Disclosed herein is a binding molecule and/or antibody referred to as 350-341.

In an aspect of a disclosed binding molecule and/or antibody, the heavy chain variable region can comprise the amino acid sequence set forth in SEQ ID NO:15. In an aspect of a disclosed binding molecule and/or antibody, the light chain variable region can comprise the amino acid sequence set forth in SEQ ID NO:27. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:15. In an aspect of a disclosed binding molecule and/or antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:27.

In an aspect of a disclosed binding molecule and/or antibody, the heavy chain variable region can comprise the amino acid sequence set forth in SEQ ID NO:15, and wherein the light chain variable region can comprise the amino acid sequence set forth in SEQ ID NO:27. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:15, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:27.

In an aspect of a disclosed binding molecule and/or antibody, the VH can comprise at least one CDR having the sequence set forth in any one of SEQ ID NO:158-SEQ ID NO:162. In an aspect of a disclosed binding molecule and/or antibody, the VL can comprise at least one CDR having the sequence set forth in any one of SEQ ID NO:163-SEQ ID NO:168. In an aspect of a disclosed binding molecule and/or antibody, the VH can comprise at least one CDR having the sequence set forth in any one of SEQ ID NO:158-SEQ ID NO:162, and wherein the VL can comprise at least one CDR having the sequence set forth in any one of SEQ ID NO:163-SEQ ID NO:168.

In an aspect of a disclosed binding molecule and/or antibody, the VH can comprise 3 CDRs selected from SEQ ID NO:158-SEQ ID NO:162, and the VL can comprise 3 CDRs selected from SEQ ID NO:163-SEQ ID NO:168. n an aspect of a disclosed binding molecule and/or antibody, a first CDR in the VH can comprise the sequence set forth in SEQ ID NO:158 or SEQ ID NO:159; a second CDR in the VH can comprise the sequence set forth in SEQ ID NO:160 or SEQ ID NO:161; and a third CDR in the VH can comprise the sequence set forth in SEQ ID NO:162. In an aspect of a disclosed binding molecule and/or antibody, a first CDR in the VL can comprise the sequence set forth in SEQ ID NO:163 or SEQ ID NO:164; a second CDR in the VL can comprise the sequence set forth in SEQ ID NO:165 or SEQ ID NO:166; and a third CDR in the VL can comprise the sequence set forth in SEQ ID NO:167 or SEQ ID NO:168.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:158-SEQ ID NO:162. In an aspect of a disclosed binding molecule and/or antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:163-SEQ ID NO:168. In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:158-SEQ ID NO:162, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:163-SEQ ID NO:168.

In an aspect of a disclosed binding molecule and/or antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:158-SEQ ID NO:162, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:163-SEQ ID NO:168.

In an aspect of a disclosed binding molecule and/or antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:158 or SEQ ID NO:159; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:160 or SEQ ID NO:161; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:162.

In an aspect of a disclosed binding molecule and/or antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:163 or SEQ ID NO:164; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:165 or SEQ ID NO:166; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:167 or SEQ ID NO:168.

TABLE 10
Exemplary VH-CDRs and VL-CDRs for 350-341

| SEQ ID # | Description | Sequence |
|---|---|---|
| 158 | VH CDR1 | GFTFTTHY |
| 159 | VH CDR1 | GFTFTTH |
| 160 | VH CDR2 | IDPSSGGT |
| 161 | VH CDR2 | DPSSGG |
| 162 | VH CDR3 | ARVRFGSGWY |
| 163 | VL CDR1 | TGPVTSGHY |
| 164 | VL CDR1 | GSTTGPVTSGHYPY |
| 165 | VL CDR2 | DTT |
| 166 | VL CDR2 | DTTNKHS |
| 167 | VL CDR3 | LLAYSGTWV |
| 168 | VL CDR3 | LLAYSGTWV |

Disclosed herein is a binding molecule and/or antibody referred to as S5V6-P7F12.

In an aspect of a disclosed binding molecule and/or antibody, the heavy chain variable region can comprise the amino acid sequence set forth in SEQ ID NO:16. In an aspect of a disclosed binding molecule and/or antibody, the light chain variable region can comprise the amino acid sequence set forth in SEQ ID NO:28. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:16. In an aspect of a disclosed binding molecule and/or antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:28.

In an aspect of a disclosed binding molecule and/or antibody, the heavy chain variable region can comprise the amino acid sequence set forth in SEQ ID NO:16, and wherein the light chain variable region can comprise the amino acid sequence set forth in SEQ ID NO:28. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:16, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:28.

In an aspect of a disclosed binding molecule and/or antibody, the VH can comprise at least one CDR having the sequence set forth in any one of SEQ ID NO:180-SEQ ID NO:184. In an aspect of a disclosed binding molecule and/or antibody, the VL can comprise at least one CDR having the sequence set forth in any one of SEQ ID NO:185-SEQ ID NO:190. In an aspect of a disclosed binding molecule and/or antibody, the VH can comprise at least one CDR having the sequence set forth in any one of SEQ ID NO:180-SEQ ID NO:184, and the VL can comprise at least one CDR having the sequence set forth in any one of SEQ ID NO:185-SEQ ID NO:190. In an aspect of a disclosed binding molecule and/or antibody, the VH can comprise 3 CDRs selected from SEQ ID NO:180-SEQ ID NO:184, and the VL can comprise 3 CDRs selected from SEQ ID NO:185-SEQ ID NO:190.

In an aspect of a disclosed binding molecule and/or antibody, a first CDR in the VH can comprise the sequence set forth in SEQ ID NO:180 or SEQ ID NO:181; a second CDR in the VH can comprise the sequence set forth in SEQ ID NO:182 or SEQ ID NO:183; and a third CDR in the VH can comprise the sequence set forth in SEQ ID NO:184.

In an aspect of a disclosed binding molecule and/or antibody, a first CDR in the VL can comprise the sequence set forth in SEQ ID NO:185 or SEQ ID NO:186; a second CDR in the VL can comprise the sequence set forth in SEQ ID NO:187 or SEQ ID NO:188; and a third CDR in the VL can comprise the sequence set forth in SEQ ID NO:189 or SEQ ID NO:190.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:180-SEQ ID NO:184. In an aspect of a disclosed binding molecule and/or antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:185-SEQ ID NO:190.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:180-SEQ ID NO:184, and the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:185-SEQ ID NO:190.

In an aspect of a disclosed binding molecule and/or antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:180-SEQ ID NO:184, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:185-SEQ ID NO:190.

In an aspect of a disclosed binding molecule and/or antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:180 or SEQ ID NO:181; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:182 or SEQ ID NO:183; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:184.

In an aspect of a disclosed binding molecule and/or antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:185 or SEQ ID NO:186; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:187 or SEQ ID NO:188; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:189 or SEQ ID NO:190.

TABLE 11
Exemplary VH-CDRs and VL-CDRs for S5V6-P7F12

| SEQ ID # | Description | Sequence |
|---|---|---|
| 180 | VH CDR1 | GVTFSGYI |
| 181 | VH CDR1 | GVTFSGY |
| 182 | VH CDR2 | IIPIFGKT |
| 183 | VH CDR2 | IPIFGK |
| 184 | VH CDR3 | ARGGCSSSGCYARPFDP |
| 185 | VL CDR1 | SDINVGTYR |
| 186 | VL CDR1 | ALRSDINVGTYRIY |
| 187 | VL CDR2 | YRSDSDK |
| 188 | VL CDR2 | YRSDSDKDLGS |
| 189 | VL CDR3 | MIWHSSAVI |
| 190 | VL CDR3 | MIWHSSAVI |

Disclosed herein is a binding molecule and/or antibody referred to as M94.

In an aspect of a disclosed binding molecule and/or antibody, the heavy chain variable region can comprise the amino acid sequence set forth in SEQ ID NO:11. In an aspect of a disclosed binding molecule and/or antibody, the light chain variable region can comprise the amino acid sequence set forth in SEQ ID NO:23. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:11. In an aspect of a disclosed binding molecule and/or antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:23.

In an aspect of a disclosed binding molecule and/or antibody, the heavy chain variable region can comprise the amino acid sequence set forth in SEQ ID NO:11, and wherein the light chain variable region can comprise the amino acid sequence set forth in SEQ ID NO:23. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:11, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:23.

In an aspect of a disclosed binding molecule and/or antibody, the VH can comprise at least one CDR having the sequence set forth in any one of SEQ ID NO:125-SEQ ID NO:129. In an aspect of a disclosed binding molecule and/or antibody, the VL can comprise at least one CDR having the sequence set forth in any one of SEQ ID NO:130-SEQ ID NO:135. In an aspect of a disclosed binding molecule and/or antibody, the VH can comprise at least one CDR having the sequence set forth in any one of SEQ ID NO:125-SEQ ID NO:129, and the VL comprise at least one CDR having the sequence set forth in any one of SEQ ID NO:130-SEQ ID NO:135.

In an aspect of a disclosed binding molecule and/or antibody, the VH can comprise 3 CDRs selected from SEQ ID NO:125-SEQ ID NO:129, and the VL can comprise 3 CDRs selected from SEQ ID NO:130-SEQ ID NO:135. In an aspect of a disclosed binding molecule and/or antibody, a first CDR in the VH can comprise the sequence set forth in SEQ ID NO:125 or SEQ ID NO:126; a second CDR in the VH can comprise the sequence set forth in SEQ ID NO:127 or SEQ ID NO:128; and a third CDR in the VH can comprise the sequence set forth in SEQ ID NO:129. In an aspect of a disclosed binding molecule and/or antibody, a first CDR in the VL can comprise the sequence set forth in SEQ ID NO:130 or SEQ ID NO:131; a second CDR in the VL can comprise the sequence set forth in SEQ ID NO:132 or SEQ ID NO:133; and a third CDR in the VL can comprise the sequence set forth in SEQ ID NO:134 or SEQ ID NO:135.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:125-SEQ ID NO:129. In an aspect of a disclosed binding molecule and/or antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:130-SEQ ID NO:135. In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:125-SEQ ID NO:129, and the VL comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:130-SEQ ID NO:135.

In an aspect of a disclosed binding molecule and/or antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:125-SEQ ID NO:129, and the VL can comprise 3 CDRs can comprise a sequence having at least about 80%, 85%, 90%, 91%, 92%, 93%, 94%, 95%, 96%, 97%, 98%, 99% or greater identity to the sequence set forth in any one of SEQ ID NO:130-SEQ ID NO:135.

In an aspect of a disclosed binding molecule and/or antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:125 or SEQ ID NO:126; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:127 or SEQ ID NO:128; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:129.

In an aspect of a disclosed binding molecule and/or antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:130 or SEQ ID NO:131; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:132 or SEQ ID NO:133; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:134 or SEQ ID NO:135.

TABLE 12
Exemplary VH-CDRs and VL-CDRs for M94

| SEQ ID # | Description | Sequence |
|---|---|---|
| 125 | VH CDR1 | EYRFAASY |
| 126 | VH CDR1 | EYRFAAS |
| 127 | VH CDR2 | INLNSGDT |
| 128 | VH CDR2 | NLNSGD |
| 129 | VH CDR3 | AKIILGGGGLDV |
| 130 | VL CDR1 | STDVGSYEF |
| 131 | VL CDR1 | SGTSTDVGSYEFVS |
| 132 | VL CDR2 | DVS |
| 133 | VL CDR2 | DVSKRPS |
| 134 | VL CDR3 | SSYADTSNTWV |
| 135 | VL CDR3 | SSYADTSNTWV |

Disclosed herein is a binding molecule and/or antibody referred to as C1_ID26079-156194.

In an aspect of a disclosed binding molecule and/or antibody, the heavy chain variable region can comprise the amino acid sequence set forth in SEQ ID NO:12. In an aspect of a disclosed binding molecule and/or antibody, the light chain variable region can comprise the amino acid sequence set forth in SEQ ID NO:24. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:12. In an aspect of a disclosed binding molecule and/or antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:24.

In an aspect of a disclosed binding molecule and/or antibody, the heavy chain variable region can comprise the amino acid sequence set forth in SEQ ID NO:12, and wherein the light chain variable region can comprise the amino acid sequence set forth in SEQ ID NO:24. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:12, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:24.

In an aspect of a disclosed binding molecule and/or antibody, the VH can comprise at least one CDR having the sequence set forth in any one of SEQ ID NO:136-SEQ ID NO:140. In an aspect of a disclosed binding molecule and/or antibody, the VL can comprise at least one CDR having the sequence set forth in any one of SEQ ID NO:141-SEQ ID NO:146. In an aspect of a disclosed binding molecule and/or antibody, the VH can comprise at least one CDR having the sequence set forth in any one of SEQ ID NO:136-SEQ ID NO:140, and the VL comprise at least one CDR having the sequence set forth in any one of SEQ ID NO:141-SEQ ID NO:146. In an aspect of a disclosed binding molecule and/or antibody, the VH can comprise 3 CDRs selected from SEQ ID NO:136-SEQ ID NO:140, and the VL can comprise 3 CDRs selected from SEQ ID NO:141-SEQ ID NO:146.

In an aspect of a disclosed binding molecule and/or antibody, a first CDR in the VH can comprise the sequence set forth in SEQ ID NO:136 or SEQ ID NO:137; a second CDR in the VH can comprise the sequence set forth in SEQ ID NO:138 or SEQ ID NO:139; and a third CDR in the VH can comprise the sequence set forth in SEQ ID NO:140.

In an aspect of a disclosed binding molecule and/or antibody, a first CDR in the VL can comprise the sequence set forth in SEQ ID NO:141 or SEQ ID NO:142; a second CDR in the VL can comprise the sequence set forth in SEQ ID NO:143 or SEQ ID NO:144; and a third CDR in the VL can comprise the sequence set forth in SEQ ID NO:145 or SEQ ID NO:146.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:136-SEQ ID NO:140. In an aspect of a disclosed binding molecule and/or antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:141-SEQ ID NO:146.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:136-SEQ ID NO:140, and the VL comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:141-SEQ ID NO:146.

In an aspect of a disclosed binding molecule and/or antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:136-SEQ ID NO:140, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:141-SEQ ID NO:146.

In an aspect of a disclosed binding molecule and/or antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:136 or SEQ ID NO:137; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:138 or SEQ ID NO:139; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:140.

In an aspect of a disclosed binding molecule and/or antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:141 or SEQ ID NO:142; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:143 or SEQ ID NO:144; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:145 or SEQ ID NO:146.

TABLE 13
Exemplary VH-CDRs and VL-CDRs for
C1_ID26079-156194

| SEQ ID # | Description | Sequence |
|---|---|---|
| 136 | VH CDR1 | GYTFTGNY |
| 137 | VH CDR1 | GYTFTGN |
| 138 | VH CDR2 | INPDSGGT |
| 139 | VH CDR2 | NPDSGG |
| 140 | VH CDR3 | ARVMLRSSGFDS |
| 141 | VL CDR1 | SSDVGTYNL |
| 142 | VL CDR1 | TGTSSDVGTYNLVS |
| 143 | VL CDR2 | EAS |
| 144 | VL CDR2 | EASERPS |
| 145 | VL CDR3 | CSYAGNSAWV |
| 146 | VL CDR3 | CSYAGNSAWV |

Disclosed herein is a binding molecule and/or antibody referred to as FDA053-87001.

In an aspect of a disclosed binding molecule and/or antibody, the heavy chain variable region can comprise the amino acid sequence set forth in SEQ ID NO:13. In an aspect of a disclosed binding molecule and/or antibody, the light chain variable region can comprise the amino acid sequence set forth in SEQ ID NO:25.

In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:13. In an aspect of a disclosed binding molecule and/or antibody, the light chain variable region can comprise a sequence having at least about 80%, 85%, 90%, 91%, 92%, 93%, 94%, 95%, 96%, 97%, 98%, 99% or greater identity to the sequence set forth in SEQ ID NO:25.

In an aspect of a disclosed binding molecule and/or antibody, the heavy chain variable region can comprise the amino acid sequence set forth in SEQ ID NO:13, and wherein the light chain variable region can comprise the amino acid sequence set forth in SEQ ID NO:25. In an aspect of a disclosed binding molecule and/or antibody, the heavy chain variable region can comprise a sequence having at least about 80%, 85%, 90%, 91%, 92%, 93%, 94%, 95%, 96%, 97%, 98%, 99% or greater identity to the sequence set forth in SEQ ID NO:13, and wherein the light chain variable region can comprise a sequence having at least about 80%, 85%, 90%, 91%, 92%, 93%, 94%, 95%, 96%, 97%, 98%, 99% or greater identity to the sequence set forth in SEQ ID NO:25.

In an aspect of a disclosed binding molecule and/or antibody, the VH can comprise at least one CDR having the sequence set forth in any one of SEQ ID NO:147-SEQ ID NO:151. In an aspect of a disclosed binding molecule and/or antibody, the VL can comprise at least one CDR having the sequence set forth in any one of SEQ ID NO:152-SEQ ID NO:157. In an aspect of a disclosed binding molecule and/or antibody, the VH can comprise at least one CDR having the sequence set forth in any one of SEQ ID NO:147-SEQ ID NO:151, and the VL comprise at least one CDR having the sequence set forth in any one of SEQ ID NO:152-SEQ ID NO:157. In an aspect of a disclosed binding molecule and/or antibody, the VH can comprise 3 CDRs selected from SEQ ID NO:147-SEQ ID NO:151, and the VL can comprise 3 CDRs selected from SEQ ID NO:152-SEQ ID NO:157.

In an aspect of a disclosed binding molecule and/or antibody, a first CDR in the VH can comprise the sequence set forth in SEQ ID NO:147 or SEQ ID NO:148; a second CDR in the VH can comprise the sequence set forth in SEQ ID NO:149 or SEQ ID NO:150; and a third CDR in the VH can comprise the sequence set forth in SEQ ID NO:151.

In an aspect of a disclosed binding molecule and/or antibody, a first CDR in the VL can comprise the sequence set forth in SEQ ID NO:152 or SEQ ID NO:153; a second CDR in the VL can comprise the sequence set forth in SEQ ID NO:154 or SEQ ID NO:155; and a third CDR in the VL can comprise the sequence set forth in SEQ ID NO:156 or SEQ ID NO:157.

In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:147-SEQ ID NO:151. In an aspect of a disclosed binding molecule and/or antibody, the VL can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:152-SEQ ID NO:157. In an aspect of a disclosed binding molecule and/or antibody, the VH can comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:147-SEQ ID NO:151, and the VL comprise at least one CDR comprising a sequence having at least about 80%, 85%, 90%, 91%, 92%, 93%, 94%, 95%, 96%, 97%, 98%, 99% or greater identity to the sequence set forth in any one of SEQ ID NO:152-SEQ ID NO:157.

In an aspect of a disclosed binding molecule and/or antibody, the VH can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:147-SEQ ID NO:151, and the VL can comprise 3 CDRs comprising a sequence having at least about 80%, 85%, 90%, 91%, 92%, 93%, 94%, 95%, 96%, 97%, 98%, 99% or greater identity to the sequence set forth in any one of SEQ ID NO:152-SEQ ID NO:157.

In an aspect of a disclosed binding molecule and/or antibody, a first CDR in the VH can comprise a sequence having at least about 80%, 85%, 90%, 91%, 92%, 93%, 94%, 95%, 96%, 97%, 98%, 99% or greater identity to the sequence set forth in SEQ ID NO:147 or SEQ ID NO:148; a second CDR in the VH can comprise a sequence having at least about 80%, 85%, 90%, 91%, 92%, 93%, 94%, 95%, 96%, 97%, 98%, 99% or greater identity to the sequence set forth in SEQ ID NO:149 or SEQ ID NO:150; and a third CDR in the VH can comprise a sequence having at least about 80%, 85%, 90%, 91%, 92%, 93%, 94%, 95%, 96%, 97%, 98%, 99% or greater identity to the sequence set forth in SEQ ID NO:151.

In an aspect of a disclosed binding molecule and/or antibody, a first CDR in the VL can comprise a sequence having at least about 80%, 85%, 90%, 91%, 92%, 93%, 94%, 95%, 96%, 97%, 98%, 99% or greater identity to the sequence set forth in SEQ ID NO:152 or SEQ ID NO:153; a second CDR in the VL can comprise a sequence having at least about 80%, 85%, 90%, 91%, 92%, 93%, 94%, 95%, 96%, 97%, 98%, 99% or greater identity to the sequence set forth in SEQ ID NO:154 or SEQ ID NO:155; and a third CDR in the VL can comprise a sequence having at least about 80%, 85%, 90%, 91%, 92%, 93%, 94%, 95%, 96%, 97%, 98%, 99% or greater identity to the sequence set forth in SEQ ID NO:156 or SEQ ID NO:157.

TABLE 14
Exemplary VH-CDRs and VL-CDRs
for FDA053-87001

| SEQ ID # | Description | Sequence |
|---|---|---|
| 147 | VH CDR1 | GYTFSGYY |
| 148 | VH CDR1 | GYTFSGY |
| 149 | VH CDR2 | IDPNSGAT |
| 150 | VH CDR2 | DPNSGA |
| 151 | VH CDR3 | ARVRYGSGFH |
| 152 | VL CDR1 | TGAVTSGHY |
| 153 | VL CDR1 | GSSTGAVTSGHYPY |
| 154 | VL CDR2 | DTG |
| 155 | VL CDR2 | DTGNKHS |
| 156 | VL CDR3 | LLSHSGPWV |
| 157 | VL CDR3 | LLSHSGPWV |

In an aspect, a disclosed binding molecule and/or antibody can confer Fc-dependent protection. In an aspect, the binding of a disclosed binding molecule and/or antibody can activate and/or elicit complement-dependent cytotoxicity (CDC), antibody-dependent cellular phagocytosis (ADCP), and antibody-dependent cellular cytotoxicity (ADCC), or any combination thereof. In an aspect, a disclosed binding molecule and/or antibody does not neutralize infectivity of the influenza virus. In an aspect, a disclosed binding molecule and/or antibody can confer protection against lethal infection of the influenza virus. In an aspect, a disclosed binding molecule and/or antibody can confer protection against mortality and/or morbidity associated with the influenza virus.

In an aspect, a disclosed binding molecule and/or antibody can bind to a sequence in a β-hairpin of the prefusion HA2. In an aspect, a disclosed binding molecule and/or antibody can bind to a sequence in a β-hairpin of the postfusion HA2. In an aspect, a disclosed postfusion HA protein2 can comprise a β-hairpin epitope. In an aspect, a disclosed postfusion HA protein2 can comprise a β-hairpin epitope comprising a consensus sequence having the sequence set forth in SEQ ID NO:191. In an aspect, a disclosed binding molecule and/or antibody can bind to HA2 via β-hairpin epitope. In an aspect, a disclosed CDR3 of a disclosed LC of a binding molecule and/or antibody can engage the β-hairpin of HA2. In an aspect, a β-hairpin of HA2 can comprise residues 75-155. In an aspect, a β-hairpin of HA2 can comprise residues 75-155 of any disclosed HA2. Exemplary HA2 sequences are set forth in Table 16. Table 16 contains full length and truncated versions of several HA2 sequences. In an aspect, a β-hairpin of HA2 can comprise about residue 75 to about residue 155 of any one of SEQ ID NO:192-SEQ ID NO:218.

In an aspect, a disclosed binding molecule and/or antibody can bind to a sequence in a β-hairpin any one of SEQ ID NO:192-218. In an aspect, a disclosed binding molecule and/or antibody can bind to a sequence in a β-hairpin any one of SEQ ID NO:29-SEQ ID NO:55.

In an aspect, a disclosed CDR3 can comprise one or more serine amino acids and/or one or more threonine amino acids. In an aspect, a disclosed CDR3 can comprise one or more serine amino acids and/or one or more threonine amino acids can engage the β-hairpin of HA2. In an aspect of a disclosed binding molecule and/or antibody, a disclosed CDR3 of a disclosed light can comprise 9 amino acids, 10 amino acids, or 11 amino acids, and have one or more serines and/or threonines. In an aspect, a LC CDR3 can comprise 9 amino acids and can comprise a serine or a threonine at residue 109 or 114 or 115 (IMGT numbering). In an aspect, a LC CDR3 can comprise 10 amino acids and can comprise a serine or a threonine at residue 113 or 114 or 115 (IMGT numbering). In an aspect, a LC CDR3 can comprise 11 amino acids and can comprise a serine or a threonine at residue 110 or 113 or 114 (IMGT numbering).

Disclosed herein is a binding molecule and/or antibody comprising a VH and/or a VL encoded by a sequence set forth in Table 15. Disclosed herein is a binding molecule and/or antibody comprising a VH and/or a VL encoded by a sequence set forth in any one of SEQ ID NO:01-SEQ ID NO:04 and SEQ ID NO:219-SEQ ID NO:236.

In an aspect, a disclosed binding molecule and/or antibody can inhibit egress of influenza virus from infected cells. In an aspect, a disclosed binding molecule and/or a disclosed antibody (i) can minimize and/or decrease the risk of the subject's mortality; (ii) can increase and/or prolong the subject's survival; (iii) can enhance and/or improve the subject's quality of life; (iv) can reduce and/or minimize the likelihood of surgical intervention; (v) can reduce and/or decrease the frequency of other treatment; (vi) can relieve and/or ameliorate one or more subject's symptoms; (vii) can reduce and/or minimize the subject's risk of hospitalization; (viii) can prevent and/or minimize the need for surgical intervention; (ix) can improve and/or restore normal metabolism of one or more of the subject's organ systems, (x) can restore and/or improve one or more aspects of cellular homeostasis and/or cellular functionality, and/or metabolic dysregulation in one or more of the subject's affected systems, or (xi) any combination thereof. In an aspect, a disclosed binding molecule and/or a disclosed antibody can treat an influenza infection.

In an aspect, a disclosed binding molecule and/or a disclosed antibody can be formulated for encapsulation in a nanoparticle and/or a lipid nanoparticle.

### Postfusion HA Protein

Disclosed herein is a postfusion HA protein or a fragment thereof. In an aspect, a postfusion HA protein or fragment thereof can be isolated from one or more subjects having an active influenza infection or having had an influenza infection. In an aspect, a disclosed postfusion HA protein or fragment thereof can comprise a β-hairpin. In an aspect, a disclosed postfusion HA protein or fragment thereof can comprise a β-hairpin capable of binding to one or more disclosed binding molecules and/or disclosed antibodies. In an aspect, a disclosed postfusion HA protein or fragment thereof can comprise a β-hairpin comprising the sequence set forth in SEQ ID NO:191. In an aspect, a disclosed postfusion HA protein or fragment thereof can comprise the sequence set forth in any one of SEQ ID NO:219-SEQ ID NO:236. In an aspect, a disclosed postfusion HA protein or fragment thereof can comprise the sequence set forth in any one of SEQ ID NO:29-SEQ ID NO:58. In an aspect, a disclosed postfusion HA protein or fragment thereof can comprise a sequence having at least about 80%, at least about 85%, at least about 90%, at least about 95%, or more than at least 95% identity to the sequence set forth in any one of SEQ ID NO:219-SEQ ID NO:236. In an aspect, a disclosed postfusion HA protein or fragment thereof can comprise a sequence having at least about 80%, at least about 85%, at least about 90%, at least about 95%, or more than at least 95% identity to the sequence set forth in any one of SEQ ID NO:29-SEQ ID NO:58.

In an aspect, a disclosed postfusion HA protein or fragment thereof can comprise a sequence having at least about 80%, at least about 85%, at least about 90%, at least about 95%, or more than at least 95% identity to the sequence set forth in Table 16. In an aspect, a disclosed postfusion HA protein or fragment thereof can comprise a sequence set forth in Table 16.

In an aspect, a disclosed postfusion HA protein or fragment thereof can be generated following an influenza infection. In an aspect, a disclosed postfusion HA protein or fragment thereof can be antigenic. In an aspect, a disclosed postfusion HA protein or fragment thereof can be antigenic. In an aspect, a disclosed postfusion HA protein or fragment thereof can be characterized including, for example, for antigenic properties. In an aspect, “antigenic characterization” can refer to the analysis of virus' antigenic properties to help assess how related it is to another virus.

In an aspect, a disclosed postfusion HA protein or fragment thereof can be used in a method of inhibiting egress of influenza virus from infected cells. In an aspect, a disclosed postfusion HA protein or fragment thereof can be used in a method that (i) can minimize and/or decrease the risk of the subject's mortality; (ii) can increase and/or prolong the subject's survival; (iii) can enhance and/or improve the subject's quality of life; (iv) can reduce and/or minimize the likelihood of surgical intervention; (v) can reduce and/or decrease the frequency of other treatment; (vi) can relieve and/or ameliorate one or more subject's symptoms; (vii) can reduce and/or minimize the subject's risk of hospitalization; (viii) can prevent and/or minimize the need for surgical intervention; (ix) can improve and/or restore normal metabolism of one or more of the subject's organ systems, (x) can restore and/or improve one or more aspects of cellular homeostasis and/or cellular functionality, and/or metabolic dysregulation in one or more of the subject's affected systems, or (xi) any combination thereof. In an aspect, a disclosed postfusion HA protein or fragment thereof can be used in a method of treating an influenza infection. In an aspect, a disclosed postfusion HA protein or fragment thereof can be used in a method of eliciting and/or provoking a protective antibody result in a subject in need thereof. In an aspect, a disclosed postfusion HA protein or fragment thereof can be generated following administration of one or more disclosed binding molecules and/or disclosed antibodies.

In an aspect, a disclosed postfusion HA protein or fragment thereof can be prepared in a disclosed pharmaceutical formulation. In an aspect, a disclosed postfusion HA protein or fragment thereof can be encoded by a disclosed nucleic acid sequence or molecule. In an aspect, a disclosed postfusion HA protein or fragment thereof can be used in a disclosed method. In an aspect, a disclosed postfusion HA protein or fragment thereof can be prepared with a disclosed adjuvant or any suitable adjuvant known to the skilled person or any commercially available adjuvant.

In an aspect, a disclosed postfusion HA or a fragment thereof can be formulated for encapsulation in a nanoparticle and/or a lipid nanoparticle.

### Nucleic Acid Molecules

Disclosed herein is a nucleic acid molecule encoding a disclosed binding molecule and/or antibody. Disclosed herein is a nucleic acid molecule encoding a disclosed antibody. Disclosed herein is a nucleic acid molecule comprising IGHV1-2*02/IGHD5-12*01/IGHJ5*02 and IGLV2-23*01/IGLJ3*02. Disclosed herein is nucleic acid molecule comprising IGHV1-2*02/IGHD5-12*01/IGHJ5*02 encoding a heavy chain, and IGLV2-23*01/IGLJ3*02 encoding a light chain. Disclosed herein is a nucleic acid molecule comprising IGHV1-2*02/IGHD5-12*01/IGHJ5*02 encoding a heavy chain, and IGLV2-23*01/IGLJ3*02 encoding a light chain, wherein the heavy chain can comprise about 1% to about 20% somatic mutations, and/or wherein the light chain can comprise about 1% to about 20% somatic mutations. Disclosed herein is a nucleic acid molecule comprising IGHV1-2*02/IGHD5-12*01/IGHJ5*02 encoding a heavy chain and IGLV2-23*01/IGLJ3*02 encoding a light chain, wherein the heavy chain can comprise about 1% to about 20% somatic mutations, and/or wherein the light chain can comprise about 1% to about 20% somatic mutations. Disclosed

Disclosed herein is a nucleic acid molecule comprising a heavy chain encoded by any gene/allele combination identified in Table 24 and a light chain encoded by any gene/allel combination identified in Table 24. Disclosed herein is a nucleic acid molecule comprising a heavy chain encoded by any gene/allele combination identified in Table 24 and a light chain encoded by any gene/allel combination identified in Table 24, wherein the heavy chain can comprise about 1% to about 20% somatic mutations, and/or wherein the light chain can comprise about 1% to about 20% somatic mutations.

TABLE 24
Gene and Allele Calls for Exemplary Disclosed Antibodies
and/or Binding Molecules

| | V-GENE | J-GENE | D-GENE |
|---|---|---|---|
| Sequence ID | and Allele | and Allele | and Allele |
| S1V2-72_IGH | IGHV1-2*02 | IGHJ5*02 | IGHD5-12*01 |
| K06.18_IGH | IGHV1-2*07 | IGHJ4*02 | IGHD6-13*01 |
| 334-100_IGH | IGHV1-2*04 | IGHJ6*02 | IGHD2-2*01 |
| 350-310-D7B_IGH | IGHV1-2*02 | IGHJ5*01 | IGHD6-13*01 |
| 350-315_IGH | IGHV1-2*02 | IGHJ5*01 | IGHD6-13*01 |
| 350-376_IGH | IGHV1-2*02 | IGHJ4*02 | IGHD3-10*03 |
| M94_IGH | IGHV1-2*04 | IGHJ6*02 | IGHD2-21*01 |
| C1_ID26079- | IGHV1-2*02 | IGHJ4*02 | IGHD3-22*01 |
| 156194_IGH | | | |
| FDA053-87001_IGH | IGHV1-2*02 | IGHJ5*02 | IGHD6-19*01 |
| 351-52_IGH | IGHV1-2*02 | IGHJ4*02 | IGHD5-24*01 ORF |
| 350-341_IGH | IGHV1-2*02 | IGHJ1*01 | IGHD6-19*01 |
| S1V2-72_IGL | IGLV2-23*01 | IGLJ3*02 | — |
| K06.18_IGL | IGLV2-23*02 | IGLJ3*02 | — |
| 334-100_IGL | IGLV2-11*01 | IGLJ3*02 | — |
| 350-310-D7B_IGL | IGLV2-23*02 | IGLJ3*02 | — |
| 350-315_IGL | IGLV2-23*02 | IGLJ2*01 | — |
| 350-376_IGL | IGLV2-11*01 | IGLJ3*02 | — |
| M94_IGL | IGLV2-11*01 | IGLJ3*02 | — |
| C1_ID26079- | IGLV2-23*01 | IGLJ3*02 | — |
| 156194_IGL | | | |
| FDA053-87001_IGL | IGLV7-46*01 | IGLJ3*02 | — |
| 351-52_IGK | IGKV3-20*01 | IGKJ1*01 | — |
| 350-341_IGL | IGLV7-46*01 | IGLJ2*01 | — |

In an aspect, a disclosed nucleic acid encoding a heavy chain can comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% somatic mutations. In an aspect, a disclosed heavy chain can comprise at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% somatic mutations. In an aspect, a disclosed nucleic acid encoding a heavy chain can comprise about 10% to about 11% somatic mutations.

In an aspect, a disclosed nucleic acid encoding a light chain can comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% somatic mutations. In an aspect, a disclosed light chain can comprise at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% somatic mutations. In an aspect, a disclosed nucleic acid encoding a light chain can comprise about 3% to about 4% somatic mutations.

Disclosed herein nucleic acid molecule encoding a binding molecule and/or antibody comprising (i) a heavy chain encoded by IGHV1-2*07/IGHD6-13*01/IGHJ4*02, and (ii) a light chain encoded by IGLV2-23*01/IGLJ3*02. Disclosed herein nucleic acid molecule encoding an antibody comprising (i) a heavy chain encoded by IGHV1-2*07/IGHD6-13*01/IGHJ4*02, and (ii) a light chain encoded by IGLV2-23*01/IGLJ3*02. Disclosed herein nucleic acid molecule encoding a binding molecule and/or antibody comprising (i) a heavy chain encoded by IGHV1-2*07/IGHD6-13*01/IGHJ4*02, and (ii) a light chain encoded by IGLV2-23*01/IGLJ3*02, wherein the heavy chain can comprise about 1% to about 20% somatic mutations, and/or wherein the light chain can comprise about 1% to about 20% somatic mutations.

Disclosed herein nucleic acid molecule encoding an antibody comprising (i) a heavy chain encoded by IGHV1-2*07/IGHD6-13*01/IGHJ4*02, and (ii) a light chain encoded by IGLV2-23*01/IGLJ3*02, wherein the heavy chain can comprise about 1% to about 20% somatic mutations, and/or wherein the light chain can comprise about 1% to about 20% somatic mutations.

In an aspect, a disclosed heavy chain can comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% somatic mutations. In an aspect, a disclosed heavy chain can comprise at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% somatic mutations. In an aspect, a disclosed heavy chain can comprise about 5.5% to about 6.5% somatic mutations.

In an aspect, a disclosed light chain can comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% somatic mutations. In an aspect, a disclosed light chain can comprise at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% somatic mutations. In an aspect, a disclosed light chain can comprise about 4% to about 5% somatic mutations.

Disclosed herein is a nucleic acid sequence encoding a heavy chain and comprising the sequence set forth in SEQ ID NO:01. Disclosed herein is a nucleic acid sequence encoding a light chain and comprising the sequence set forth in SEQ ID NO:02. Disclosed herein is a nucleic acid sequence encoding a heavy chain and comprising the sequence set forth in SEQ ID NO:03. Disclosed herein is a nucleic acid sequence encoding a light chain and comprising the sequence set forth in SEQ ID NO:04. Disclosed herein is a nucleic acid sequence encoding a heavy chain and comprising the sequence set forth in SEQ ID NO:219. Disclosed herein is a nucleic acid sequence encoding a light chain and comprising the sequence set forth in SEQ ID NO:220. Disclosed herein is a nucleic acid sequence encoding a heavy chain and comprising the sequence set forth in SEQ ID NO:221. Disclosed herein is a nucleic acid sequence encoding a light chain and comprising the sequence set forth in SEQ ID NO:222. Disclosed herein is a nucleic acid sequence encoding a heavy chain and comprising the sequence set forth in SEQ ID NO:223. Disclosed herein is a nucleic acid sequence encoding a light chain and comprising the sequence set forth in SEQ ID NO:224. Disclosed herein is a nucleic acid sequence encoding a heavy chain and comprising the sequence set forth in SEQ ID NO:225. Disclosed herein is a nucleic acid sequence encoding a light chain and comprising the sequence set forth in SEQ ID NO:226. Disclosed herein is a nucleic acid sequence encoding a heavy chain and comprising the sequence set forth in SEQ ID NO:227. Disclosed herein is a nucleic acid sequence encoding a light chain and comprising the sequence set forth in SEQ ID NO:228. Disclosed herein is a nucleic acid sequence encoding a heavy chain and comprising the sequence set forth in SEQ ID NO:229. Disclosed herein is a nucleic acid sequence encoding a light chain and comprising the sequence set forth in SEQ ID NO:230. Disclosed herein is a nucleic acid sequence encoding a heavy chain and comprising the sequence set forth in SEQ ID NO:231. Disclosed herein is a nucleic acid sequence encoding a light chain and comprising the sequence set forth in SEQ ID NO:232. Disclosed herein is a nucleic acid sequence encoding a heavy chain and comprising the sequence set forth in SEQ ID NO:233. Disclosed herein is a nucleic acid sequence encoding a light chain and comprising the sequence set forth in SEQ ID NO:234. Disclosed herein is a nucleic acid sequence encoding a heavy chain and comprising the sequence set forth in SEQ ID NO:235. Disclosed herein is a nucleic acid sequence encoding a light chain and comprising the sequence set forth in SEQ ID NO:236.

TABLE 15
Exemplary Nucleic Acid Sequences for VL and VH Regions

| Description | Sequence |
|---|---|
| S1V2-72 | caggtgcagctggtgcagtctggggctgaactgaagaagcctggggcgtcagtgaaggtctcatgca |
| (VH) | aggcttctggatacactttcaccggcaactatatccactggatgcgacaggtccctggacaagggct |
| | tgagtggatggggtggatcaacccaagaactggtgacacacatcatgcacagaagtttcagggcagg |
| | gtcgacatgaccagggacacctccatcaacacagcctacctggaactgaccaggctggaatctgacg |
| | acacagccctttattactgtgcgcgatgtgtcttcgcaacttcgcaattcgacccctggggccaggg |
| | aaccctggtcaccgtctcctca (SEQ ID NO: 01) |
| S1V2-72 | cagtctgccctgactcagcctgcctccgtgtctgggtctcctggacagtcgatcaccatctcctgca |
| (VL) | ctggaaccaacagtgatattgggagtcataaccttgtctcctggtaccaacaacacccaggcaaagc |
| | ccccaaagtcatgatttatgacgacagtaagcggccctcaggagtttctaatcgcttctctggctcc |
| | aagtctggcagcacggcctccctgacaatctctgggctccagtctgaggacgaggctgattattatt |
| | gctgctcatatgcaggtagtagcaactgggtgttcggcggagggaccaagctgacactccta (SEQ |
| | ID NO: 02) |
| K06.18 (VH) | caggtgcagctggtgcagtctggggctgaggtgaagaagcctggggcctcagtgagggtctcctgca |
| | gggcctctggatacagcttcaccgacaactatattcactgggtgcggcaggcccctggacaagggct |
| | tgagtggatgggatggatcaaccctgacagtggtgccacaagctacgaacataagtttcagggcagg |
| | gtcaccatgaccagggacacgtccatcagcacagccttcatggaactgggcagtctgagatctgacg |
| | acacggccgtttattactgtgcgagggtgcggagggggacagcaggacttgactactggggccaggg |
| | aaccctggtcaccgtctcctca (SEQ ID NO: 03) |
| K06.18 (VL) | cagtctgtgctgactcagcctgcctccgtgtctgggtctcttggacagtcgatcaccatctcctgca |
| | ctggaaccagcagtgatattgggaattataaccttgtctcctggtaccaacagcacccaggccaagc |
| | ccccaaactcatcatttatgaggtcaataggcggccctcaggggtttctgatcgcttctctgactcc |
| | aagtctggcaacacggcctccctgacaatctctgggctccaggctgaggacgagggtgattattact |
| | gcttttcatatgcaggtggtagtacttgggtgttcggcggagggaccaaggtgaccgtcctc (SEQ |
| | ID NO: 04) |
| 334-100 (VH) | caggtgcagctggtgcagtctggggctgaggtgaagaagcctggggcctcagtgaaggtctcctgca |
| | aggcttctggatacaccttcaccggctactatatgcactgggtgcgacaggcccctggacaagggct |
| | tgagtggatgggatggatcaaccctaatactggtgacacaaagtatgcacagaagtttcaggactgg |
| | gtcaccatgaccagggacacgtccatcagtacagcctacatggagctgaacaggctgagatctggcg |
| | acacggccgtctattattgtgcgaggataattagcaggtccagcggtctggacgtctggggccaagg |
| | gaccacggtcaccgtctcctcag (SEQ ID NO: 219) |
| 334-100 (VL) | cagtctgccctgactcagcctcgctcagtgtccgggtctcctggacagtcagtcaccatctcctgcg |
| | ctggaaccagcagtgatgttggtggttataactatgtctcctggtaccaacaccaccccggcaaagc |
| | ccccaaactcatgatttatgatgtctttaagcggccctcaggggtccctgatcgcttctctggctcc |
| | aagtctggcaacgcggcctccctgatcatctctgggctccaggctgaggatgaggctgattattact |
| | gctactcatattcaggcagctacactttttgggtgttcggcggagggaccaagctgaccgtcctag |
| | (SEQ ID NO: 220) |
| 350-310-D7B | caggtgcagctggtgcagtctggggctgaggtgaagaagcctggggcctcagtgaaggtgtcctgca |
| (VH) | gggcttctggatacaccttcaccgccaattatatgcactgggtgcgacaggcccctggacaagggct |
| | tgagtggatgggatggatcaaccctaaaagtggtgacacaagctctacagagaagtttcagggcagg |
| | gtcaccatgaccagggacacgtccatcaccacagcctacatggaactgagtaggttgagatctgacg |
| | acacggccgtgtattactgtgcgagagtcccctatggcagcagccccgacttttggggccagggaac |
| | cctggtcaccgtctcctcag (SEQ ID NO: 221) |
| 350-310-D7B | cagtctgccctgactcagcctgcctccgtgtctgggtctcctggacagtcgatcaccatctcctgca |
| (VL) | ctggaaccagcagtgatgttgggaggtataagtttgtctcctggtaccaacagcacccaggcaaagc |
| | ccccaaagtcatgatttttgaggtcactaagcggccctcaggggtttctgatcgcttctctggctcc |
| | aagtctgccaacacggcctccctgacaatctctgggctccaggctgaggacgaggctgattattact |
| | gcttgtcatatgccggcagtagcaattgggtcttcggcggagggaccaggctgaccgtcctag |
| | (SEQ ID NO: 222) |
| 350-315 (VH) | caggtgcagctggtgcagtctggggctgaggtgaagaagcctggggcctcagtgaaggtctcctgca |
| | gggcttctggatacaccttcaccgccaattatatgcactgggtgcgacaggcccctggacaagggct |
| | tgagtggatgggatggatcaaccctaacagtggtgacacaagctctgcacagaagtttcagggcagg |
| | gtcaccatgaccagggacacgtccatcagcacagcctacatggagctgagtagactgagatctgacg |
| | acacggccgtgtattactgtgcgagagtcccctatggcagcagccccgacttttggggccagggaac |
| | cctggtcaccgtctcctcag (SEQ ID NO: 223) |
| 350-315 (VL) | cagtctgccctgactcagcctgcctccgtgtctgggtctcctggacagtcgatcaccatctcctgca |
| | ctggaaccagcagtgatgttgggagttataagtttgtctcctggtaccaacagcacccaggcaaagc |
| | ccccaaagtcatgatttttgaggtcactaagcggccctcaggggtttctgatcgcttctctggctcc |
| | aagtctgccaacacggcctccctgacaatctctgggctccaggctgaggacgaggctgattattact |
| | gcttctcatatgcaggtagtagcacttgggtattcggcggagggaccaggctgaccgtcctag |
| | (SEQ ID NO: 224) |
| 350-376_IGH | caggtgcagctggtgcaatctggggctgaggtgaagaagcctggggcctcaatgaaggtctcctgca |
| | aggcttctggatacaccttcacccgctactatgtgcactgggtgcgacaggcccctggacaagggct |
| | tgagtggatgggatggatggaccctaatagtggtggcacaaactctgcacagaactttcagggtagg |
| | gtaaccatgaccagggacacgtccatcaacacggcctacatggaggtgagcgggctgagatctgacg |
| | acacgggcgtttattactgtgcgaggattacagtgggttatttcccatactttgacttctggggcca |
| | gggaaccctggtcaccgtctcctcag (SEQ ID NO: 225) |
| 350-376 (VH) | cagtctgccctgattcagcctcgctcagtgtccgggtctcctggacagtcaatcaccatctcctgca |
| | gtggaaccagcagtgatgttggtaattataactatatttcctggtaccaacagcacccaggcaaagc |
| | ccccaaagtcatgatttatgatgtcaataagcggccctcaggggtccctgatcgcttctctggctcc |
| | aagtctggcaacacggcctccctgaccatctctgggctccaggctgaagatgaggctgattactact |
| | gctgctcatatgcaggcagctcctcttgggtgttcggcggagggaccaaactgaccgtcctag |
| | (SEQ ID NO: 226) |
| M94 (VH) | caggtgcagctggtggagtctggggctgaggtgaagaggcctggggcgtcagtgaaggtcgcctgca |
| | aggcttctgaatacagatttgccgcctcctatatacactgggtgcgacaggccccaggacaagggct |
| | tgagtggatgggctggataaacctcaacagtggtgacacaaagtcttcacagaagtttcagggctgg |
| | gtcaccctgacccgcgacacgtccatcaacacagtctacatggagatgactaatctgaaatttgacg |
| | acacggccatttattactgtgcgaagatcatcctgggggggggcggtctagacgtctggggccaagg |
| | gaccacggtcaccgtctcctcagcctccaccaagggcccat (SEQ ID NO: 227) |
| M94 (VL) | gctagcccagtctgttctgactcagcctcgctcagtgtccgggtctcttggacagtcagtcaccatc |
| | tcctgcagtggaaccagcactgatgttggtagttatgagtttgtctcctggtaccaacaccacccag |
| | gcaaagcccccaaactcattatttatgatgtctctaagcggccctcaggagtccctgatcgcttctc |
| | tggctcaaagtctggcaacacggcctccctggccgtctctgggctccagtctgacgatgaggctgac |
| | tattattgctcgtcatatgctgacacctccaatacttgggtgttcggcggagggacgaagctgaccg |
| | tcctaggtcagcccaaggctgccccctcggtcactctgttcccaccctcgcggccgc (SEQ ID |
| | NO: 228) |
| C1_ID26079- | caggtgcagctggtgcagtctggggctgaggtgaagaagcctggggcctcagtgaaggtctcctgca |
| 156194 (VH) | aggcttctggatacaccttcaccggcaactatatgcactgggtgcgacaggcccctggacaagggct |
| | tgagtggatgggctggatcaaccctgacagtggtggcacaaactatgcacagaggtttcagggcagg |
| | gtcaccatgaccagggacacgtccatcagcacagcctacatggagctgagcaggctgacatctgacg |
| | acacggccatgtattactgtgcgagagtgatgctgaggagtagtggatttgactcctggggccaggg |
| | aaccctggtcaccgtctcctcagcctccaccaagggcccatcccagggcccat (SEQ ID NO: |
| | 229) |
| C1_ID26079- | gctagcccagtctgccctgattcagcctgcctccgtgtctgggtctcctggacagtcgatcaccatc |
| 156194 (VL) | tcctgcactggaaccagcagtgatgtcgggacttataaccttgtctcctggtatcaacagcacccag |
| | gcaaagtccccaaactcatgatttatgaggccagtgagcggccctcaggggtttctaatcgcttctc |
| | tggctccaagtctggcaacacggcctccttgacaatctctgggctccaggctgaggacgaggctgac |
| | tattactgctgctcatatgcagggaatagcgcttgggtgttcggcggagggaccaagctgaccgtcc |
| | taggtcagcccaaggctgccccctcggtcactctgttcccaccctcgcggcccgcac (SEQ ID |
| | NO: 230) |
| FDA053- | caggtgcagctggtgcagtctggggctgagatgaagaaccctggggcctcagtgaaggtctcctgca |
| 87001 (VH) | aggcttctggatacaccttcagcggctactatatacactgggtgcgacaggcccctggacaagggct |
| | tgagtggatggcatggatcgaccctaatagtggtgccacaaactatccagagaagtttcagggcagg |
| | gtcaccatgaccacggacacgtccatcaccacaacctacatggagctgaccagcctgagttctgacg |
| | acacggccgtgtattactgtgcgagagttcgatatggcagtggcttccactggggccagggaaccct |
| | ggtcaccgtctcctcag (SEQ ID NO: 231) |
| FDA053- | caggctgtggtgactcaggagccctcactgactgtgtccccaggagggacagtcactctcacctgtg |
| 87001 (VL) | gctccagcactggagctgtcaccagtggtcattatccctactggttccagcagaaggctggccaagc |
| | ccccaggacactgatttatgatacaggcaacaaacactcctcgacacctgcccggttctcaggctcc |
| | ctccttgggggcaaagctgccctgaccctttcgggtgcgcagcctgaggatgaggctgaatattact |
| | gcttgctctcccatagtggtccgtgggtgttcggcggagggaccaagctgaccgtcctag (SEQ |
| | ID NO: 232) |
| 351-52 (VH) | caggtgcagctggcgcagtctggggctgaggtgaggaagcctggggcctcagtgaaagtctcctgca |
| | aggcttctggatacaccttcaccgactactatatacactgggtgcgacaggcccctggacaagggct |
| | tgagtggatgggatggatcaaccctgacagtggcaggacaaactatgcacagagatttcagggcagg |
| | gtcagtatgaccagcgacacgtccatcagcacagcctacatggacctgagcagactgagatctgacg |
| | acacggccgtgtatttctgtgcgagagagggggctcataattaccaacactttgaccactggggcca |
| | gggaaccctggtcaccgtgtccgca (SEQ ID NO: 233) |
| 351-52 (VK) | gaaattgtgttgacgcagtctccagacaccctgtctttgtctccaggggaaagcgccaccctcttct |
| | gcagggccagtcagagtgttaggagcagttacttagcctggtaccagcagacacctggccaggctcc |
| | caggctcctcatctatggtgcatccagcagggccactggcatcccagacaggttcagtggcagtggg |
| | tctgggacagacttcactctcaccatcagcagactggagcctgaagattttgcagtgtattactgtc |
| | agcagtttggtaggtcgtcgtggacgttcggccaagggaccaaggtggaaatcaaa (SEQ ID |
| | NO: 234) |
| 350-341 (VH) | caggtgcaactggtgcagtctggggctgaggtgaagaagcctggggcctcagtgagggtctcctgca |
| | aggcttctggattcaccttcaccacacactacatacactgggtgcgacaggcccctggacaagggct |
| | tgagtggatgggatggatcgaccctagcagtggtggcacaaagtttgcacagcactttcagggcagg |
| | gtcaccatgaccacggacgcgtccatcaacacagccttcatggacctgaacaggctgagatctgacg |
| | acacggccatgtattactgtgcgagagtgcgctttggaagtggctggtactggggccagggaaccct |
| | ggtcaccgtctcctca (SEQ ID NO: 235) |
| 350-341 (VL) | caggctgtggtgactcaggagccctcactgactgtgtccccaggagggacggtcactctcacctgtg |
| | gttccaccactggacctgtcaccagtggtcattatccctactggttccaacagaagcctggccaagc |
| | ccccaggacagtgatttatgatacaaccaacaaacactcctggacacctgcctggttctcaggctcc |
| | ctccttgggggcaaagctgccctgaccctttcgggtgcacagcctgaggatgaggctgagtattact |
| | gcttgctcgcctatagtggtacttgggtattcggcggagggaccaagctgaccgtcctt (SEQ ID |
| | NO: 236) |

Disclosed herein is a nucleic acid sequence encoding a heavy chain and comprising the sequence set forth in SEQ ID NO:01, wherein the heavy chain can comprise about 1% to about 20% somatic mutations. Disclosed herein is a nucleic acid sequence encoding a light chain and comprising the sequence set forth in SEQ ID NO:02, wherein the light chain can comprise about 1% to about 20% somatic mutations. Disclosed herein is a nucleic acid sequence encoding a heavy chain and comprising the sequence set forth in SEQ ID NO:03, wherein the heavy chain can comprise about 1% to about 20% somatic mutations. Disclosed herein is a nucleic acid sequence encoding a light chain and comprising the sequence set forth in SEQ ID NO:04, wherein the light chain can comprise about 1% to about 20% somatic mutations. Disclosed herein is a nucleic acid sequence encoding a heavy chain and comprising the sequence set forth in SEQ ID NO:219, wherein the heavy chain can comprise about 1% to about 20% somatic mutations. Disclosed herein is a nucleic acid sequence encoding a light chain and comprising the sequence set forth in SEQ ID NO:220, wherein the light chain can comprise about 1% to about 20% somatic mutations. Disclosed herein is a nucleic acid sequence encoding a heavy chain and comprising the sequence set forth in SEQ ID NO:221, wherein the heavy chain can comprise about 1% to about 20% somatic mutations. Disclosed herein is a nucleic acid sequence encoding a light chain and comprising the sequence set forth in SEQ ID NO:222, wherein the light chain can comprise about 1% to about 20% somatic mutations. Disclosed herein is a nucleic acid sequence encoding a heavy chain and comprising the sequence set forth in SEQ ID NO:223, wherein the heavy chain can comprise about 1% to about 20% somatic mutations. Disclosed herein is a nucleic acid sequence encoding a light chain and comprising the sequence set forth in SEQ ID NO:224, wherein the light chain can comprise about 1% to about 20% somatic mutations. Disclosed herein is a nucleic acid sequence encoding a heavy chain and comprising the sequence set forth in SEQ ID NO:225, wherein the heavy chain can comprise about 1% to about 20% somatic mutations. Disclosed herein is a nucleic acid sequence encoding a light chain and comprising the sequence set forth in SEQ ID NO:226, wherein the light chain can comprise about 1% to about 20% somatic mutations. Disclosed herein is a nucleic acid sequence encoding a heavy chain and comprising the sequence set forth in SEQ ID NO:227, wherein the heavy chain can comprise about 1% to about 20% somatic mutations. Disclosed herein is a nucleic acid sequence encoding a light chain and comprising the sequence set forth in SEQ ID NO:228, wherein the light chain can comprise about 1% to about 20% somatic mutations. Disclosed herein is a nucleic acid sequence encoding a heavy chain and comprising the sequence set forth in SEQ ID NO:229, wherein the heavy chain can comprise about 1% to about 20% somatic mutations. Disclosed herein is a nucleic acid sequence encoding a light chain and comprising the sequence set forth in SEQ ID NO:230, wherein the light chain can comprise about 1% to about 20% somatic mutations. Disclosed herein is a nucleic acid sequence encoding a heavy chain and comprising the sequence set forth in SEQ ID NO:231, wherein the heavy chain can comprise about 1% to about 20% somatic mutations. Disclosed herein is a nucleic acid sequence encoding a light chain and comprising the sequence set forth in SEQ ID NO:232, wherein the light chain can comprise about 1% to about 20% somatic mutations. Disclosed herein is a nucleic acid sequence encoding a heavy chain and comprising the sequence set forth in SEQ ID NO:233, wherein the heavy chain can comprise about 1% to about 20% somatic mutations. Disclosed herein is a nucleic acid sequence encoding a light chain and comprising the sequence set forth in SEQ ID NO:234, wherein the light chain can comprise about 1% to about 20% somatic mutations. Disclosed herein is a nucleic acid sequence encoding a heavy chain and comprising the sequence set forth in SEQ ID NO:235, wherein the heavy chain can comprise about 1% to about 20% somatic mutations. Disclosed herein is a nucleic acid sequence encoding a light chain and comprising the sequence set forth in SEQ ID NO:236, wherein the light chain can comprise about 1% to about 20% somatic mutations.

Disclosed herein is a nucleic acid sequence encoding a light chain set forth in Table 2. Disclosed herein is a nucleic acid sequence encoding a heavy chain set forth in Table 1.

Disclosed herein is a nucleic acid sequence encoding one or more disclosed CDRs.

Disclosed herein is a nucleic acid sequence encoding one or more disclosed CDRs including those, for example, in Table 1, Table 2, Table 3, Table 4, Table 5, Table 6, Table 7, Table 8, Table 9, Table 10, Table 11, Table 12, Table 13, and Table 14.

Disclosed herein is a nucleic acid sequence encoding a light chain set forth in Table 2 and a heavy chain set forth in Table 1. Disclosed herein is a nucleic acid sequence encoding a light chain set forth in Table 2 and a heavy chain set forth in Table 1, wherein the light chain can comprise about 1% to about 20% somatic mutations, and/or wherein the heavy chain can comprise about 1% to about 20% somatic mutations. Disclosed herein is a nucleic acid sequence as set forth in Table 15.

Disclosed herein is a nucleic acid sequence encoding any HA or fragment thereof set forth in Table 16. Disclosed herein is a nucleic acid sequence encoding the HA sequence set forth in any one of SEQ ID NO:29-SEQ ID NO:58. Disclosed herein is a nucleic acid sequence encoding the HA sequence set forth in any one of SEQ ID NO:192-SEQ ID NO:218.

Disclosed herein is a nucleic acid sequence encoding a disclosed postfusion HA protein or a fragment thereof. Disclosed herein is a nucleic acid sequence encoding a disclosed postfusion

HA protein or fragment thereof having a β-hairpin comprising the sequence set forth in SEQ ID NO:191. Disclosed herein is a nucleic acid sequence encoding a disclosed postfusion HA protein or fragment thereof having the sequence set forth in any one of SEQ ID NO:219-SEQ ID NO:236. Disclosed herein is a nucleic acid sequence encoding a disclosed postfusion HA protein or fragment thereof having the sequence set forth in any one of SEQ ID NO:29-SEQ ID NO:58. Disclosed herein is a nucleic acid sequence encoding a disclosed postfusion HA protein or fragment thereof having a sequence having at least about 80%, at least about 85%, at least about 90%, at least about 95%, or more than at least 95% identity to the sequence set forth in any one of SEQ ID NO:219-SEQ ID NO:236. Disclosed herein is a nucleic acid sequence encoding a disclosed postfusion HA protein or fragment thereof having a sequence having at least about 80%, at least about 85%, at least about 90%, at least about 95%, or more than at least 95% identity to the sequence set forth in any one of SEQ ID NO:29-SEQ ID NO:58. Disclosed herein is a nucleic acid sequence encoding a disclosed postfusion HA protein or fragment thereof having a sequence having at least about 80%, at least about 85%, at least about 90%, at least about 95%, or more than at least 95% identity to the sequence set forth in Table 16. Disclosed herein is a nucleic acid sequence encoding a disclosed postfusion HA protein or fragment thereof having a sequence set forth in Table 16.

In an aspect, a disclosed nucleic acid molecule can encode a binding molecule and/or a disclosed antibody that can confer Fc-dependent protection. In an aspect, a disclosed nucleic acid molecule can encode a binding molecule and/or a disclosed antibody that can activate and/or elicit complement-dependent cytotoxicity (CDC), antibody-dependent cellular phagocytosis (ADCP), and antibody-dependent cellular cytotoxicity (ADCC), or any combination thereof. In an aspect, a disclosed nucleic acid molecule can encode a binding molecule and/or a disclosed antibody that does not neutralize infectivity of the influenza virus. In an aspect, a disclosed nucleic acid molecule can encode a binding molecule and/or a disclosed antibody that can confer protection against lethal infection of the influenza virus. In an aspect, a disclosed nucleic acid molecule can encode a binding molecule and/or a disclosed antibody that can confer protection against mortality and/or morbidity associated with the influenza virus.

In an aspect, a disclosed method can further comprise characterizing one or more disclosed binding molecules and/or disclosed antibodies or disclosed pharmaceutical formulations. In an aspect, characterizing one or more disclosed binding molecules can comprise analyzing and/or determining and/or quantifying the efficacy of one or more disclosed binding molecules and/or disclosed antibodies or disclosed pharmaceutical formulations. In an aspect, efficacy can comprise the ability of one or more disclosed binding molecules and/or disclosed antibodies or disclosed pharmaceutical formulations to reduce and/or decrease and/or minimize symptoms of an influenza infection, the risk of the subject's morbidity and/or mortality, the duration that the subject remains symptomatic, and any combination thereof. In an aspect, characterizing one or more disclosed binding molecules can comprise analyzing and/or determining and/or quantifying the efficacy of one or more disclosed binding molecules and/or disclosed antibodies or disclosed pharmaceutical formulations.

In an aspect, a disclosed nucleic acid molecule and/or a disclosed nucleic acid sequence can be formulated for encapsulation in a nanoparticle and/or a lipid nanoparticle.

### Universal Influenza Antigen

Disclosed herein is a universal influenza vaccine antigen. Disclosed herein is a universal influenza vaccine antigen comprising a β-hairpin epitope of HA2. Disclosed herein is a universal influenza vaccine antigen comprising a β-hairpin epitope of prefusion HA2. Disclosed herein is a universal influenza vaccine antigen comprising a β-hairpin epitope of postfusion HA protein2. Disclosed herein is a composition comprising a universal influenza vaccine antigen. Disclosed herein is a composition comprising a universal influenza vaccine antigen and a vaccine adjuvant.

In an aspect, a disclosed universal influenza vaccine antigen can comprise a sequence corresponding to a portion of a β-hairpin of the prefusion HA2. In an aspect, a disclosed universal influenza vaccine antigen can comprise a sequence corresponding to a portion of a β-hairpin of the postfusion HA protein2. In an aspect, a disclosed prefusion HA2 can comprise a β-hairpin pitope. In an aspect, a disclosed postfusion HA protein2 can comprise a β-hairpin epitope.

In an aspect, a disclosed β-hairpin epitope can comprise a sequence having the sequence set forth in SEQ ID NO:191. In an aspect, a disclosed universal influenza vaccine antigen can comprise the sequence set forth in SEQ ID NO:191. In an aspect, a disclosed β-hairpin epitope can comprise a consensus sequence having the sequence set forth in SEQ ID NO:191. In an aspect, a disclosed universal influenza vaccine antigen can comprise a conserved Gly-Asn-Gly sequence. In an aspect, a β-hairpin of HA2 can comprise residues 75-155.

In an aspect, a disclosed universal influenza vaccine antigen can be used to produce a binding molecule and/or a disclosed antibody that binds to a disclosed β-hairpin in a prefusion HA. In an aspect, a disclosed universal influenza vaccine antigen can be used to produce a binding molecule and/or a disclosed antibody that binds to a disclosed β-hairpin in a postfusion HA protein.

In an aspect, a disclosed universal influenza vaccine antigen can be used to produce a disclosed binding molecule and/or a disclosed antibody. In an aspect, a disclosed universal influenza vaccine antigen can be used to produce a disclosed binding molecule and/or a disclosed antibody comprising a heavy chain variable region set forth in Table 1. In an aspect, a disclosed universal influenza vaccine antigen can be used to produce a disclosed binding molecule and/or a disclosed antibody comprising a light chain variable region set forth in Table 2.

In an aspect, a disclosed universal influenza vaccine antigen can be used to produce a disclosed S1V2-72 binding molecule and/or a disclosed antibody, a disclosed K06.18 binding molecule and/or a disclosed antibody, a disclosed 334-100 binding molecule and/or a disclosed antibody, a disclosed 350-310-D7B binding molecule and/or a disclosed antibody, a disclosed 350-315 binding molecule and/or a disclosed antibody, a disclosed 350-376 binding molecule and/or a disclosed antibody, a disclosed 351-52 antibody, a disclosed 350-341 binding molecule and/or a disclosed antibody, a disclosed S5V6-P7F12 binding molecule and/or a disclosed antibody, a disclosed M94 binding molecule and/or a disclosed antibody, a disclosed C1_ID26079-156194 binding molecule and/or a disclosed antibody, a disclosed FDA053-87001 binding molecule and/or a disclosed antibody, or any combination thereof. In an aspect, a disclosed universal influenza vaccine antigen can produce a recombinant S1V2-72 binding molecule and/or a disclosed antibody, a recombinant K06.18 binding molecule and/or a disclosed antibody, a recombinant 334-100 binding molecule and/or a disclosed antibody, a recombinant 350-310-D7B binding molecule and/or a disclosed antibody, a recombinant 350-315 binding molecule and/or a disclosed antibody, a recombinant 350-376 binding molecule and/or a disclosed antibody, a recombinant 351-52 binding molecule and/or a disclosed antibody, a recombinant 350-341 binding molecule and/or a disclosed antibody, a recombinant S5V6-P7F12 binding molecule and/or a disclosed antibody, a recombinant M94 binding molecule and/or a disclosed antibody, a recombinant C1_ID26079-156194 binding molecule and/or a disclosed antibody, a recombinant FDA053-87001 binding molecule and/or a disclosed antibody, or any combination thereof.

In an aspect, a disclosed universal influenza vaccine antigen can be used to produce a disclosed binding molecule and/or a disclosed antibody capable of specifically binding to postfusion hemagglutinin (HA) of influenza. In an aspect, a disclosed universal influenza vaccine antigen can be used to produce a disclosed binding molecule and/or a disclosed antibody capable of specifically binding to postfusion hemagglutinin (HA) of influenza A virus strains. In an aspect, a disclosed universal influenza vaccine antigen can be used to produce a disclosed binding molecule and/or a disclosed antibody capable of specifically binding to postfusion hemagglutinin (HA) of influenza B virus strains. In an aspect, a disclosed universal influenza vaccine antigen can be used to produce a disclosed binding molecule and/or a disclosed antibody capable of specifically binding to postfusion hemagglutinin (HA) of influenza A virus strains and influenza B virus strains.

In an aspect, a disclosed universal influenza vaccine antigen can be used to produce a binding molecule and/or a disclosed antibody that can bind to the stem region of the HA protein.

TABLE 16
Exemplary EHA2 Sequences and EHA2 Core Sequences
(Conserved Gly-Asn-Gly Underlined)

| Description | Sequence (SEQ ID NO) |
|---|---|
| A/USSR/90/1977 (H1N1) | nrmenlnkkvddgfldiwtynaellvllenertldfhdsnvknlyekvksq |
| | lknnakeigngsfefyhkcnnecmesvkng (SEQ ID NO: 29) |
| A/Solomon Islands/3/2006 | nrmenlnkkvddgfidiwtynaellvllenertldfhdsnvknlyekvksq |
| (H1N1) | lknnakeigngsfefyhkcndecmesvkng (SEQ ID NO: 30) |
| A/California/07/2009 (H1N1) | nrienlnkkvddgfldiwtynaellvllenertldyhdsnvknlyekvrsq |
| | lknnakeigngsfefyhkcdntcmesvkng (SEQ ID NO: 31) |
| A/Michigan/45/2015 (H1N1) | nrienlnkkvddgfldiwtynaellvllenertldyhdsnvknlyekvrnq |
| | lknnakeigngsfefyhkcdntcmesvkng (SEQ ID NO: 32) |
| A/Wisconsin/67/2022 (H1N1) | nrienlnkkvddgfldvwtynaellvllenertldyhdsnvknlyekvrhq |
| | lknnakeigngsfefyhkcdntcmesvkng (SEQ ID NO: 33) |
| A/Japan/305/1957 (H2N2) | nrlenlnkkmedgfldvwtynaellvlmenertldfhdsnvknlydkvrmq |
| | lrdnvkelgngsfefyhkcddecmnsvkng (SEQ ID NO: 34) |
| A/Aichi/2/1968 (H3N2) | nriqdlekyvedtkidlwsynaellvalenqhtidltdsemnklfektrrq |
| | lrenaedmgngsfkiyhkcdnaciesirng (SEQ ID NO: 35) |
| A/Texas/50/2012 (H3N2) | nriqdlekyvedtkidlwsynaellvalenqhtidltdsemnklfektkkq |
| | lrenaedmgngsfkiyhkcdnacigsirng (SEQ ID NO: 36) |
| A/Massachusetts/18/2022 | nrvqdlekyvedtkidlwsynaellvalenqhtidltdsemnklfektkkq |
| (H3N2) | Irenaedmgngsfkiyhkcdnacigsirne (SEQ ID NO: 37) |
| A/mallard/New Brunswick/ | nriqdlekyvedtkidlwsynaellvalenqhtidvtdsemnklfervrrq |
| 00389/2010 (H4N6) | lrenaedkgngsfeifhqcdnnciesirng (SEQ ID NO: 38) |
| A/Texas/37/2024 (H5N1) | nrienlnkkmedgfldvwtynaellvlmenertldfhdsnvknlydkvrlq |
| | lrdnakelgngcfefyhkcdnecmesvrng (SEQ ID NO: 39) |
| A/chicken/Taiwan/2759/2012 | nrignlnkrmedgfldvwtynaellvllenertldlhdanvknlyekvksq |
| (H6N1) | Irdnandlgngsfefwhkcdnecmesvkng (SEQ ID NO: 40) |
| A/chicken/Guangdong/J1/2017 | nqignvinwtrdsitevwsynaellvamenqhtidladsemdklyervkrq |
| (H7N9) | lrenaeedgtgsfeifhkcdddcmasirnn (SEQ ID NO: 41) |
| A/northern shoveler/ | nrinmindkiddqieglwaynaellvllenqktldehdsnvknlfdevkrr |
| California/HKWF1204/2007 | Istnamdagngsfdilhkcnnecmetikng (SEQ ID NO: 42) |
| (H8N4) | |
| A/duck/Beijing/MG0617/2005 | nrlnminnkiddqiqdiwaynaellvllenqktldehdanvnnlynkvkra |
| (H9N2) | lgsnavedgkgsfelyhkcddqcmetirng (SEQ ID NO: 43) |
| A/chicken/Jiangxi/18933/2013 | nqignvinwtkdsitdiwtyqaellvamenqhtidmadsemlnlyervrkq |
| (H10N8) | lrqnaeedgkgsfeiyhacddscmesirnn (SEQ ID NO: 44) |
| A/common teal/Netherlands | nrinqlskhvddsvvdiwsynaqllvllenektldlhdsnvrnlhekvrrm |
| /1/2015 (H11N1) | lkdnakdegngsftfyhkcdnecierving (SEQ ID NO: 45) |
| A/Northern Shoveler/Nevada | nrinminskvddqitdiwaynaellvllenqktldehdanvrnlhdrvrrv |
| /D1516557/2015 (H12N2) | lrenaidtgdgsfeilhkcdnncmdtirng (SEQ ID NO: 46) |
| A/Armenian gull/Republic of | nrinmlanriddavtdvwsynakllvllendktldmhdanvrnlhdqvrrv |
| Georgia/2/2012 (H13N2) | lktnaidegngsfellhkcndscmetirng (SEQ ID NO: 47) |
| A/blue-winged teal/TX/AI13- | nriqdlekyvedtkidlwsynaellvalenqhtidvtdsemnklfervrrq |
| 1028/2013 (H14N5) | lrenaedqgngsfeifhqcdnnciesirng (SEQ ID NO: 48) |
| A/shearwater/Australia/2576/ | nqignvinwtrdslteiwsynaellvamenqhtidladsemnklyervrrq |
| 1979 (H15N6) | lrenaeedgtgsfeifhrcddqcmesirnn (SEQ ID NO: 49) |
| A/shorebird/Delaware/172/ | nrinmladrvddavtdvwsynakllvllengrtldlhdanvrnlhdqikrt |
| 2006 (H16N3) | lknnaidegdgsfnllhkcndscmeairng (SEQ ID NO: 50) |
| A/little yellow-shouldered | nriqhlsdrvddalldiwsyntellvllenertldfhdanvknlfekvkaq |
| bat/Guatemala/060/2010 | lkdnaidegngsflllhkcnnscmddikng (SEQ ID NO: 51) |
| (H17N10) | |
| A/flat-faced bat/Peru/033/ | nrikhlsdrvddgfldvwsynaellvllenertldfhdanvnnlyqkvkvq |
| 2010 (H18N11) | lkdnaidmgngsfkilhkcnntcmddikng (SEQ ID NO: 52) |
| B/Malaysia/2506/2004 | neileldekvddlradtissqielavllsnegiinsedehllalerklkkm |
| | lgpsaveigngsfetkhkcnqtcldriaag (SEQ ID NO: 53) |
| B/Phuket/3073/2013 | neileldekvddlradtissqielavllsnegiinsedehllalerklkkm |
| | lgpsavdigngsfetkhkcnqtcldriaag (SEQ ID NO: 54) |
| B/Wisconsin/22/2024 | neileldekvddlradtissqielavllsnegiinsedehllalerklkkm |
| | lgpsaveigngsfetkhkcnqtcldriaag (SEQ ID NO: 55) |
| A/USSR/90/1977 (H1N1) | gyhhqneqgsgyaadqkstqnaingitnkvnsviekmntqftavgkefnkl |
| | ekrmenlnkkvddgfldiwtynaellvllenertldfhdsnvknlyekvks |
| | qlknnakeigngsfefyhkcnnecmesvkngtydypkyseeskInrekidg |
| | vklesm (SEQ ID NO: 192) |
| A/Solomon Islands/3/2006 | gyhhqneqgsgyaadqkstqnaingitnkvnsviekmntqftavgkefnkl |
| (H1N1) | errmenlnkkvddgfidiwtynaellvllenertldfhdsnvknlyekvks |
| | qlknnakeigngsfefyhkcndecmesvkngtydypkyseeskInrekidg |
| | vklesm (SEQ ID NO: 193) |
| A/California/07/2009 (H1N1) | gyhhqneqgsgyaadlkstqnaideitnkvnsviekmntqftavgkefnhl |
| | ekrienlnkkvddgfldiwtynaellvllenertldyhdsnvknlyekvrs |
| | qlknnakeigngsfefyhkcdntcmesvkngtydypkyseeaklnreeidg |
| | vklestr (SEQ ID NO: 194) |
| A/Michigan/45/2015 (H1N1) | gyhhqneqgsgyaadlkstqnaidkitnkvnsviekmntqftavgkefnhl |
| | ekrienlnkkvddgfldiwtynaellvllenertldyhdsnvknlyekvrn |
| | qlknnakeigngsfefyhkcdntcmesvkngtydypkyseeaklnrekidg |
| | vklestr (SEQ ID NO: 195) |
| A/Wisconsin/67/2022 (H1N1) | gyhhqndqgsgyaadlkstqnaidkitnkvnsviekmntqftavgkefnhl |
| | ekrienlnkkvddgfldvwtynaellvllenertldyhdsnvknlyekvrh |
| | qlknnakeigngsfefyhkcdntcmesvkngtydypkyseeakInrekidg |
| | vkldstriyq (SEQ ID NO: 196) |
| A/Japan/305/1957 (H2N2) | gyhhsndqgsgyaadkestqkafdgitnkvnsviekmntqfeavgkefsnl |
| | errlenlnkkmedgfldvwtynaellvlmenertldfhdsnvknlydkvrm |
| | qlrdnvkelgngsfefyhkcddecmnsvkngtydypkyeeesklnrneikg |
| | vklssmgvyq (SEQ ID NO: 197) |
| A/Aichi/2/1968 (H3N2) | gfrhqnsegtgqaadlkstqaaidqingklnrviektnekfhqiekefsev |
| | egriqdlekyvedtkidlwsynaellvalenqhtidltdsemnklfektrr |
| | qlrenaedmgngsfkiyhkcdnaciesirngtydhdvyrdealnnrfqikg |
| | velksgykd (SEQ ID NO: 198) |
| A/Texas/50/2012 (H3N2) | gfrhqnsegrgqaadlkstqaaidqingklnrligetnekfhqiekefsev |
| | egriqdlekyvedtkidlwsynaellvalenqhtidltdsemnklfektkk |
| | qlrenaedmgngsfkiyhkcdnacigsirngtydhdvyrdealnnrfqikg |
| | velksgykd (SEQ ID NO: 199) |
| A/Massachusetts/18/2022 | gfrhqnsegrgqaadlkstqaaidqisgklnrligktnekfhqiekefsev |
| (H3N2) | egrvqdlekyvedtkidlwsynaellvalenqhtidltdsemnklfektkk |
| | qlrenaedmgngsfkiyhkcdnacigsirnetydhnvyrdealnnrfqikg |
| | velksgykd (SEQ ID NO: 200) |
| A/mallard/New | gfrhqnaegtgtaadlkstqaaidqingklnrliektnekyhqiekefeqv |
| Brunswick/00389/2010 (H4N6) | egriqdlekyvedtkidlwsynaellvalenqhtidvtdsemnklfervrr |
| | qlrenaedkgngsfeifhqcdnnciesirngtydhdiyrdeainnrfqiqg |
| | vkltqgykd (SEQ ID NO: 201) |
| A/Texas/37/2024 (H5N1) | gyhhsneqgsgyaadkestqkaidgvtnkvnsiidkmntqfeavgrefnnl |
| | errienlnkkmedgfldvwtynaellvlmenertldfhdsnvknlydkvrl |
| | qlrdnakelgngcfefyhkcdnecmesvrngtydypqyseearlkreeisg |
| | vklesvgtyq (SEQ ID NO: 202) |
| A/chicken/Taiwan/2759/2012 | gyhhensqgsgyaadrestqkaidgitnkvnsiinkmntqfeavdhefsnl |
| (H6N1) | errignlnkrmedgfldvwtynaellvllenertldlhdanvknlyekvks |
| | qlrdnandlgngsfefwhkcdnecmesvkngtydypkyqkesklnrqgies |
| | vklenlgvyq (SEQ ID NO: 203) |
| A/chicken/Guangdong/J1/2017 | gfrhqnaqgegtaadykstqsaidqitgklnrliaktnqqfklidnefnev |
| (H7N9) | ekqignvinwtrdsitevwsynaellvamenqhtidladsemdklyervkr |
| | qlrenaeedgtgsfeifhkcdddcmasirnntydhrkyreeamqnriqidp |
| | vklssgykd (SEQ ID NO: 204) |
| A/northern | gfhhsnsegtgmaadqkstqeaidkitnkvnnivdkmnrefevvnhefsev |
| shoveler/California/ | ekrinmindkiddqieglwaynaellvllenqktldehdsnvknlfdevkr |
| HKWF1204/2007 (H8N4) | rlstnamdagngsfdilhkcnnecmetikngtynhkeyeceaklersking |
| | vkleenttyk (SEQ ID NO: 205) |
| A/duck/Beijing/MG0617/2005 | gfqhsndqgvgmaadrdstqkaidkitskvnnivdkmnkqyeiidhefsev |
| (H9N2) | etrlnminnkiddqiqdiwaynaellvllenqktldehdanvnnlynkvkr |
| | algsnavedgkgsfelyhkcddqcmetirngtynrrkyqeesklerqkieg |
| | vklesegtyk (SEQ ID NO: 206) |
| A/chicken/Jiangxi/18933/2013 | gfrhqnaqgtgqaadykstqaaidqitgklnrlvektntefesiesefsei |
| (H10N8) | ehqignvinwtkdsitdiwtyqaellvamenqhtidmadsemlnlyervrk |
| | qlrqnaeedgkgsfeiyhacddscmesirnntydhsqyreeallnrlninp |
| | vtlssgykd (SEQ ID NO: 207) |
| A/common | gfqhrndegtgiaadkestqkaidqitskvnnivdrmntnfesvqhefsei |
| teal/Netherlands/1/2015 | eerinqlskhvddsvvdiwsynaqllvllenektldlhdsnvrnlhekvrr |
| (H11N1) | mlkdnakdegngsftfyhkcdneciervrngtydhkefeeeskinrqeieg |
| | vkldssgnvyk (SEQ ID NO: 208) |
| A/Northern | gfqhqnaegtgiaadrdstqkaidnmqnklnnvidkmnkqfdvvnhefsev |
| Shoveler/Nevada/D1516557/ | esrinminskvddqitdiwaynaellvllenqktldehdanvrnlhdrvrr |
| 2015 (H12N2) | vlrenaidtgdgsfeilhkcdnncmdtirngtydhkeyeeeskierqking |
| | vkleenstyk (SEQ ID NO: 209) |
| A/Armenian gull/Republic of | gfqhqneqgvgiaadkestqkainqittkinniiekmngnydsirgefnqv |
| Georgia/2/2012 (H13N2) | eqrinmlanriddavtdvwsynakllvllendktldmhdanvrnlhdqvrr |
| | vlktnaidegngsfellhkcndscmetirngtynhteyeeesklkrqeieg |
| | iklksddnvyk (SEQ ID NO: 210) |
| A/blue-winged teal/TX/AI13- | gfrhqnaegtgtaadlkstqaaidqingklnrliektnekyhqiekefeqv |
| 1028/2013 (H14N5) | egriqdlekyvedtkidlwsynaellvalenqhtidvtdsemnklfervrr |
| | qlrenaedqgngsfeifhqcdnnciesirngtydhniyrdeainnrikinp |
| | vnltmgykd (SEQ ID NO: 211) |
| A/shearwater/Australia/2576/ | gfrhqnaqgqgtaadykstqaaidqitgklnrliektnkqfelidneftev |
| 1979 (H15N6) | eqqignvinwtrdslteiwsynaellvamenqhtidladsemnklyervrr |
| | qlrenaeedgtgsfeifhrcddqcmesirnntynhteyrqealqnriminp |
| | vklssgykd (SEQ ID NO: 212) |
| A/shorebird/Delaware/172/ | gfqhqneqgtgiaadkastqkaideittkinniiekmngnydsirgefnqv |
| 2006 (H16N3) | ekrinmladrvddavtdvwsynakllvllengrtldlhdanvrnlhdqikr |
| | tlknnaidegdgsfnllhkcndscmeairngtynhedykeesqlkrqeieg |
| | iklktednvyk (SEQ ID NO: 213) |
| A/little yellow-shouldered | gyhhenqegsgyaadkeatqkavdaitnkvnsiidkmnsqfesnikefnrl |
| bat/Guatemala/060/2010 | elriqhlsdrvddalldiwsyntellvllenertldfhdanvknlfekvka |
| (H17N10) | qlkdnaidegngsflllhkcnnscmddikngtykymdyreeshiekqkidg |
| | vkltdysryytmt (SEQ ID NO: 214) |
| A/flat-faced bat/Peru/033/ | gyhhqnsegsgyaadkeatqkavdaittkvnniidkmntqfestakefnki |
| 2010 (H18N11) | emrikhlsdrvddgfldvwsynaellvllenertldfhdanvnnlyqkvkv |
| | qlkdnaidmgngsfkilhkcnntcmddikngtynyyeyrkeshlekqkidg |
| | vklsenssykim (SEQ ID NO: 215) |
| B/Malaysia/2506/2004 | gytshgahgvavaadlkstqeainkitknlnslselevknlqrlsgamdel |
| | hneileldekvddlradtissqielavllsnegiinsedehllalerklkk |
| | mlgpsaveigngsfetkhkcnqtcldriaagtfdagefslptfdslnitaa |
| | slnddgldnhti (SEQ ID NO: 216) |
| B/Phuket/3073/2013 | gytshgahgvavaadlkstqeainkitknlnslselevknlqrlsgamdel |
| | hneileldekvddlradtissqielavllsnegiinsedehllalerklkk |
| | mlgpsavdigngsfetkhkcnqtcldriaagtfnagefslptfdslnitaa |
| | slnddgldnht (SEQ ID NO: 217) |
| B/Wisconsin/22/2024 | gytshgahgvavaadlkstqeainkitknlnslselevknlqrlsgamdel |
| | hneileldekvddlradtissqielavllsnegiinsedehllalerklkk |
| | mlgpsaveigngsfetkhkcnqtcldriaagtfdagefslptfdslnitaa |
| | slnddgldnhti (SEQ ID NO: 218) |

In an aspect, a disclosed universal influenza vaccine antigen can be used to produce a binding molecule and/or a disclosed antibody comprising (i) a variable light chain region (VL) comprising 3 complementarity determining regions (CDRs), and (ii) a variable heavy chain region (VH) comprising 3 CDRs and a constant heavy chain region.

In an aspect, a disclosed universal influenza vaccine antigen can be used to produce a binding molecule and/or a disclosed antibody comprising a VH having at least one CDR having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect, a disclosed universal influenza vaccine antigen can be used to produce a binding molecule and/or a disclosed antibody comprising a VH having at least two CDRs having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect, a disclosed universal influenza vaccine antigen can be used to produce a binding molecule and/or a disclosed antibody comprising VH having at least three CDRs having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect, a disclosed universal influenza vaccine antigen can be used to produce a binding molecule and/or a disclosed antibody comprising a VL having at least one CDR having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a VL having at least two CDRs having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a VL having at least three CDRs having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a VH having at least three CDRs having the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184, and a VL having at least three CDRs having the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a V having at least one CDR having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect, a disclosed universal influenza vaccine antigen can be used to produce a binding molecule and/or a disclosed antibody comprising a VH having at least two CDRs having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect, a disclosed universal influenza vaccine antigen can be used to produce a binding molecule and/or a disclosed antibody comprising a VH having at least three CDRs having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184.

In an aspect, a disclosed universal influenza vaccine antigen can be used to produce a binding molecule and/or a disclosed antibody comprising a VL having at least one CDR having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a VL having at least two CDRs having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a VL having at least three CDRs having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used to produce a binding molecule and/or a disclosed antibody comprising a VH having at least three CDRs having a sequence having at least about 80%, 85%, 90%, 91%, 92%, 93%, 94%, 95%, 96%, 97%, 98%, 99% or greater identity to the sequence set forth in any one of SEQ ID NO:59-SEQ ID NO:63, SEQ ID NO:70-SEQ ID NO:74, SEQ ID NO:81-SEQ ID NO:85, SEQ ID NO:92-SEQ ID NO:96, SEQ ID NO:103-SEQ ID NO:107, SEQ ID NO:114-SEQ ID NO:118, SEQ ID NO:125-SEQ ID NO:129, SEQ ID NO:136-SEQ ID NO:140, SEQ ID NO:147-SEQ ID NO:151, SEQ ID NO:158-SEQ ID NO:162, SEQ ID NO:169-SEQ ID NO:173, and SEQ ID NO:180-SEQ ID NO:184, and a VL having at least three CDRs having a sequence having at least about 80%, 85%, 90%, 91%, 92%, 93%, 94%, 95%, 96%, 97%, 98%, 99% or greater identity to the sequence set forth in any one of SEQ ID NO:64-SEQ ID NO:69, SEQ ID NO:75-SEQ ID NO:80, SEQ ID NO:86-SEQ ID NO:91, SEQ ID NO:97-SEQ ID NO:102, SEQ ID NO:108-SEQ ID NO:113, SEQ ID NO:119-SEQ ID NO:124, SEQ ID NO:130-SEQ ID NO:135, SEQ ID NO:141-SEQ ID NO:146, SEQ ID NO:152-SEQ ID NO:157, SEQ ID NO:163-SEQ ID NO:168, SEQ ID NO:174-SEQ ID NO:179, and SEQ ID NO:185-SEQ ID NO:190.

In an aspect, a disclosed universal influenza vaccine antigen can be used with a vaccine adjuvant. In an aspect, a disclosed vaccine adjuvant can comprise a substance having an immune-enhancing effect (e.g., enhancing the immunogenicity of an antigen by being added to the antigen). In an aspect, a vaccine adjuvant can comprise alum, emulsions (e.g., Freund's adjuvant, MF59 AddaVax™, AS03, etc.), a substance that enhance the physiological activity of NOD receptors, a substance that enhance the physiological activity of RIG-I receptors, a substance that enhance the physiological activity of C-type lectin receptors, a substance that enhance the physiological activity of cytosolic DNA receptors, a substance that enhance the physiological activity of STING, etc.).

In an aspect, a disclosed universal influenza antigen can be formulated for encapsulation in a nanoparticle and/or a lipid nanoparticle.

### Pharmaceutical Formulations

Disclosed herein is a pharmaceutical formulation comprising a disclosed binding molecule and/or antibody and one or more pharmaceutically acceptable carriers and/or excipients. Disclosed herein is a pharmaceutical formulation comprising a disclosed antibody and one or more pharmaceutically acceptable carriers and/or excipients. Disclosed herein is a pharmaceutical formulation comprising a disclosed universal influenza antigen and one or more pharmaceutically acceptable carriers and/or excipients. Disclosed herein is a pharmaceutical formulation comprising a disclosed nucleic acid molecule and one or more pharmaceutically acceptable carriers and/or excipients. Disclosed herein is a pharmaceutical formulation comprising one or more disclosed binding molecules and/or antibodies and one or more pharmaceutically acceptable carriers and/or excipients. Disclosed herein is a pharmaceutical formulation comprising one or more disclosed antibodies and one or more pharmaceutically acceptable carriers and/or excipients. Disclosed herein is a pharmaceutical formulation comprising one or more disclosed universal influenza antigens and one or more pharmaceutically acceptable carriers and/or excipients. Disclosed herein is a pharmaceutical formulation comprising one or more disclosed nucleic acid molecules and one or more pharmaceutically acceptable carriers and/or excipients.

Disclosed herein is a pharmaceutical formulation comprising a disclosed postfusion HA protein or a fragment thereof and one or more pharmaceutically acceptable carriers and/or excipients. Disclosed herein is a pharmaceutical formulation comprising one or more disclosed postfusion HA proteins or fragments thereof and one or more pharmaceutically acceptable carriers and/or excipients. Disclosed herein is a pharmaceutical formulation comprising a disclosed postfusion HA protein or a fragment thereof, a disclosed binding molecule and/or antibody, and one or more pharmaceutically acceptable carriers and/or excipients. Disclosed herein is a pharmaceutical formulation comprising one or more disclosed postfusion HA proteins or fragments thereof, one or more disclosed binding molecules and/or disclosed antibodies, and one or more pharmaceutically acceptable carriers and/or excipients.

In an aspect, a disclosed pharmaceutical formulation can be administered to a subject in need thereof. In an aspect, a disclosed pharmaceutical formulation can be formulated for administration and/or can be administered via one or more routes. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration of a disclosed pharmaceutical formulation can comprise administration directly into the CNS (e.g., intraparenchymal, intracerebroventricular, intrathecal cisternal, intrathecal (lumbar), deep gray matter delivery, convection-enhanced delivery to deep gray matter) or the PNS. Administration can be continuous or intermittent.

In an aspect, a disclosed pharmaceutical formulation comprising a disclosed binding molecule and/or antibodies and/or antibody can be used in a disclosed method. In an aspect, a disclosed pharmaceutical formulation comprising a disclosed nucleic acid molecule can be used in a disclosed method. In an aspect, a disclosed pharmaceutical formulation comprising a disclosed binding molecule and/or antibodies and/or antibody can be used in a disclosed method of treating a subject having an influenza infection.

In an aspect, a disclosed pharmaceutical formulation (i) can minimize and/or decrease the risk of the subject's mortality; (ii) can increase and/or prolong the subject's survival; (iii) can enhance and/or improve the subject's quality of life; (iv) can reduce and/or minimize the likelihood of surgical intervention; (v) can reduce and/or decrease the frequency of other treatment; (vi) can relieve and/or ameliorate one or more subject's symptoms; (vii) can reduce and/or minimize the subject's risk of hospitalization; (viii) can prevent and/or minimize the need for surgical intervention; (ix) can improve and/or restore normal metabolism of one or more of the subject's organ systems, (x) can restore and/or improve one or more aspects of cellular homeostasis and/or cellular functionality, and/or metabolic dysregulation in one or more of the subject's affected systems, or (xi) any combination thereof. In an aspect, a disclosed pharmaceutical formulation can treat an influenza infection.

In an aspect, a disclosed pharmaceutical formulation can be used with one or more additional therapeutic agents. In an aspect, a disclosed pharmaceutical formulation comprising can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed pharmaceutical formulation can be used with one or more targeted therapies. In an aspect, a disclosed pharmaceutical formulation can comprise one or more anti-viral agents. In an aspect, anti-viral agents can comprise oseltamivir phosphate, zanamivir, peramivir, baloxavir marboxil, or any combination thereof.

In an aspect, a disclosed pharmaceutical formulation can be used to improve and/or can be used to enhance the quality of the subject's life when compared to a pre-treatment level.

In an aspect, a disclosed pharmaceutical formulation can be used to improve the subject's quality of life by at least 50% when compared to the subject's pre-treatment quality of life.

In an aspect, a disclosed pharmaceutical formulation can be used to diminish and/or decrease one or more symptoms associated with and/or related to the subject's an influenza infection.

In an aspect, a disclosed pharmaceutical formulation can be used to prevent an undesired physiological change, disease, pathological condition, or disorder from occurring in the subject having an influenza infection. In an aspect, a disclosed pharmaceutical formulation can be used to inhibit a physiological change, disease, pathological condition, or disorder, i.e., arresting its development, in the subject. In an aspect, a disclosed pharmaceutical formulation can be used to relieve a physiological change, disease, pathological condition, or disorder, i.e., causing regression of the disease, in the subject.

In an aspect, one or more disclosed pharmaceutical formulations can be subjected to one or more validating and/or characterizing steps and/or protocols. For example, in an aspect, validating and/or characterizing one or more disclosed pharmaceutical formulations can comprise measuring, ascertaining, and/or determining the purity and/or efficacy of the one or more disclosed pharmaceutical formulations.

In an aspect, a disclosed pharmaceutical formulation can confer Fc-dependent protection. In an aspect, a disclosed pharmaceutical formulation can activate and/or elicit complement-dependent cytotoxicity (CDC), antibody-dependent cellular phagocytosis (ADCP), and antibody-dependent cellular cytotoxicity (ADCC), or any combination thereof. In an aspect, a disclosed pharmaceutical formulation does not neutralize infectivity of the influenza virus. In an aspect, a disclosed pharmaceutical formulation can confer protection against lethal infection of the influenza virus. In an aspect, a disclosed pharmaceutical formulation can confer protection against mortality and/or morbidity associated with the influenza virus.

In an aspect, a disclosed pharmaceutical formulation can be formulated for encapsulation in a nanoparticle and/or a lipid nanoparticle.

### D. Methods

Disclosed herein is a method of treating a subject having an influenza infection, the method comprising administering to a subject in need thereof a therapeutically effective amount of a disclosed binding molecule and/or a disclosed antibody. Disclosed herein is a method of treating a subject having an influenza infection, the method comprising administering to a subject in need thereof a therapeutically effective amount of one or more disclosed binding molecules and/or disclosed antibodies. Disclosed herein is a method of treating a subject having an influenza infection, the method comprising administering to a subject in need thereof a therapeutically effective amount of a disclosed pharmaceutical formulation. Disclosed herein is a method of treating a subject having an influenza infection, the method comprising administering to a subject in need thereof a therapeutically effective amount of one or more disclosed pharmaceutical formulations.

Disclosed herein is a method of slowing progression of an influenza infection, the method comprising administering to a subject in need thereof a therapeutically effective amount of a disclosed binding molecule and/or a disclosed antibody. Disclosed herein is a method of slowing progression of an influenza infection, the method comprising administering to a subject in need thereof a therapeutically effective amount of one or more disclosed binding molecules and/or disclosed antibodies. Disclosed herein is a method of slowing progression of an influenza infection, the method comprising administering to a subject in need thereof a therapeutically effective amount of a disclosed pharmaceutical formulation. Disclosed herein is a method of slowing progression of an influenza infection, the method comprising administering to a subject in need thereof a therapeutically effective amount of one or more disclosed pharmaceutical formulations.

Disclosed herein is a method of reducing the symptoms of an influenza infection, the method comprising administering to a subject in need thereof a therapeutically effective amount of a disclosed binding molecule and/or a disclosed antibody. Disclosed herein is a method of reducing the symptoms of an influenza infection, the method comprising administering to a subject in need thereof a therapeutically effective amount of one or more disclosed binding molecules and/or disclosed antibodies. Disclosed herein is a method of reducing the symptoms of an influenza infection, the method comprising administering to a subject in need thereof a therapeutically effective amount of a disclosed pharmaceutical formulation. Disclosed herein is a method of reducing the symptoms of an influenza infection, the method comprising administering to a subject in need thereof a therapeutically effective amount of one or more disclosed pharmaceutical formulations.

Disclosed herein is a method of minimizing the morbidity and/or the risk of mortality due to an influenza infection, the method comprising administering to a subject in need thereof a therapeutically effective amount of a disclosed binding molecule and/or a disclosed antibody. Disclosed herein is a method of minimizing the morbidity and/or the risk of mortality due to an influenza infection, the method comprising administering to a subject in need thereof a therapeutically effective amount of one or more disclosed binding molecules and/or disclosed antibodies. Disclosed herein is a method of minimizing the morbidity and/or the risk of mortality due to an influenza infection, the method comprising administering to a subject in need thereof a therapeutically effective amount of a disclosed pharmaceutical formulation. Disclosed herein is a method of minimizing the morbidity and/or the risk of mortality due to an influenza infection, the method comprising administering to a subject in need thereof a therapeutically effective amount of one or more disclosed pharmaceutical formulations. Disclosed herein is a method of treating a subject having an influenza infection, the method comprising administering to a subject in need thereof a therapeutically effective amount of a binding molecule and/or a disclosed antibody, wherein the binding molecule and/or a disclosed antibody can comprise (i) a VL comprising the sequence set forth in any one of SEQ ID NO:17-SEQ ID NO:28, and (ii) a VH comprising the sequence set forth in any one of SEQ ID NO:05-SEQ ID NO:16.

In an aspect of a disclosed method, a disclosed binding molecule and/or a disclosed antibody comprising a VL can comprise any disclosed CDR including, for example, those listed in Table 3-Table 14. In an aspect of a disclosed method, a disclosed binding molecule and/or a disclosed antibody comprising a VH can comprise any disclosed CDR including, for example, those listed in Table 3-Table 14.

In an aspect of a disclosed method, a disclosed binding molecule and/or a disclosed antibody can comprise a VH having the sequence set forth in Table 1. In an aspect of a disclosed method, a disclosed binding molecule and/or a disclosed antibody can comprise a VL having the sequence set forth in Table 2.

In an aspect of a disclosed method, a disclosed binding molecule and/or a disclosed antibody can comprise a VH having the sequence set forth in Table 1 and can comprise a VL having the sequence set forth in Table 2.

Disclosed herein is a method of treating a subject having an influenza infection, the method comprising administering to a subject in need thereof a therapeutically effective amount of a disclosed postfusion HA protein or a fragment thereof. Disclosed herein is a method of treating a subject having an influenza infection, the method comprising administering to a subject in need thereof a therapeutically effective amount of one or more disclosed postfusion HA proteins or fragments thereof. Disclosed herein is a method of treating a subject having an influenza infection, the method comprising administering to a subject in need thereof a therapeutically effective amount of a disclosed pharmaceutical formulation comprising a disclosed postfusion HA protein or a fragment thereof. Disclosed herein is a method of treating a subject having an influenza infection, the method comprising administering to a subject in need thereof a therapeutically effective amount of one or more disclosed pharmaceutical formulations one or more disclosed postfusion HA protein or fragments thereof.

Disclosed herein is a method of slowing progression of an influenza infection, the method comprising administering to a subject in need thereof a therapeutically effective amount of a disclosed postfusion HA protein or a fragment thereof. Disclosed herein is a method of slowing progression of an influenza infection, the method comprising administering to a subject in need thereof a therapeutically effective amount of one or more a disclosed postfusion HA proteins or fragments thereof. Disclosed herein is a method of slowing progression of an influenza infection, the method comprising administering to a subject in need thereof a therapeutically effective amount of a disclosed pharmaceutical formulation comprising a disclosed postfusion HA protein or a fragment thereof. Disclosed herein is a method of slowing progression of an influenza infection, the method comprising administering to a subject in need thereof a therapeutically effective amount of one or more disclosed pharmaceutical formulations comprising one or more disclosed postfusion HA proteins or fragments thereof.

Disclosed herein is a method of reducing the symptoms of an influenza infection, the method comprising administering to a subject in need thereof a therapeutically effective amount of a disclosed postfusion HA protein or a fragment thereof. Disclosed herein is a method of reducing the symptoms of an influenza infection, the method comprising administering to a subject in need thereof a therapeutically effective amount of one or more disclosed postfusion HA proteins or fragments thereof. Disclosed herein is a method of reducing the symptoms of an influenza infection, the method comprising administering to a subject in need thereof a therapeutically effective amount of a disclosed pharmaceutical formulation comprising a disclosed postfusion HA protein or a fragment thereof. Disclosed herein is a method of reducing the symptoms of an influenza infection, the method comprising administering to a subject in need thereof a therapeutically effective amount of one or more disclosed pharmaceutical formulations comprising one or more a disclosed postfusion HA proteins or fragments thereof.

Disclosed herein is a method of minimizing the morbidity and/or the risk of mortality due to an influenza infection, the method comprising administering to a subject in need thereof a therapeutically effective amount of a disclosed postfusion HA protein or a fragment thereof. Disclosed herein is a method of minimizing the morbidity and/or the risk of mortality due to an influenza infection, the method comprising administering to a subject in need thereof a therapeutically effective amount of one or more disclosed postfusion HA proteins or fragments thereof. Disclosed herein is a method of minimizing the morbidity and/or the risk of mortality due to an influenza infection, the method comprising administering to a subject in need thereof a therapeutically effective amount of a disclosed pharmaceutical formulation comprising a disclosed postfusion HA protein or a fragment thereof. Disclosed herein is a method of minimizing the morbidity and/or the risk of mortality due to an influenza infection, the method comprising administering to a subject in need thereof a therapeutically effective amount of one or more disclosed pharmaceutical formulations comprising one or more disclosed postfusion HA proteins or fragments thereof. Disclosed herein is a method of treating a subject having an influenza infection, the method comprising administering to a subject in need thereof a therapeutically effective amount of a disclosed postfusion HA protein or a fragment thereof, such as those, for example, set forth in Table 16.

In an aspect, a disclosed postfusion HA protein or fragment thereof can comprise a β-hairpin. In an aspect, a disclosed postfusion HA protein or fragment thereof can comprise a β-hairpin capable of binding to one or more disclosed binding molecules and/or disclosed antibodies. In an aspect, a disclosed postfusion HA protein or fragment thereof can comprise a β-hairpin comprising the sequence set forth in SEQ ID NO:191. In an aspect, a disclosed postfusion HA protein or fragment thereof can comprise the sequence set forth in any one of SEQ ID NO:219-SEQ ID NO:236. In an aspect, a disclosed postfusion HA protein or fragment thereof can comprise the sequence set forth in any one of SEQ ID NO:29-SEQ ID NO:58. In an aspect, a disclosed postfusion HA protein or fragment thereof can comprise a sequence having at least about 80%, at least about 85%, at least about 90%, at least about 95%, or more than at least 95% identity to the sequence set forth in any one of SEQ ID NO:219-SEQ ID NO:236. In an aspect, a disclosed postfusion HA protein or fragment thereof can comprise a sequence having at least about 80%, at least about 85%, at least about 90%, at least about 95%, or more than at least 95% identity to the sequence set forth in any one of SEQ ID NO:29-SEQ ID NO:58.

In an aspect, a disclosed postfusion HA protein or fragment thereof can comprise a sequence having at least about 80%, at least about 85%, at least about 90%, at least about 95%, or more than at least 95% identity to the sequence set forth in Table 16. In an aspect, a disclosed postfusion HA protein or fragment thereof can comprise a sequence set forth in Table 16.

In an aspect, a disclosed postfusion HA protein or fragment thereof can be generated following an influenza infection. In an aspect, a disclosed postfusion HA protein or fragment thereof can be antigenic. In an aspect, a disclosed postfusion HA protein or fragment thereof can be antigenic. In an aspect, a disclosed postfusion HA protein or fragment thereof can be characterized including, for example, for antigenic properties. In an aspect, “antigenic characterization” can refer to the analysis of virus' antigenic properties to help assess how related it is to another virus.

In an aspect, a disclosed postfusion HA protein or fragment thereof can be used in a method of inhibiting egress of influenza virus from infected cells. In an aspect, a disclosed postfusion HA protein or fragment thereof can be used in a method that (i) can minimize and/or decrease the risk of the subject's mortality; (ii) can increase and/or prolong the subject's survival; (iii) can enhance and/or improve the subject's quality of life; (iv) can reduce and/or minimize the likelihood of surgical intervention; (v) can reduce and/or decrease the frequency of other treatment; (vi) can relieve and/or ameliorate one or more subject's symptoms; (vii) can reduce and/or minimize the subject's risk of hospitalization; (viii) can prevent and/or minimize the need for surgical intervention; (ix) can improve and/or restore normal metabolism of one or more of the subject's organ systems, (x) can restore and/or improve one or more aspects of cellular homeostasis and/or cellular functionality, and/or metabolic dysregulation in one or more of the subject's affected systems, or (xi) any combination thereof. In an aspect, a disclosed postfusion HA protein or fragment thereof can be used in a method of treating an influenza infection. In an aspect, a disclosed postfusion HA protein or fragment thereof can be used in a method of eliciting and/or provoking a protective antibody result in a subject in need thereof. In an aspect, a disclosed postfusion HA protein or fragment thereof can be generated following administration of one or more disclosed binding molecules and/or disclosed antibodies.

In an aspect, a disclosed postfusion HA protein or fragment thereof can be prepared in a disclosed pharmaceutical formulation. In an aspect, a disclosed postfusion HA protein or fragment thereof can be encoded by a disclosed nucleic acid sequence or molecule. In an aspect, a disclosed postfusion HA protein or fragment thereof can be used in a disclosed method. In an aspect, a disclosed postfusion HA protein or fragment thereof can be prepared with a disclosed adjuvant or any suitable adjuvant known to the skilled person or any commercially available adjuvant.

In an aspect, a disclosed method can comprise administering to a subject (i) one or more disclosed binding molecules and/or disclosed antibodies, and (ii) one or more disclosed postfusion HA protein and/or fragments thereof. In an aspect, a disclosed method can comprise administering to a subject (i) a disclosed pharmaceutical formulation comprising one or more disclosed binding molecules and/or disclosed antibodies, and (ii) a disclosed pharmaceutical formulation comprising one or more disclosed postfusion HA protein and/or fragments thereof. In an aspect, a disclosed method can comprise administering to a subject a pharmaceutical formulation comprising (i) one or more disclosed binding molecules and/or disclosed antibodies, and (ii) one or more disclosed postfusion HA protein and/or fragments thereof.

In an aspect, a disclosed method can comprise repeating one or more times the administering to a subject (i) a disclosed pharmaceutical formulation comprising one or more disclosed binding molecules and/or disclosed antibodies, and (ii) a disclosed pharmaceutical formulation comprising one or more disclosed postfusion HA protein and/or fragments thereof.

In an aspect, a disclosed method can comprise repeating one or more times the administering to a subject a pharmaceutical formulation comprising (i) one or more disclosed binding molecules and/or disclosed antibodies, and (ii) one or more disclosed postfusion HA protein and/or fragments thereof.

In an aspect, a disclosed method can further comprise administering to the subject one or more additional therapies.

In an aspect, following the administering step, (i) the risk of the subject's mortality can be minimized and/or decreased; (ii) the subject's survival can be increased and/or prolonged; (iii) the subject's quality of life can be enhanced and/or improved; (iv) the likelihood of surgical intervention can be reduced and/or minimized; (v) the frequency of other treatment can be reduced and/or decreased; (vi) one or more subject's symptoms can be relieved and/or ameliorated; (vii) the subject's risk of hospitalization can be reduced and/or minimized; (viii) the need for surgical intervention can be prevented and/or minimized; (ix) normal metabolism of one or more of the subject's organ systems can be improved and/or restored, (x) one or more aspects of cellular homeostasis and/or cellular functionality, and/or metabolic dysregulation in one or more of the subject's affected systems can be restored and/or improved, or (xi) any combination thereof.

In an aspect, an affected system can be the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect, following the administering step, one or more aspects of the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof can be improved, treated, repaired, and/or restored to wellness.

In an aspect of a disclosed method, administering one or more disclosed binding molecules and/or disclosed antibodies can comprise a single dose or multiple doses (such as 2, 3, 4, 5, 6, 7, 8, 9, 10, or more than 10 doses). In an aspect of a disclosed method, administering one or more disclosed binding molecules and/or disclosed antibodies can comprise a single dose or multiple doses (such as 2, 3, 4, 5, 6, 7, 8, 9, 10, or more than 10 doses). In an aspect of a disclosed method, administering one or more disclosed binding molecules and/or disclosed antibodies can comprise a single dose or multiple doses (such as 2, 3, 4, 5, 6, 7, 8, 9, 10, or more than 10 doses).

In an aspect, a therapeutically effective amount of a disclosed binding molecule and/or a disclosed antibody can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed binding molecule and/or a disclosed antibody can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed subject has already received or is actively receiving one or more therapies and/or treatments. In an aspect, a subject is treatment-naïve.

In an aspect of a disclosed method of treating an influenza infection, administering a disclosed treatment regimen can comprise intravenous administration. In an aspect, a disclosed treatment regimen can be administered to a subject intravenously using, for example, a dual-channel infusion pump or two single channel pumps and central venous catheter. In an aspect, a disclosed IV administration of a disclosed treatment regimen can occur once every four hours at the infusion rate of from about 50 mL/hr to about 250 mL/hr (e.g., about 50, 75, 100, 125, 150, 175, 200, 225, 250 mL/hr) depending on the subject's age and condition/tolerance. In an aspect, one or more disclosed binding molecules and/or disclosed antibodies or disclosed pharmaceutical formulations can be delivered via an ambulatory infusion pump and subclavian catheter.

In an aspect, a disclosed method can comprise titrating the dose of a disclosed treatment regimen. In an aspect, a disclosed method can comprise titrating the dose of one or more disclosed binding molecules and/or disclosed antibodies or disclosed pharmaceutical formulations. In an aspect, a disclosed method can comprise titrating the dose of one or more disclosed binding molecules and/or disclosed antibodies or disclosed pharmaceutical formulations, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof to identify an effective dose and/or to identify an effective dose eliciting only mild adverse and/or side effects.

In an aspect, a disclosed method can comprise titrating the dose of a disclosed treatment regimen in a specific or disclosed subject. In an aspect, a disclosed method can comprise titrating the dose of one or more disclosed binding molecules and/or disclosed antibodies or disclosed pharmaceutical formulations in a specific or disclosed subject.

In an aspect, a disclosed method can comprise titrating the dose of a disclosed binding molecule and/or a disclosed antibody, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof to identify an effective dose and/or to identify an effective dose eliciting only mild adverse and/or side effects for a specific or disclosed subject.

In an aspect, administering can comprise administering to the subject the maximum tolerated dose of the one or more disclosed binding molecules and/or disclosed antibodies or disclosed pharmaceutical formulations. In an aspect, administering can comprise administering to the subject less than the maximum tolerated dose of the one or more disclosed binding molecules and/or disclosed antibodies or disclosed pharmaceutical formulations.

In an aspect, IV administration of a disclosed treatment regimen can comprise an outpatient setting. In an aspect, one or more disclosed binding molecules and/or disclosed antibodies or disclosed pharmaceutical formulations can be administering prior to, concurrent with, or after administering of a disclosed therapy and/or anti-viral agent. In an aspect, the order of administering the one or more disclosed binding molecules and/or disclosed antibodies or disclosed pharmaceutical formulations thereof and the administering of a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof can change during a treatment regimen. In an aspect, anti-viral agents can comprise oseltamivir phosphate, zanamivir, peramivir, baloxavir marboxil, or any combination thereof.

In an aspect, a disclosed method of can further comprise obtaining a biological sample from the subject prior to administering a disclosed treatment regimen. In an aspect, a disclosed method can further comprise obtaining a biological sample from the subject after administering a disclosed treatment regimen. In an aspect, a disclosed method can further comprise subjecting the biological sample to a cell-free DNA (cfDNA) analysis. cfDNA analyses are known to the skilled person in the art. In an aspect, a disclosed cfDNA analysis can be repeated one or more times. In an aspect, a disclosed obtaining step can be repeated one or more times.

In an aspect, a disclosed method can comprise generating a control sample and/or a pooled control sample. In an aspect, a disclosed method can comprise generating a reference sample and/or a pooled reference sample. In an aspect, a disclosed method can comprise generating a range of control samples and/or pooled control samples. In an aspect, a disclosed method can comprise generating a range of reference samples and/or pooled reference samples.

In an aspect, a complete response can be defined as complete disappearance of all tumors with no recurrence of tumors for at least four weeks. In an aspect, a partial response can be defined as a 50% reduction in total tumor size with such reduction lasting at least four weeks. In an aspect, stable disease can be defined as less than 50% reduction in size but no more than 25% increase in size of the tumor mass lasting for at least twelve weeks.

In an aspect, a disclosed method can further comprise measuring the subject's molecular response to a disclosed treatment regimen. In an aspect, a disclosed molecular response can comprise a decrease in the number of somatic genomic aberrations in a disclosed biological sample obtained from the subject. In an aspect, disclosed somatic genomic aberrations can comprise mutations, insertions, deletions, chromosomal rearrangements, copy number aberrations, fusions, or any combination thereof.

In an aspect, a disclosed molecular marker that can determine one or more suitable precision an influenza infection treatments in one or more disclosed methods can be measured from a sample by high-density expression array, DNA microarray, polymerase chain reaction (PCR), reverse transcriptase PCR (RT-PCR), real-time quantitative reverse transcription PCR (qRT-PCR), serial analysis of gene expression (SAGE), spotted cDNA arrays, GeneChip, spotted oligo arrays, bead arrays, RNA Seq, tiling array, northern blotting, hybridization microarray, in situ hybridization, whole-exome sequencing, whole-genome sequencing, liquid biopsy, next-generation sequencing, or any combination thereof.

In an aspect, a disclosed molecular marker can determine one or more treatments for use in a disclosed method of treating and/or preventing an influenza infection can determined from the nucleic acid sequence of the at least one of circulating DNA and/or RNA. In an aspect, a disclosed molecular marker can be assessed from circulating tumor DNA and/or RNA (ctDNA and/or ctRNA); circulating cell-free DNA and/or RNA (cfDNA, cfRNA); or any combination thereof. ctDNA/ctRNA refers to tumor-derived fragmented DNA in the bloodstream that is not associated with cells. cfDNA/cfRNA refers to DNA that is freely circulating in the bloodstream, but is not necessarily of tumor origin. In an aspect, cfDNA/ctDNA can include any whole or fragmented genomic DNA, or mitochondrial DNA, and/or cfRNA/ctRNA can include mRNA, tRNA, microRNA, small interfering RNA, long non-coding RNA (1 ncRNA). In an aspect, cfDNA and/or ctDNA can be a fragmented DNA with a length of at least about 50 base pair (bp), about 100 bp, about 200 bp, about 500 bp, or about 1 kbp. In an aspect, cfRNA and/or ctRNA can be a full length or a fragment of mRNA (e.g., at least 70% of full-length, at least 50% of full length, at least 30% of full length, etc.). In an aspect, a disclosed molecular marker can be directed against any an influenza infection-related gene disclosed herein.

In an aspect, a disclosed method can further comprise surgical intervention. In an aspect, a disclosed method can further comprise subjecting the subject to one or more invasive or non-invasive diagnostic assessments. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination. In an aspect, a disclosed invasive diagnostic assessment can comprise a tissue biopsy.

For example, in an aspect, repeating one or more disclosed steps of a disclosed method can comprise repeating the administering to the subject of a disclosed binding molecule and/or a disclosed antibody, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof, repeating the measuring of the subject's response, repeating the obtaining of a biological sample from the subject, repeating the subjecting the biological sample to cfDNA analysis, repeating the administering of one or more additional therapeutic agents, or any combination thereof.

In an aspect, a disclosed molecular marker can be detected, quantified, and/or analyzed over time (at different time points) to determine the effectiveness of a disclosed treatment regimen (e.g., one or more disclosed binding molecules and/or disclosed antibodies or disclosed pharmaceutical formulations) to the subject and/or to determine the response of a subject or subject's response to treatment. In an aspect, a disclosed method can comprise obtaining multiple measurements over time from the same subject and same sample may be quantified at a single time point or over time. In an aspect, a disclosed treatment regimen treatment (e.g., a disclosed treatment regimen comprising one or more disclosed binding molecules and/or disclosed antibodies or disclosed pharmaceutical formulations) can be designed and/or determined based on the influenza infection status and/or the changes/types of one or more molecular markers. In an aspect, the likelihood of success of a disclosed treatment regimen can be determined based on the influenza infection status and the type/quantity of one or more molecular markers.

In an aspect, a subject can be a human patient. In an aspect a subject can be any age (e.g., geriatric, adult, young adult, teenager, tween, adolescent, child, toddler, baby, or infant), can be male or female, can be any nationality, can be of any ethnicity, and/or can be of any race. In an aspect, a subject can be immune compromised. In an aspect, a disclosed subject can be geriatric and/or elderly. In an aspect, a disclosed subject be treatment-naive.

In an aspect, a disclosed method can be modified or one or more steps of a disclosed method can be modified. In an aspect, a disclosed method of treating and/or preventing an influenza infection can comprise modifying or changing one or more features or aspects of one or more steps. In an aspect, a method can be altered by changing the amount of a disclosed treatment regimen, a disclosed binding molecule and/or a disclosed antibody, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof administered to a subject, or by changing the frequency of administration of disclosed treatment regimen, a disclosed binding molecule and/or a disclosed antibody, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof to a subject, by changing the duration of time that disclosed treatment regimen, a disclosed binding molecule and/or a disclosed antibody, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof is administered to a subject, or by substituting for one or more of the disclosed components and/or reagents with a similar or equivalent component and/or reagent. The same applies to all disclosed treatment regimens, disclosed binding molecule and/or disclosed antibodies, disclosed targeted therapies, disclosed pharmaceutical formulations, disclosed anti-chemokines, disclosed anti-cancer agents, disclosed chemotherapeutic agents, or combinations thereof.

In an aspect, a disclosed method can improve and/or extend the survivability of the subject, can improve a subject's quality of life, can increase and/or prolong a subject's life span, or any combination thereof. In an aspect, the subject's life expectancy can be compared to the life expectancy of a control (i.e., no treatment with a disclosed binding molecule and/or a disclosed antibody). In an aspect, a control can be a subject not receiving a disclosed pharmaceutical composition. In an aspect, a control can be a pooled number of subjects not receiving a disclosed pharmaceutical composition. In an aspect, a control is one or more subjects having the same type of influenza infection as the subject. In an aspect, life expectancy can be defined as the time at which 50 percent of subjects are alive and 50 percent have passed away.

In an aspect, patient life expectancy can be indefinite following treatment with a disclosed method. In an aspect, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with the identical or near identical viral infection and the identical or near identical predicted outcome.

In an aspect, a disclosed method can further comprise monitoring the subject for adverse effects. In an aspect, in the absence of adverse effects, a disclosed method can further comprise continuing to treat the subject. In an aspect, continuing to treat the subject can comprise continuing to administer a disclosed composition (i.e., one or more disclosed binding molecules and/or disclosed antibodies or disclosed pharmaceutical formulations).

In an aspect, in the presence of adverse effects, a disclosed method can further comprise modifying one or more steps of the method. In an aspect, modifying one or more steps of a disclosed method can comprise modifying one or more administering steps. In an aspect, modifying one or more disclosed administering steps can comprise changing the amount of a disclosed composition (i.e., one or more disclosed binding molecules and/or antibodies or disclosed pharmaceutical formulations) administered to the subject, changing the frequency of a disclosed composition (i.e., one or more disclosed binding molecules and/or disclosed antibodies or disclosed pharmaceutical formulations) administration, changing the duration of a disclosed composition (i.e., one or more disclosed binding molecules and/or disclosed antibodies or disclosed pharmaceutical formulations) administration, changing the route of a disclosed composition (i.e., one or more disclosed binding molecules and/or disclosed antibodies or disclosed pharmaceutical formulations) administration, or any combination thereof. In an aspect, modifying one or more disclosed administering steps can comprise changing the amount a disclosed composition (i.e., one or more disclosed binding molecules and/or disclosed antibodies or disclosed pharmaceutical formulations) administered to the subject, changing the frequency of a disclosed composition (i.e., one or more disclosed binding molecules and/or disclosed antibodies d pharmaceutical formulations) administration, changing the duration of a disclosed composition (i.e., one or more disclosed binding molecules and/or disclosed antibodies or disclosed pharmaceutical formulations) administration, changing the route of a disclosed composition (i.e., one or more disclosed binding molecules and/or disclosed antibodies or disclosed pharmaceutical formulations), or any combination thereof.

In an aspect, a disclosed method can reduce the risk of developing an influenza infection. A reduction in the risk of developing an influenza infection can comprise a reduction of 10%, 20%, 30%, 40%, 50%, 60%, 70%, 80%, 90%, 100%, or any amount in the risk of an influenza infection when compared to a control subject (such as, for example, a subject that has not received one or more disclosed binding molecules and/or disclosed antibodies or disclosed pharmaceutical formulations).

In an aspect, a disclosed method can comprise improving and/or enhancing the subject's quality of life and/or movement. In an aspect of a disclosed method, a disclosed improvement and/or enhancement (such as, for example, in the subject's quality of life and/or movement) can comprise a 10%, 20%, 30%, 40%, 50%, 60%, 70%, 80%, 90%, 100%, or any amount of improvement and/or enhancement when compared to a control subject (such as, for example, a subject that has not received one or more disclosed binding molecules and/or disclosed antibodies or disclosed pharmaceutical formulations). In an aspect, a disclosed improvement and/or enhancement (such as, for example, in the subject's quality of life and/or movement) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not received one or more disclosed binding molecules and/or disclosed antibodies or disclosed pharmaceutical formulations).

In an aspect, a disclosed diminishment (such as, for example, the size of one or more tumors) can comprise a 10%, 20%, 30%, 40%, 50%, 60%, 70%, 80%, 90%, 100%, or any amount of a decrease when compared to a control subject (such as, for example, a subject that has not received one or more disclosed binding molecules and/or disclosed antibodies or disclosed pharmaceutical formulations). In an aspect, a disclosed diminishment (such as, for example, the size of one or more tumors) can comprise a 10-20%, 20-30%, 30-40%, 40-50%, 50-60%, 60-70%, 70-80%, 80-90%, or 90-100% or any amount of a decrease when compared to a control subject (such as a subject that has not received one or more disclosed binding molecules and/or disclosed antibodies or disclosed pharmaceutical formulations).

In an aspect, a disclosed method can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect of a disclosed method, administering one or more disclosed binding molecules and/or disclosed antibodies or disclosed pharmaceutical formulations can be daily, weekly, bi-weekly, monthly, or yearly. In an aspect, one or more disclosed binding molecules and/or disclosed antibodies or disclosed pharmaceutical formulations can be administered to a subject one or times per day (e.g., 1× per day, 2× per day, 3× per day, more than 3× per day, etc.), one or times per week, one or more times per month, or one or more times per year.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer disclosed binding molecule and/or disclosed antibodies or disclosed pharmaceutical formulations. In an aspect, metabolic and/or physiologic data can inform the clinician when make subsequent treatment decisions.

In an aspect, a disclosed method can further comprise repeating one or more disclosed steps of a disclosed method. In an aspect, a disclosed method can further comprise continuing to administer to the subject a therapeutically effective amount of one or more disclosed binding molecules and/or disclosed antibodies or disclosed pharmaceutical formulations.

In an aspect, a disclosed method can further comprise generating and/or making one or more disclosed binding molecules and/or disclosed antibodies or disclosed pharmaceutical formulations. In an aspect, a disclosed method can further comprise validating and/or characterizing one or more disclosed binding molecules and/or disclosed antibodies or disclosed pharmaceutical formulations. For example, in an aspect, validating and/or characterizing one or more disclosed binding molecules and/or disclosed antibodies or disclosed pharmaceutical formulations can comprise measuring, ascertaining, and/or determining the purity and/or efficacy of the one or more disclosed binding molecules and/or disclosed antibodies or disclosed pharmaceutical formulations.

In an aspect, a disclosed method can further comprise characterizing one or more disclosed binding molecules and/or disclosed antibodies or disclosed pharmaceutical formulations. In an aspect, characterizing one or more disclosed binding molecules can comprise analyzing and/or determining and/or quantifying the efficacy of one or more disclosed binding molecules and/or disclosed antibodies or disclosed pharmaceutical formulations. In an aspect, characterizing one or more disclosed binding molecules can comprise analyzing and/or determining and/or quantifying the efficacy of one or more disclosed binding molecules and/or disclosed antibodies or disclosed pharmaceutical formulations. In an aspect, efficacy can comprise the ability of one or more disclosed binding molecules and/or disclosed antibodies or disclosed pharmaceutical formulations to reduce and/or decrease and/or minimize symptoms of an influenza infection, the risk of the subject's morbidity and/or mortality, the duration that the subject remains symptomatic, and any combination thereof. In an aspect, efficacy can comprise the ability of one or more disclosed binding molecules and/or disclosed antibodies or disclosed pharmaceutical formulations (i) to minimize and/or decrease the risk of the subject's mortality; (ii) to increase and/or prolong the subject's survival; (iii) to enhance and/or improve the subject's quality of life; (iv) to reduce and/or minimize the likelihood of surgical intervention; (v) to reduce and/or decrease the frequency of other treatment; (vi) to relieve and/or ameliorate one or more subject's symptoms; (vii) to reduce and/or minimize the subject's risk of hospitalization; (viii) to prevent and/or minimize the need for surgical intervention; (ix) to improve and/or restore normal metabolism of one or more of the subject's organ systems, (x) to restore and/or improve one or more aspects of cellular homeostasis and/or cellular functionality, and/or metabolic dysregulation in one or more of the subject's affected systems, or (xi) any combination thereof.

### E. Kits

Disclosed herein is a kit comprising one or more disclosed binding molecules and/or disclosed antibodies or disclosed pharmaceutical formulations.

Disclosed herein is a kit comprising one or more disclosed postfusion HA proteins and/or fragments thereof. Disclosed herein is a kit comprising one or more disclosed binding molecules and/or disclosed antibodies or disclosed pharmaceutical formulations and one or more additional therapies. Disclosed herein is a kit comprising one or more disclosed postfusion HA proteins and/or fragments thereof and one or more additional therapies. Disclosed herein is a kit comprising one or more disclosed binding molecules and/or disclosed antibodies or disclosed pharmaceutical formulations used to treat a subject having an influenza infection. Disclosed herein is a kit comprising one or more disclosed binding molecules and/or disclosed antibodies or disclosed pharmaceutical formulations and one or more additional therapies used to treat a subject having an influenza infection. Disclosed herein is a kit for making and/or generating one or more disclosed binding molecules and/or disclosed antibodies or disclosed pharmaceutical formulations. Disclosed herein is a kit for making and/or generating one or more disclosed binding molecules and/or disclosed antibodies or disclosed pharmaceutical formulations for use in a method of treating a subject having an influenza infection. Disclosed herein is a kit for validating one or more disclosed binding molecules and/or disclosed antibodies or disclosed pharmaceutical formulations.

In an aspect, by using a disclosed kit, (i) the risk of the subject's mortality can be minimized and/or decreased; (ii) the subject's survival can be increased and/or prolonged; (iii) the subject's quality of life can be enhanced and/or improved; (iv) the likelihood of surgical intervention can be reduced and/or minimized; (v) the frequency of other treatment can be reduced and/or decreased; (vi) one or more subject's symptoms can be relieved and/or ameliorated; (vii) the subject's risk of hospitalization can be reduced and/or minimized; (viii) the need for surgical intervention can be prevented and/or minimized; (ix) normal metabolism of one or more of the subject's organ systems can be improved and/or restored, (x) one or more aspects of cellular homeostasis and/or cellular functionality, and/or metabolic dysregulation in one or more of the subject's affected systems can be restored and/or improved, or (xi) any combination thereof.

In an aspect, by using a disclosed kit, an influenza infection can be treated.

In an aspect, a disclosed kit can comprise one or more additional and/or therapeutic agents (e.g., anti-viral agents). Agents and therapeutic agents are known to the art and are described herein. In an aspect, anti-viral agents can comprise oseltamivir phosphate, zanamivir, peramivir, baloxavir marboxil, or any combination thereof.

In an aspect, a disclosed kit can comprise at least two components constituting the kit. Together, the components constitute a functional unit for a given purpose (such as, for example, treating a subject diagnosed with or suspected of having a disease or disorder such as an influenza infection). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

In an aspect, a disclosed kit for use in a disclosed method can comprise one or more containers holding a disclosed treatment regimen, a disclosed binding molecule and/or a disclosed antibody, a disclosed targeted therapy, a disclosed anti-viral agent, a disclosed pharmaceutical formulation, a disclosed anti-chemokine, a disclosed anti-cancer agent, a disclosed chemotherapeutic agent, or a combination thereof, and a label or package insert with instructions for use. In an aspect, suitable containers include, for example, bottles, vials, syringes, blister pack, etc. The containers can be formed from a variety of materials such as glass or plastic. The container can hold one or more disclosed binding molecules and/or disclosed antibodies, one or more disclosed pharmaceutical formulations comprising one or more disclosed binding molecules and/or disclosed antibodies or disclosed pharmaceutical formulations, or any combination thereof, and can have a sterile access port (for example the container may be an intravenous solution bag or a vial having a stopper pierceable by a hypodermic injection needle). The label or package insert can indicate one or more disclosed binding molecules and/or disclosed antibodies, disclosed pharmaceutical formulations, or any combination thereof can be used for treating, preventing, inhibiting, and/or ameliorating a disease or disorder or complications and/or symptoms associated with an influenza infection. A kit can comprise additional components necessary for administration such as, for example, other buffers, diluents, filters, needles, and syringes.

### EXAMPLES

The Examples that follow are illustrative of specific aspects of the invention, and various uses thereof. The Examples set forth for explanatory purposes only and are not to be taken as limiting the invention.

Influenza A and B viruses (IAVs and IBVs) comprise multiple phylogenetically and antigenically distinct groups, of which two A lineages (H1N1 and H3N2) and two B lineages (Victoria and Yamagata) currently circulate among humans (Krammer F, et al. (2018) Nat Rev Dis Primers 4:3). Antibody (Ab) responses to IAV/IBV infection or vaccination are largely directed toward the viral hemagglutinin (HA) glycoprotein, which mediates viral attachment and membrane fusion with the host cell (Krammer F, et al. (2018) Nat Rev Dis Primers 4:3). Population-level immune pressure drives selection of HA mutations that promote viral escape from immune control (Drake J W. (1993) Proc Natl Acad Sci USA. 90:4171-4175; Cobey S, et al. (2017) Curr Opin Virol. 22:105-111). Consequently, annually updated, licensed influenza vaccines typically protect only against seasonal strains and closely related subtypes (Krammer F, et al. (2015) Nat Rev Drug Discov. 14:167-182; Gerdil C, et al. (2003) Vaccine. 21:1776-1779).

The HA trimer exposes its three, independently folded, receptor-binding “head” domains at the membrane-distal end of an elongated “stem”. Mutations in the immunodominant head are responsible for annual antigenic drift. Absence of immune pressure probably accounts for conservation of the stem, sequestered by tight HA packing on the virion surface from exposure to B-cell receptors and Abs, but functional requirements for fusion may also limit its mutational possibilities (Knossow M, et al. (2006) Immunology. 119:1-7). Abs to conserved stem epitopes, as well as those to an epitopic region on the head interface or to the receptor site, protect against infection or disease by diverse subtypes and lineages of IAVs or IBVs (Dreyfus C, et al. (2012) Science. 337:1343-1348; Ekiert D C, et al. (2012) Nature. 489:526-532; Whittle J R, et al. (2011) Proc Natl Acad Sci USA. 108:14216-14221; Watanabe A, et al. (2019) Cell. 177:1124-1135 e1116), but such broadly protective HA Abs are rare. The only example of a protective monoclonal Ab (mAb) that cross-reacts with both IAV and IBV HAs is CR9114, a stem Ab isolated from a phage-displayed combinatorial Ab library (Dreyfus C, et al. (2012) Science. 337:1343-1348). Human Abs with naturally paired heavy (H)- and light (L)-chains that protect against both IAV and IBV infection or disease have yet to be detected.

The presence of neutralizing Abs—as measured by hemagglutination inhibition (HAI) titer-is the only commonly accepted correlate of protection against influenza infection (Paules C I, et al. (2017) Immunity. 47:599-603). Accordingly, research on IAV/IBV vaccines has focused largely on eliciting Abs with HAI activity and/or those that directly block viral entry (Krammer F, et al. (2016) Curr Opin Virol. 17:95-103; Laursen N S, et al. (2013) Antiviral Res. 98:476-483; Wei C J, et al. (2020) Nat Rev Drug Discov. 19:239-252).

Although an Ab response that prevents infection is strictly necessary for prophylaxis, even non-neutralizing Abs can effectively mediate protection against morbidity by promoting clearance of virus and infected cells, through complement-dependent cytotoxicity (CDC), Ab-dependent cellular phagocytosis (ADCP), and Ab-dependent cellular cytotoxicity (ADCC). Indeed, these mechanisms are the principal means by which many broadly reactive HA Abs confer protection against lethal infection (Watanabe A, et al. (2019) Cell. 177:1124-1135 e1116; DiLillo D J, et al. (2016) J Clin Invest. 126:605-610; DiLillo D J, et al. (2014) Nat Med. 20:143-151). Examples of protective, CDC/ADCP/ADCC-dependent Abs include those that bind HA in its prefusion conformation as well as those that preferentially bind HA in its post-fusion state. The relevant epitopes of the latter Abs are probably occluded in the prefusion structure, and exposed in the postfusion state, or else absent in the prefusion conformation, and then generated by fusogenic conformational rearrangement.

Research on vaccines for influenza and many other viruses concentrates largely on eliciting neutralizing Abs, which prevent infection and often have greater protective potency than non-neutralizing Abs. But a central conundrum of neutralizing Abs is their tendency to select for viral mutations that enable escape from immune control. Because the number of neutralizing epitopes is generally a modest fraction of the total number of potential epitopes on viral proteins, relatively few escape mutations are needed to evade neutralizing humoral responses. Non-neutralizing, HA-directed Abs with potent Fc effector activities protect mice from morbidity and mortality after receiving an otherwise lethal dose of influenza virus. Therefore, to confer immunity that resists antigenic drift, an influenza vaccine might need to broaden its epitopic coverage beyond neutralization and include additional tiers of control, including eliciting Abs to conserved, non-neutralizing epitopes.

Detailed herein is the identification and characterization of B cells secreting IgGs that bind cell-surface expressed HA from various IAV subtypes and from both IBV lineages. The IgGs bind the corresponding postfusion HA2s in vitro, as well as the recombinant, soluble, HA0 trimeric ectodomains in Luminex and ELISA assays. The structure of a Fab from one of them (S1V2-72) bound with the “EHA2” recombinant mimic (Chen J, et al. (1999) Proc Natl Acad Sci USA. 96:8967-8972) of postfusion HA2 from B/Malaysia/2506/2004, defined the epitope as a β-hairpin loop also present on the prefusion HA trimer, but occluded at the membrane-proximal end of the molecule. The fusogenic conformational change translocated this loop to the membrane-distal end of the postfusion form. S1V2-72 IgG did not neutralize infectivity in vitro, but conferred Fc-dependent protection from lethal challenge by either an IAV or an IBV.

### Materials and Methods

The full protocols for studies involving human subjects were approved by the Duke University Institutional Review Board and Boston University Institutional Review Board. Peripheral blood mononuclear cells (PBMCs) were obtained from human donors KEL01 (male, age 39), KEL03 (female, age 39), and KEL06 (female, age 35), S1 (female, age 51-55), S5 (male, age 21-25), S8 (female, age 26-30), S9 (female, age 51-55), and S12. Written informed consent was obtained from all subjects.

Soluble rHAs were expressed in baculovirus-infected insect cells, E. coli cells, or Expi293F cells. LAH-Fc fusion proteins and rigGs were expressed in Expi293F cells. IgG⁺ Bmem cells that bound fluorescently tagged rHA were isolated from healthy human donors before and 1 wk-2 wk after immunization with seasonal influenza vaccine. IgG-containing supernatants from cultures of individual Bmem cells were screened for reactivity to H1, H5, H3, and B HAs in Luminex assays, and V(D)J rearrangements encoding clonal IgGs of interest were recovered from cultured B cells by RT-PCR. Broadly binding clonal IgGs were expressed as rigGs for further characterization of binding breadth by ELISA, Luminex assay, flow cytometry, and BLI assays. The protective activity of S1V2-72 was tested in vitro by standard microneutralization assays with H1N1, H3N2, and IBV, and by an ADCC assay comprising co-incubated S1V2-72 or control mAbs (as mouse rIgG2c) plus HA-expressing target cells and effector cells in which luciferase activity was stimulated by mouse FcγRIV activation. In vivo protection was determined by i.p. injecting mouse IgG2c or IgG1 versions of S1V2-72 or control mAbs into female C57BL6/J mice 3 h prior to lethal intranasal challenge with H3N2 or IBV. Animals were weighed and monitored for survival daily. The immunogenicity of the S1V2-72 epitope was determined by footpad immunization of female C57BL6/J mice with B/MY04 EHA2 plus alum. Some mice were boosted with homologous antigen in the hock 4-5 weeks after priming. Eighteen days post-prime or 8 d post-boost, immune responses in the draining LN were analyzed by flow cytometry and single B-cell culture. Immune sera were also collected for analysis by ELISA and Luminex. The specificity of mouse GC B cells and serum IgG for the S1V2-72 epitope was determined by Luminex competitive binding assay. A competitive binding assay was also used to screen supernatants from cultured human Bmem cells, to identify S1V2-72-competing IgGs. For cryo-EM structural analysis, S1V2-72 Fab was incubated with B/MY04 EHA2 trimer in a 9:1 ratio to achieve 3 Fabs per trimer, and the complex was purified by gel filtration. S1V2-72: EHA2 complex was deposited onto 300 mesh Quantifoil Au 0.6/1.0 grids, blotted, and vitrified in liquid ethane. Micrographs were recorded on a 300 kV Titan Krios G3i microscope with a K3 direct electron detector. Dose-fractionated images were gain normalized, aligned, dose-weighted and summed using MotionCor2. CryoSPARC was used for contrast transfer function and defocus value estimation, particle picking, 2D classification, 3D reconstruction, and refinement. Atomic models of B/MY04 EHA2 and S1V2-72 Fab were predicted with AF2 and fit to the cryoEM map with UCSF ChimeraX and Phenix. V(D)J sequences for S1V2-72 and K06.18 are available at GenBank accession numbers OR542561 (SEQ ID NO:01), OR5425462 (SEQ ID NO:02), OR5425463 (SEQ ID NO:03), and OR542564 (SEQ ID NO:04). The map of the cryo-EM reconstruction of S1V2-72 Fab bound to EHA2 is available at the Electron Microscopy Data Bank (accession number EMD-42149). Coordinates for the docked model of S1V2-72 bound to EHA2 is available at the Protein Data Bank, accession number 8UDG.

Human Subjects. KEL01 and KEL03 received the trivalent inactivated seasonal influenza vaccine (TIV) 2014-2015 Fluvirin, which contained A/Christchurch/16/2010, NIB-74 (H1N1), A/Texas/50/2012, NYMC X-223 (H3N2), and B/Massachusetts/2/2012, NYMC BX-51B. KEL06 received the TIV 2015-2016 Flucelvax, which contained A/Brisbane/10/2010 (H1N1), A/South Australia/55/2014 (H3N2), and B/Utah/9/2014. Blood was drawn on day 14 post-vaccination, and PBMCs isolated by centrifugation over Ficoll density gradients (SepMate-50 tubes, StemCell Tech) were frozen and kept in liquid nitrogen until use.

Subjects S1, S5, S8, S9, and S12 met all of the following inclusion criteria for a study approved by the Boston University Institutional Review Board: between 18 and 65 years of age; in good health, as determined by vital signs [heart rate (<100 bpm), blood pressure (systolic≤140 mm Hg and ≥90 mm Hg, diastolic≤90 mm Hg), oral temperature (<100.0° F.)] and medical history to ensure existing medical diagnoses/conditions are not clinically significant; can understand and comply with study procedures, and; provided written informed consent prior to initiation of the study. Exclusion criteria included: (1) life-threating allergies, including an allergy to eggs; (2) have ever had a severe reaction after influenza vaccination; (3) a history of Guillain-Barre Syndrome; (4) a history of receiving immunoglobulin or other blood product within the 3 months prior to vaccination in this study; (5) received an experimental agent (vaccine, drug, biologic, device, blood product, or medication) within 1 month prior to vaccination in this study or expect to receive an experimental agent during this study; (6) have received any live licensed vaccines within 4 weeks or inactivated licensed vaccines within 2 weeks prior to the vaccination in this study or plan receipt of such vaccines within 2 weeks following the vaccination; (7) have an acute or chronic medical condition that might render vaccination unsafe, or interfere with the evaluation of humoral responses (includes, but is not limited to, known cardiac disease, chronic liver disease, significant renal disease, unstable or progressive neurological disorders, diabetes mellitus, autoimmune disorders and transplant recipients); (8) have an acute illness, including an oral temperature greater than 99.9° F., within 1 week of vaccination; (9) active HIV, hepatitis B, or hepatitis C infection; (10) a history of alcohol or drug abuse in the last 5 years; and (11) a history of a coagulation disorder or receiving medications that affect coagulation. Subjects S1, S5, S8, S9, and S12 received seasonal influenza vaccination during three consecutive North American flu seasons (2015-2016, 2016-2017, 2017-2018), and had blood drawn on day 0 (pre-vaccination) and day 7 (post-vaccination) each year. During the 2015-2016 season, the subjects received the TIV Fluvirin, which contained A/reassortant/NYMC X-181 (California/07/2009×NYMC X-157) (H1N1), A/South Australia/55/2014 IVR-175 (H3N2), and B/Phuket/3073/2013. During the 2016-2017 season, the subjects received the quadrivalent inactivated vaccine Flucelvax, containing A/Brisbane/10/2010 (H1N1), A/Hong Kong/4801/2014 (H3N2), B/Utah/9/2014, and B/Hong Kong/259/2010. During the 2017-2018 season, the subjects received the quadrivalent inactivated vaccine Flucelvax, containing A/Singapore/GP1908/2015 IVR-180 (H1N1), A/Singapore/GP2050/2015 (H3N2), B/Utah/9/2014, and B/Hong Kong/259/2010.

Mice. Female C57BL/6J mice (#000664) were obtained from the Jackson Laboratory and maintained under specific pathogen-free conditions at the Duke University Animal Care Facility. HA β-hairpin antibody-knockin mice harboring unrearranged, human germline IGHV1-2, IGHD3-22, and IGHJ4 gene segments knocked into the mouse Igh locus (β-hairpin HC^{+/+}), and the unrearranged, human germline IGLV2-23 and IGLJ3 and IGLC gene segments knocked into the mouse Igk locus (β-hairpin LC^{+/+}), and a human terminal deoxynucleotidyl transferase (hTDT) transgene, were housed in specific pathogen-free conditions at the Boston Children's Hospital animal care facility. Eight-week-old to 12-week-old mice were immunized in the hind footpad (prime) or ipsilateral hock (boost) with 10 μg of B/MY04 EHA2 adjuvanted with Alhydrogel® (Invivogen). All experiments involving animals were approved by the Duke University Institutional Animal Care and Use Committee or Boston Children's Hospital Institutional Animal Care and Use Committee.

Cell Line Culture. Unless otherwise noted, mammalian cell lines were maintained in static cultures at 37° C. with 5% CO₂ in a humidified incubator, and culture reagents were from Gibco. MS40L-low feeder cells (Mus musculus) (Luo X M, et al. (2009) Blood. 113:1422-1431; Su KY, (2016) J Immunol. 197:4163-4176) were expanded from frozen aliquots in Iscove's Modified Dulbecco's Medium containing 10% HyClone FBS (Cytiva), 2-mercaptoethanol (55 μM), penicillin (100 units/mL), and streptomycin (100 μg/mL). NB21.2D9 cells (Mus musculus) (Kuraoka M, et al. (2016) Immunity. 44:542-552) were cultured in Dulbecco's Modified Eagle Medium (DMEM) plus 10% FBS, penicillin, streptomycin, and 1×MEM non-essential amino acids. Expi293F cells (Homo sapiens; Thermo Fisher) were cultured in Expi293 Expression Medium plus penicillin and streptomycin, at 8% CO₂ with shaking. K530-derived cell lines (Homo sapiens) (Song S, et al. (2021) Commun Biol. 4:1338) were cultured in B-cell medium (BCM: RPMI-1640 medium plus 10% FBS, 2-mercaptoethanol (55 μM), penicillin, streptomycin, HEPES (10 mM), sodium pyruvate (1 mM), and MEM nonessential amino acids). Madin-Darby canine kidney (MDCK) cells (Canis lupus familiaris) were maintained in DMEM plus 10% FBS. High Five cells (BTI-TN-5B1-4; Trichoplusia ni; Thermo Fisher) were maintained in ESF 921 medium (Expression Systems) at 28° C. in spinner flasks in air. Cell lines were not subject to authentication.

Recombinant HA Expression and Purification. Except where explicitly noted otherwise in the figures or text, all recombinant HAs were full-length soluble ectodomain (FLSE) trimers. Recombinant HAs (except for EHA2s) were expressed by infection of insect cells with recombinant baculovirus as described (Whittle J R, et al. (2011) Proc Natl Acad Sci USA. 108:14216-14221; Xu H, et al. (2015) Proteins. 83:771-780; Schmidt A G, et al. (2013) Proc Natl Acad Sci USA. 110:264-269; Raymond D D, et al. (2016) Nat Med. 22:1465-1469). In brief, synthetic DNA corresponding to the full-length ectodomain or globular HA-head were subcloned into a pFastBac vector modified to encode a C-terminal thrombin cleavage site, a T4 fibritin (foldon) trimerization tag, and a hexa-His tag. The resulting baculoviruses produce HA trimers and trimeric HA heads. Supernatant from recombinant baculovirus-infected High Five cells was harvested 72 h post-infection and clarified by centrifugation. Proteins were purified by adsorption to cobalt-nitrilotriacetic acid (Co-NTA) agarose resin (Takara), followed by a wash in buffer A (10 mM Tris, 150 mM NaCl, pH 7.5) plus 5 mM imidazole, elution in buffer A plus 350 mM imidazole (pH 8), and gel filtration chromatography on a Superdex 200 column (GE Healthcare) in buffer A.

The following HAs were thus produced: H1N1 A/USSR/90/1977 (H1/USSR77), H1N1 A/Massachusetts/1/1990 (H1/MA90), H1N1 A/Florida/2/1993 (H1/FL93), H1N1 A/Solomon Islands/03/2006 (H1/SI06), H1N1 A/California/04/2009 (H1/CA09), H1N1 A/reassortant/NYMC X-181 (California/07/2009 NYMC X-157) (H1/X181), H1N1 A/Michigan/45/2015 X-275 (H1/MI15 X-275), H1N1 A/Brisbane/2/2018 IVR-190 (H1/BN18 IVR-190), H3N2 A/Aichi/2/1968 (H3/X31), H3N2 A/Texas/1/1977 (H3/TX77), H3N2 A/Bangkok/01/1979 (H3/BK79), H3N2 A/Philippines/2/1982 (H3/PH82), H3N2 A/Leningrad/360/1986 (H3/LG86), H3N2 A/Wisconsin/67/2005 (H3/WI05), H3N2 A/South Australia/55/2014 IVR-175 (H3/IVR175), H3N2 A/Hong Kong/4801/2014 (H3/HK14), H3N2 A/Singapore/INFIMH-16-0019/2016 (H3/SP16), H3N2 A/Kansas/14/2017 X-327 (H3/KS17) and point mutants thereof (9), H4 A/American black duck/New Brunswick/00464/2010 (H4/NB10), H5 A/Vietnam/1203/2004 (H5/VN04), B/Malaysia/2506/2004 (B/MY04), B/Brisbane/60/2008 (B/BN08), B/Phuket/3073/2013 (B/PK13), B/Florida/84/2017 (B/FL17); and trimeric, head-only constructs rHA of H3/WI05 (H3/WI05-h), H3 A/Johannesburg/33/1994 (H3/J′burg-h) and H3/X31 (H3/X31-h).

EHA2 Expression and Purification. Synthetic DNA encoding recombinant post-fusion HA2 (Chen J, et al. (1999) Proc Natl Acad Sci USA. 96:8967-8972) from IBV or various IAVs (Table 16) was cloned into a pET vector modified to include an N-terminal hexa-His tag and tobacco etch virus (TEV) protease cleavage site. Plasmid-transformed Rosetta (DE3) E. coli clones (Novagen) were inoculated into 50 mL cultures of 2×YT or LB medium and grown overnight at 37° C. with shaking. The following morning, the 50 mL cultures were used to inoculate 1 L cultures of 2×YT or LB medium. The 1 L cultures were incubated at 37° C. with shaking until the OD600 reached 0.6-0.8, at which point EHA2 expression was induced by addition of isopropyl β-D-1-thiogalactopyranoside (1 mM final). The cultures were then incubated overnight at 18° C. with shaking. Cells were harvested by centrifugation, suspended in lysis buffer (40 mM Tris pH 8, 500 mM NaCl, 2 mM 2-mercaptoethanol, 10 mM imidazole), and disrupted by ultrasonication on ice. The EHA2-containing lysate was clarified by centrifugation at 40,000×g for 30 min at 4° C., then EHA2 was purified from the supernatant fluid by adsorption to Co-NTA agarose resin, followed by extensive washing in 40 mM Tris pH 8 plus 100 mM NaCl, 10% glycerol, 2 mM 2-mercaptoethanol, and 30 mM imidazole. EHA2 was eluted in buffer A plus 400 mM imidazole. Alternatively, synthetic DNA encoding recombinant post-fusion HA2 was cloned into pVRC with an N-terminal secretion signal, hexa-His tag, and HRV3C protease cleavage site. The resultant vector was transfected into Expi293F cells, and HA2-containing culture supernatant was harvested five days post-transfection and clarified by centrifugation. EHA2 was purified from the supernatant by adsorption to Co-NTA agarose resin, as described above. For immunization and cryo-EM studies, the hexa-His tag was then removed by incubating the EHA2 with TEV protease or HRV3C protease, and the tag-less EHA2 was isolated by gel filtration chromatography on a Superdex 200 column in PBS.

Generation of HA-Expressing K530 Cell Lines. Monoclonal, fluorescent protein-barcoded K530 cell lines stably expressing HAs from A/California/04/2009 (H1/CA09; GenBank accession FJ966082), B/Malaysia/2506/2004 (B/MY04; GenBank CY038287), or B/reassortant/NYMC BX-51B (Massachusetts/2/2012×NYMC BX-46) (B/MA12; GenBank KF752446) were prepared by lentiviral transduction and single-cell sorting, as described (Song S, et al. (2021) Commun Biol. 4:1338).

Flow Cytometry. Human Bmem cells were isolated by flow cytometry as described (McCarthy K R, et al. Immunity. 48:174-184 e179). PBMCs in DMEM containing 10% FBS were blocked with mouse IgG1 (MG1K; Rockland) and then labeled with anti-human IgM-FITC (MHM-88), anti-CD3-PE-Cy5 (UCHT1), anti-CD14-Tri (TuK4), anti-CD16-PE-Cy5 (3G8), anti-CD19-PE-Cy7 (HIB19), anti-IgG-APC (G18-145), anti-IgD-APC-Cy7 (IA6-2), anti-CD27-BV421 (M-T271), and anti-CD24-BV510 (ML5), purchased from BD Biosciences, BioLegend, or Thermo Scientific. To isolate HA-binding Bmem cells from donors KEL01, KEL03, and KEL06, PE-labeled H3/WI05 was prepared using R-Phycoerythrin Labeling Kit-NH2 (Dojindo). To isolate HA-binding Bmem cells from donors S1, S5, S8, S9, S12, and in some experiments KEL01 and KEL03, a mixture of PE-labeled H1/X181, PE-labeled H3/IVR175, and PE-labeled B/PK13 was used. HA-binding (PE+) CD19+CD27+CD24hi IgD-IgM-IgG+ cells were sorted on a FACS Vantage cytometer with Diva software (BD Biosciences). Flow cytometric data were analyzed with FlowJo software (Treestar Inc.). Doublets were excluded by FSC-A vs. FSC-H gating. Cells positive for 7-AAD (BD Bioscience) or for CD3, CD14, or CD16 expression were also excluded.

Mouse popliteal LNs were dispersed by gentle grinding between frosted glass slides. Dispersed cells were suspended in DMEM plus 10% FBS, 2-mercaptoethanol (55 μM), 2 mM additional L-glutamine, penicillin, and streptomycin, then labeled with fluorophore-conjugated Abs: anti-B220 BV785 (RA3-6B2, BioLegend), anti-CD38 PE-Cy7 (90, BioLegend), GL7 FITC (BD Biosciences), anti-CD138 BV605 (281-2, BD), and anti-IgD BV480 (11-26c.2a, BD). GC B cells were identified as B220hi CD138-CD38lo GL-7+IgD-, mature follicular B cells as B220hi CD138-CD38hiGL-7-IgD+, and PCs as B220-CD138+.

Dead cells and doublets were excluded from analysis based on propidium iodide (Sigma-Aldrich) staining and FSC-A/FSC-H gating, respectively. Labeled cells were analyzed or sorted into culture plates with a BDFACSymphony A5 analyzer or BD FACSymphony S6 sorter. K530-derived cell lines were thawed from cryopreserved aliquots and expanded in culture for ≥3 days. Pooled K530 cells were incubated at room temperature (RT) for 25-30 min with 2 μg/mL anti-HA rIgGs or irrelevant human IgG (151L, Southern Biotech) diluted in PBS plus 2% fetal bovine serum. After washing, cells were labeled with 2 μg/mL PE-conjugated goat anti-human IgG (Southern Biotech) for 20-30 min at RT. Cells were then washed and analyzed with a BD FACS Canto II, a BD FACSymphony A5, or a BD LSRII flow cytometer.

Human Single B-Cell Culture (Nojima Culture). Human Bmem cells were expanded in the presence of MS40L-low feeder cells as described (Luo X M, et al. (2009) Blood. 113:1422-1431; Su K Y, (2016) J Immunol. 197:4163-4176; McCarthy K R, et al. Immunity. 48:174-184 e179). Single human Bmem cells were directly sorted into separate wells of 96-well plates and cultured with MS40L-low feeder cells in BCM supplemented with exogenous recombinant human IL-2 (50 ng/ml), IL-4 (10 ng/ml), IL-21 (10 ng/mL) and BAFF (10 ng/ml; all Peprotech). Cultures were maintained at 37° C. with 5% CO₂. Half of the culture medium was replaced twice weekly with fresh BCM plus cytokines. On culture day 25, culture supernatants were harvested for screening the reactivity of secreted clonal IgGs. Expanded clonal B cells were frozen for subsequent V(D)J sequence analysis.

Mouse Single B-Cell Culture (Nojima Culture). Mouse GC B or MF B cells were expanded in the presence of NB21.2D9 feeder cells essentially as described (Song S, et al. (2021) Commun Biol. 4:1338). Single B cells were directly sorted into separate wells of 96-well plates pre-seeded with NB21.2D9 cells in 200 μL BCM supplemented with recombinant murine IL-4 (2 ng/ml; Peprotech). Cultures were maintained at 37° C. with 5% CO₂. After two days of culture, 100 μL of culture medium was removed and replaced with 200 μL of fresh BCM (without IL-4). On culture days 3, 5, 6, 7 and 8, 200 μl of culture medium was replaced with fresh BCM. On culture day 10, culture supernatants were harvested for screening the reactivity of secreted clonal IgGs.

Ab V(D)J Rearrangement Amplification and Analysis. Rearranged V(D)J gene sequences for human Bmem cells from single-cell cultures were obtained by RT-PCR as described (McCarthy K R, et al. Immunity. 48:174-184 e179; Kuraoka M, et al. (2022) mBio 13: e0254622), DNA sequencing was performed at the Duke University DNA Analysis Facility. V(D)J rearrangements were identified with Cloanalyst (Kepler T B, et al. (2013) F1000Research. 2:103) and IMGT/V-QUEST (Brochet X, et al. (2008) Nucleic Acids Res. 36: W503-508).

Recombinant IgG Expression and Purification. DNA encoding H- or L-chain variable domains was cloned into expression vectors harboring the constant regions of mouse or human IgG1, Igκ, Igλ, or mouse IgG2c. IgGs were produced by transient transfection of Expi293F cells with the Expifectamine 293 transfection kit (Thermo Fisher), according to the manufacturer's instructions. Five days post-transfection, supernatants were harvested, clarified by low-speed centrifugation, mixed 1:1 with Protein G binding buffer (for mouse IgG1) or Protein A binding buffer (for human IgG1 or mouse IgG2c), and incubated overnight with Pierce Protein G or Protein A agarose resin (Thermo Fisher). The resin was collected in a chromatography column, washed with binding buffer, eluted in Pierce6 IgG Elution Buffer (Thermo Fisher), neutralized by 1M Tris (pH 9), and dialyzed into PBS. IgG concentrations were determined with a NanoDrop spectrophotometer (Thermo Fisher).

Recombinant Fab Purification. S1V2-72 Fab was generated from recombinant S1V2-72 human IgG1 with the Pierce Fab Preparation Kit (Thermo Fisher), according to the manufacturer's instructions.

LAH Fusion Proteins. LAH peptides were expressed as human IgG1 Fc fusion proteins in Expi293F cells and purified with Protein A resin (Thermo Fisher) according to the manufacturer's protocol. Purified LAH-Fc fusion proteins were stored in PBS at 4° C.

TABLE 17
Exemplary LAH Peptides

| LAH | |
|---|---|
| Peptide | Sequence (Sequence Identifier) |
| H3/X31 | RIQDLEKYVEDTKIDLWSYNAELLVALENQHTIDLTDSEM |
| | NKLFEKTRRQLRENA (SEQ ID NO: 56) |
| H5/VN04 | ERRIENLNKKMEDGFLDVWTYNAELLVLMENERTLDFHDS |
| | NVKNLYDKVRLOLRDNA (SEQ ID NO: 57) |
| B/MY04 | HNEILELDEKVDDLRADTISSQIELAVLLSNEGIINSEDE |
| | HLLALERKLKKMLGPS (SEQ ID NO: 58) |

Multiplex Bead Assay. The specificity and avidity of rIgGs and clonal IgGs in culture supernatants were determined by Luminex multiplex assay (Luminex Corp.) (McCarthy K R, et al. Immunity. 48:174-184 e179; Watanabe A, et al. (2019) Cell. 177:1124-1135 e1116). Culture supernatants or rIgGs were diluted in Luminex assay buffer (PBS plus 1% BSA, 0.05% NaN3 and 0.05% Tween20) plus 1% non-fat milk and incubated for 2 h at RT (or overnight at 4° C.) with a mixture of antigen-coupled microsphere beads in 96-well filter-bottom plates (Millipore). After washing with assay buffer, beads were incubated at RT for 1 h (or overnight at 4° C.) with PE-conjugated rat anti-mouse IgG1 (SB77e), mouse anti-human IgG Fc (JDC-10), goat anti-human IgG, goat anti-mouse IgG (all from Southern Biotech), or rat anti-mouse IgG2a (RMG2a-62, BioLegend). After washing, the beads were suspended in assay buffer and analyzed on a Bio-Plex 3D Suspension Array System (Bio-Rad). The following antigens were coupled with carboxylated beads (Luminex Corp): BSA, goat anti-human Igk, goat anti-human Ig) (both Southern Biotech), goat anti-human IgG Fc (Jackson ImmunoResearch), and rHAs.

ELISA. High-binding 96- or 384-well microplates were coated with 2 μg/mL protein in 0.1 M sodium carbonate buffer (pH 9) overnight at 4° C. For quantitation of secreted IgG in B-cell culture supernatants, the coating proteins were goat anti-human Igk plus goat anti-human Igλ, or goat anti-mouse Igk plus goat anti-mouse Ig), or goat anti-mouse Igλ plus goat anti-human Igλ (all Southern Biotech). Otherwise, the coating proteins were rHA constructs. After washing with PBS plus 0.1% Tween20, the plates were blocked with PBS plus 0.5% BSA at RT for ≥30 min, then culture supernatants or rIgGs diluted in PBS plus 0.5% BSA and 0.1% Tween20 were added to the plates and incubated for 2 h at RT, or overnight at 4° C. After extensive washing, bound Abs were detected with goat anti-human IgG-HRP, goat anti-mouse IgG-HRP, or a mixture of goat anti-human Igκ-HRP and goat anti-human Igλ-HRP (all Southern Biotech) diluted in PBS plus 0.5% BSA and 0.1% Tween20. After washing, HRP activity was detected with the TMB substrate kit (BioLegend) and a Spectramax plate reader (Molecular Devices). Background signal at 650 nm was subtracted from the signal at 450 nm to calculate the OD450.

Competitive Inhibition Assay. Ab epitope mapping was performed by competitive binding inhibition in ELISA or Luminex assay (McCarthy K R, et al. Immunity. 48:174-184 e179; Watanabe A, et al. (2019) Cell. 177:1124-1135 e1116). In all experiments, competitor IgGs were from a different species than the analyte Ab (mouse vs. human, or vice versa). Generally, serially diluted competitor rIgGs or mouse sera were incubated with HA-coated substrate (either Luminex microspheres or ELISA plates) for 2 h at RT or overnight at 4° C. Analyte Ab (either K06.18 rIgG or S1V2-72 rIgG) was then added at a fixed concentration to each well, and incubated with the competitor Abs and HA substrate for 2 h at RT. After washing, bound analyte Ab was detected either by mouse anti-human IgG Fc-PE (JDC-10, SouthernBiotech), rat anti-mouse IgG2a-PE (RMG2a-62, BioLegend) or goat anti-mouse IgG Fc-HRP (SouthernBiotech).

In some experiments, the competitor Igs were diluted (1:10) supernatants from cultures of human Bmem cells, rather than rIgGs. Culture supernatants containing HA-nonbinding IgG were used as non-inhibiting controls.

To map the epitopes of mouse GC B cells elicited by immunization with B/MY04 EHA2, S1V2-72 human rIgG or irrelevant human IgG (151L, SouthernBiotech) was incubated with B/MY04 EHA2-conjugated microspheres for 3 2 h, then diluted (1:10 final) supernatants from cultures of mouse GC B cells were added to each well and incubated for 2 h at RT. The final concentration of S1V2-72 or irrelevant IgG was 10 μg/mL. After washing, bound mouse Ab was detected by rat anti-mouse IgG1-PE (SB77e, SouthernBiotech). To calculate binding as a percent of control, GC B-cell IgG binding signal in the presence of S1V2-72 was divided by GC B-cell IgG binding signal in the presence of irrelevant IgG, then multiplied by 100.

Biolayer Interferometry (BLI). BLI experiments were performed on a BLItz label-free protein analysis system (ForteBIO). All measurements were in Luminex assay buffer at RT. Purified B/MY04 FLSE or EHA2 was immobilized on Ni-NTA biosensors (Sartorius), and Fabs or IgGs were titrated to determine relative association rates. Alternatively, IgGs were immobilized on Protein A biosensors (Sartorius), and B/MY04 FLSE was titrated to determine relative association rates.

Mouse ADCC Reporter Assay. The potential for mAbs to mediate ADCC activity was determined using the Mouse FcγRIV ADCC Bioassay (Promega) according to manufacturer's instructions. Briefly, cloned H3/X31- or B/MY04-expressing K530 cells (target cells) were dispensed at 2.5×104 cells/well into white, flat-bottom 96-well assay plates (Corning #3917). Serially diluted mouse IgG2c Abs were added to the target cells and incubated at RT for 15 min. Effector cells expressing mouse FcgRIV were added to the wells for an effector: target ratio of 3:1, then the plate was incubated at 37° C., 5% CO₂ for 6 h. Finally, Bio-Glo Reagent was added to the plate and incubated for 15 min at RT. Luminescence was detected with a Synergy HTX multimode plate reader (BioTek).

Background signal from wells containing no cells was subtracted from the data, and fold-induction was calculated as the quotient of signal in wells containing Ab divided by the signal in wells containing no Ab. Antibodies were assayed in duplicate.

Virus Propagation. Influenza viruses A/Solomon Islands/3/2006 (H1N1), A/Aichi/2/68, X-31 (H3N2), and B/Malaysia/2506/2004 were propagated in embryonated, specific-pathogen-free, chicken eggs (Sunrise Farms Inc., Catskill, NY or Charles River Avian Vaccine Services, Norwich, CT) or MDCK cells (NBL-2; ATCC® CCL-34™), as described (McCarthy K R, et al. Immunity. 48:174-184 e179). Stocks were harvested as pooled and clarified cell culture supernatant/lysate or egg allantoic/amniotic fluid and stored at −80° C. Stocks were titered by tissue culture infectious dose 50 percent (TCID₅₀) assay as described in standard protocols.

In Vivo Protection Experiments. Mice were injected i.p. with 3-100 μg of rIgGs diluted to 200 μL in PBS. Three hours later, mice were anesthetized by i.p. injection of ketamine (85 mg/mL) and xylazine (13 mg/mL) and infected intranasally by either ≥10×LD₅₀ (5×10⁴ PFU; the LD50 was determined to be between 1.5×10³ PFU and 5×10³ PFU) of B/Malaysia/2506/2004, or 3×LD₅₀ (1.5×10⁴ PFU) or 10×LD₅₀ (5×10⁴ PFU) of A/Aichi/2/1968 X-31 (H3N2) in 40 μL volumes. X-31 typically was used at a lower dose than B/Malaysia due to the former's greater virulence, demonstrated by more rapid weight loss after infection. Mice were monitored daily for survival and body weight loss until 13 days post-challenge. The humane endpoint was set at 20% body weight loss relative to the initial body weight at the time of infection.

Influenza Microneutralization Assay. Virus neutralization endpoint titers were determined using the influenza microneutralization assay on Madin-Darby Canine Kidney (MDCK) cells (London Line, FR-58) as described (McCarthy K R, et al. (2021) Proc Natl Acad Sci USA. 118: e2026752118; CDC. (2007) Influenza virus microneutralization assay; CDC. (2009) Influenza Virus Microneutralization Assay H1N1 Pandemic Response; WHO. (2010) Serological diagnosis of influenza by microneutralization assay; WHO. (2011) Manual for the laboratory diagnosis and virological surveillance of influenza). Monoclonal Abs were diluted to 100 μg/mL in assay diluent and then serially two-fold diluted. Neutralizing antisera served as neutralization controls. Diluted Abs were mixed with 100 TCID₅₀ of influenza virus, dispensed to microtiter plates, and incubated for 60 min at 37° C., 5% CO₂. MDCK cells (1.5×10⁴) were added to each well, then incubated overnight at 37° C., 5% CO₂. The cells were washed once in PBS, fixed with pre-chilled (−20° C.) 80% acetone and incubated at RT for 10 min, then air-dried. After washing the fixed cells, primary antibody (mouse anti-influenza A nucleoprotein, Millipore MAB8251; or mouse anti-influenza B nucleoprotein, Millipore MAB8661) diluted 1:4,000 was incubated with the cells for 60 min at RT, followed by extensive washing. Secondary antibody (HRP-conjugated goat anti-mouse IgG; KPL 474-1802; 1:4000) was incubated with cells for 60 min at RT, followed by extensive washing and HRP substrate development at RT. The HRP reaction was stopped with 0.5 N sulfuric acid. Absorbance was measured at 490 nM wavelength in a Synergy H1 automated microplate reader (BioTek Instruments). Wells with absorbance values≤50% of virus-only control wells were scored as neutralization positive. Data were expressed as the mean of the reciprocal of the final dilution that was positive for neutralization. All samples were assayed in duplicate.

Reagents obtained through BEI Resources, NIAID, NIH include: influenza A virus A/Aichi/2/1968 (H3N2), NR-3177; polyclonal influenza virus A/Aichi/2/1968 (H3N2) serum (guinea pig), NR-3126. Influenza A virus A/Solomon Islands/3/2006 (H1N1; FR-331) and MDCK London cells (FR-58) were obtained through the International Reagent Resource (formerly the Influenza Reagent Resource), Influenza Division, WHO Collaborating Center for Surveillance, Epidemiology and Control of Influenza, Centers for Disease Control and Prevention, Atlanta, GA, USA.

Cryo-EM Sample Preparation. B/MY04 EHA2 was incubated with 3-fold molar excess (per EHA2 polypeptide chain) of S1V2-72 Fab at 4° C. for 60 min. Fab: EHA2 complex was isolated by size-exclusion chromatography over a Superdex 200 Increase 10/300 GL column (GE Healthcare) equilibrated (McCarthy K R, et al. (2021) Proc Natl Acad Sci USA. 118: e2026752118) in PBS. Pooled fractions containing the Fab: EHA2 complex were concentrated to 3 mg/mL. 3.0 μL of sample was deposited onto 300 mesh Quantifoil Au 0.6/1.0 grids that had been glow discharged in a PELCO easiGLOW (Ted Pella) at 0.39 mBar, 15 mA for 30 s. Samples were vitrified in 100% liquid ethane using a Vitrobot Mark IV (Thermo Fisher Scientific), with a wait time of 10 s, blot time of 8 s and a blot force of 6 at 100% humidity.

Cryo-EM Data Collection and Processing. Cryo-EM data were recorded on a 300 kV Titan Krios G3i Microscope (Thermo Fisher Scientific) equipped with a K3 direct electron detector (Gatan) and a GIF quantum energy filter (20 eV; Gatan) at the Harvard Cryo-Electron Microscopy Center for Structural Biology at Harvard Medical School. Data were acquired in counting mode, using image shift and real-time coma correction by beamtilt with the automated data collection software SerialEM (Mastronarde D N, et al. (2005) J Struct Biol. 152:36-51); nine holes were visited per stage position, acquiring one movie per hole. Details of the data collection and dataset parameters are summarized Table 19. Dose-fractionated images were gain normalized, aligned, dose-weighted and summed using MotionCor2 (Zheng S Q, et al. (2017) Nat Methods. 14:331-332). Patch contrast transfer function (CTF) and defocus value estimation were performed using CryoSPARC (Punjani S Q, (2017) Nat Methods. 14:290-296). Details of the data processing strategy are shown in McCarthy K R, et al. (2021) Proc Natl Acad Sci USA. 118: e2026752118. In short, particle picking in a subset of micrographs was carried out using CryoSPARC, followed by 2D classification and culling of junk classes within CryoSPARC. Selected 2D classes were used as templates to pick particles from a larger subset of micrographs, which, after 2D classification, were culled to 609,447 particles. Ab-initio reconstruction within CryoSPARC yielded an initial model (from 246,000 particles) that was used to create 2D templates for another round of particle picking. This time, particles were picked from all 14,273 micrographs; these particles were culled by multiple rounds of 2D classification to select the best-looking classes of particles appearing to include 2 Fabs bound to an EHA2 rod (282,599 particles). After Ab-Initio reconstruction and heterogeneous refinement of five 3D classes from these particles, the 135,398 particles in the three best-looking classes were used for another round of ab-initio reconstruction with three 3D classes. The largest and best-looking 3D class (59,527 particles) was subjected to homogeneous refinement and local refinement within CryoSPARC to produce the final ˜5 Å reconstruction. UCSF ChimeraX (Goddard T D, et al. (2018) Protein Sci. 27:14-25; Pettersen E F, et al. (2021) Protein Sci. 30:70-82) was used to fit the AF2-predicted models of EHA2 and S1V2-72 to cryo-EM map. Structural biology applications used in this project were compiled and configured by SBGrid (Morin A, et al. (2013) Elife. 2: e01456).

Statistical Analyses. All statistical analyses were performed using GraphPad Prism (v10) software. Statistical significance of differences in mouse survival after lethal influenza challenge was calculated by the log-rank (Mantel-Cox) test, with Bonferroni's correction for multiple comparisons applied. For flow cytometry and serological analyses in EHA2-immunized mice, data were log-transformed to achieve normal distribution, then statistically significant differences between groups were detected by Welch's version of one-way ANOVA. Where ANOVA detected significant differences in means (P<0.05), the multiplicity-adjusted P-values of Dunnett's T3 post-test were reported with asterisks on the corresponding graphs. *, P<0.05; **, P<0.01; ***, P<0.001.

### Example 1

### Identification of IAV and IBV Cross-Reactive HA Antibodies

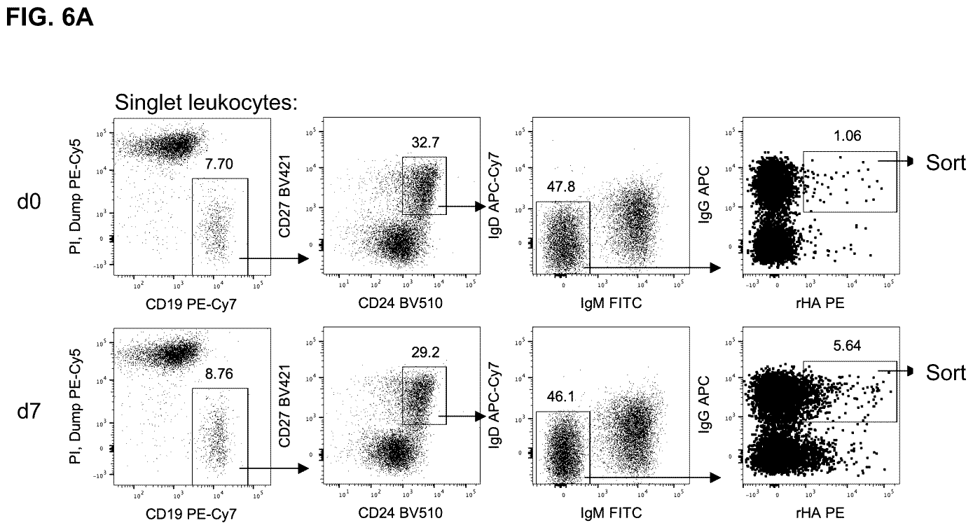

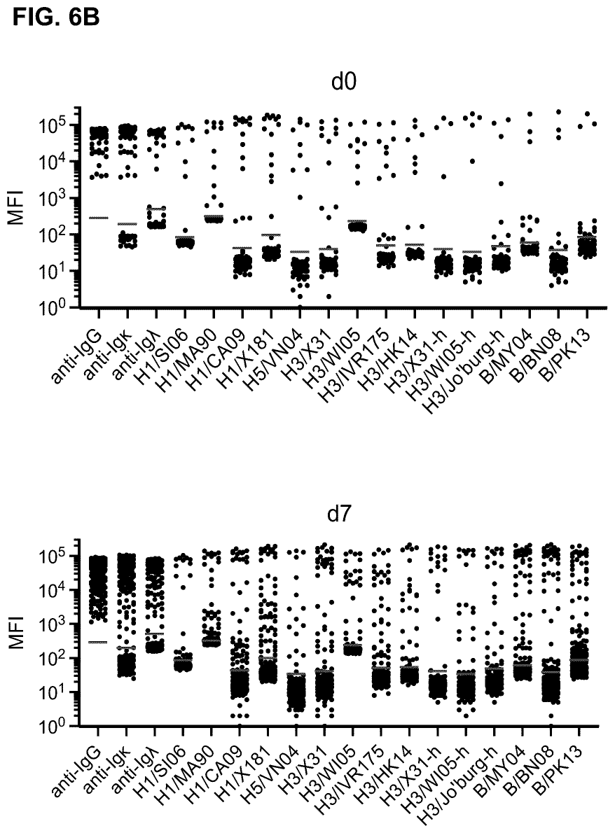

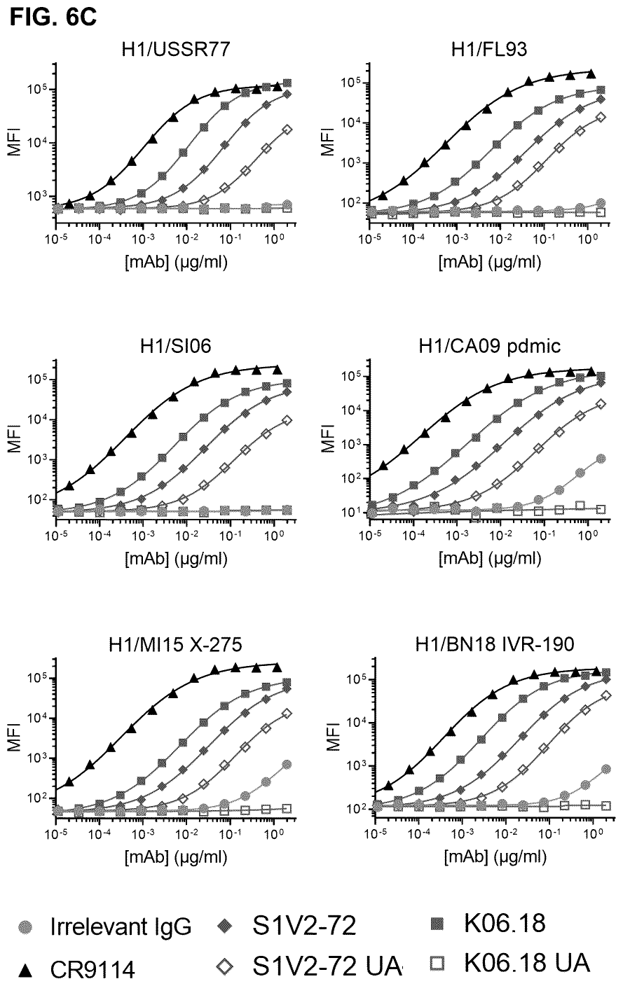

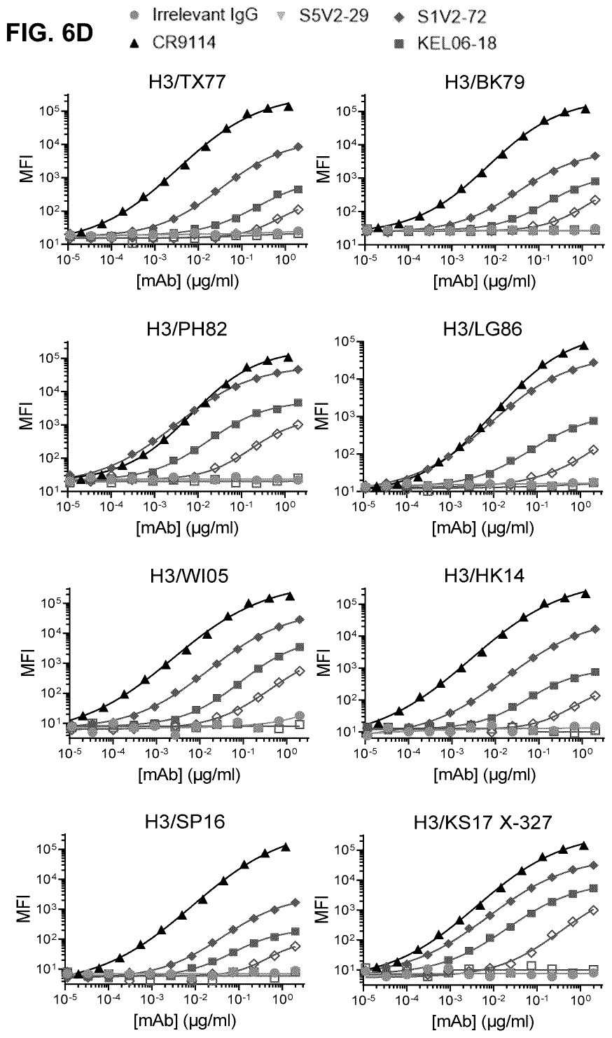

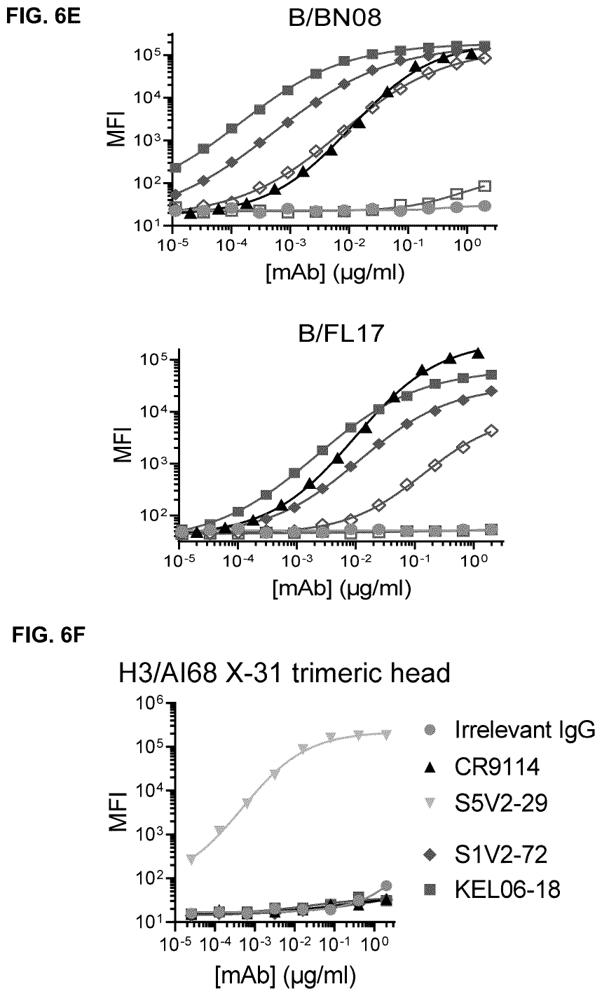

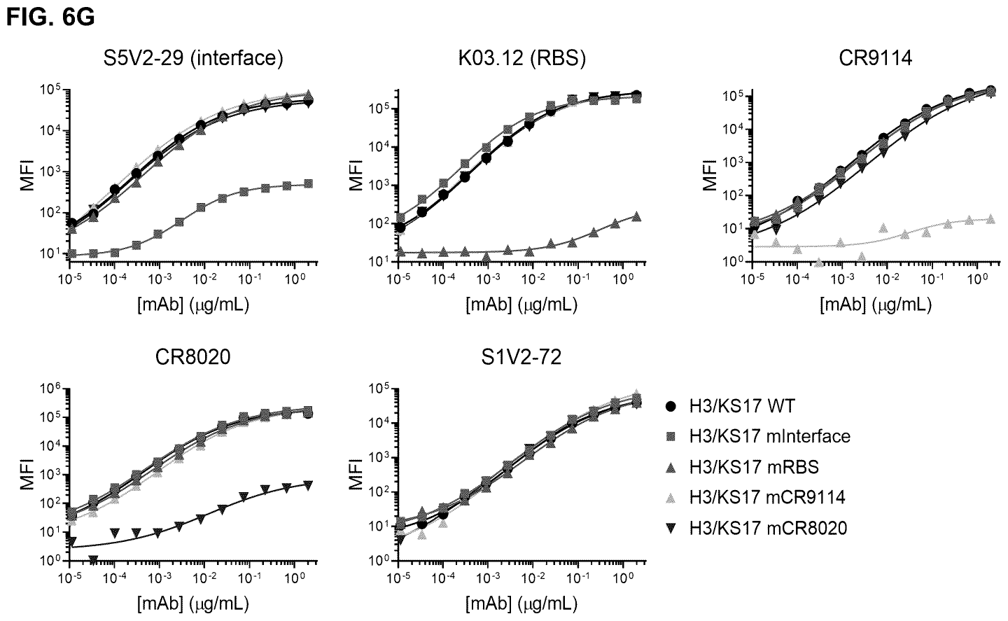

To identify IAV- and IBV-cross-reactive HA Abs, individual HA-binding IgG⁺ memory B (Bmem) cells sorted from peripheral blood mononuclear cells (PBMCs) of eight human donors before or 1-2 weeks after seasonal influenza vaccination were cultured. (FIG. 6A) (Watanabe A, et al. (2019) Cell. 177:1124-1135 e1116; McCarthy K R, et al. (2018) Immunity 48:174-184 e179). A multiplex binding assay was used to screen the secreted clonal IgGs for reactivity against a panel of recombinant Has (rHAs) (FIG. 6B and Table 17)

TABLE 18
Summary of Culture Screening Results for New^{a} Human Nojima.

| Donor ID | No. Wells Screened | No. IgG^{+b} | No. rHA^{+c} |
|---|---|---|---|
| KEL01 | 96 | 79 | 13 |
| KEL03 | 384 | 336 | 84 |
| S1 | 1,572 | 1,139 | 221 |
| S5 | 1,152 | 880 | 87 |
| S8 | 1,788 | 1,310 | 208 |
| S9 | 338 | 221 | 34 |
| S12 | 1,116 | 765 | 80 |
| Total | 6,446 | 4,730 | 727 |

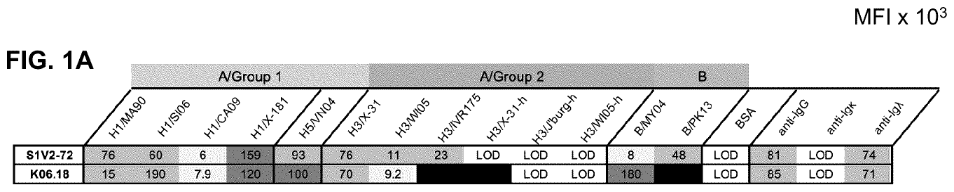

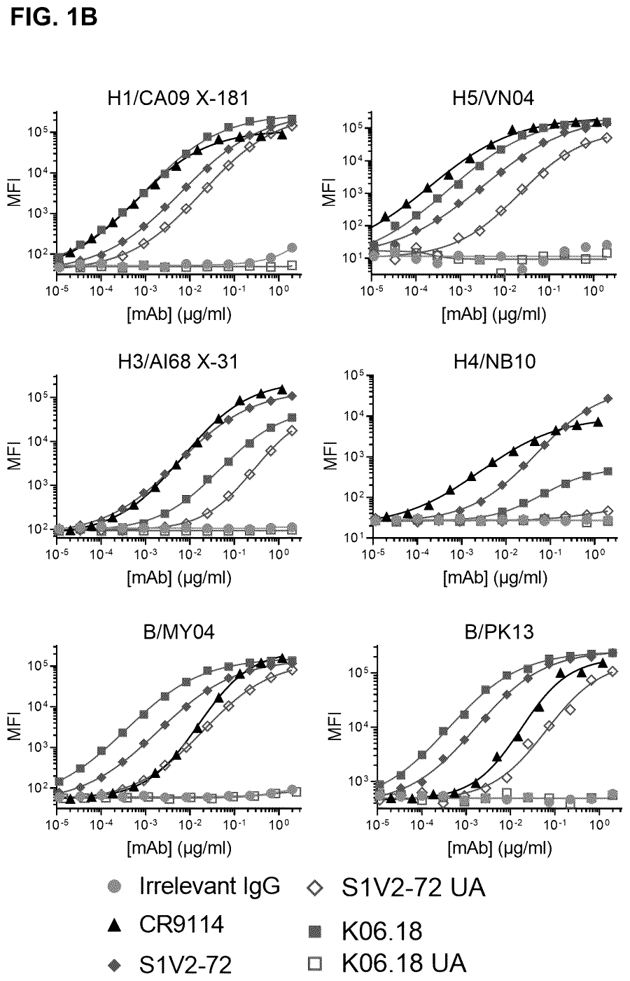

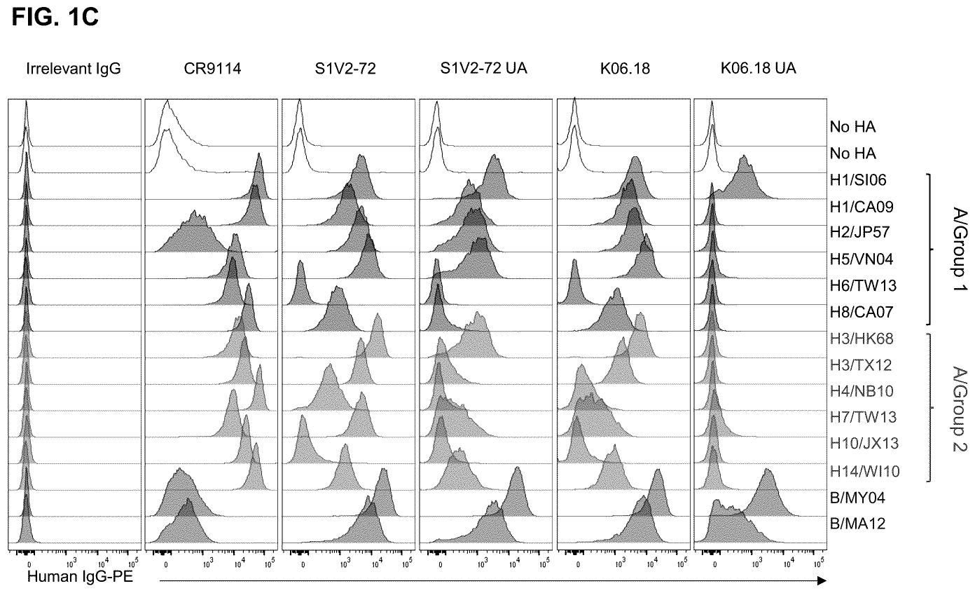

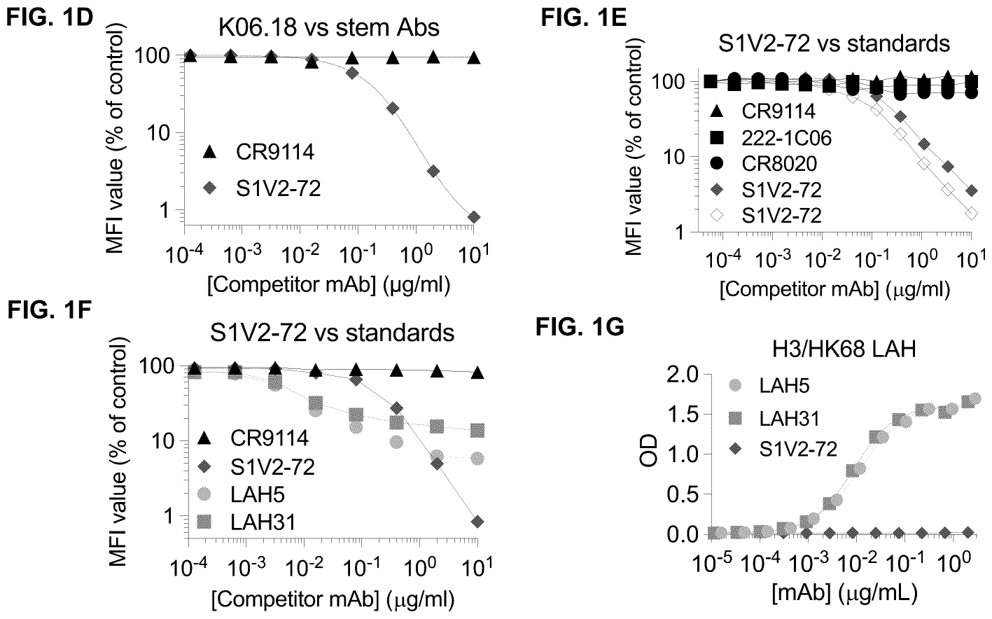

In Table 18, the following applies: (i) superscript a (a) means additional (n=915) HA-binding culture supernatant IgGs used in this study have been reported already (11, 15); (ii) superscript b (b) means the number of IgG-positive wells, determined by Luminex binding assay; and (iii) superscript c (c) means the number of HA-binding IgGs, determined by Luminex binding assay. From eight donors, 1,642 IgG+ HA-binding cultures were recovered. Two clonal Abs (S1V2-72 and K06.18), from unrelated donors, were identified that bound rHAs from group 1 (H1 and H5) and group 2 (H3) IAV strains, as well as from the Victoria and Yamagata lineages of IBV (FIG. 1A).

S1V2-72 is encoded by IGHV1-2*02/IGHD5-12*01/IGHJ5*02 and IGLV2-23*01/IGLJ3*02, with 10.5% and 3.8% somatic mutations in the heavy (H) and light (L) chain variable genes, respectively. K06.18 is encoded by similar V(D)J rearrangements (IGHV1-2*07/IGHD6-13*01/IGHJ4*02 and IGLV2-23*01/IGLJ3*02) and is also substantially mutated in the H-(6.0%) and L-chain (4.7%) variable regions (McCarthy K R, et al. (2018) Immunity 48:174-184 e179). No other Bmem cells clonally related to S1V2-72 or K06.18 were recovered from donors S1 or KEL06.

Recombinant S1V2-72 and K06.18 bound H1 HAs spanning ≥41 years of antigenic evolution, H3 HAs spanning ≥49 years, H4/NB10, H5/VN04, and HAs from the Yamagata and Victoria lineages of IBV (FIG. 1B and FIG. 6C-FIG. 6E). The inferred unmutated ancestor (UA) (Bonsignori M, et al. (2011) J Virol. 85:9998-10009) of S1V2-72 also bound many of these HAs with substantial avidity, whereas the K06.18 UA did not bind any of the rHAs screened by Luminex assays. In addition to binding bead-conjugated rHAs, S1V2-72 and K06.18 rAbs bound IAV and IBV rHAs expressed on the surface of the K530 cell line (FIG. 1C) (Song S, et al. (2021) Commun Biol. 4:1338). The S1V2-72 UA and K06.18 UA Abs also bound cell-surface rHAs, although less avidly than their mutated descendants, indicating that selection for enhanced binding to HA encountered by infection or vaccination drove affinity maturation of S1V2-72 and K06.18. That the K06.18 UA Ab bound to some cell-surface HAs, but not to the corresponding HAs conjugated to Luminex beads, indicates that cell-surface display better exposes the relevant epitope. S1V2-72 and K06.18 bound full-length HA trimers, but not “head-only” trimer constructs lacking the HA stem region (FIG. 1A-FIG. 1B, FIG. 6F) (Watanabe A, et al. (2019) Cell. 177:1124-1135 e1116), indicating these Abs engage stem epitopes.

Competitive binding assays showed that S1V2-72 strongly inhibits K06.18 binding (FIG. 1D), indicating the two Abs recognize the same or overlapping epitopes. S1V2-72 does not, however, compete with standard Abs that define known conserved HA stem epitopes, including CR9114 (Dreyfus C, et al. (2012) Science. 337:1343-1348), CR8020 (Friesen R H, et al. (2014) Proc Natl Acad Sci USA. 111:445-450), and 222-1C06 (Guthmiller J J, et al. (2022) Nature. 602:314-320) (FIG. 1E). The novelty of the S1V2-72/K06.18 epitope was corroborated by mutating conserved head and stem epitopes in rH3/KS17 to abolish the binding of epitope-defining standard Abs (McCarthy K R, et al. (2021) Proc Natl Acad Sci USA. 118::e202675211). There was no defining set of mutations strongly affected S1V2-72 reactivity (FIG. 6G).

S1V2-72 competed modestly with LAH5 and LAH31 (FIG. 1F), two cross-reactive mAbs with epitopes contained in the long a helix of the HA2 subunit (Adachi Y, et al. (2019) Nat Commun. 10:3883; Tonouchi K, et al. (2023) PLOS Pathog. 19: e1011554). LAH5 and LAH31 bound avidly to an HA peptide comprising solely the long a helix of prefusion HA2, but S1V2-72 did not (FIG. 1G). Therefore, the epitope of S1V2-72 is substantively distinct from those of the long a helix mAbs.

### Example 2

### Determination of Cryo-EM Structure of S1V2-72 in Complex with Postfusion HA2

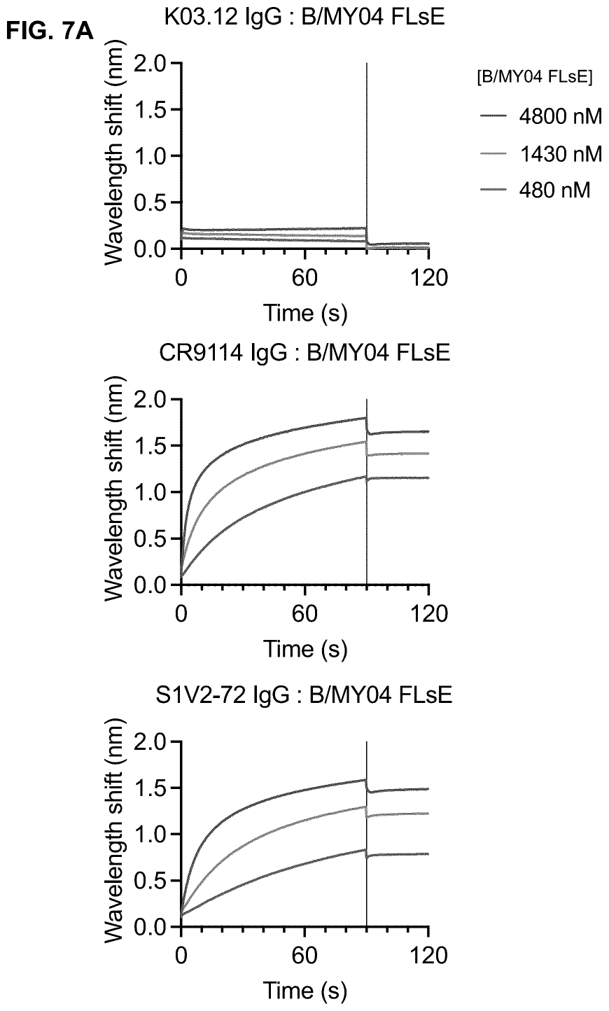

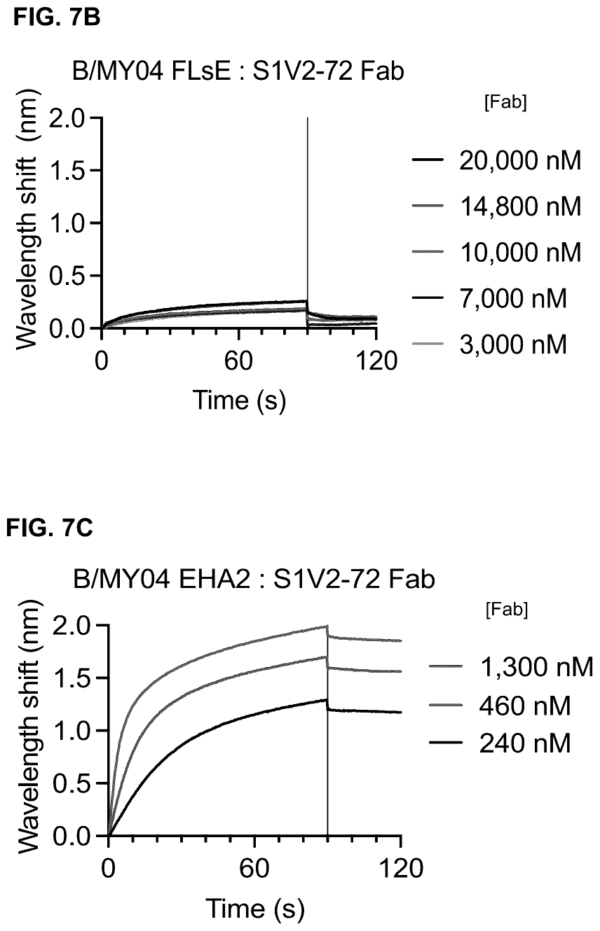

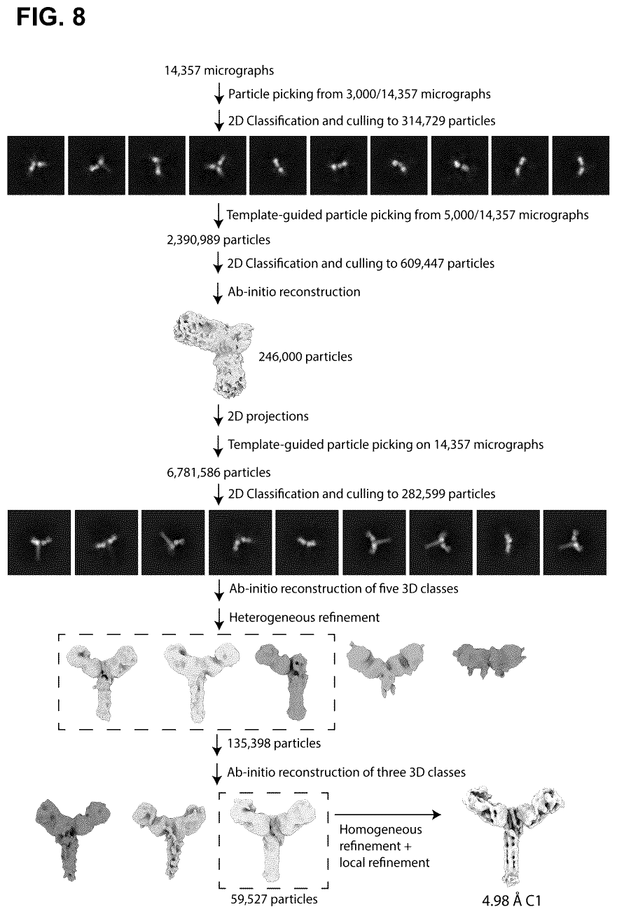

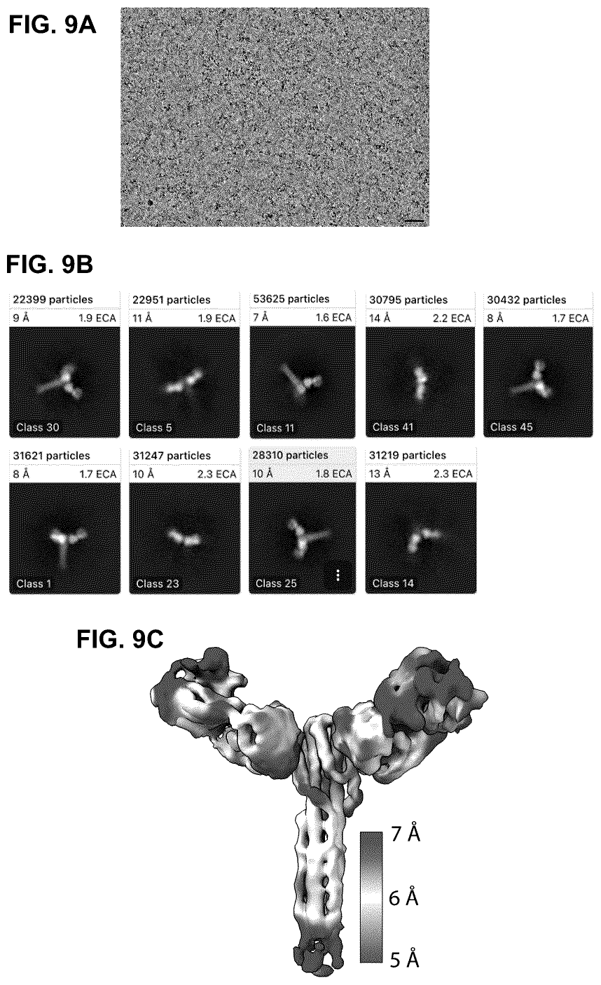

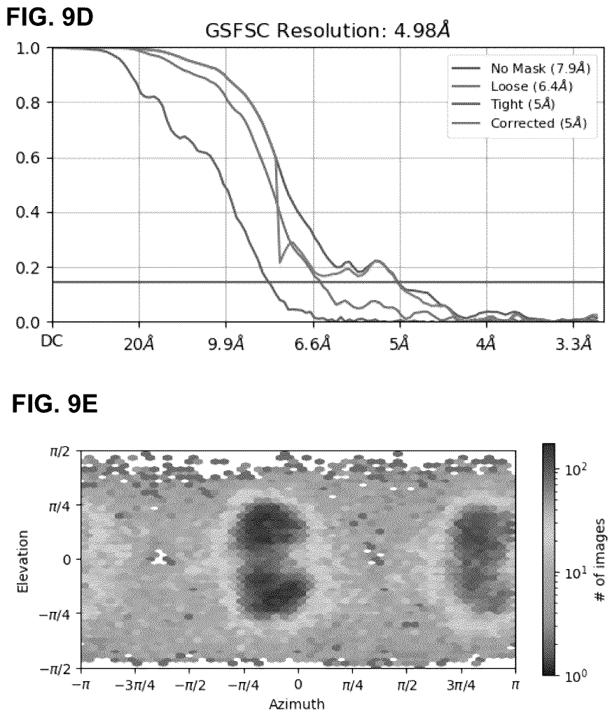

S1V2-72 Fab bound to trimeric B/MY04 HA0 ectodomain were not isolated. Nonetheless, he S1V2-72 epitope is at least partially exposed in native HA0, because S1V2-72 IgG bound HA0s conjugated to Luminex beads (FIG. 1B and FIG. 6C-FIG. 6E) and because S1V2-72 IgG immobilized on a biolayer interferometry (BLI) sensor captured soluble trimers of B/MY04 HA0 (FIG. 7A). In the converse experiment, however, HA0 immobilized on the sensor did not capture S1V2-72 Fab (FIG. 7B). Thus, low-avidity binding between Fab and HA0 was apparently insufficient for detectable complex formation. However, S1V2-72 Fab avidly bound B/MY04 EHA2, a postfusion HA2 construct (FIG. 7C) (Chen J, et al. (1999) Proc Natl Acad Sci USA. 96:8967-8972). The resulting complex was used for single-particle cryo electron microscopy analysis. 14,357 images were recorded with a Titan Krios electron microscope and a Gatan K3 detector. Two-dimensional class averages from a total of ˜6,780,000 particles, calculated in CryoSPARC (Punjani A, et al. (2017) Nat Methods. 14:290-296), showed that most of the complexes had shed one or more of their bound Fabs (FIG. 8, FIG. 9A-FIG. 9B).

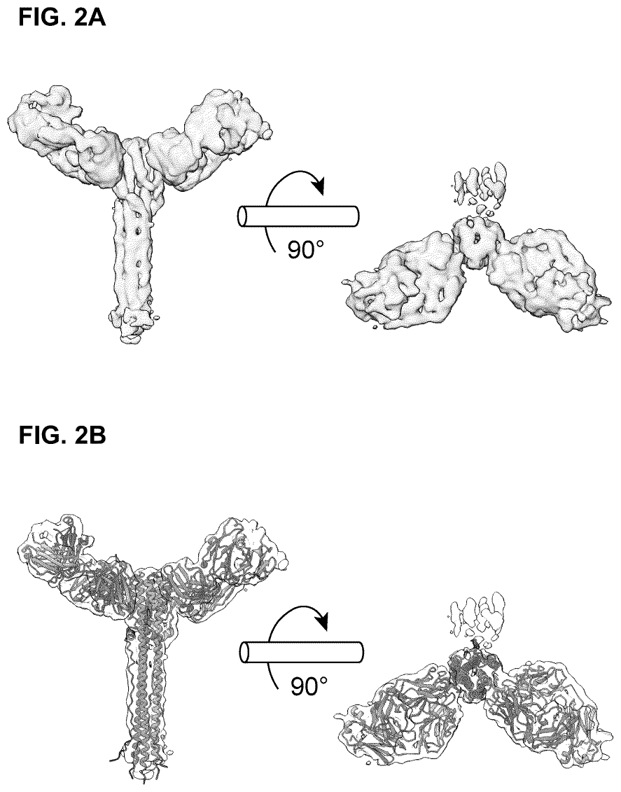

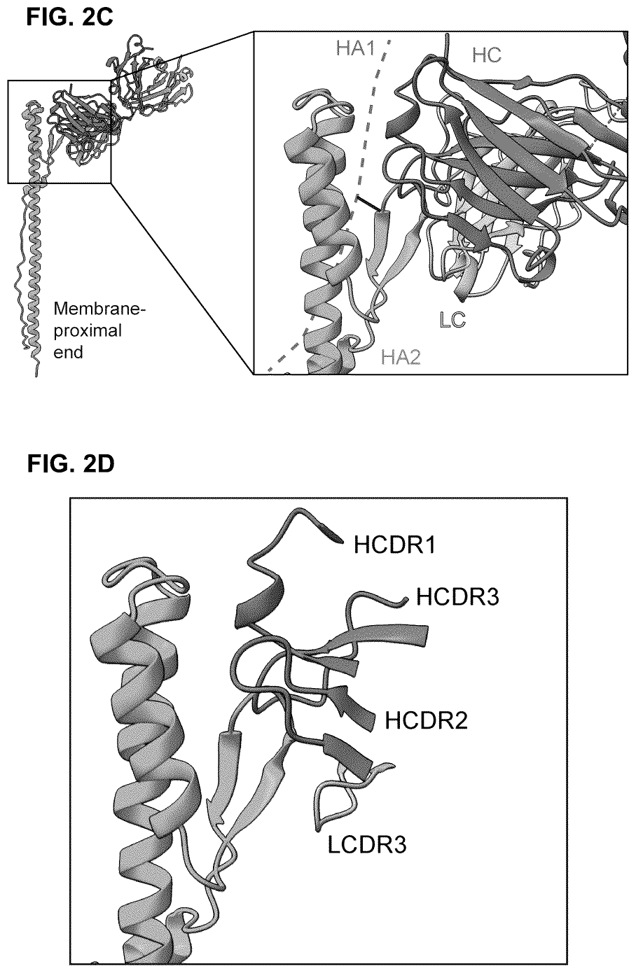

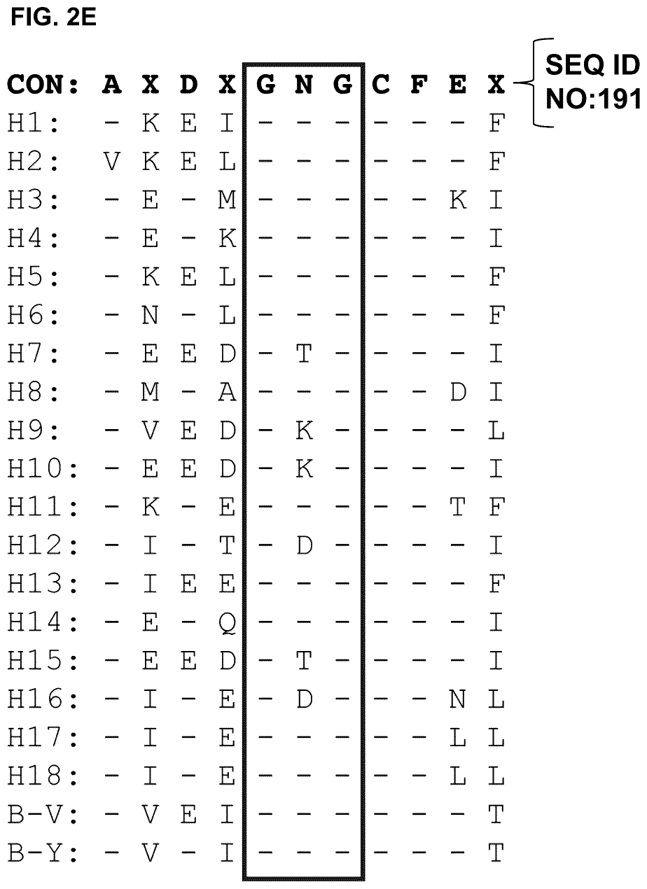

Classes corresponding to both one bound Fab and two bound Fab were selected. Here, the latter refined more readily in three dimensions than did the former, and the corresponding three-dimensional reconstruction (from about 60,000 contributing particles) was chosen as reference for further refinement in CryoSPARC (to a final resolution of about 5 Å). (FIG. 2A, FIG. 8, FIG. 9A-FIG. 9E, Table 18).

TABLE 19
Cryo-EM Data Collection and Processing Statistics.

| Data Collection and Processing | Value |
|---|---|
| Magnification | 105,000 |
| Voltage (kV) | 300 |
| Electron exposure (e⁻/Å⁻²) | 76.12 |
| Defocus range (um) | 0.8, 2.2 |
| Pixel size (Å) | 0.825 |
| Symmetry imposed | C1 |
| Initial particle images (no.) | 6,781,586 |
| Final particle images (no.) | 59,527 |
| Map Resolution (Å) | 4.98 |
| FSC threshold | 0.143 |
| Map resolution range (Å) | 5.0-7.0 |

Alphafold2 (AF2) (Jumper J, et al. (2021) Nature. 596:583-589) was used to prepare a model of EHA2 with the sequence of the B/Malaysia/2506/2004 HA2 ectodomain (residues 23-181, H3 numbering) in its postfusion conformation. AF2 was used to model the variable module of S1V2-72 and this model was used to compare with the structure of the inferred germline Fab from the DH270 lineage (Bonsignori M, et al. (2017) Sci Transl Med. 9:eaai7514), which derives from precisely the same VH and VL as S1V2-72. The AF2 and DH270 variable modules were nearly identical, except for the HCDR3, which is 20 residues in DH270 but only 12 in S1V2-72. Independent docking of the three modules (EHA2 and variable modules from AF2, plus the constant-region dimers from the DH270 UCA) produced an excellent fit to the map (FIG. 2B).

The center of the epitope is a β-hairpin, residues 129-141, at the membrane-distal end of the postfusion HA2 rod (FIG. 2C). In the Fab complex, the tip of the hairpin fits into a shallow cavity lined by the three heavy-chain CDRs, with additional contact from the light-chain CDR3 (FIG. 2D). The apparent interactions outside the β-hairpin are between the tips of both HCDR1 and HCDR2 and residues around the position at which the HA2 polypeptide chain folds back on itself, when the extended intermediate collapses into the postfusion conformation (FIG. 2D). The flexibility of the hairpin in the recombinant EHA2 construct makes more precise modeling difficult at this stage.

In the prefusion HA trimer, the β-hairpin is part of a small, 5-strand sheet, which fixes its conformation more firmly than in EHA2. The central strand of that sheet comprises residues from HA1, including Cys14, which forms a disulfide link with HA2 Cys137 in the flanking strand—the descending strand of the β-hairpin epitope. The full postfusion structure thus retains HA1, flexibly tethered to the HA2 hairpin through that disulfide. The fit to the density map indicates that the interaction of S1V2-72 with the β-hairpin can accommodate that tether (FIG. 2C). The hairpin's conformation in the prefusion structure is nearly the same as its average conformation in postfusion HA2, but the trimer interface in prefusion HA would prevent Ab access. Nonetheless, conformational fluctuations at the membrane-proximal end of prefusion HA might expose that loop, allowing transient access. The Luminex and BLI data (FIG. 1A-FIG. 1B, FIG. 6C-FIG. 6E, FIG. 7A) indicate that such fluctuations may indeed occur, at least in the context of the soluble, trimeric ectodomain.

The breadth of S1V2-72 binding can probably be attributed in part to the conserved primary structure of the β-hairpin (FIG. 2E). The bend of the hairpin encompasses Gly134-Asn135-Gly136, a sequence conserved in HAs from both lineages of IBV, plus all IAV HA subtypes except H7, H9, H10, H12, H15, and H16. The descending β strand continues with the absolutely conserved pair of Cys137 (and its disulfide linkage to Cys14 in HA1) and Phe 138. Further study is needed to determine whether the amino acid substitutions in H9, H12, H15, and H16 prohibit S1V2-72 binding. S1V2-72 accommodates substitution of Thr for Asn135 in H7 (FIG. 1C), whereas substitution of Lys for Asn135 in H10 might account for its lack of S1V2-72 binding (FIG. 1C).

### Example 3

### Examination of Protection by S1V2-72

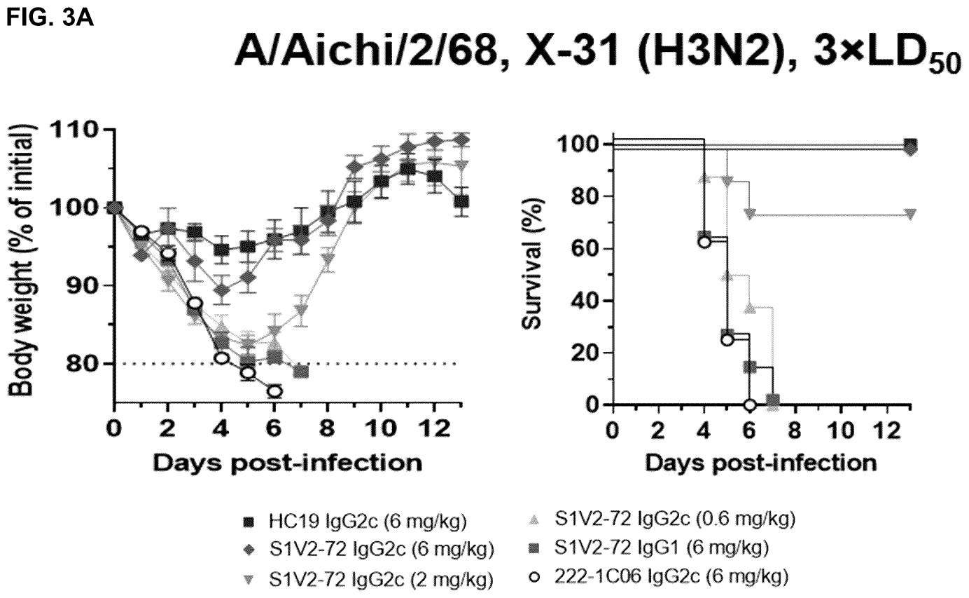

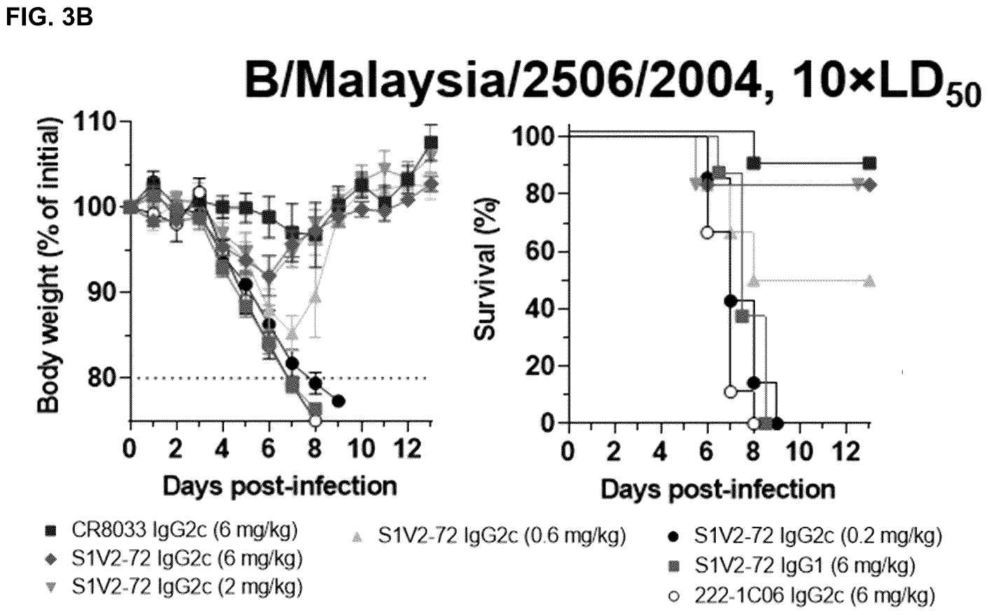

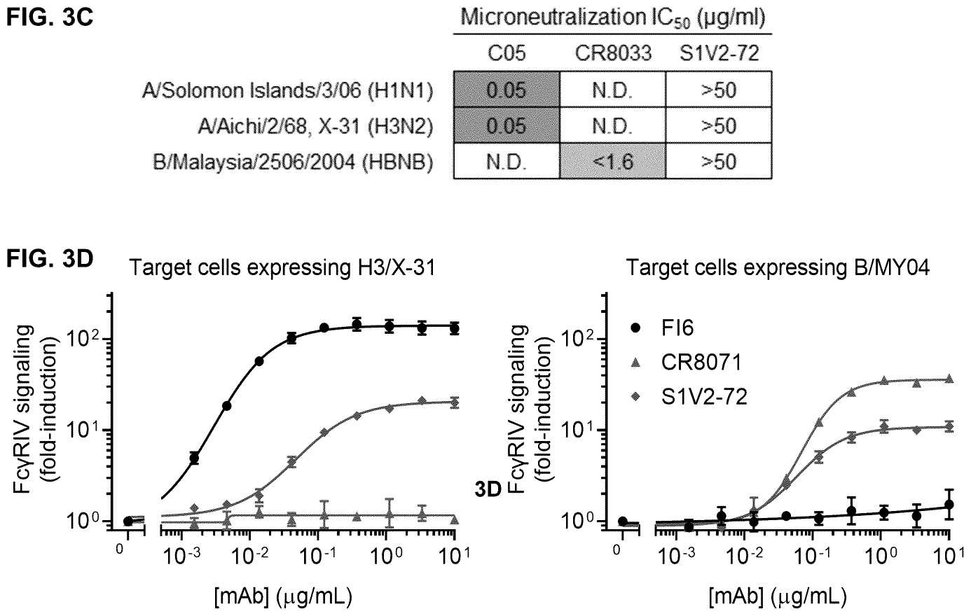

To determine whether S1V2-72 can protect against lethal IAV or IBV infections, the following IgGs were passively transferred to mice: (i) S1V2-72, (ii) the potent neutralizing HA Abs HC19 (Bizebard T, et al. (1995) Nature. 376:92-94) or CR8033 (Dreyfus C, et al. (2012) Science. 337:1343-1348), and (iii) an H1-specific Ab (222-1C06) (Guthmiller J J, et al. (2022) Nature. 602:314-320). When administered as IgG2c, each mAb was injected at 6 mg Ab/kg mouse. Alternatively, S1V2-72 IgG2c was transferred at doses lower than 6 mg/kg or as S1V2-72 IgG1 (at 6 mg/kg). Three hours after mAb infusion, mice were infected intranasally with H3N2 IAV (A/Aichi/2/1968, X-31) or IBV (B/Malaysia/2506/2004). All animals treated with the irrelevant 222-1C06 IgG2c Ab showed losses in body weight that required ethical euthanasia by 4-8 days post-infection, whereas H3N2- and IBV-neutralizing Abs were potently protective (FIG. 3A, FIG. 3B).

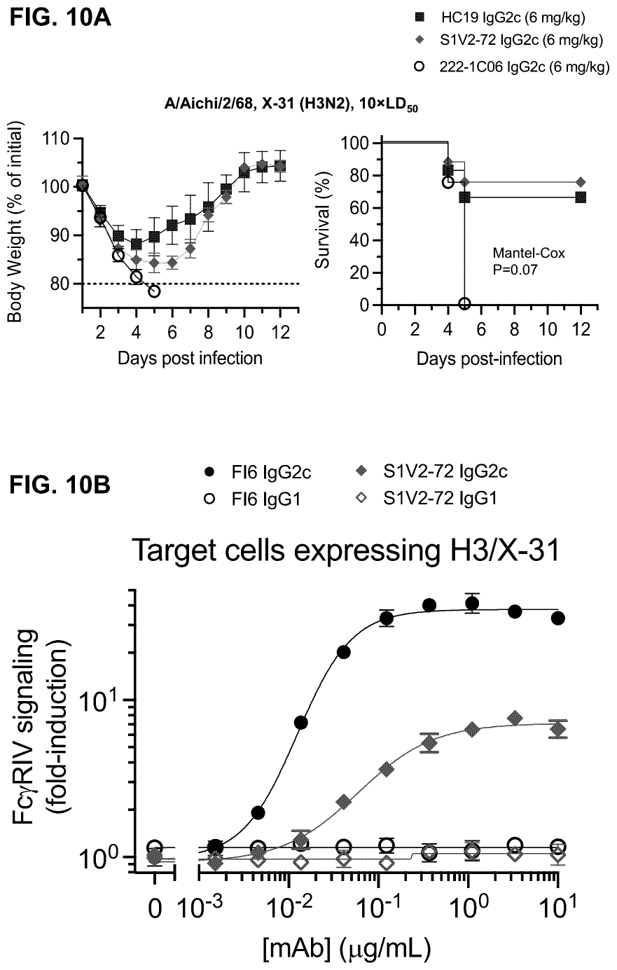

S1V2-72 protected efficiently against infection-induced weight loss when administered as mouse IgG2c, but failed to protect when administered as mouse IgG1 (FIG. 3A, FIG. 3B, and FIG. 10A). In an in vitro influenza microneutralization assay, S1V2-72 neutralized neither IAV nor IBV, in contrast to receptor binding site Abs C05 (Ekiert D C, et al. (2012) Nature. 489:526-532) and CR8033 (FIG. 3C). As an IgG2c, S1V2-72 induced FcγRIV signaling in an Ab-dependent cell-mediated cytotoxicity (ADCC) assay (FIG. 3D). As mouse IgG1, S1V2-72 did not induce FcγRIV signaling in the same assay (FIG. 10B), as expected (Nimmerjahn F, et al. (2005) Immunity. 23:41-51). Based on this data, Abs to the S1V2-72 epitope protected against influenza-driven pathology by Fcγ-receptor-dependent mechanisms, rather than by neutralization.

### Example 4

### Identification of a Shared Genetic Signature for S1V2-72-Like Abs

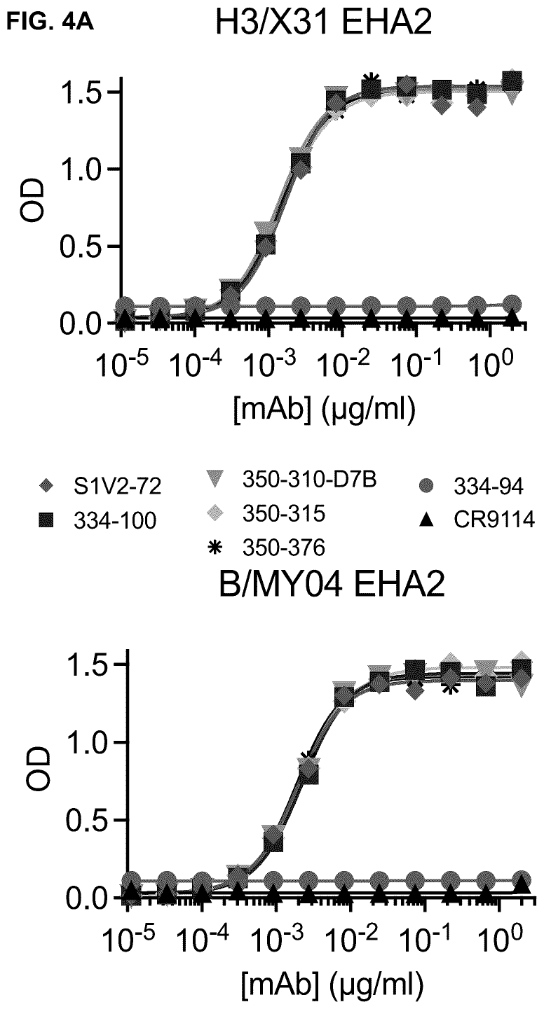

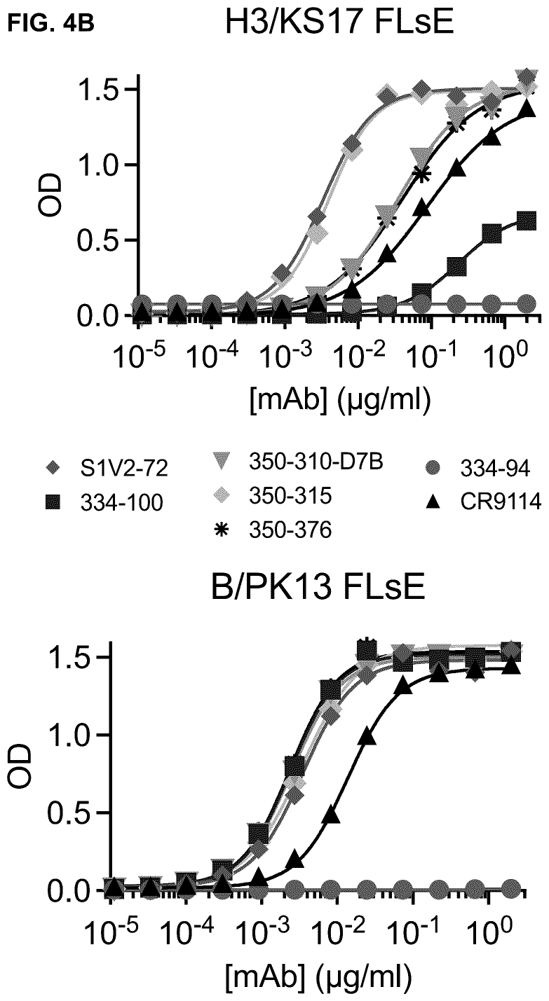

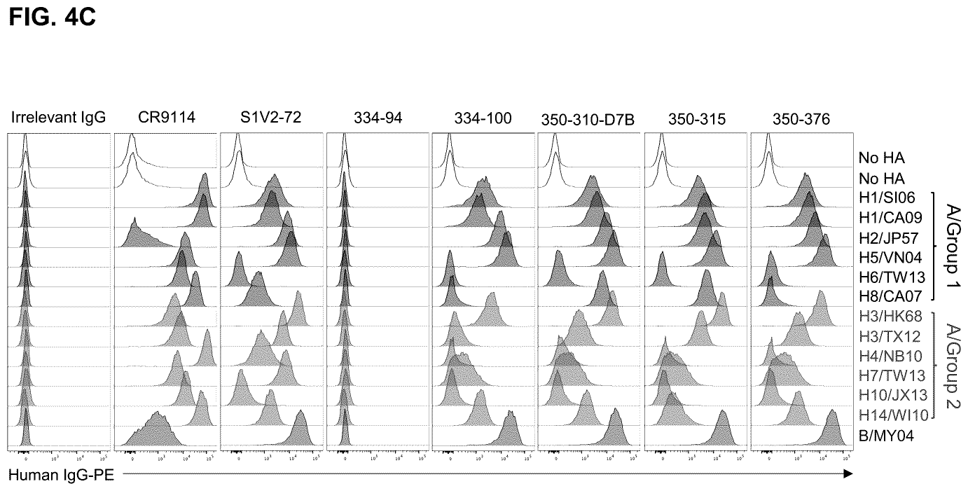

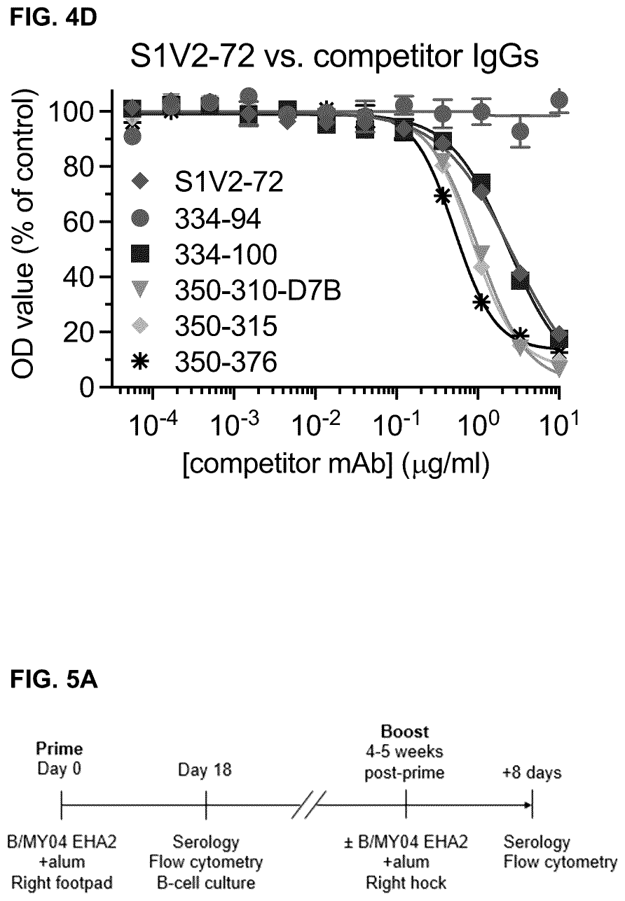

S1V2-72 and K06.18 are from different donors, but they compete for the same epitope, have similar breadth, and come from similar V(D)J rearrangements comprising a IGHV1-2-derived H chain with a 12 amino acid HCDR3, and a IGLV2-family L chain. To test whether such V(D)J rearrangements constitute a genetic signature for an S1V2-72-like public Ab response, a dataset of 1,952 mAbs generated from putative HA-binding Bmem cells isolated from human donors 28 days after immunization with a chimeric HA vaccine was mined (Guthmiller J J, et al. (2022) Nature. 602:314-320). Initially, five (5) mAbs with HCs encoded by IGHV1-2, LCs encoded by IGLV2-family genes, and HCDR3s encompassing 11-14 amino acids were selected. The five mAbs (i.e., named 334-94, 334-100, 350-310-D7B, 350-315, and 350-376), were from two donors (334 and 350), and included three singletons and a clonal dyad. Recombinant IgG versions of each mAb, except 334-94 (a singleton), bound H3/X31 and B/MY04 EHA2s (FIG. 4A), as well as B/PK13 and H3/KS17 HA0s (FIG. 4B) in ELISAs. Moreover, except for 334-94, the mAbs bound B/MY04 plus many IAV Group 1 and 2 rHAs expressed on the surface of K530 cell lines (FIG. 4C), although no mAb could match the full breadth of S1V2-72 for IAV HA subtypes (e.g., only S1V2-72 bound H4/NB10). Like S1V2-72, none of the new mAbs bound H6/TW13 or H10/JX13. Finally, the four new HA-reactive mAbs potently competed with S1V2-72 for EHA2 binding (FIG. 4D).

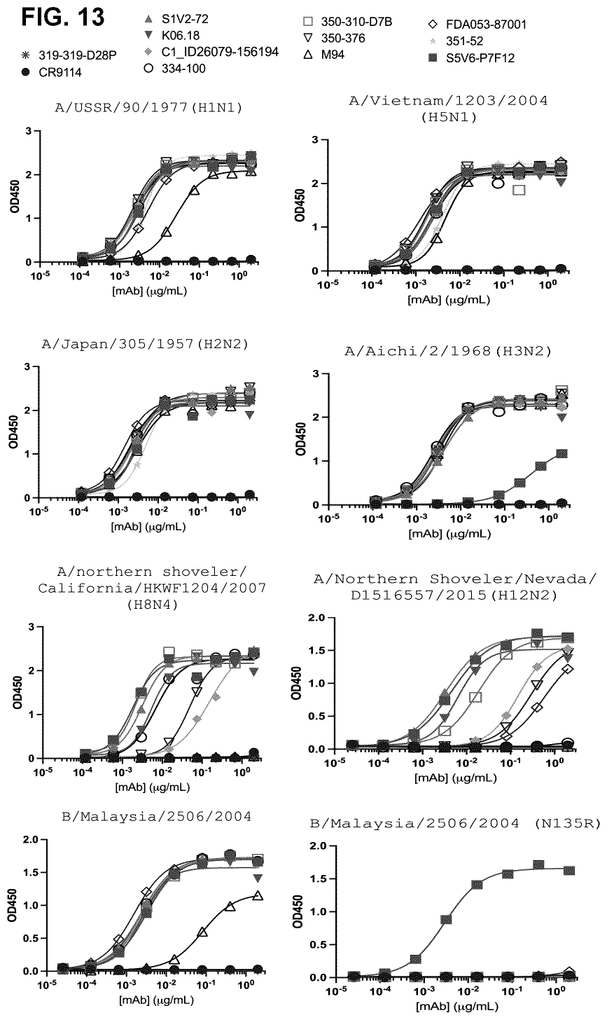

Subsequently, five more potential S1V2-72-like mAbs were identified, either in the same published dataset (Guthmiller J J, et al. (2022) Nature. 602:314-320), or from other sources. These mAbs (i.e., named M94, C1_ID26079-156194, 351-52, FDA053-87001, and 350-341) were from donor 350 and four new donors. Each of the potential S1V2-72-like mAbs has a HC encoded by human IGHV1-2 and an HCDR3 encompassing 10-13 amino acids (Table 20). M94 and C1_ID26079-156194 have LCs encoded by IGLV2-family genes, while the remaining three mAbs have LCs encoded by IGLV7-46 or IGKV3-20. Like other S1V2-72 antibodies, recombinant IgG versions of M94, C1_ID26079-156194, 351-52, and FDA053-87001 bound various EHA2s representing several IAV subtypes (FIG. 13). Each mAb also bound wildtype (WT) B/MY04 EHA2. None of the mAbs bound mutant B/MY04 EHA2 containing an Asn135 to Arg135 substitution (N135R) in the β-hairpin epitope (FIG. 13), indicating these antibodies all target the β-hairpin epitope.

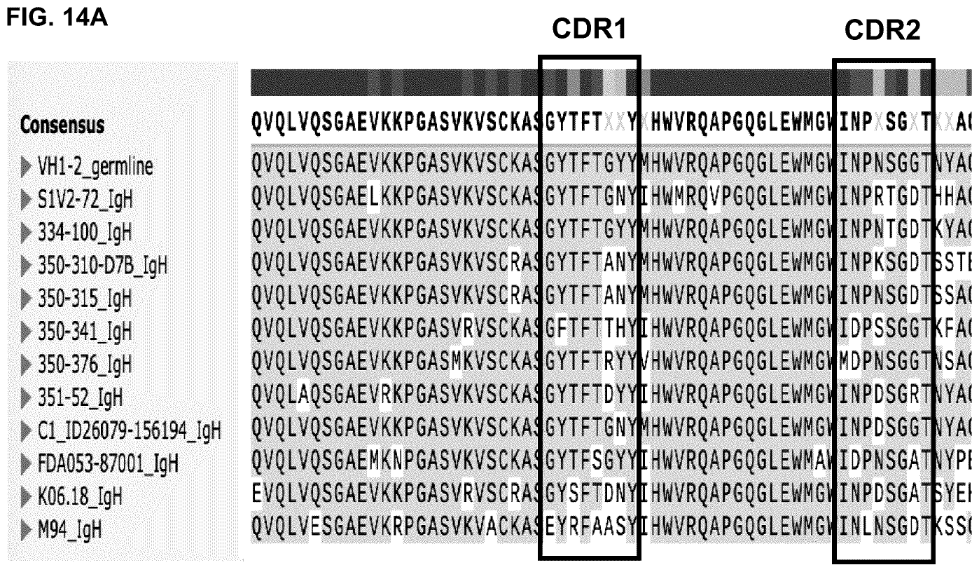

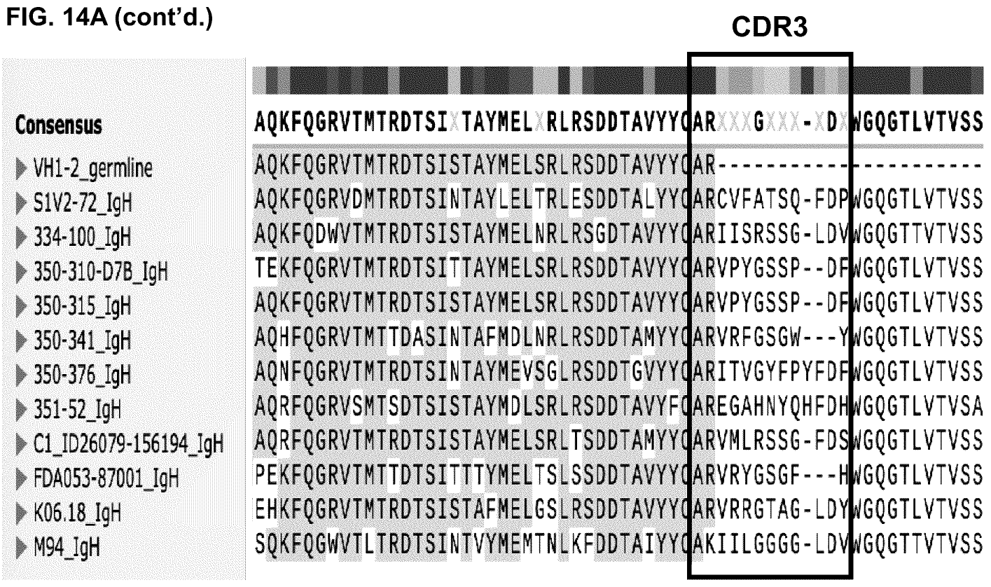

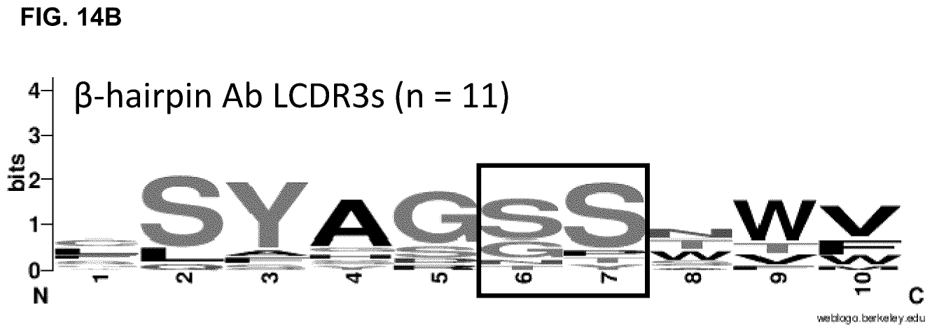

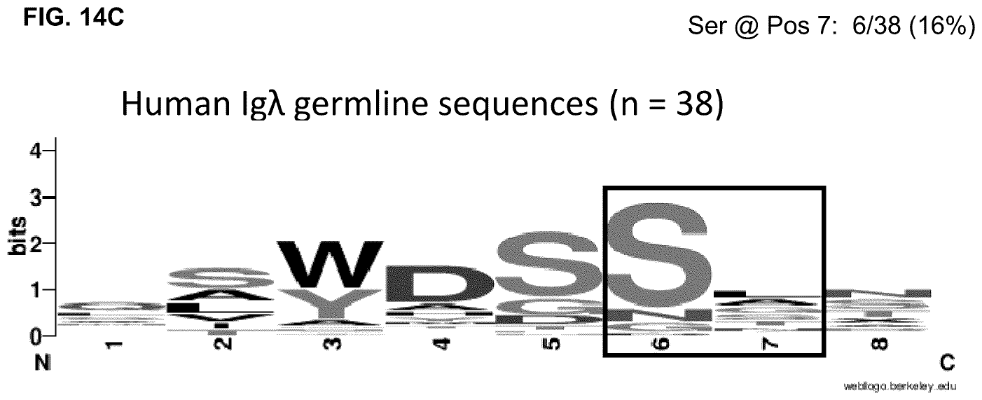

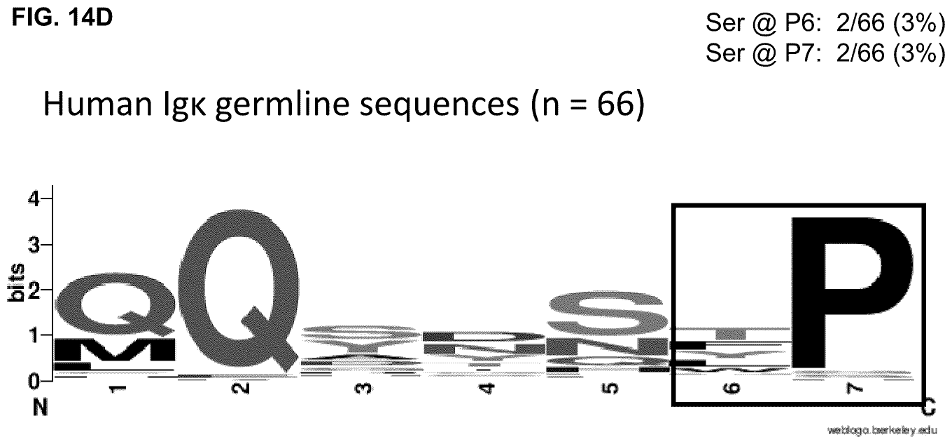

In summary, ten clonal HA2 β-hairpin antibodies from eight genetically unrelated donors each have heavy chains constructed from IGHV1-2 (Table 20), indicating this human V-gene segment uniquely encodes features necessary for β-hairpin binding. Multiple-sequence alignment of the amino acid sequences of the β-hairpin antibody HCs shows many universally or widely conserved residues, including within HCDR1, HCDR2, and HCDR3 (FIG. 14A), each of which is positioned to interact with HA2 (FIG. 2D). Likewise, that β-hairpin antibody LCs are so frequently assembled from IGLV genes, particularly IGLV2-family genes, implies these genes encode functionally important features. The tip of the LCDR3 loop is the only region of the S1V2-72 LC in position to bind HA2 (FIG. 2D), and the amino acids at the tip of the loop are most frequently serines (FIG. 14B). These serine residues are germline-encoded in many IGLV genes, especially IGLV2 genes (FIG. 14C). In contrast, germline IGKV genes rarely encode serines in these positions (FIG. 14D), although IGKV3-20, the gene that encodes the 351-52 antibody LC, is an exception.

TABLE 20
Exemplary Genetic Features of S1V2-72-Like Antibodies

| | | HCDR3 | | LCDR3 |
|---|---|---|---|---|
| | | Length | | Length |
| Antibody | IGHV | (AA) | IGLV | (AA) |
| S1V2-72 | IGHV1-2*02 | 12 | IGLV2-23*01 | 10 |
| K06.18 | IGHV1-2*07 | 12 | IGLV2-23*02 | 10 |
| 334-100 | IGHV1-2*04 | 12 | IGLV2-11*01 | 11 |
| 350-310-D7B | IGHV1-2*02 | 11 | IGLV2-23*02 | 10 |
| 350-315 | IGHV1-2*02 | 11 | IGLV2-23*02 | 10 |
| 350-376 | IGHV1-2*02 | 13 | IGLV2-11*01 | 10 |
| M94 | IGHV1-2*04 | 12 | IGLV2-11*01 | 11 |
| C1_ID26079- | IGHV1-2*02 | 12 | IGLV2-23*01 | 10 |
| 156194 | | | | |
| 351-52 | IGHV1-2*02 | 13 | IGKV3-20*01 | 9 |
| FDA053-87001 | IGHV1-2*02 | 10 | IGLV7-46*01 | 9 |
| 350-341 | IGHV1-2*02 | 10 | IGLV7-46*01 | 9 |

### Example 5

### Frequency of S1V2-72-Competing Bmem in Human Donors

Whereas Bmem with breadth for Group 1 and Group 2 IAV plus IBV are rare (2/1,642 HA⁺ Nojima cultures; 0.1%), Bmem with epitopes that overlap the S1V2-72 epitope and hence compete, but bind with more restricted breadth, may be more frequent. To find Bmem of this kind, Nojima culture supernatants containing HA-binding IgG (n=860, from 7 donors) were screened for competition with S1V2-72.

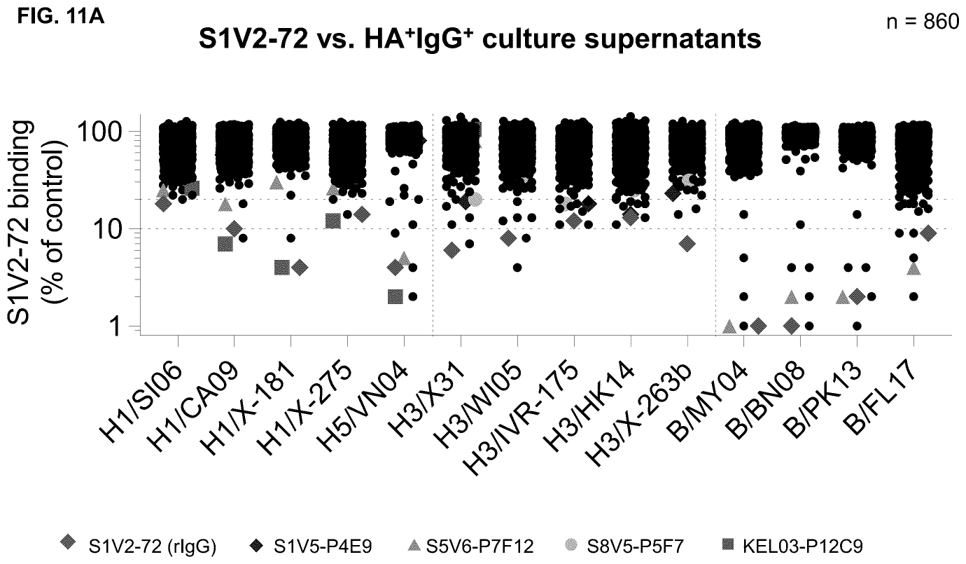

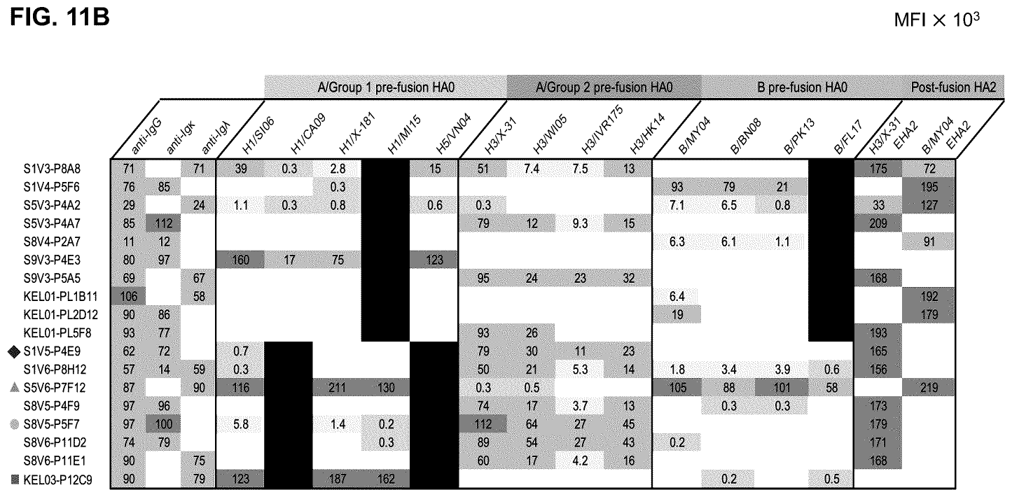

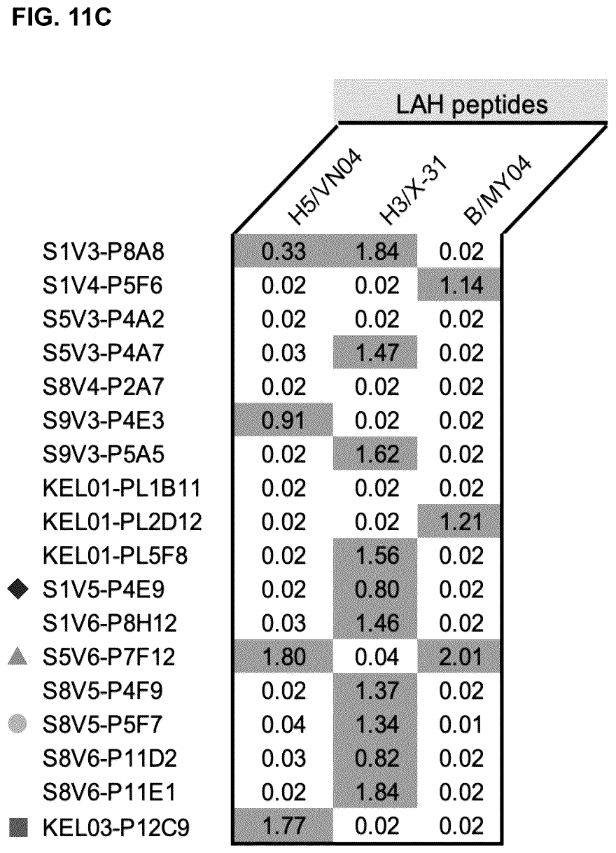

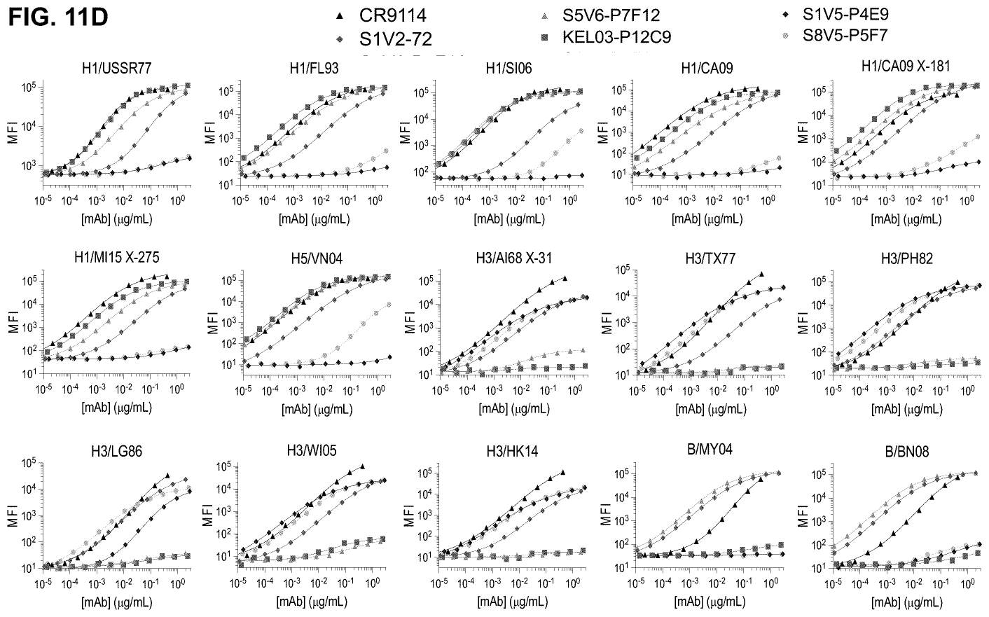

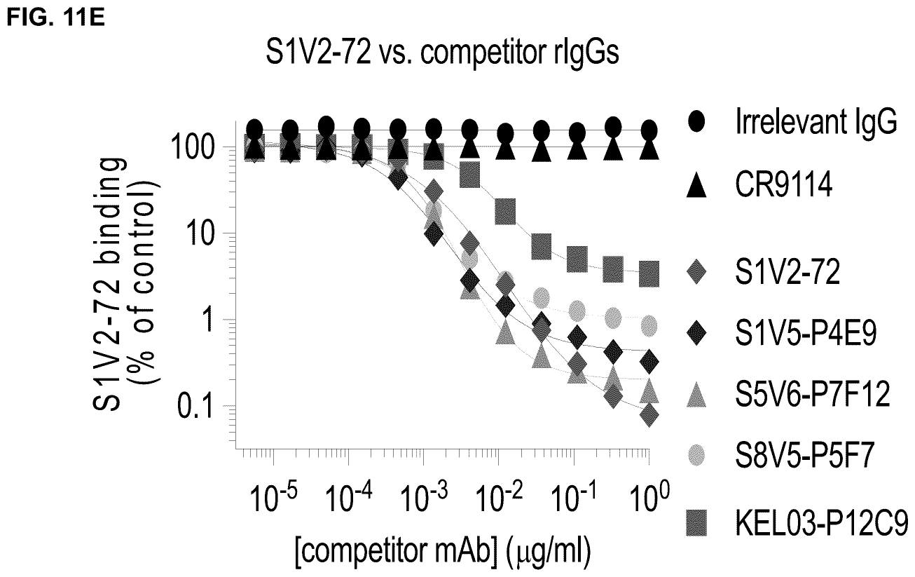

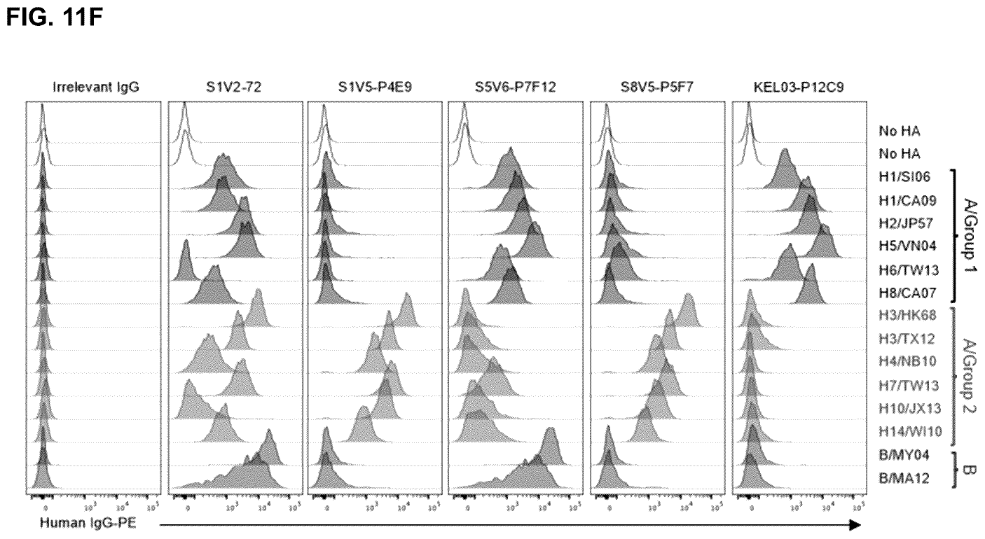

About 4% (33/860) of culture supernatant IgGs, including some from each donor, inhibited by ≥80% the binding of S1V2-72 to one or more HAs (FIG. 11A). Six of the 7 donors had Bmem (18/860) that inhibited S1V2-72 binding by ≥90%. The 18 most potent S1V2-72 competitors had substantial intra-subtypic breadth for historical H1, H3, or B HAs, and some even had heterosubtypic or cross-group breadth (FIG. 11B). Sixteen of the 18 Bmem bound EHA2 forms of H3/X31 or B/MY04 (FIG. 11B). The remaining two Bmem might also bind postfusion HA, but they are specific for Group 1 HAs, which were not represented by any EHA2s in this screen. Nearly all (15/18) of the S1V2-72 competitors also reacted with LAH peptides from H5/VN04, H3/X31, and/or B/MY04 (FIG. S6C), although S1V2-72 did not (FIG. 1G). Four broadly binding S1V2-72-competing mAbs from different donors were selected for further characterization as rIgGs. The rigGs recapitulated the breadth of HA binding with the corresponding Nojima culture supernatant Abs (FIG. 11D) and also substantially inhibited S1V2-72 binding to rHAs on Luminex beads (FIG. 11E). Each S1V2-72 competitor had detectable heterosubtypic reactivity with the broadest (5V6-P7F12), avidly binding HAs from most IAV Group 1 subtypes and from both lineages of IBV, but interacted with IAV Group 2 HAs weakly, if at all (FIG. 11D, FIG. 11F).

### Example 6

### Immunogenicity of the S1V2-72 Epitope in Mice

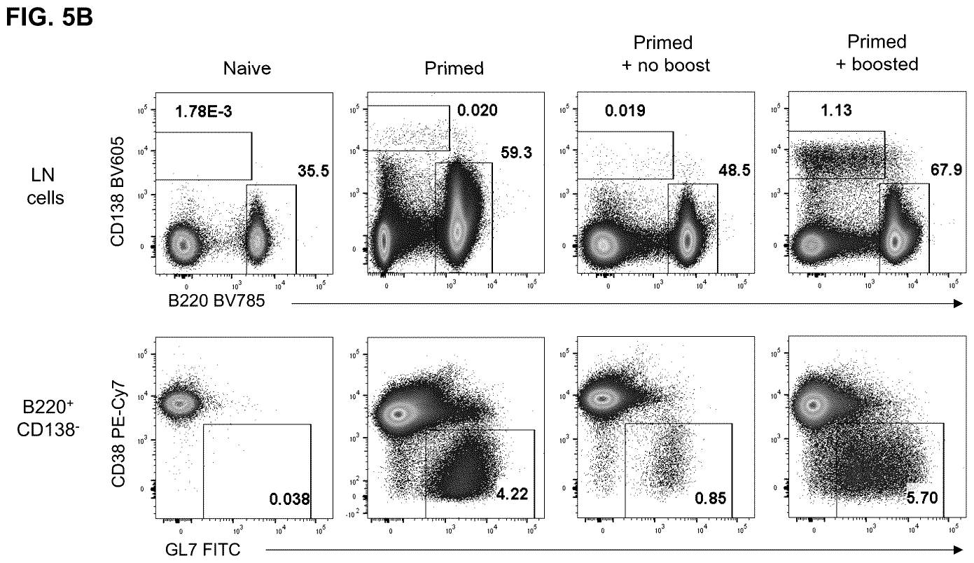

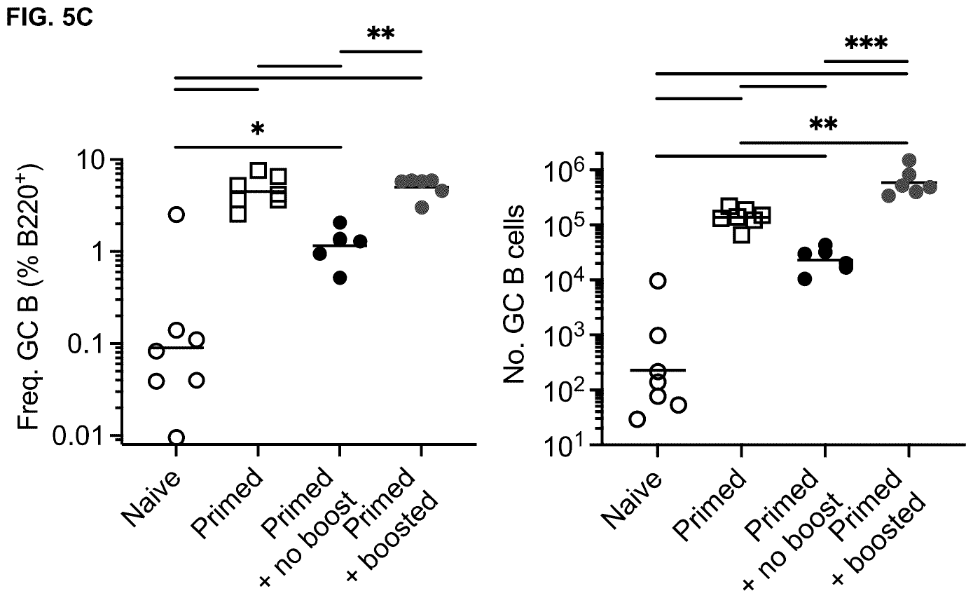

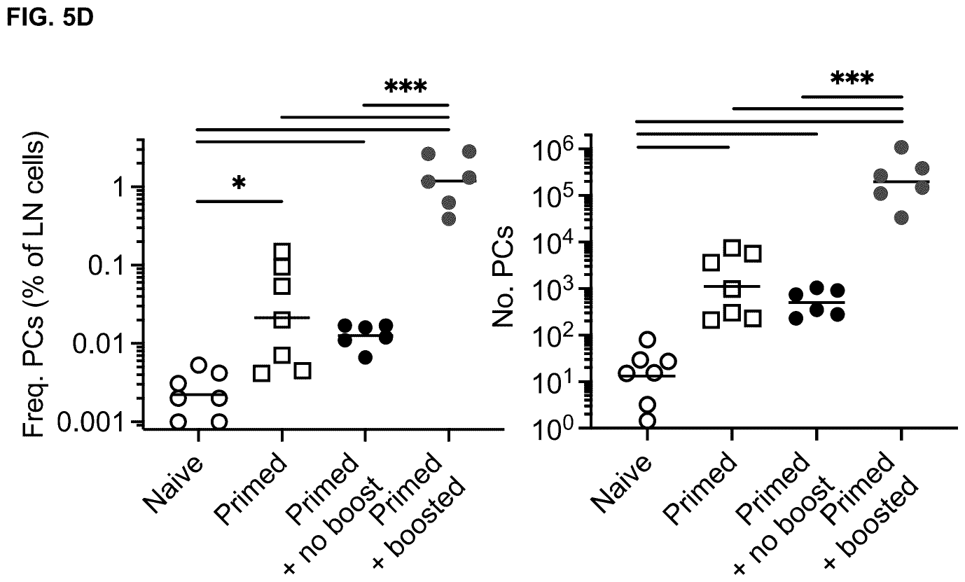

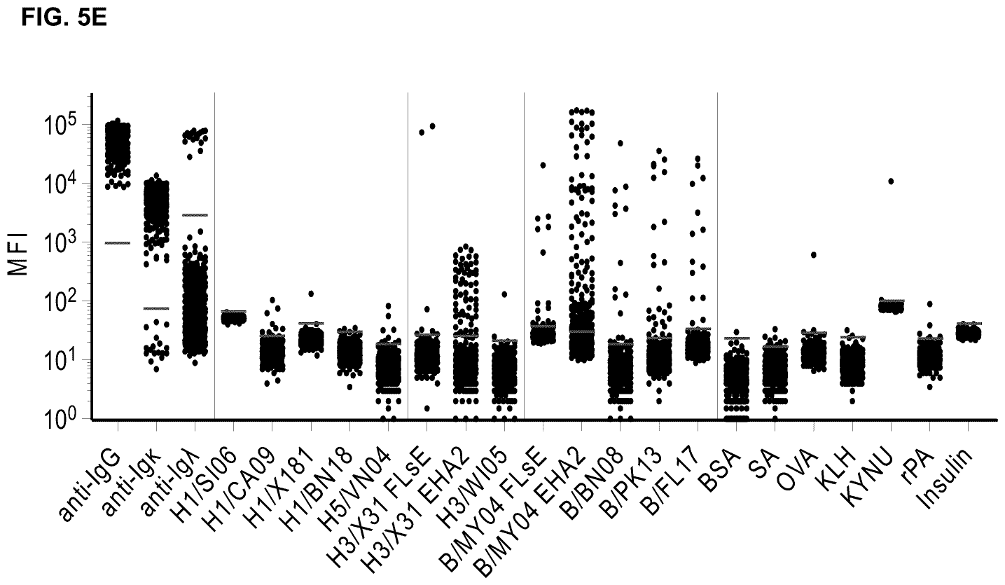

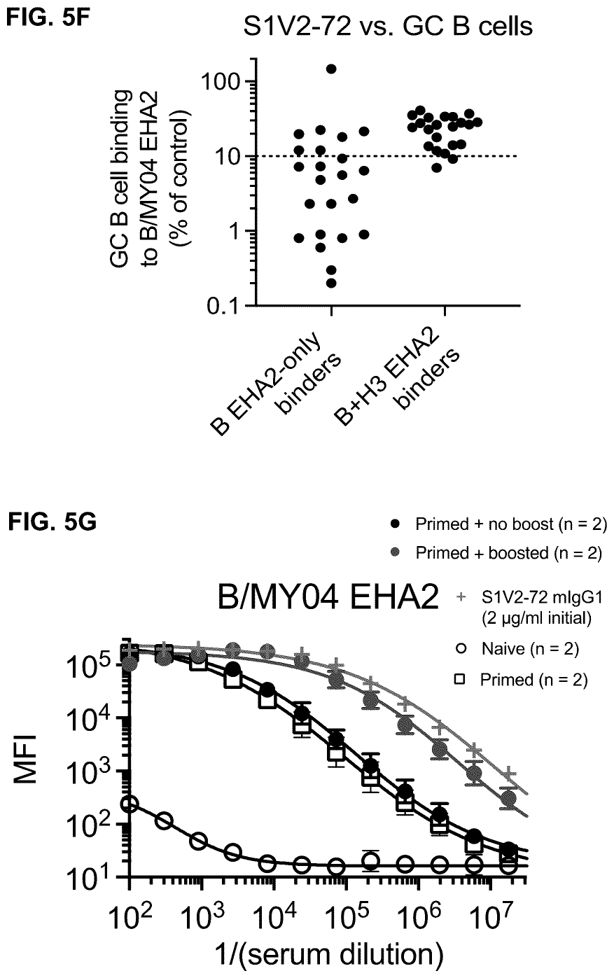

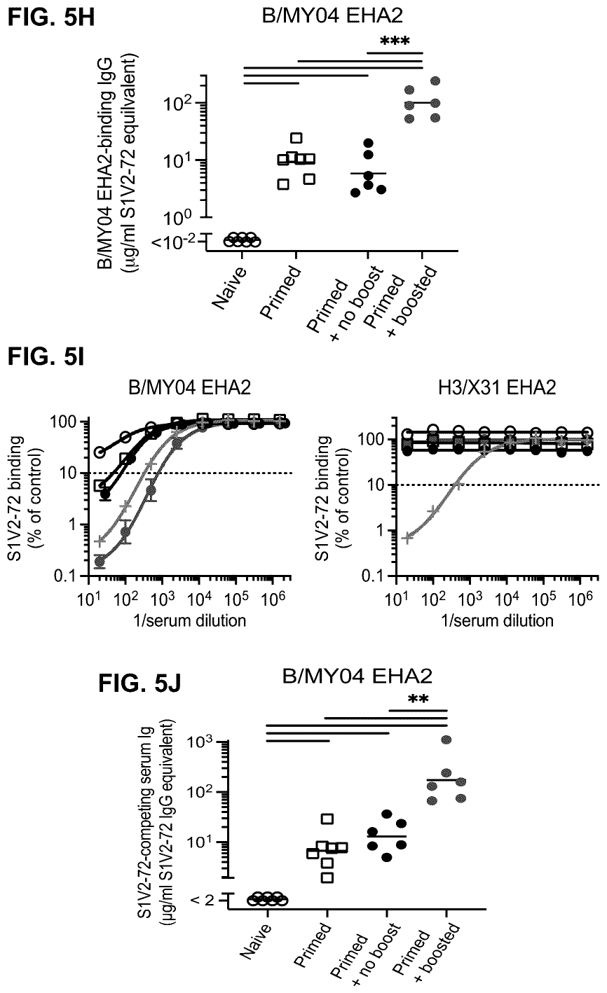

To determine whether vaccination with postfusion HA2 can elicit S1V2-72-like Abs, C57BL/6J mice were immunized with B/MY04 EHA2 in alum adjuvant. Serum Ab and draining lymph node (LN) germinal center (GC) B cell and plasma cell (PC) responses were analyzed 18 days later, or 8 days after ipsilaterally boosting with homologous antigen (FIG. 5A).

At 18 days post-prime, draining LN GC B cell (B220⁺CD138⁻CD38^{1o}GL7⁺IgD⁻) numbers were ≥30-fold greater in B/MY04 EHA2-vaccinated mice than in naïve controls, and remained ≥10-fold above background for at least 5-6 weeks in primed animals (FIG. 5B-FIG. 5C). LN PC (B220-CD138+) numbers were also modestly greater in most B/MY04 EHA2-primed animals than in naïve controls, both at 18 days and 5-6 weeks (FIG. 5B, FIG. 5D). Eight days after boosting with homologous antigen, GC B cell and PC numbers were ˜10- and ˜100-fold greater, respectively, than in unboosted mice (FIG. 5B-FIG. 5D).

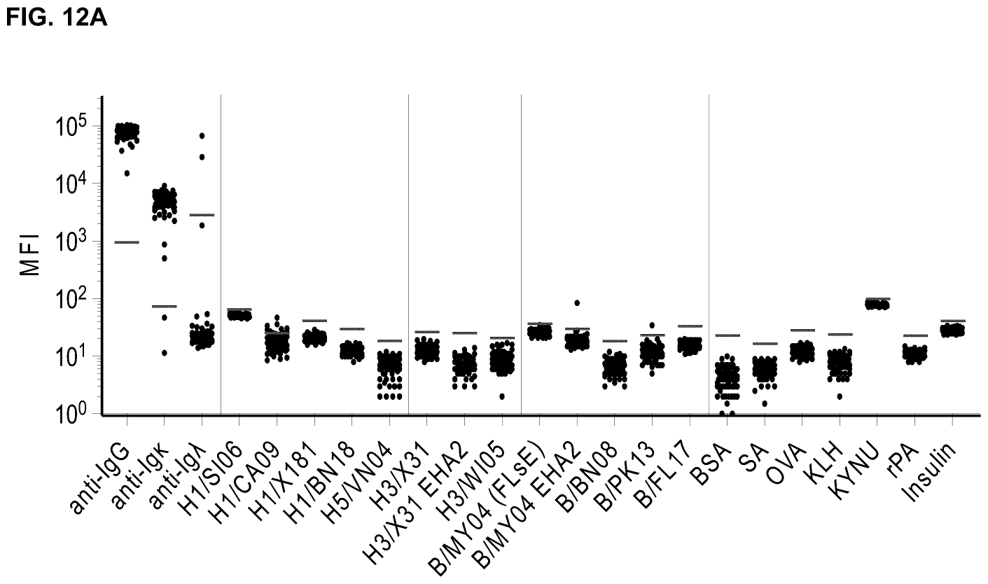

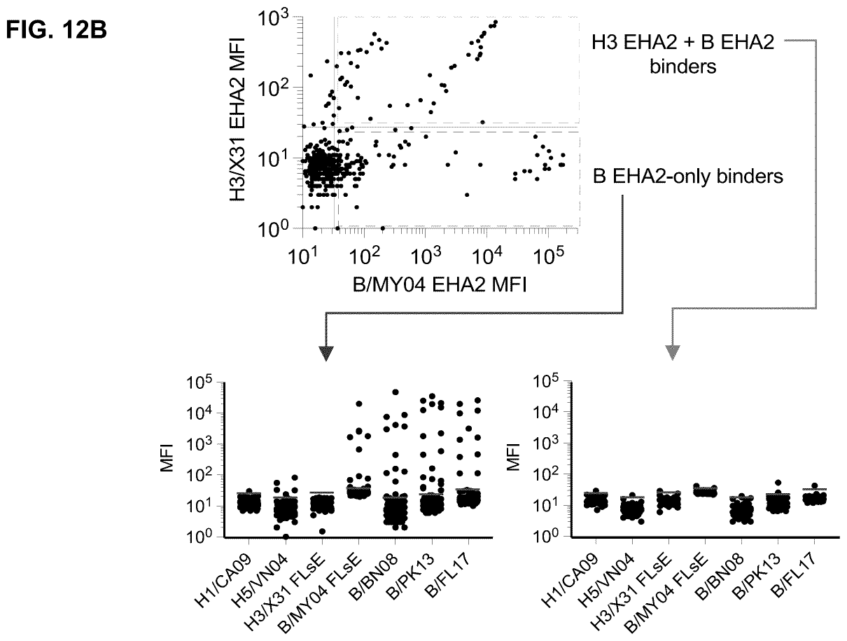

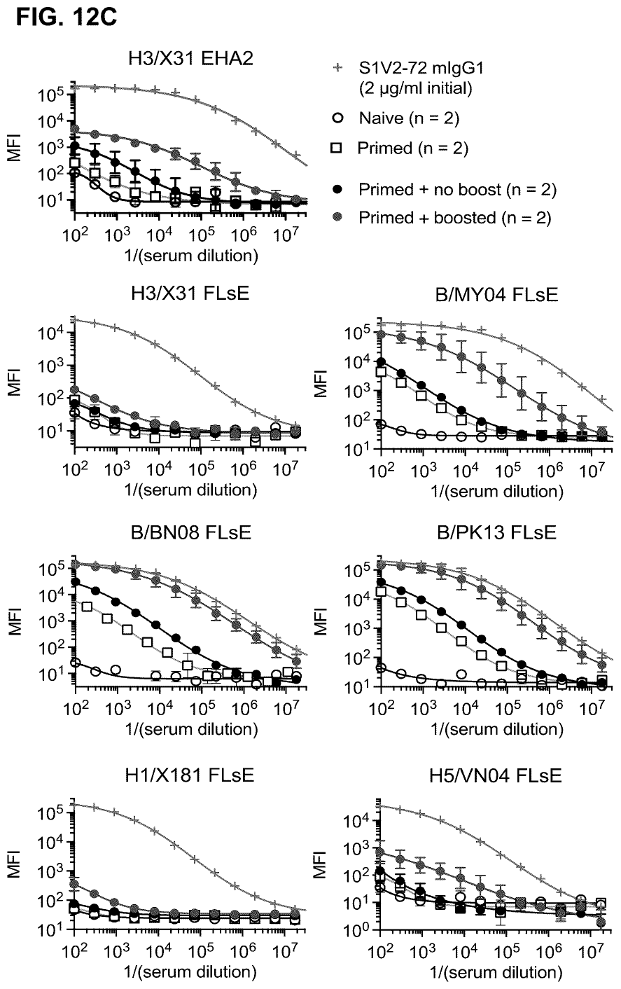

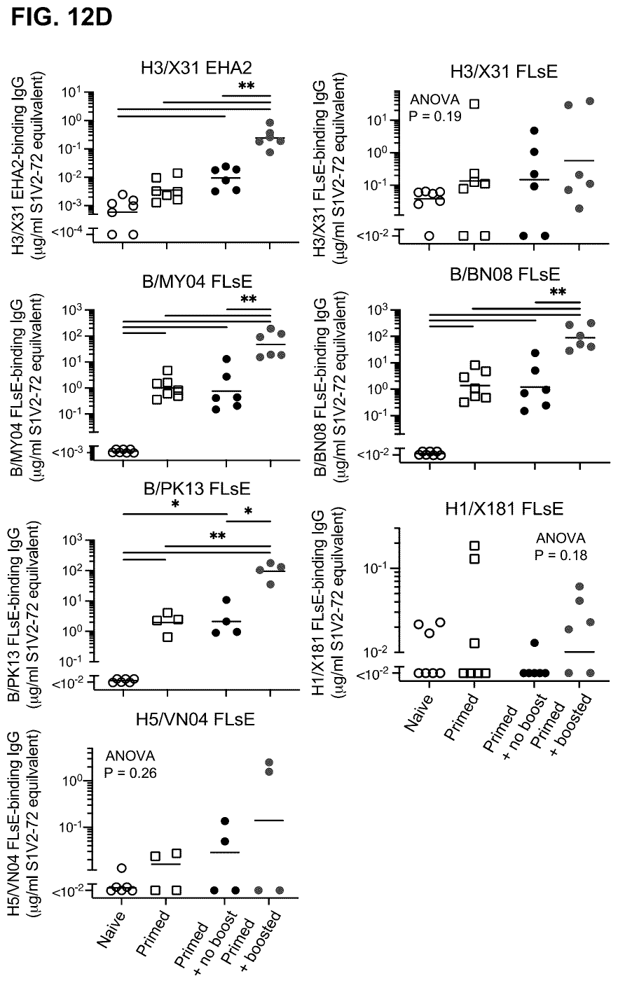

To determine the antigen-specificity of GC B cells responding to primary immunization with B/MY04 EHA2, individual GC B cells 18 days post-prime were sorted into single-cell Nojima cultures and the secreted clonal IgGs were subsequently analyzed (Kuraoka M, et al. (2016) Immunity. 44:542-552). In contrast to resting mature follicular B cells, of which ≤5% (7/151) bound HAs (and even then, only weakly), ˜25% of primary GC B cells bound B/MY04 EHA2 (FIG. 5E, FIG. 12A, Table 21). Of the B/MY04 EHA2-binders in the G C, ˜25% also bound H3/X31 EHA2, while a separate subpopulation cross-reacted with IBV prefusion HAs (FIG. 12B, Table 21). None of the GC B cells bound strongly to Group 1 prefusion HA0s (FIG. 5E). S1V2-72 competed strongly with many IBV-restricted GC B cells for HA binding (FIG. 5F), indicating substantial epitope overlap, while most B+H3 cross-reactive cells were less sensitive to S1V2-72 competition (FIG. 5F). These results indicate distinct epitopes.

Thus, immunization with IBV EHA2 elicits GC B cell responses targeting multiple epitopes: some cells bind at least parts of the S1V2-72 epitope, but only for IBV. Other GC B cells target one or more epitopes that do not overlap with that of S1V2-72, yet are present in the postfusion HA2 of H3/X31, B/MY04, and possibly other HAs not tested.

In Table 21, the following apply: Superscript a (^{a}) means resting mature follicular B cells; Superscript b (^{b}) is the number of IgG-positive samples divided by the number of samples screened; Superscript c (^{c}) is the number of all B/MY04 EHA2⁺ samples divided by the number of IgG⁺ samples; Superscript d (^{d}) is the samples bound B/MY04 EHA2 just above the limit of detection; Superscript e (^{e}) is the number of samples that cross-reacted with B/MY04 EHA2 and another antigen, divided by the total number of B/MY04 EHA2-reactive samples; and Superscript f (^{f}) is the number of samples that cross-reacted with B/MY04 EHA2 and H1/X181 FLsE and/or H5/VN04 FLsE, divided by the total number of B/MY04 EHA2-reactive samples.

In addition to provoking substantial GC B cell responses, priming and boosting with B/MY04 EHA2 also elicited a high concentration of serum IgG that bound B/MY04 EHA2 and prefusion IBV HAs (FIG. 5G-FIG. 5H, FIG. 12C-FIG. 12D). Vaccination also modestly but reproducibly raised the titer of H3/X31 EHA2-reactive serum IgG (FIG. 12C-FIG. 12D), which may have derived from the many GC B cells of low avidity for H3/X31 EHA2 (FIG. 5E).B/MY04 EHA2 immunization rarely yielded enhanced IgG titers against prefusion H3/X31, although a few animals developed detectable anti-H3/X31 FLsE responses (FIG. 12C-FIG. 12D).Serum IgG titers against prefusion Group 1 HAs (H1/X181, H5/VN04) were no different from those in naïve controls with a few exceptions (FIG. 12C-FIG. 12D). In most cases, the immune serum potently inhibited S1V2-72 binding to B/MY04 EHA2, but not H3/X31 EHA2 (FIG. 5I-FIG. 5J).

### Example 7

### Immunogenicity of the S1V2-72 Epitope in Humanized Mice

To ascertain further whether vaccination with postfusion HA2 can elicit broadly cross-reactive, S1V2-72-like Abs, experiments with HA β-hairpin antibody-knockin mice were conducted. These mice have unrearranged, human germline IGHV1-2, IGHD3-22, and IGHJ4 gene segments knocked into the mouse Igh locus (β-hairpin HC^{+/+}), and the unrearranged, human germline IGLV2-23 and IGLJ3 gene segments knocked into the mouse Igk locus (β-hairpin LC⁺⁺). Additionally, the mice express a human terminal deoxynucleotidyl transferase (hTDT) transgene, allowing for human-like diversification of light chain (LC) V-J junctions. During B-cell development, these genetic modifications collectively permit β-hairpin HC^{+/+} β-hairpin LC⁺⁺ (hereafter denoted as β-hairpin HC^{+/+}LC⁺⁺, for simplicity) B cells to assemble B-cell antigen receptors with S1V2-72-like human variable regions, or, alternatively, endogenous mouse variable regions.

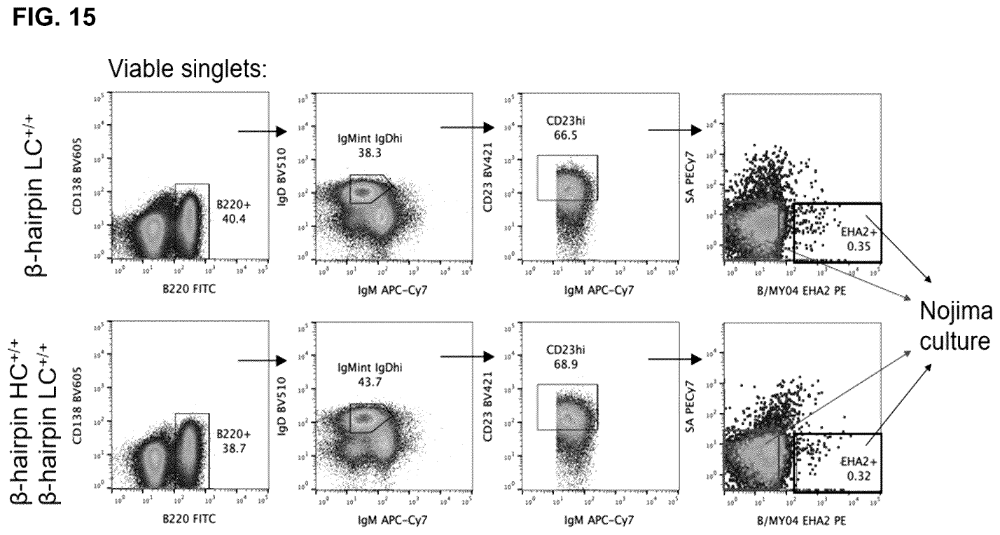

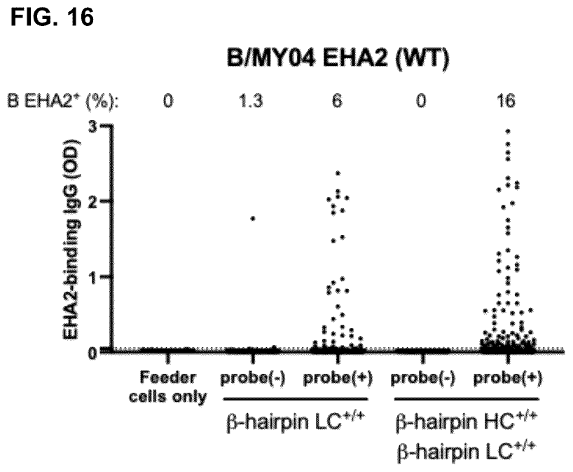

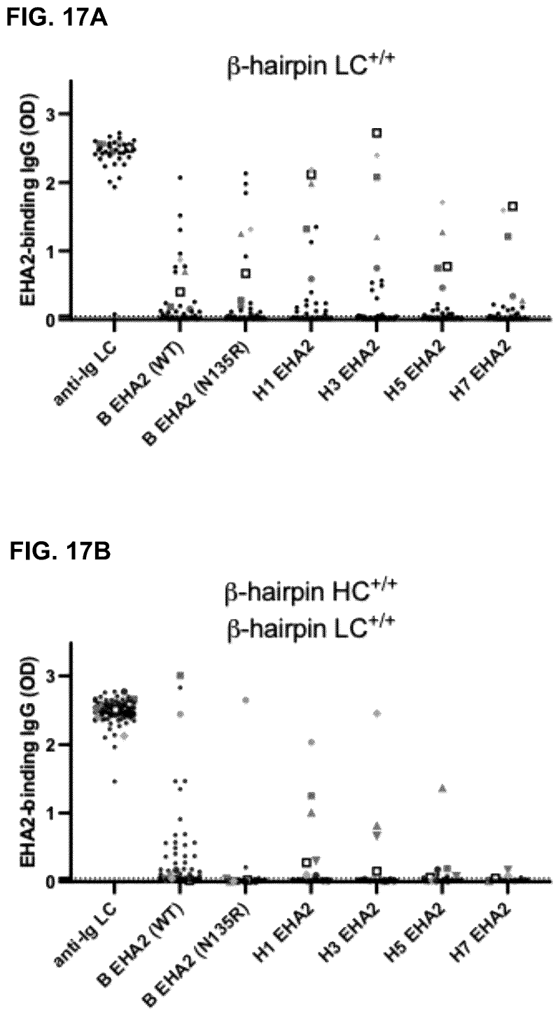

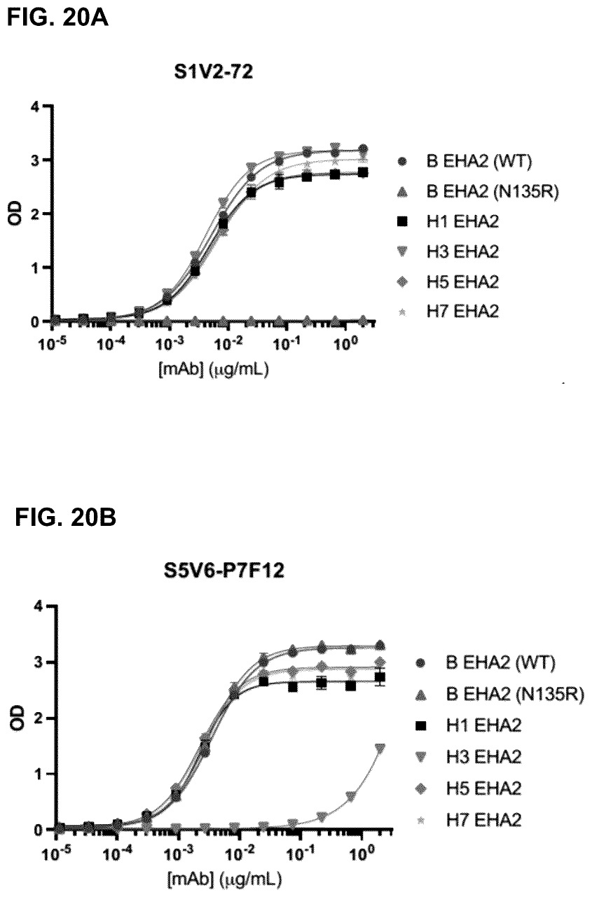

To determine the antigen-specificity of resting mature follicular (MF) B cells in β-hairpin HC^{+/+}LC⁺⁺ mice and control animals bearing only the knockin LC (β-hairpin LC⁺⁺), B/MY04 EHA2-binding or -nonbinding splenic MF B cells (FIG. 15) were sorted into single-cell Nojima cultures, and the secreted clonal IgGs were subsequently analyzed by ELISA (Kuraoka M, et al. (2016) Immunity. 44:542-552) (Table 22). Control supernatants, from cultures containing feeder cells but no B cells, did not react with B/MY04 EHA2 (FIG. 16 and Table 22). Likewise, MF B cells that did not bind fluorescent EHA2 antigen (probe [-]) during cell sorting rarely, if ever, produced IgGs that bound EHA2 in ELISA. In contrast, in both β-hairpin HC^{+/+}LC⁺⁺ and β-hairpin LC⁺⁺ mice, the population of MF B cells that bound fluorescent EHA2 antigen (probe [+]) during cell sorting was enriched for cells that secreted B/MY04 EHA2-binding IgG (FIG. 16 and Table 22). Like S1V2-72 IgG, most (44/46) β-hairpin HC^{+/+}LC⁺⁺ MF B cells that bound wildtype B/MY04 EHA2 did not bind B/MY04 N135R (FIG. 17B and FIG. 20A), indicating these B cells target the β-hairpin epitope. A subset (7/44) of these cells cross-reacted with one or more EHA2s representing IAV HA subtypes (H1 A/USSR/90/1977, H3 A/Aichi/2/1968, H5 A/Vietnam/1203/2004, or H7 A/chicken/Guangdong/J1/2017) (FIG. 17B). In contrast, most (15/24) β-hairpin LC⁺⁺ MF B cells that bound wildtype B/MY04 EHA2 also bound B/MY04 N135R EHA2 (FIG. 17A and Table 22), indicating the epitopes of these cells are substantially different from that of S1V2-72, and may instead be more similar to that of S5V6-P7F12, which does not require HA2 Asn135 to bind (FIG. 20B). However, 9/24 β-hairpin LC⁺⁺ MF B cells required Asn135 to bind B/MY04 EHA2, and five of these cells cross-reacted with ≥1 IAV EHA2 (Table 22).

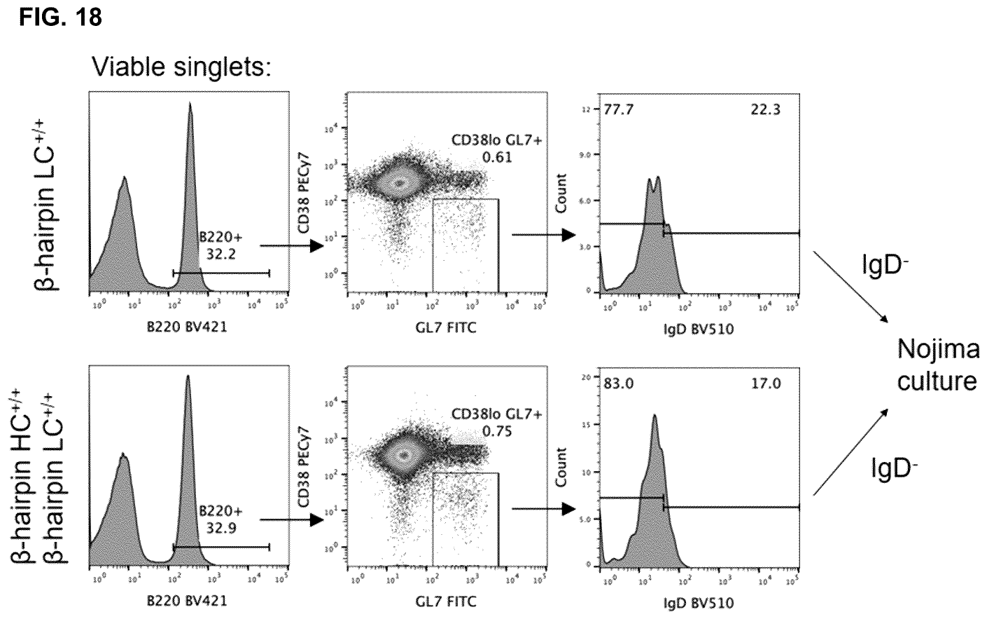

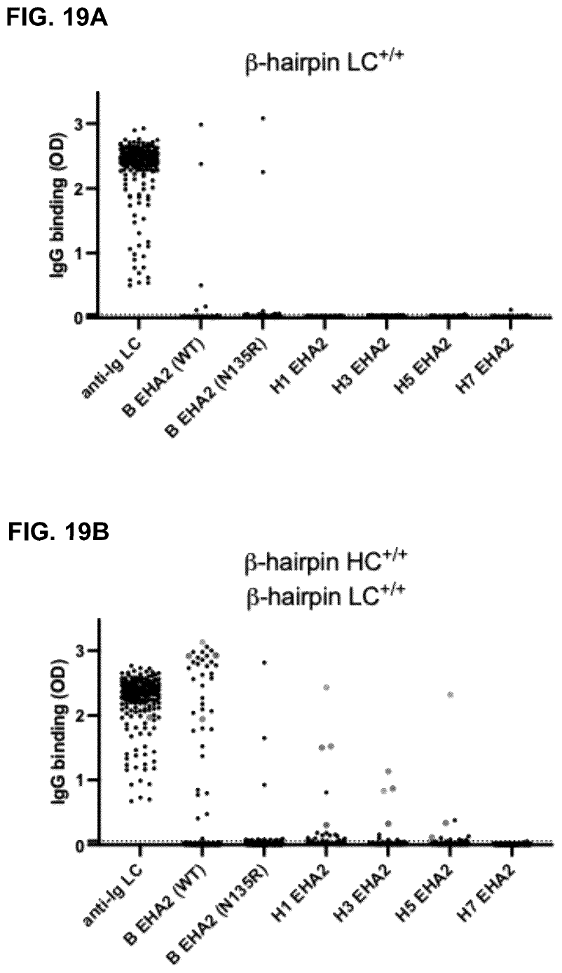

To determine the antigen-specificity of GC B cells responding to primary immunization with EHA2, β-hairpin HC^{+/+}LC⁺⁺ and β-hairpin LC⁺⁺ animals were immunized with B/MY04 EHA2 in alum adjuvant. Eight days post-immunization, GC B cells from the draining popliteal lymph node (FIG. 18) were sorted for Nojima culture. About 18% (39/219) of β-hairpin HC^{+/+}LC^{+/+}GC B cells reacted with B/MY04 EHA2, and ˜90% of these required HA2 Asn135 for binding (FIG. 19B and Table 22). More than half (18/25) of the Asn135-dependent GC B cells cross-reacted with ≥1 IAV EHA2, and 5/35 B cells bound ≥3 IAV EHA2s, including representatives from both IAV HA Groups 1 and 2. Only 2% (5/213) of β-hairpin LC⁺⁺ GC B cells bound B/MY04 EHA2 (FIG. 19A).

Analysis of the V(D)J rearrangements from a subset of the MF and GC B cells (Table 23) revealed that each of the cells expressed a LC encoded by the knockin human IGLV2-23 and IGLJ3 genes. Most of the sampled cells also expressed heavy chains encoded by the knockin human IGHV1-2 and IGHJ4 genes, although a few were encoded by endogenous mouse VH genes. Among the cells that expressed both human HC and human LC were two GC B cells and one MF B cell that cross-reacted with IAV and IBV EHA2s and required Asn135 for EHA2 binding.

That B/MY04 EHA2 immunogen binds resting MF B cells expressing humanized, S1V2-72-like B-cell receptors and recruits these B cells into the GC reaction indicates that vaccinating humans with EHA2 is likely to elicit broadly protective β-hairpin humoral responses, i.e., EHA2 is a plausible universal influenza vaccine immunogen.

In Table 22, the following apply: Superscript a (^{a}) means resting mature follicular B cells that did not bind fluorophore-conjugated EHA2, as determined by flow cytometric analysis; Superscript b (^{b}) means resting mature follicular B cells that did bind fluorophore-conjugated EHA2, as determined by flow cytometric analysis; Superscript c (^{c}) is the number of IgG-positive samples divided by the number of samples screened; Superscript d (^{d}) is the number of all B/MY04 wildtype EHA2⁺ samples divided by the number of IgG⁺ samples; Superscript e (^{e}) is the number of samples that reacted with B/MY04 wildtype EHA2 and B/MY04 N135R mutant EHA2, divided by the total number of B/MY04 wildtype EHA2-reactive samples; Superscript f (^{f}) is the number of samples that reacted with B/MY04 wildtype EHA2 but not B/MY04 N135R mutant EHA2, divided by the total number of B/MY04 wildtype EHA2-reactive samples; Superscript g (^{g}) is the number of B/MY04 EHA2+B/MY04 N135R EHA2⁻ samples that cross-reacted with none of the IAV EHA2s tested, divided by the total number of B/MY04 wildtype EHA2-reactive samples; Superscript h (^{h}) is the number of B/MY04 EHA2⁺ B/MY04 N135R EHA2⁻ samples that cross-reacted with any one of the IAV EHA2s tested, divided by the total number of B/MY04 wildtype EHA2-reactive samples; Superscript i (ⁱ) is the number of B/MY04 EHA2⁺ B/MY04 N135R EHA2⁻ samples that cross-reacted with any two of the IAV EHA2s tested, divided by the total number of B/MY04 wildtype EHA2-reactive samples; Superscript j (J) is the number of B/MY04 EHA2⁺ B/MY04 N135R EHA2⁻ samples that cross-reacted with any three of the IAV EHA2s tested, divided by the total number of B/MY04 wildtype EHA2-reactive samples; Superscript k (^{k}) is the number of B/MY04 EHA2⁺ B/MY04 N135R EHA2⁻ samples that cross-reacted with all four of the IAV EHA2s tested, divided by the total number of B/MY04 wildtype EHA2-reactive samples “n.d.” means not determined.

In Table 23, the following apply: Superscript a (^{a}) means IgG binding to EHA2 antigens in ELISA; Superscript b (^{b}) means binding to B/MY04 wildtype (WT) EHA2; Superscript c (^{c}) means binding to B/MY04 N135R EHA2; Superscript d (^{d}) is the number somatic nucleotide mutations in the recovered LC V-gene; Superscript e (^{e}) is the number of somatic nucleotide mutations in the recovered HC V-gene; “H.s.” means Homo sapiens; “M.m.” means Mus musculus; “n.d.” means not determined; “+” indicates binding, i.e., an ELISA result higher than 6 standard deviations above the arithmetic mean signal from the negative control wells lacking IgG; “−” indicates no binding, i.e., an ELISA result less than 6 standard deviations above the arithmetic mean signal from the negative control wells.

TABLE 21
Summary of Mouse Nojima Culture Screening Results

| | Experiment 1 | | Experiment 2 | | Total | |
|---|---|---|---|---|---|---|
| | Day 18 | MF | Day 18 | MF | Day 18 | MF |
| | GCB | B^{a} | GCB | B^{a} | GCB | B^{a} |
| IgG⁺/total screened^{b} | 546/4,032 | 73/96 | 736/4,032 | 78/96 | 1,282/8,064 | 151/192 |
| Cloning efficiency | 14 | 76 | 18 | 81 | 16 | 79 |
| (%) | | | | | | |
| Total B/MY04 | 172/546 | 1/76^{d} | 182/736 | 6/78^{d} | 354/1,282 | 7/151^{d} |
| EHA2⁺ cultures^{c} | | | | | | |
| B/MY04 EHA²⁺ + | 12/172 | 0/1 | 49/182 | 0/6 | 61/354 | 0/7 |
| B/MY04 FLsE⁺ | | | | | | |
| cross-reactive | | | | | | |
| cultures^{e} | | | | | | |
| B/MY04 EHA²⁺ + | 16/172 | 0/1 | 53/182 | 0/6 | 69/354 | 0/7 |
| B/BN08 FLsE⁺ cross- | | | | | | |
| reactive cultures^{e} | | | | | | |
| B/MY04 EHA²⁺ + | 20/172 | 0/1 | 65/182 | 0/6 | 85/354 | 0/7 |
| B/PK13 FLsE⁺ cross- | | | | | | |
| reactive cultures^{e} | | | | | | |
| B/MY04 EHA²⁺ + | 46/172 | 0/1 | 49/182 | 0/6 | 95/354 | 0/7 |
| H3/X31 EHA²⁺ | | | | | | |
| cross-reactive | | | | | | |
| cultures^{e} | | | | | | |
| B/MY04 EHA²⁺ + | 9/172 | 0/1 | 11/182 | 0/6 | 20/354 | 0/7 |
| Group 1 HA⁺ cross- | | | | | | |
| reactive cultures^{f} | | | | | | |

### Example 8

### Truncating Postfusion HA2 Antigen to Focus the Humoral Response on the β-Hairpin Epitope

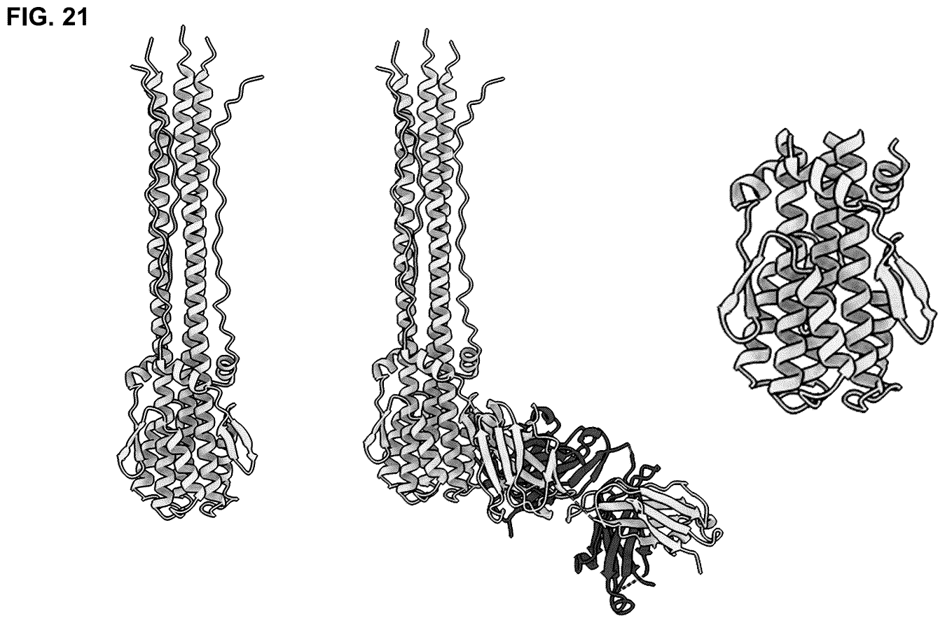

Immunizing animals with full-length, postfusion HA2 antigen presumably elicits antibodies to several epitopes on the antigen. Antibodies to certain epitopes bind only postfusion HA2 from a single HA subtype or influenza virus lineage, e.g., antibodies S1V4-P5F6 (FIG. 11B) and 20B10 (Table 23) bind only IBV HA2, whereas S1V2-72-like β-hairpin antibodies bind HA2s from numerous IAV subtypes and IBV (FIG. 4). Antibodies to different epitopes on the same molecule are not expected to contribute equally to universal influenza immunity: antibody S1V4-P5F6 will not prevent disease caused by H1N1 influenza infection, whereas S1V2-72-like antibodies that bind H1 HA2 may reduce the severity of symptoms after H1N1 infection. Because elicitation of less desirable antibodies may interfere with elicitation of more desirable β-hairpin antibodies, it may be advantageous to use a truncated postfusion HA2 antigen as a vaccine immunogen, to focus the antibody response on the β-hairpin epitope. A proposed truncated HA2 antigen comprises HA2 residues 75-155. This truncated antigen includes the complete epitope of β-hairpin antibodies, but eliminates ˜50% of the HA2 residues not bound by β-hairpin antibodies (FIG. 21).

TABLE 22
Summary of Mouse Nojima Culture Screening Results

| | β-hairpin HC^{+/+}LC^{+/+} | | | β-hairpin LC^{+/+} | | |
|---|---|---|---|---|---|---|
| | Probe⁻ | Probe⁺ | Day 8 | Probe⁻ | Probe⁺ | Day 8 |
| | MF B^{a} | MF B^{b} | GC B | MF B^{a} | MF B^{b} | GCB |
| IgG⁺/total screened^{c} | 120/192 | 633/768 | 219/960 | 152/192 | 622/768 | 213/960 |
| Cloning efficiency (%) | 62.5 | 82.4 | 22.8 | 79.2 | 81.0 | 22.2 |
| Total B/MY04 EHA2⁺ | 0/120 | 46/633 | 39/219 | 2/152 | 24/622 | 5/213 |
| cultures^{d} | | | | | | |
| B/MY04 EHA2⁺ | n.d. | 2/46 | 4/39 | n.d. | 15/24 | 3/5 |
| B/MY04 N135R EHA2⁺ | | | | | | |
| cultures^{e} | | | | | | |
| B/MY04 EHA2⁺ | n.d. | 44/46 | 35/39 | n.d. | 9/24 | 2/5 |
| B/MY04 N135R EHA2⁻ | | | | | | |
| cultures^{f} | | | | | | |
| B/MY04 EHA2⁺ | n.d. | 37/44 | 17/35 | n.d. | 4/9 | 1/2 |
| B/MY04 N135R EHA2⁻ | | | | | | |
| cultures that bind only | | | | | | |
| IBV EHA2^{g} | | | | | | |
| B/MY04 EHA2⁺ | n.d. | 3/44 | 7/35 | n.d. | 2/9 | 1/2 |
| B/MY04 N135R EHA2⁻ | | | | | | |
| cultures that bind one | | | | | | |
| IAV EHA2^{h} | | | | | | |
| B/MY04 EHA2⁺ | n.d. | 2/44 | 10/35 | n.d. | 2/9 | 0/2 |
| B/MY04 N135R EHA2⁻ | | | | | | |
| cultures that bind two | | | | | | |
| IAV EHA2sⁱ | | | | | | |
| B/MY04 EHA2⁺ | n.d. | 2/44 | 4/35 | n.d. | 1/9 | 0/2 |
| B/MY04 N135R EHA2⁻ | | | | | | |
| cultures that bind three | | | | | | |
| IAV EHA2s^{j} | | | | | | |
| B/MY04 EHA2⁺ | n.d. | 0/44 | 1/35 | n.d. | 0/9 | 0/2 |
| B/MY04 N135R EHA2⁻ | | | | | | |
| cultures that bind four | | | | | | |
| IAV EHA2s^{k} | | | | | | |

TABLE 23
EHA2 Binding Data and V-Gene Use
for Selected MF B and GC B Cells

| | | | EHA2 binding^{a} | | | | | |
|---|---|---|---|---|---|---|---|---|
| Cell | | Cell | B | B | | | | |
| ID | Genotype | type | (WT)^{b} | (N135R)^{c} | H1 | H3 | H5 | H7 |
| 12B06 | β-hairpin | GC B | + | − | + | + | − | − |
| | HC^{+/+}LC^{+/+} | | | | | | | |
| 14E04 | β-hairpin | GC B | + | − | + | + | + | − |
| | HC^{+/+}LC^{+/+} | | | | | | | |
| 14G03 | β-hairpin | GC B | + | − | − | − | − | − |
| | HC^{+/+}LC^{+/+} | | | | | | | |
| 18B09 | β-hairpin | GC B | + | − | + | + | + | − |
| | HC^{+/+}LC^{+/+} | | | | | | | |
| 18C09 | β-hairpin | GC B | + | − | + | − | − | − |
| | HC^{+/+}LC^{+/+} | | | | | | | |
| 18E04 | β-hairpin | GC B | − | − | − | − | − | − |
| | HC^{+/+}LC^{+/+} | | | | | | | |
| 20B10 | β-hairpin | GC B | + | + | − | − | − | − |
| | HC^{+/+}LC^{+/+} | | | | | | | |
| 24A05 | β-hairpin | MF B | + | + | + | + | + | + |
| | LC^{+/+} | | | | | | | |
| 39A02 | β-hairpin | MF B | − | n.d. | n.d. | n.d. | n.d. | n.d. |
| | HC^{+/+}LC^{+/+} | | | | | | | |
| 39A05 | β-hairpin | MF B | − | n.d. | n.d. | n.d | n.d | n.d. |
| | HC^{+/+}LC^{+/+} | | | | | | | |
| 39C03 | β-hairpin | MF B | + | − | − | − | − | − |
| | HC^{+/+}LC^{+/+} | | | | | | | |
| 39D04 | β-hairpin | MF B | + | − | + | + | + | − |
| | HC^{+/+}LC^{+/+} | | | | | | | |
| 39E04 | β-hairpin | MF B | − | − | + | + | + | + |
| | HC^{+/+}LC^{+/+} | | | | | | | |
| 39H06 | β-hairpin | MF B | − | − | + | + | + | + |
| | HC^{+/+}LC^{+/+} | | | | | | | |

### Summary of Examples

Of previously studied HA Abs, CR9114 is the only one that protects broadly against both IAV- and IBV-driven disease. Isolation of CR9114 from a combinatorial display library raised the question of whether any definitively native human Ab can bind diverse IAV and IBV HAs.

Here, 10 novel human Abs were characterized as having broad cross-reactivity for IAV and IBV HAs. These 10 Abs bind overlapping epitopes centered on a previously undescribed HA2 β-hairpin motif that is conserved among most IAV and IBV HA subtypes. All these HA2 β-hairpin Abs have heavy chains constructed from IGHV1-2, and all have short HCDR3s (10-13 amino acids). Additionally, the β-hairpin Abs typically use light chains encoded by IGLV2-family genes or other genes whose germline versions encode Ser at the positions that will form the tip of the LCDR3 loop. Thus, S1V2-72-like β-hairpin Abs collectively constitute a public Ab response. S1V2-72 protected against both IAV and IBV when constituted with an FcγR-activating constant region, and immunization of human β-hairpin antibody knockin mice with EHA2 (i.e., postfusion HA2) recruited broadly cross-reactive S1V2-72-like B cells into germinal center responses. Moreover, that S1V2-72-like Abs recognize not only HAs from IAV and IBV strains currently circulating in human populations, but also HAs from subtypes that currently circulate solely among animals, indicates that a vaccine provoking an S1V2-72-like humoral response may impart pre-pandemic immunity as well. Collectively, these results support the use of EHA2 as a universal influenza vaccine antigen, and indicate that therapeutic administration of β-hairpin antibodies to influenza virus-infected patients may lessen morbidity and mortality.

The experiments described in the Examples show that S1V2-72 can bind its epitope as present on solubilized prefusion ectodomains, indicating that conformational fluctuations may transiently expose the otherwise occluded epitope, analogous to the molecular “breathing” that allows HA head-interface Abs access to their epitopes (Watanabe A, et al. (2019) Cell. 177:1124-1135 e1116). S1V2-72 might be able to direct immune clearance of virions and infected cells by recognizing its epitope on HA in the prefusion conformation.

Presence of postfusion HA on the surface of infected cells might also account for at least part of the protective efficacy of postfusion-HA directed Abs. The recently identified mAb, LAH31 (Tonouchi K, et al. (2023) PLOS Pathog. 19: e1011554), binds a conformational epitope unique to postfusion HA, yet LAH31 avidly binds cell-surface HA on IAV-infected cell lines in vitro, and protects mice against IAV disease, indicating that there is postfusion HA on infected cells in vivo. Because of its avidity for postfusion HA2, S1V2-72 could also confer in vivo protection by recognizing postfusion HA directly. Like S1V2-72 and LAH31, several HA mAbs with cryptic epitopes exposed at pH<5.5 also protect against influenza-associated weight loss by FcγR-dependent mechanisms (Henry Dunand C J, et al. (2016) Cell Host Microbe. 19:800-813), providing further evidence that humoral responses to postfusion HA epitopes can be important contributors to immune control of IAV and IBV infections.

How can postfusion HA appear on the cell surface, since HA transitions to the postfusion form in acidifying (pH<5.5) endosomes and thereafter presumably is degraded when the endosome merges with a lysosome? There are three non-mutually exclusive hypotheses for how extracellular postfusion HA might arise: (i) release from lysed infected cells, (ii) conformational transition during inflammation-driven extracellular acidification, and (iii) conformational transition during endosomal recycling in follicular dendritic cells. All three hypotheses explain how the immune system could be sensitized to postfusion HA, and the second mechanism could account for how postfusion HA Abs such as LAH31 could control an influenza infection, if the postfusion HA decorated the surface of infected cells. However, extracellular acidification cannot explain why LAH31 brightly labels cells infected with IAV in vitro where the extracellular pH is buffered ≥6 (Tonouchi K, et al. (2023) PLOS Pathog. 19: e1011554). Instead, postfusion HA may regularly appear on the surface of infected cells after escaping from acidified early endosomes via endocytic recycling pathways (Cullen P J, et al. (2018) Nat Rev Mol Cell Biol. 19:679-696). In this way, expression of postfusion HA on cell membranes would be independent of extracellular pH and would identify infected cells for immune lysis.

The breadth of inferred germline human β-hairpin Abs and of the precursor B cells in β-hairpin antibody knockin mice, paired with the contributions of HCDR1 and HCDR2 to the paratope, implies that the capacity of S1V2-72-like Abs to bind both IAV and IBV HAs may be germline-encoded. In contrast, the CR9114 germline precursor binds H1s, but needs 5 specific amino acid substitutions to bind H3s, and 9 substitutions to bind B HAs (Phillips A M, et al. (2021) Elife. 10: e71393). Therefore, the vaccination regimen required to elicit broadly protective S1V2-72-like β-hairpin Abs is likely to be much simpler than the regimen needed to elicit CR9114-like antibodies of equally broad protective efficacy; indeed a single immunization with B/MY04 EHA2 elicits S1V2-72-like GC B cells that recognize HA2 from IBV and several medically relevant IAVs.

What is claimed is:

1. A binding molecule capable of specifically binding to postfusion hemagglutinin (HA),
wherein the binding molecule binds to the stem region of HA; and
wherein the stem region of HA comprises an epitope comprises a β-hairpin.

2. The binding molecule of claim 1, wherein the epitope comprises the sequence set forth in SEQ ID NO:191.

3. The binding molecule of claim 1, comprising:
(i) a variable light chain region (VL) comprising 3 complementarity determining regions (CDRs), and
(ii) a variable heavy chain region (VH) comprising 3 CDRs and a constant heavy chain region.

4. The binding molecule of claim 3, wherein the VH comprises a sequence having at least 80% identity to the sequence set forth in any one of SEQ ID NO:05-SEQ ID NO:16.

5. The binding molecule of claim 3, wherein the VL comprises a sequence having at least 80% identity to the sequence set forth in any one of SEQ ID NO:17-SEQ ID NO:28.

6. The binding molecule of claim 4, wherein the VH comprises a sequence having at least 80% identity to the sequence set forth in SEQ ID NO:05 or SEQ ID NO:06.

7. The binding molecule of claim 5, wherein the VL comprises a sequence having at least 80% identity to the sequence set forth in SEQ ID NO:17 or SEQ ID NO:18.

8. The binding molecule of claim 3, wherein the VL comprises at least one CDR having the sequence set forth in any one of SEQ ID NO:64-SEQ ID NO:69; and wherein the VH comprises at least one CDR having the sequence set forth in any one of SEQ ID NO:59-SEQ ID NO:63.

9. The binding molecule of claim 3, wherein the first CDR in the VL comprises a sequence having at least about 80% identity to the sequence set forth in SEQ ID NO:64 or SEQ ID NO:65; the second CDR in the VL comprises a sequence having at least 80% identity to the sequence set forth in SEQ ID NO:66 or SEQ ID NO:67; and the third CDR in the VL comprises a sequence having at least about 80% identity to the sequence set forth in SEQ ID NO:68 or SEQ ID NO:69.

10. The binding molecule of claim 3, wherein the first CDR in the VH comprises a sequence having at least about 80% identity to the sequence set forth in SEQ ID NO:59 or SEQ ID NO:60; the second CDR in the VH comprises a sequence having at least about 80% identity to the sequence set forth in SEQ ID NO:61 or SEQ ID NO:62; and the third CDR in the VH comprises a sequence having at least about 80% identity to the sequence set forth in SEQ ID NO:63.

11. The binding molecule of claim 3, wherein the VL comprise at least one CDR having the sequence set forth in any one of SEQ ID NO:75-SEQ ID NO:80; and wherein the VH comprises at least one CDR having the sequence set forth in any one of SEQ ID NO:70-SEQ ID NO:74.

12. The binding molecule of claim 3, wherein the first CDR in the VH comprises a sequence having at least about 80% identity to the sequence set forth in SEQ ID NO:70 or SEQ ID NO:71; the second CDR in the VH can comprise a sequence having at least about 80% identity to the sequence set forth in SEQ ID NO:72 or SEQ ID NO:73; and the third CDR in the VH comprises a sequence having at least about 80% identity to the sequence set forth in SEQ ID NO:74.

13. The binding molecule of claim 3, wherein the first CDR in the VL comprises a sequence having at least about 80% identity to the sequence set forth in SEQ ID NO:75 or SEQ ID NO:76; the second CDR in the VL comprises a sequence having at least about 80% identity to the sequence set forth in SEQ ID NO:77 or SEQ ID NO:78; and the third CDR in the VL comprises a sequence having at least about 80% identity to the sequence set forth in SEQ ID NO:79 or SEQ ID NO:80.

14. The binding molecule of claim 3, wherein the VL is encoded by IGLV2-23*01/IGLJ3*02, wherein the VL comprises about 1% to about 20% somatic mutations; and wherein the VH is encoded by IGHV1-2*02/IGHD5-12*01/IGHJ5*02, wherein the VL comprises about 1% to about 20% somatic mutations.

15. The binding molecule of claim 3, wherein the VL is encoded by IGLV2-23*01/IGLJ3*02, wherein the VL comprises about 1% to about 20% somatic mutations; and wherein the VH is encoded by IGHV1-2*07/IGHD6-13*01/IGHJ4*02, wherein the VL comprises about 1% to about 20% somatic mutations.

16. A pharmaceutical formulation comprising the binding molecule of claim 1.

17. A method of treating a subject, the method comprising:
administering to a subject in need thereof a therapeutically effective amount of the pharmaceutical formulation of claim 16.

18. The method of claim 17, wherein following the administering step, (i) the risk of the subject's mortality is minimized and/or decreased; (ii) the subject's survival is increased and/or prolonged; (iii) the subject's quality of life is enhanced and/or improved; (iv) the likelihood of surgical intervention is reduced and/or minimized; (v) the frequency of other treatment is reduced and/or decreased; (vi) one or more subject's symptoms is relieved and/or ameliorated; (vii) the subject's risk of hospitalization is reduced and/or minimized; (viii) the need for surgical intervention is prevented and/or minimized; (ix) normal metabolism of one or more of the subject's organ systems is improved and/or restored, (x) one or more aspects of cellular homeostasis and/or cellular functionality, and/or metabolic dysregulation in one or more of the subject's affected systems is restored and/or improved, or (xi) any combination thereof.

19. The method of claim 17, further comprising repeating the administering to the subject of the pharmaceutical formulation.

20. The method of claim 17, further comprising administering to the subject one or more postfusion hemagglutinin (HA) proteins, wherein the one or more proteins comprise the sequence set forth in any one of SEQ ID NO:29-SEQ ID NO:58 and SEQ ID NO:192-SEQ ID NO:218.
